(19) United States
(12) Patent Application Publication
(10) Pub. No.: US 2014/0221354 A1
(43) Pub. Date: Aug. 7, 2014

(54) IMIDAZO [1,2-a]PYRIDINE COMPOUNDS FOR USE IN THERAPY

(71) Applicants: Marcel Mulbaier, Meckesheim (DE); Jorge Alonso, Mannheim (DE); Douglas Thomson, Heidelberg (DE); Bernd Janssen, Harvard, MA (US); Arantxa Encinas Lopez, Karlsruhe (DE); Bernd Wendt, Schorndorf (DE); Christoph Schultes, Mannheim (DE)

(72) Inventors: Marcel Mulbaier, Meckesheim (DE); Jorge Alonso, Mannheim (DE); Douglas Thomson, Heidelberg (DE); Bernd Janssen, Harvard, MA (US); Arantxa Encinas Lopez, Karlsruhe (DE); Bernd Wendt, Schorndorf (DE); Christoph Schultes, Mannheim (DE)

(73) Assignee: EMBLEM Technology Transfer GmbH, Heidelberg (DE)

(21) Appl. No.: 14/007,613

(22) PCT Filed: Mar. 31, 2011
(86) PCT No.: PCT/EP11/55059

§ 371 Date: Apr. 3, 2014

(51) Int. Cl. C07D 471/04; A61K 31/496; A61K 45/06; A61K 31/551

(52) U.S. Cl. CPC C07D 471/04; A61K 31/551; A61K 31/496; A61K 45/06
USPC 514/218; 514/253.04

(57) ABSTRACT

The present invention provides novel imidazo[1,2-a]pyridine compounds that are useful in therapy of diseases and disorders. The novel compounds inhibit the activation of Hypoxia Inducible Factor (HIF)-mediated transcription and signaling under hypoxic conditions. In one aspect, the compounds of the present invention are useful for the preparation of a medicament for the treatment or prevention of a disease or disorder selected from the group consisting of an inflammatory disease, a hyperproliferative disease or disorder, a hypoxia-related pathology and a disease characterized by excessive vascularization. Also provided is a pharmaceutical composition, comprising a compound of the invention and a second therapeutic agent or radiation, useful for the treatment or prevention of the mentioned diseases or disorders, wherein X is CH₂, CH₂CH₂ or C═O; R¹ is phenyl or C-bound monocyclic 5- or 6-membered heteroaryl, wherein phenyl and monocyclic 5- or 6-membered heteroaryl are unsubstituted or carry 1, 2, 3, 4 or 5 radicals R^{1a} which are identical or different; R² is phenyl or C-bound monocyclic 5- or 6-membered heteroaryl, wherein phenyl and monocyclic 5- or 6-membered heteroaryl are unsubstituted or carry 1, 2, 3, 4 or 5 radicals R^{2a} which are identical or different; R³ is hydrogen, C₁-C₆-alkyl, C₁-C₄-alkoxy-C₁-C₄-alkyl, fluorinated C₁-C₂-alkyl C(O)R⁴; where R^{1a}, R^{2a} and R⁴ are as defined in the claims and the specification.

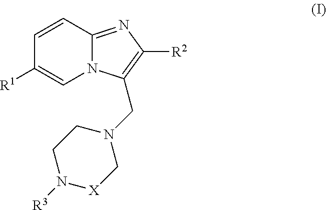

The present invention provides novel imidazo[1,2-a]pyridine compounds that are useful in therapy of diseases and disorders. The novel compounds inhibit the activation of Hypoxia Inducible Factor (HIF)-mediated transcription and signaling under hypoxic conditions. In one aspect, the compounds of the present invention are useful for the preparation of a medicament for the treatment or prevention of a disease or disorder selected from the group consisting of an inflammatory disease, a hyperproliferative disease or disorder, a hypoxia-related pathology and a disease characterized by excessive vascularization. Also provided is a pharmaceutical composition, comprising a compound of the invention and a second therapeutic agent or radiation, useful for the treatment or prevention of the mentioned diseases or disorders.

### BACKGROUND OF THE INVENTION

The normal response of cells to inadequate oxygen supply is mediated by the hypoxia signaling pathway. This response is important for a number of physiological functions such as tumor development and metastasis, resistance to apoptosis, induction of new blood vessel formation, and shift towards anaerobic metabolism amongst others. For a general review on hypoxia signaling see e.g. Qingdong Ke and Max Costa, Molecular Pharmacology (2006), vol. 70, no. 5.

As a result of hypoxia, augmented levels of a heterodimeric complex of transcription factors (Hypoxia Inducible Factor, HIF), most notably HIF-1α and HIF-113, are observed in e.g. tumors to compensate in cooperation with additional co-factors for the reduced availability of oxygen and nutrients in this fast growing tissue type. Under anaerobic conditions, homeostasis of HIF-1α is imbalanced by its reduced degradation, thus enabling enhanced signaling through the Hypoxia Responsive Element (HRE) and resulting in increased expression of a large number of survival and growth factors.

Hypoxic conditions are also found in non-tumor tissue. For example, retinopathy is a general term that refers to non-inflammatory damage to the retina of the eye. This condition is most commonly caused by an insufficient blood supply leading to hypoxia. Particularly people with diabetes mellitus are at risk of retinopathy. The lack of oxygen in the retina of diabetics causes fragile, new blood vessels to grow along the retina and in the clear, gel-like vitreous humor that fills the inside of the eye. Without timely treatment, these new blood vessels can bleed, cloud vision, and destroy the retina. Fibrovascular proliferation can also cause fractional retinal detachment. The new blood vessels can also grow into the anterior chamber of the eye and cause neovascular glaucoma.

Recently, evidence has accumulated that inhibition of HIF-1 activity could also act to prevent inflammation, by virtue of its role in the activation and infiltration of macrophages and neutrophils into affected tissues (see e.g. Giaccia et al., Drug Discovery, vol. 2, October 2003).

For the above-outlined reasons, compounds that inhibit HIF function are valuable medicaments for the treatment or prevention of a disease or disorder selected from the group consisting of an inflammatory disease, a hyperproliferative disease or disorder, a hypoxia-related pathology and a disease characterized by excessive vascularization.

Because of the importance of HIF-1 in tumor development, progression and metastasis, a considerable amount of effort has been devoted to identify HIF-1 inhibitors for cancer therapy. A number of small molecules and RNA constructs, like siRNA, have been reported to exhibit inhibition of the HIF-1 pathway, e.g. Kung A L et al., Cancer Cell (2004), vol. 6, p. 33 ff; Rapisarda A., et al. Cancer Res. (2002), vol. 62, p. 4316 ff.; Tan C. et al., Cancer Res. (2005), vol. 65, p. 605 ff.; Mabjeesh N J et al., Cancer Cell, (2003), vol. 3, p. 363ff;. Kong X, et al., Mol Cell Biol (2006), vol. 26, p. 2019 ff.; Kong D., et al., Cancer Res. (2005), vol. 65, p. 9047 ff.; Chau N. et al., Cancer Res. (2005), vol. 65, p. 4918 ff.; Welsh S., et al., Mol. Cancer. Ther. (2004), vol. 3, p. 233 ff. However, these compounds often have activities other than HIF-1 inhibition, and most of them lack the desired pharmacokinetic properties or toxicity profiles required for a useful pharmaceutical agent. Furthermore, some of the compounds have the disadvantage that they can not be administered orally, such as the HIF-1 inhibitor EZN-2968, which is a locked nucleic acid antisense oligonucleotide.

WO 2010/085968 discloses N-phenyl (monocyclic heteroaryl)sulfonamide compounds which inhibit cell proliferation cell division and which inhibit the activation of Hypoxia Inducible Factor (HIF)-mediated transcription and signaling under hypoxic conditions.

WO 2010/075869 discloses N-phenyl benzenesulfonamide compounds which inhibit cell proliferation cell division and which inhibit the activation of Hypoxia Inducible Factor (HIF)-mediated transcription and signaling under hypoxic conditions.

U.S. 61/359,119 (unpublished) discloses N-phenylsulfonamide compounds of the formula B

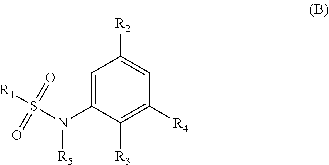

wherein R¹ is optionally substituted naphthyl or C-bound bicyclic heterocyclyl, R² is substituted phenyl or optionally substituted C- or N-bound monocyclic 5- or 6-membered heteroaryl; R³ is i.a. hydrogen, ethynyl, methyl, CH₂OH, CH₂O—C₁-C₄-alkyl, or CH₂O—C₁-C₄-alkoxy-C₁-C₄-alkyl; R⁴ is hydrogen, ethynyl or methyl and R⁵ is hydrogen or C₁-C₄-alkyl. The compounds are useful for the treatment or prevention of a disease or disorder selected from the group consisting of an inflammatory disease, a hyperproliferative disease or disorder, a hypoxia related pathology and a disease characterized by pathophysiological hypervascularization.

PCT/EP2009/064138 (unpublished) discloses 2,3-dihydrobenzazine compound of the formula C

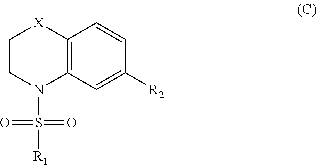

where X is O, S, SO or SO₂, R¹ is phenyl or C-bound monocyclic 5- or 6-membered heteroaryl where the two last-mentioned radicals may be unsubstituted or carry 1, 2 or 3 radicals. Two of said radicals which are bound to adjacent carbon atoms of phenyl or 5- or 6-membered heteroaryl, may also form a bridging moiety O—CH₂—O, O—CHF—O, O—CF₂—O or O—CH₂—CH₂—O. R² is phenyl or C- or N-bound monocyclic 5- or 6-membered heteroaryl, phenyl and monocyclic 5- or 6-membered heteroaryl carrying a CN radical and optionally further substituents. The compounds are useful for the treatment or prevention of a disease or disorder selected from the group consisting of an inflammatory disease, a hyperproliferative disease or disorder, a hypoxia related pathology and a disease characterized by pathophysiological hyper-vascularisation.

U.S. 61/422,586 (unpublished) discloses tetrahydroquinoxaline compounds of the formula D

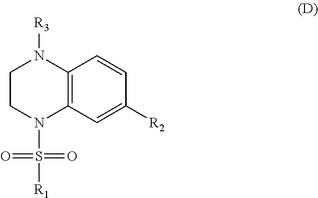

wherein R¹ is an optionally substituted aromatic radical selected from phenyl, C-bound monocyclic 5- or 6-membered heteroaryl or C-bound bicyclyl comprising a first ring which is a benzene ring or a 5- or 6-membered heteroaromatic ring and a second ring, which is fused to the first ring, where the second ring is a 5-, 6- or 7-membered carbocyclic ring or a 5-, 6- or 7-membered heterocyclic ring, where the saturated or unsaturated carbocyclic or heterocyclic second ring may also have 1 or 2 carbonyl groups or thiocarbonyl groups as ring members; R₂ is an optionally substituted aromatic radical selected from phenyl, C-bound monocyclic 5- or 6-membered heteroaryl or C-bound bicyclyl comprising a first ring which is a benzene ring or a 5- or 6-membered heteroaromatic ring and a second ring, which is fused to the first ring, where the second ring is a 5-, 6- or 7-membered carbocyclic ring or a 5-, 6- or 7-membered heterocyclic ring, where the saturated or unsaturated carbocyclic or heterocyclic second ring may also have 1 or 2 carbonyl groups or thiocarbonyl groups as ring member; R₃ is hydrogen, C₁-C₆-alkyl, fluorinated C₁-C₂-alkyl, or a radical C(X)Rₓ, wherein X is O or S and Rₓ is C₁-C₆-alkyl, fluorinated C₁-C₂-alkyl, C₁-C₆-alkoxy, C₃-C₈-cycloalkoxy, benzyloxy or fluorenylmethoxy. The compounds inhibit cell proliferation and cell division and they also inhibit the activation of Hypoxia Inducible Factor (HIF)-mediated transcription and signaling under hypoxic conditions.

The scientific literature cited above emphasizes the high medical need for new therapeutic agents to provide more efficient treatment of different proliferative and inflammatory diseases or disorders, hypoxia-related pathologies and diseases characterized by excessive vascularization. It is an object of the present invention to provide novel compounds which can be used as very potent inhibitors of (i) the activation of HIF mediated transcription under hypoxic conditions and of (ii) cell proliferation.

### SUMMARY OF THE INVENTION

The present invention provides novel compounds capable of prevention or treatment of a disease or disorder. Data presented herein establish that compounds according to the present invention are surprisingly very potent inhibitors of (i) the activation of HIF mediated transcription under hypoxic conditions and of (ii) cell proliferation.

In a first aspect the present invention relates to imidazo[1,2-a]pyridine compounds of the formula (I) for use in therapy

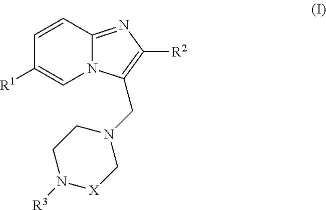

wherein
X is CH₂, CH₂CH₂ or C═O;
R¹ is phenyl or C-bound monocyclic 5- or 6-membered heteroaryl, wherein phenyl and monocyclic 5- or 6-membered heteroaryl are unsubstituted or carry 1, 2, 3, 4 or 5 radicals R^{1a} which are identical or different;
   R^{1a} is selected from the group consisting of halogen, CN, NO₂, NH₂, OH, SH, C₁-C₁₀-alkyl, C₂-C₁₀-alkenyl, C₂-C₁₀-alkynyl, C₁-C₆-alkoxy, C₁-C₆-alkylthio, hydroxy-C₁-C₆-alkyl, C₁-C₄-alkoxy-C₁-C₄-alkyl, fluorinated C₁-C₂-alkyl, SF₅, fluorinated C₁-C₂-alkoxy, fluorinated C₁-C₂-alkylsulfonyl, fluorinated C₁-C₂-alkylthio, C(O)R⁴, NR⁵R⁶, C(O)NR⁷R⁸ and C(O)OR⁹, or two radicals R^{1a}, which are bound to adjacent carbon atoms, may also form a bridging moiety O-Alk-O, wherein Alk is selected from CH₂, CH₂CH₂, CHF, CF₂ and CF₂CF₂;
R² is phenyl or C-bound monocyclic 5- or 6-membered heteroaryl, wherein phenyl and monocyclic 5- or 6-membered heteroaryl are unsubstituted or carry 1, 2, 3, 4 or 5 radicals R^{2a} which are identical or different;
   R^{2a} is selected from the group consisting of halogen, CN, NO₂, NH₂, OH, SH, C₁-C₁₀-alkyl, C₂-C₁₀-alkenyl, C₂-C₁₀-alkynyl, C₁-C₆-alkoxy, C₁-C₆-alkylthio, hydroxy-C₁-C₆-alkyl, C₁-C₄-alkoxy-C₁-C₄-alkyl, fluorinated C₁-C₂-alkyl, SF₅, C₁-C₄-alkyl-sulfonyl, fluorinated C₁-C₂-alkoxy, C(O)R⁴, NR⁵R⁶, C(O)NR⁷R⁸ and C(O)OR⁹, or two radicals R^{2a}, which are bound to adjacent carbon atoms, may also form a bridging moiety O-Alk′-O, wherein Alk′ is selected from CH₂, CH₂CH₂, CHF, CF₂ and CF₂CF₂;
R³ is hydrogen, C₁-C₆-alkyl, C₁-C₄-alkoxy-C₁-C₄-alkyl, fluorinated C₁-C₂-alkyl or C(O)R⁴;
R⁴ is selected from the group consisting of hydrogen, C₁-C₄-alkyl, C₁-C₄-alkoxy-C₁-C₄-alkyl, C₃-C₇-cycloalkyl, fluorinated C₁-C₂-alkyl, C₁-C₄-alkoxy, C₁-C₄-alkoxy-C₁-C₄-alkoxy, C₃-C₇-cycloalkoxy and fluorinated C₁-C₂-alkoxy;
R⁵ is hydrogen or C₁-C₆-alkyl;
R⁶ is C₁-C₆-alkyl, hydroxy-C₂-C₆-alkyl, C₁-C₄-alkoxy-C₂-C₄-alkyl OH, C₁-C₄-alkoxy or a radical C(O)R^{x}, wherein Rₓ is C₁-C₄-alkyl; or
R⁵, R⁶ together with the nitrogen atom, to which they are bound, form an N-bound, 5- or 6-membered saturated nitrogen heterocycle;
R⁷ and R⁸ independently of one another are hydrogen, OH, C₁-C₄-alkoxy or C₁-C₆-alkyl;
R⁹ is hydrogen, C₁-C₆-alkyl, hydroxy-C₂-C₆-alkyl or C₁-C₄-alkoxy-C₂-C₄-alkyl;
and the pharmaceutically acceptable salts thereof, the N-oxides thereof and the pharmaceutically acceptable salts of said N-oxides.

The invention relates in particular to use of the compounds of the formula (I), the pharmaceutically acceptable salts thereof, and, the N-oxides thereof or the pharmaceutically acceptable salts of said N-oxides in therapy of a disease or disorder selected from the group consisting of inflammatory diseases, a hyperproliferative disease or disorders, a hypoxia related pathology and a disease characterized by pathophysiological hypervascularization.

In a further aspect, the present invention relates to a pharmaceutical composition comprising at least one compound of the formula (I), the pharmaceutically acceptable salts thereof, and the N-oxides thereof or the pharmaceutically acceptable salts of said N-oxides optionally together with at least one physiologically acceptable carrier or auxiliary substance.

In a further aspect, the present invention relates to a method for treatment or prevention of a disease or disorder selected from the group consisting of an inflammatory disease, a hyperproliferative disease or disorder, a hypoxia related pathology and a disease characterized by pathophysiological hypervascularization, said method comprising administering an effective amount of at least one compound of the formula (I), the pharmaceutically acceptable salts thereof, and the N-oxides thereof or the pharmaceutically acceptable salts of said N-oxides to a subject in need thereof.

In a further aspect, the present invention relates to the use of a compound of the formula (I), the pharmaceutically acceptable salts thereof, and the N-oxides thereof or the pharmaceutically acceptable salts of said N-oxides in the manufacture of a medicament for therapy of a disorder or disease wherein the disorder or disease is selected from the group consisting of an inflammatory disease, a hyperproliferative disease or disorder, a hypoxia related pathology and a disease characterized by pathophysiological hypervascularization.

### DETAILED DESCRIPTION OF THE INVENTION

It is to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W., Nagel, B. and Klbl, H. Eds. (1995), Helvetica Chimica Acta, CH-4010 Basel, Switzerland).

Throughout this specification and the claims which follow, unless the context requires otherwise, the word “comprise”, and variations, such as “comprises” and “comprising”, will be understood to imply the inclusion of a stated integer or step or group of integers or steps but not the exclusion of any other integer or step or group of integers or steps.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

Provided the compounds of the formula (I) of a given constitution may exist in different spatial arrangements, for example, if they possess one or more centers of asymmetry, polysubstituted rings or double bonds, or as different tautomers, the invention also relates to enantiomeric mixtures, in particular racemates, diastereomeric mixtures and tautomeric mixtures, preferably, however, the respective essentially pure enantiomers (enantiomerically pure), diastereomers and tautomers of the compounds of formula (I) and/or of their salts and/or their N-oxides. Racemates obtained can be resolved into the isomers mechanically or chemically by methods known per se. Diastereomers are preferably formed from the racemic mixture by reaction with an optically active resolving agent. Examples of suitable resolving agents are optically active acids, such as the D and L forms of tartaric acid, diacetyltartaric acid, dibenzoyltartaric acid, mandelic acid, malic acid, lactic acid or the various optically active camphorsulfonic acids, such as D- or L-camphorsulfonic acid. Also advantageous is enantiomer resolution with the aid of a column filled with an optically active resolving agent (for example dinitrobenzoylphenylglycine); an example of a suitable eluent is a hexane/isopropanol/acetonitrile mixture. The diastereomer resolution can also be carried out by standard purification processes, such as, for example, chromatography or fractional crystallization. It is also possible to obtain optically active compounds of formula (I) by the methods described below by using starting materials which are already optically active.

The invention also relates to “pharmaceutically acceptable salts” of the compounds of the formula (I), especially acid addition salts with physiologically tolerated, i.e. pharmaceutically acceptable acids. Examples of suitable physiologically tolerated organic and inorganic acids include, but are not limited to, hydrochloric acid, hydrobromic acid, phosphoric acid, sulfuric acid, C₁-C₄-alkylsulfonic acids, such as methanesulfonic acid, aromatic sulfonic acids, such as benzenesulfonic acid and toluenesulfonic acid, carboxylic acids such as oxalic acid, malic acid, maleic acid, fumaric acid, lactic acid, tartaric acid, adipic acid, mandelic acid, salicylic acid, phenylpropionic acid, nicotinic acid, benzoic acid acetate, alginic acid, ascorbic acid, aspartic acid, tannic acid, butyric acid, camphoric acid, citric acid, clavulanic acid, cyclopentanepropionic acid, gluconic acid, formic acid, acetic acid, propionic acid, pivalic acid, valeric acid, hexoic acid, heptoic acid, oleic acid, palmitic acid, pantothenic acid, pectinic acid, stearic acid, hexylresorcinic acid, hydroxynaphthoic acid, lactobionic acid and mucic acid. Other utilizable acids are described in Fortschritte der Arzneimittelforschung [Advances in drug research], Volume 10, pages 224 ff., Birkhäuser Verlag, Basel and Stuttgart, 1966 and in Berge, S. M., et al., “Pharmaceutical Salts”, Journal of Pharmaceutical Science, 1977, 66, 1-19. Illustrative examples of pharmaceutically acceptable salts include but are not limited to: acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formiate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalene-sulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like. Certain specific compounds of the present invention contain both basic and acidic functionalities that allow the compounds to be converted into either base or acid addition salts. Furthermore, where the compound of the invention carries an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counteranions such as halide, hydroxide, carboxylate, sulfate, phosphate, nitrate, alkyl sulfonate and aryl sulfonate).

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

The invention also relates to N-oxides of the compounds of the formula (I) which are characterized in that one or several nitrogen atoms of the compounds of the formula (I) are oxidized to the so-called N-oxide.

In addition to salt forms, the N-oxides and the salts of the N-oxides, the present invention provides compounds which are in a prodrug form. Prodrugs of the compounds described herein are those compounds that readily undergo chemical changes under physiological conditions to provide a compound of general formula (I). A prodrug is a pharmacologically active or inactive compound that is modified chemically through in vivo physiological action, such as hydrolysis, metabolism and the like, into a compound of this invention following administration of the prodrug to a patient. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme. The suitability and techniques involved in making and using prodrugs are well known by those skilled in the art. For a general discussion of prodrugs involving esters, see Svensson and Tunek, Drug Metabolism Reviews 16.5 (1988), and Bundgaard, Design of Prodrugs, Elsevier (1985). Examples of a masked acidic anion include a variety of esters, such as alkyl (for example, methyl, ethyl), cycloalkyl (for example, cyclohexyl), aralkyl (for example, benzyl, p-methoxybenzyl), and alkylcarbonyloxyalkyl (for example, pivaloyloxymethyl). Amines have been masked as arylcarbonyloxymethyl substituted derivatives which are cleaved by esterases in vivo releasing the free drug and formaldehyde (Bungaard J. Med. Chem. 2503 (1989)). Also, drugs containing an acidic NH group, such as imidazole, imide, indole and the like, have been masked with N-acyloxymethyl groups (Bundgaard Design of Prodrugs, Elsevier (1985)). Hydroxy groups have been masked as esters and ethers. EP 0 039 051 (Sloan and Little, Apr. 11, 1981) discloses Mannich-base hydroxamic acid prodrugs, their preparation and use.

Certain compounds of the present invention can exist in unsolvated forms as well as in solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

The compounds of the present invention may also contain unnatural proportions of atomic isotopes at one or more of the atoms that constitute such compounds. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulfur, fluorine and chlorine such as ²H, ³H, ¹³C, ¹⁴C, ¹⁵N, ¹⁷O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes, such as deuterium, i.e., ²H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents. All isotopic variations of the compounds and compositions of the present invention, whether radioactive or not, are intended to be encompassed within the scope of the present invention.

In the following definitions of the terms: alkyl, heteroaryl, alkenyl, alkynyl, hydroxyalkyl and alkoxyalkyl are provided. These terms will in each instance of its use in the remainder of the specification have the respectively defined meaning and preferred meanings. Nevertheless, in some instances of their use throughout the specification preferred meanings of these terms are indicated.

The organic moieties mentioned in the above definitions of the variables are—like the term halogen—collective terms for individual listings of the individual group members. The prefix Cₙ-Cₘ indicates in each case the possible number of carbon atoms in the group. If two or more radicals can be selected independently from each other, then the term “independently” means that the radicals may be the same or may be different.

The term “halogen” denotes in each case fluorine, bromine, chlorine or iodine, in particular fluorine, chlorine or bromine.

The term “C₁-C₁₀-alkyl” denotes a straight-chain or branched alkyl group having from 1 to 10 carbon atoms, preferably 1 to 4 carbon atoms. Examples of an alkyl group are methyl, ethyl, n-propyl, iso-propyl, n-butyl, 2-butyl, iso-butyl, tert-butyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, octyl, nonyl and decyl.

The term “fluorinated C₁-C₂ alkyl” denotes an alkyl group having 1 or 2 carbon atoms as defined above, wherein at least one hydrogen atom, e.g. 1, 2, 3, 4 or 5 hydrogen atoms, are replaced by fluorine. Examples of such a group include fluoromethyl, difluoromethyl, trifluoromethyl, 2-fluoroethyl, 2,2-difluoroethyl, 2,2,2-trifluoroethyl, 1,1,2,2-tetrafluoroethyl and 1,1,2,2,2-pentafluoroethyl.

The term “pentafluorosulfanyl” (also referred to as pentafluorothio) relates to the radical SF₅.

The term “hydroxy-C₁-C₆-alkyl” denotes a straight-chain or branched alkyl group having from 1 to 6, especially 1 to 4 carbon atoms (=hydroxy-C₁-C₄ alkyl), wherein one of the hydrogen atoms is replaced by a hydroxy group, such as in hydroxymethyl, 1-hydroxyethyl, 2-hydroxyethyl, 3-hydroxypropyl, hydroxy-iso-propyl, 1-hydroxybutyl or 2-hydroxybutyl.

The term “hydroxy-C₂-C₆-alkyl” denotes a straight-chain or branched alkyl group having from 2 to 6, especially 2 to 4 carbon atoms (=hydroxy-C₂-C₄ alkyl), wherein one of the hydrogen atoms, which is preferably not located at C1, is replaced by a hydroxy group, such as in 2-hydroxyethyl or 3-hydroxypropyl. The term “C1” refers to the carbon atom by which hydroxy-C₂-C₆-alkyl is bound to the remainder of the molecule.

The term “C₁-C₆-alkoxy” denotes a straight-chain or branched alkyl group having 1, 2, 3, 4, 5 or 6 carbon atoms, which is bound to the remainder of the molecule via an oxygen atom. Examples of an alkoxy group are methoxy, ethoxy, n-propoxy, iso-propoxy, n-butyloxy, 2-butyloxy, iso-butyloxy, tert-butyloxy, pentyloxy, 1-methylbutyloxy, 2-methylbutyloxy, 3-methylbutyloxy, 2,2-dimethylpropyloxy, 1-ethylpropyloxy, hexyloxy, 1,1-dimethylpropyloxy, 1,2-dimethylpropyloxy, 1-methylpentyloxy, 2-methylpentyloxy, 3-methylpentyloxy, 4-methylpentyloxy, 1,1-dimethylbutyloxy, 1,2-dimethylbutyloxy, 1,3-dimethylbutyloxy, 2,2-dimethylbutyloxy, 2,3-dimethylbutyloxy, 3,3-dimethylbutyloxy, 1-ethylbutyloxy, 2-ethylbutyloxy, 1,1,2-trimethylpropyloxy, 1,2,2-trimethylpropyloxy, 1-ethyl-1-methylpropyloxy and 1-ethyl-2-methylpropyloxy.

The term “fluorinated C₁-C₂-alkoxy” denotes an alkoxy group having 1 or 2 carbon atoms, wherein at least one hydrogen atom, e.g. 1, 2, 3, 4 or 5 hydrogen atoms, are replaced by fluorine. Examples of such a group include fluoromethoxy, difluoromethoxy, trifluoromethoxy, 2-fluoroethoxy, 2,2-difluoroethoxy, 2,2,2-trifluoroethoxy, 1,1,2,2-tetrafluoroethoxy and 1,1,2,2,2-pentafluoroethoxy.

The term “C₁-C₄-alkoxy-C₁-C₄-alkyl” denotes a straight-chain or branched alkyl group having from 1 to 4 carbon atoms, wherein one of the hydrogen atoms is replaced by a C₁-C₄ alkoxy group, such as in methoxymethyl, ethoxymethyl, propoxymethyl, 1-methoxyethyl, 1-ethoxyethyl, 2-methoxyethyl, 2-ethoxyethyl, 2-methoxypropyl, 2-ethoxypropyl, 3-methoxypropyl or 3-ethoxypropyl.

The term “C₁-C₄-alkoxy-C₂-C₄-alkyl” denotes a straight-chain or branched alkyl group having from 2 to 4 carbon atoms, wherein one of the hydrogen atoms, which is preferably not located at C1, is replaced by a C₁-C₄ alkoxy group, such as in 2-methoxyethyl, 2-ethoxyethyl, 2-methoxypropyl, 2-ethoxypropyl, 3-methoxypropyl or 3-ethoxypropyl. The term “C1” refers to the carbon atom by which C₁-C₄-alkoxy-C₂-C₄-alkyl is bound to the remainder of the molecule.

The term “C₁-C₆-alkylthio” (also referred to as alkylsulfanyl) denotes a straight-chain or branched alkyl groups having 1 to 6 carbon atoms (as mentioned above) which are attached to the skeleton via a sulfur atom (—S—).

The term “fluorinated C₁-C₂-alkylthio” denotes an alkylsulfanyl group having 1 or 2 carbon atoms, wherein at least one hydrogen atom, e.g. 1, 2, 3, 4 or 5 hydrogen atoms, are replaced by fluorine. Examples of such a group include fluoromethylsulfanyl, difluoromethylsulfanyl, trifluoromethylsulfanyl, 2-fluoroethylsulfanyl, 2,2-difluoroethylsulfanyl, 2,2,2-trifluoroethylsulfanyl, 1,1,2,2-tetrafluoroethylsulfanyl and 1,1,2,2,2-pentafluoroethylsulfanyl.

The term “C₁-C₄-alkylsulfonyl” refers to straight-chain or branched alkyl group having 1 to 4 carbon atoms (as defined above) bonded through a —S(═O)₂ moiety, at any position in the alkyl group, for example methylsulfonyl, ethylsulfonyl, n-propylsulfonyl, isopropylsulfonyl and t-butylsulfonyl.

The term “fluorinated C₁-C₂-alkylsulfonyl” denotes an alkylsulfonyl group having 1 or 2 carbon atoms, wherein at least one hydrogen atom, e.g. 1, 2, 3, 4 or 5 hydrogen atoms, are replaced by fluorine. Examples of such a group include fluoromethanesulfonyl, difluoromethanesulfonyl, trifluoromethanesulfonyl, 2-fluoroethanesulfonyl, 2,2-difluoroethanesulfonyl, 2,2,2-trifluoroethanesulfonyl, 1,1,2,2-tetrafluoroethanesulfonyl and 1,1,2,2,2-pentafluoroethanesulfonyl.

The term “C₂-C₁₀-alkenyl” denotes a straight-chain or branched hydrocarbon groups having 2 to 4, 6, 8 or 10 carbon atoms and one or more, e.g. 2 or 3, carbon-carbon double bonds in any position, preferably one carbon-carbon double bond, e.g. vinyl, allyl(2-propen-1-yl), 1-propen-1-yl, 2-propen-2-yl, methallyl(2-methylprop-2-en-1-yl), 2-buten-1-yl, 3-buten-1-yl, 2-penten-1-yl, 3-penten-1-yl, 4-penten-1-yl, 1-methyl-but-2-en-1-yl, 2-ethylprop-2-en-1-yl, hexenyl, heptenyl or octenyl.

The term “C₂-C₁₀ alkynyl” denotes a straight-chain or branched hydrocarbon groups having 2 to 4, 6, 8 or 10 carbon atoms and one or more, e.g. 2 or 3, carbon-carbon triple bonds in any position, preferably one carbon-carbon triple bond, for example ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl, heptynyl, octynyl, nonynyl or decynyl.

The term “C₃-C₇-cycloalkyl” as used herein denotes in each case a monocyclic radical having from 3 to 7 carbon atoms as ring members, such as cyclopropyl, cyclobutyl, cyclopentyl, cyclohexyl or cycloheptyl.

The term “C₃-C₇-cycloalkoxy” refers to C₃-C₇-cycloalkyl radical having 3 to 7 carbon atoms (as defined above), which is bound to the remainder of the molecule via an oxygen atom. Examples of a cycloalkoxy group are cyclopropoxy, cyclobutoxy, cyclopentoxy, cyclohexoxy and cycloheptoxy.

The term “C-bound monocyclic 5- or 6-membered heteroaryl” (also referred to as C-bound monocyclic 5- or 6-membered hetaryl) denotes a monocyclic 5- or 6-membered heteroaromatic radical comprising as ring members in addition to carbon atom(s) in general 1, 2, 3 or 4 heteroatoms independently of each other selected from N, O and S, wherein the a heteroaromatic radical is bound via a carbon ring atom to the skeleton.

Examples of 5- or 6-membered heteroaromatic radicals include 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl, 1,2,4-triazin-3-yl, 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl and 1H- or 2H-tetrazolyl. Preference is given to 5- to 6-membered heteroaryl radicals having one or two heteroatoms independently of each other selected from of N, O and S, for example furyl, thienyl, thiazolyl, oxazolyl, isothiazolyl, isoxazolyl, imidazolyl, pyridyl, pyrimidinyl, pyridazinyl and pyrazinyl.

The term “N-bound, 5- or 6-membered saturated nitrogen heterocycle” denotes a saturated heteromonocyclic radical containing one nitrogen atom as a ring member, which is attached to the remainder of the molecule, and optionally one or more, e.g. 1 or 2 further heteroatoms, such as O, S or N as ring member, having a total of 5 or 6 ring member atoms. Examples for “N-bound, 5- or 6-membered saturated nitrogen heterocycles” are pyrrolidin-1-yl, piperidin-1-yl, piperazin-1-yl, 4-methylpiperazin-1-yl, morpholin-4-yl, thiomorpholin-4-yl, imidazolidin-1-yl, oxazolidin-3-yl or thiazolidin-3-yl, especially pyrrolidin-1-yl, piperazin-1-yl, 4-methylpiperazin-1-yl, piperidin-1-yl and morpholin-4-yl.

Hereinafter, particular embodiments of the compounds according to the present invention are explained by giving particular or preferred meanings of the variables X, R¹, R^{1a}, R², R^{2a}, R³, R⁴, R⁵, R⁶, R⁷, R⁸ and R⁹. It is well understood by a skilled person that any particular or preferred meaning of any of these variables can be combined with the general definition or a particular or preferred meaning of any other variables, e.g. any definition of a particular or preferred embodiment of R¹ can be combined with any particular or preferred meaning of R².

According to a first embodiment, the present invention relates to the compounds of the formula I, to their salts, to their N-oxides and to the salts of the N-oxides, wherein X is CH₂. These compounds are hereinafter also denominated as compounds of the formula I.1.

According to a second embodiment, the present invention relates to the compounds of the formula I, to their salts, to their N-oxides and to the salts of the N-oxides, wherein X is CH₂CH₂. These compounds are hereinafter also denominated as compounds of the formula I.2.

According to a third embodiment, the present invention relates to the compounds of the formula I, to their salts, to their N-oxides and to the salts of the N-oxides, wherein X is C═O. These compounds are hereinafter also denominated as compounds of the formula I.3.

Particular preference is given to the second embodiment, i.e. to compounds of the formula I.2.

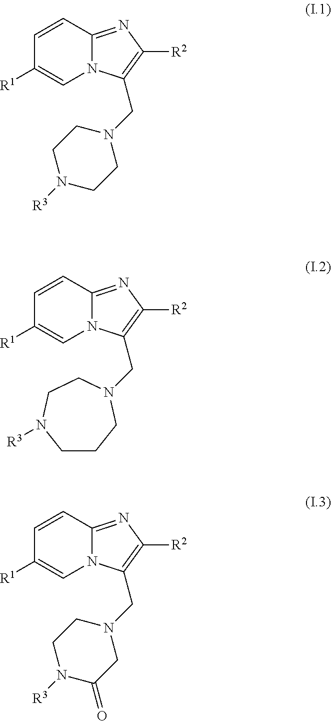

According to a particular embodiment, the present invention relates to the compounds of the formulae I, I.1, I.2 and I.3, to their salts, to their N-oxides and to the salts of the N-oxides, wherein R¹ is phenyl, which is unsubstituted or which carries 1, 2, 3, 4 or 5 and preferably 1, 2 or 3 radicals R^{1a} which are identical or different. Amongst these compounds, those are preferred, wherein the carbon ring atoms which are located in the ortho position with respect to the imidazo[1,2-a]pyridine moiety do not carry a radical R. Preferably, one of the radicals R^{1a}, if present, is located in the para-position with respect to the imidazo[1,2-a]pyridine moiety.

According to another particular embodiment, the present invention relates to the compounds of the formulae I, I.1, I.2 and I.3, to their salts, to their N-oxides and to the salts of the N-oxides, wherein R¹ is 6-membered heteroaryl, which is unsubstituted or which carries 1, 2, 3, 4 or 5 and preferably 1, 2 or 3 radicals R^{1a} which are identical or different. Amongst these compounds, those are preferred, wherein the one or more hetero ring atom(s) are not located in the ortho position with respect to the imidazo[1,2-a]pyridine moiety. Amongst these compounds, those are preferred, wherein the carbon ring atom(s) which are located in the ortho position with respect to the imidazo[1,2-a]pyridine moiety do not carry a radical R. Preferably, one of the radicals R^{1a}, if present, is located in the para-position with respect to the imidazo[1,2-a]pyridine moiety.

According to a further particular embodiment, the present invention relates to the compounds of the formulae I, I.1, I.2 and I.3, to their salts, to their N-oxides and to the salts of the N-oxides, wherein R¹ is 5-membered C-bound heteroaryl, which is unsubstituted or which carries 1, 2 or 3 and preferably 1 or 2 radicals R^{1a} which are identical or different. Amongst these compounds, those are preferred, wherein the carbon ring atom(s) which are located in the ortho position with respect to the imidazo[1,2-a]pyridine moiety do not carry a radical R.

In these embodiments, the radicals R^{1a}, if present, have the aforementioned meanings and are preferably selected from halogen, OH, SH, CN, NO₂, C₁-C₄ alkyl, especially methyl, ethyl or isopropyl, C₂-C₄ alkenyl, especially ethenyl, C₂-C₄ alkynyl, especially ethynyl, C₁-C₄-alkoxy, especially methoxy or ethoxy, C₁-C₄-alkylthio, especially methylthio or ethylthio, fluorinated C₁-C₂-alkyl, especially difluoromethyl or trifluoromethyl, SF₅, fluorinated C₁-C₂-alkoxy, especially difluoromethoxy or trifluoromethoxy, fluorinated C₁-C₂-alkylsulfonyl, especially trifluoromethylsulfonyl, fluorinated C₁-C₂-alkylthio, especially trifluoromethylsulfanyl, NH₂, hydroxy-C₁-C₄-alkyl, especially hydroxymethyl, 1-hydroxyethyl or 2-hydroxyethyl, C₁-C₄-alkoxy-C₁-C₄-alkyl, especially methoxymethyl, ethoxymethyl, 1-methoxyethyl or 2-methoxyethyl, C(O)R⁴, especially acetyl or propionyl, NR⁵R⁶ especially methylamino or dimethylamino, C(O)NR⁷R⁸, especially aminocarbonyl, methylaminocarbonyl or dimethylaminocarbonyl, and C(O)OR⁸, especially methoxycarbonyl, ethoxycarbonyl or carboxy, or two radicals R^{1a}, which are bound to adjacent carbon atoms, together may also form a moiety O-Alk-O, wherein Alk is selected from CH₂, CH₂CH₂, CHF and CF₂. Especially, the radicals R^{1a}, if present, are selected from the group consisting of halogen, in particular fluorine or chlorine, CN (=cyano), NO₂ (nitro), OH, SH, C₁-C₄-alkyl, in particular methyl, ethyl or isopropyl, C₁-C₄-alkoxy, in particular methoxy or ethoxy, NH₂, NR⁴R⁵, in particular NH(CH₃) or N(CH₃)₂, fluorinated C₁-alkyl, in particular difluoromethyl or trifluoromethyl, SF₅, fluorinated C₁-alkoxy, in particular difluoromethoxy or trifluoromethoxy, aminocarbonyl, acetyl or two radicals R^{1a}, which are bound to adjacent carbon atoms, together may also form a moiety O-Alk-O, wherein Alk is selected from CH₂, CH₂CH₂, CHF and CF₂. In this embodiment, if the radical R^{1a} is fluorinated C₁-C₂-alkylsulfonyl, fluorinated C₁-C₂-alkylthio, R^{1a} is preferably located in the para-position with respect to the point of attachment to the imidazo[1,2-a]pyridine core of (I).

Amongst the compounds of the present invention, where R¹ in the formulae I, I.1, I.2 or I.3 is phenyl or 6-membered heteroaryl, which are unsubstituted or which carry 1, 2, 3, 4 or 5 and preferably 1, 2 or 3 radicals R^{1a} which are identical or different, preference is given to those compounds, wherein R¹ in the formulae I, I.1, I.2 or I.3 is a radical of the formula Ar1:

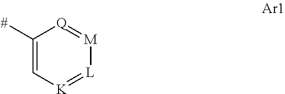

wherein # indicates the point of attachment to the imidazo[1,2-a]pyridine core of (I),
K is N or C—R¹¹,
L is N or C—R¹²,
M is N or C—R¹³,
Q is N or C—R¹⁴,
wherein R¹¹, R¹², R¹³ and R¹⁴, independently of each other, are hydrogen or have one of the meanings given for R^{1a}, in particular one of the preferred meanings.

Amongst the compounds of the present invention, where R¹ in the formulae I, I.1, I.2 or I.3 is phenyl or 6-membered heteroaryl, which are unsubstituted or which carry 1, 2, 3, 4 or 5 and preferably 1, 2 or 3 radicals R^{1a} which are identical or different, preference is given to those compounds, wherein R¹ in the formulae I, I.1, I.2 or I.3 is selected from radicals of the formulae Ar1.1 to Ar1.7, with particular preference given to the formula Ar1.1:

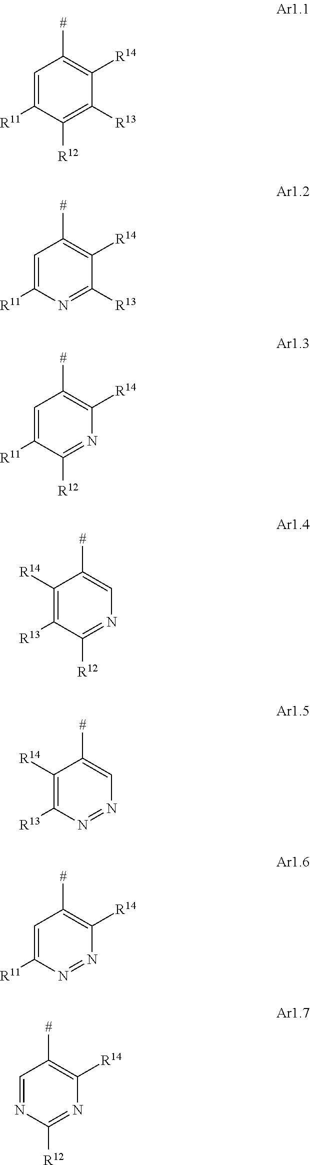

wherein # indicates the point of attachment to the imidazo[1,2-a]pyridine core, and wherein R¹¹, R¹², R¹³ and R¹⁴, independently of each other, are hydrogen or have one of the meanings given for R^{1a}, in particular one of the preferred meanings.

In the compounds of the formulae I, I.1, I.2 or I.3, where R¹ is a radical of the formulae Ar1, Ar1.1, Ar1.2, Ar1.3, Ar1.4, Ar1.5, Ar1.6 or Ar1.7, particular embodiments of the invention relate to compounds, wherein, the variables R¹¹, R¹², R¹³ and R¹⁴, if present, individually or in particular in combination have the following meanings:

R¹¹, R¹³ are independently of each other selected from the group consisting of hydrogen, halogen, especially fluorine and chlorine, OH, CN, NO₂, SH, C₁-C₄ alkyl, especially methyl, ethyl or isopropyl, C₂-C₄ alkenyl, especially ethenyl, C₂-C₄ alkynyl, especially ethynyl, C₁-C₄-alkoxy, especially methoxy or ethoxy, C₁-C₄-alkylthio, especially methylthio or ethylthio, fluorinated C₁-alkyl, especially difluoromethyl or trifluoromethyl, fluorinated C₁-alkoxy, especially difluoromethoxy or trifluoromethoxy, NH₂, NR⁵R⁶ such as NHCH₃ or N(CH₃)₂, hydroxy-C₁-C₄-alkyl, especially hydroxymethyl, 1-hydroxyethyl or 2-hydroxyethyl, C₁-C₄-alkoxy-C₁-C₄-alkyl, especially methoxymethyl, ethoxymethyl, 1-methoxyethyl or 2-methoxyethyl;

R¹² is selected from the group consisting of hydrogen, halogen, especially fluorine and chlorine, OH, SH, CN, NO₂, C₁-C₄ alkyl, especially methyl, ethyl or isopropyl, C₂-C₄ alkenyl, especially ethenyl, C₂-C₄ alkynyl, especially ethynyl, C₁-C₄-alkoxy, especially methoxy or ethoxy, C₁-C₄-alkylthio, especially methylthio or ethylthio, fluorinated C₁-C₂-alkyl, especially difluoromethyl or trifluoromethyl, SF₅, fluorinated C₁-C₂-alkoxy, especially difluoromethoxy or trifluoromethoxy, fluorinated C₁-C₂-alkylsulfonyl, especially trifluoromethylsulfonyl, fluorinated C₁-C₂-alkylthio, especially trifluoromethylsulfanyl, NH₂, hydroxy-C₁-C₄-alkyl, especially hydroxymethyl, 1-hydroxyethyl or 2-hydroxyethyl, C₁-C₄-alkoxy-C₁-C₄-alkyl, especially methoxymethyl, ethoxymethyl, 1-methoxyethyl or 2-methoxyethyl, C(O)R⁴, especially acetyl or propionyl, NR⁵R⁶ especially methylamino or dimethylamino, aminocarbonyl and C(O)OR⁹, especially methoxycarbonyl or ethoxycarbonyl. In particular, R¹² is selected from hydrogen, halogen, especially fluorine or chlorine, CN (=cyano), NO₂ (nitro), OH, SH, C₁-C₄-alkyl, especially methyl, ethyl or isopropyl, C₁-C₄-alkoxy, especially methoxy or ethoxy, NH₂, NR⁵R⁶, in particular NH(CH₃), or N(CH₃)₂, fluorinated C₁-alkyl, especially difluoromethyl or trifluoromethyl, SF₅, fluorinated C₁-alkoxy, especially difluoromethoxy or trifluoromethoxy and aminocarbonyl.

R¹⁴ is selected from the group consisting of hydrogen, halogen, especially fluorine and chlorine, OH, C₁-C₄-alkyl, especially methyl, ethyl or isopropyl, C₂-C₄ alkenyl, especially ethenyl, C₂-C₄ alkynyl, especially ethynyl, C₁-C₄-alkoxy, especially methoxy or ethoxy, fluorinated C₁-C₂-alkyl, especially difluoromethyl or trifluoromethyl, and fluorinated C₁-C₂-alkoxy, especially difluoromethoxy or trifluoromethoxy. In particular, R¹⁴ is selected from hydrogen, halogen, especially fluorine or chlorine, C₁-C₂-alkyl, especially methyl or ethyl, C₁-C₂-alkoxy, especially methoxy or ethoxy, fluorinated C₁-alkyl, especially difluoromethyl or trifluoromethyl and fluorinated C₁-alkoxy, especially difluoromethoxy or trifluoromethoxy.

In the compounds of the formulae I, I.1, I.2 or I.3, where R¹ is a radical of the formulae Ar1, Ar1.1, Ar1.2, Ar1.3, Ar1.4, Ar1.5, Ar1.6 or Ar1.7, more preferred embodiments of the invention relate to compounds, wherein, the variables R¹¹, R¹², R¹³ and R¹⁴, if present, individually or in particular in combination have the following meanings:

R¹¹, R¹³, are independently of each other selected from the group consisting of hydrogen, halogen, OH, methyl, difluoromethyl, trifluoromethyl, methoxy, difluoromethoxy and trifluoromethoxy;

In case of formulae Ar1.1 and Ar1.2 and in case of formula Ar1 with K being C—R¹¹ and M being C—R¹³, preferably one of R¹¹ and R¹³ is selected from hydrogen, halogen, OH, methyl, difluoromethyl, trifluoromethyl, methoxy, difluoromethoxy and trifluoromethoxy and the other one is hydrogen;

R¹² is selected from the group consisting of halogen, especially fluorine or chorine, OH, SH, CN, NO₂, C₁-C₄ alkyl, especially methyl, ethyl or isopropyl, C₂-C₄ alkenyl, especially ethenyl, C₂-C₄ alkynyl, especially ethynyl, C₁-C₄-alkoxy, especially methoxy or ethoxy, C₁-C₄-alkylthio, especially methylthio or ethylthio, fluorinated C₁-alkyl, especially difluoromethyl or trifluoromethyl, SF₅, fluorinated C₁-alkoxy, especially difluoromethoxy or trifluoromethoxy, NH₂, hydroxy-C₁-C₄-alkyl, especially hydroxymethyl, 1-hydroxyethyl or 2-hydroxyethyl, C₁-C₄-alkoxy-C₁-C₄-alkyl, especially methoxymethyl, ethoxymethyl, 1-methoxyethyl or 2-methoxyethyl, C(O)R⁴, especially acetyl or propionyl, aminocarbonyl, NR⁵R⁶, especially methylamino or dimethylamino; and

R¹⁴ is selected from hydrogen, halogen, especially fluorine or chlorine, C₁-C₂-alkyl, especially methyl or ethyl, C₁-C₂-alkoxy, especially methoxy or ethoxy, fluorinated C₁-alkyl, especially difluoromethyl or trifluoromethyl and fluorinated C₁-alkoxy, especially difluoromethoxy or trifluoromethoxy.

Amongst the compounds of the present invention, where R¹ in the formulae I, I.1, I.2 or I.3 is C-bound 5-membered heteroaryl, which is unsubstituted or which carries 1, 2 or 3 radicals R^{1a} which are identical or different, preference is given to those compounds, wherein R¹ in the formulae I, I.1, I.2 or I.3 is a radical of the formulae Ar2 or Ar2′:

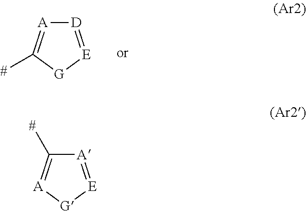

wherein # indicates the point of attachment of R¹ to the imidazo[1,2-a]pyridine core of (I),
A is N or C—R¹⁵,
A′ is N or C—R¹⁶,
D is N or C—R¹⁷,
E is N or C—R¹⁸,
G is O, S or N—R¹⁹,
G′ is O, S or N—R²⁰,

wherein R¹⁵, R¹⁶, R¹⁷ and R¹⁸, independently of each other, are hydrogen or have one of the meanings given for R^{1a}, and wherein R¹⁹ and R²⁰ are hydrogen, cyano, NH₂, OH, C₁-C₁₀-alkyl, in particular C₁-C₄-alkyl, especially methyl, ethyl or isopropyl, C₂-C₁₀-alkenyl, in particular C₂-C₄-alkenyl, especially ethenyl or 3-propenyl, C₂-C₁₀-alkynyl, in particular C₂-C₄-alkynyl, especially ethynyl or 3-propynyl, C₁-C₆-alkoxy, in particular C₁-particular C₂-C₄-alkynyl, especially ethynyl or 3-propynyl, C₁-C₆-alkoxy, in particular C₁-C₄-alkoxy, especially methoxy or ethoxy, hydroxy-C₂-C₆-alkyl, in particular hydroxy-C₂-C₄-alkyl, especially 2-ethoxyethyl, C₁-C₄-alkoxy-C₂-C₄-alkyl, especially 1-methoxyethyl or 2-methoxyethyl, fluorinated C₁-C₂-alkyl, in particular fluorinated C₁-alkyl, fluorinated C₁-C₂-alkoxy, in particular fluorinated C₁-alkoxy, especially difluoromethoxy or trifluoromethoxy, C(O)R⁴, in particular acetyl or propionyl, NR⁵R⁶, especially methylamino or dimethylamino, and C(O)OR⁸, especially methoxycarbonyl or ethoxycarbonyl.

Amongst the compounds of the present invention, where R¹ in the formulae I, I.1, I.2 or I.3 is a C-bound 5-membered heteroaryl, which is unsubstituted or which carries 1, 2 or 3 radicals R^{1a} which are identical or different, preference is given to those compounds, wherein R¹ in the formulae I, I.1, I.2 or I.3 is selected from radicals of the formulae Ar2.1, Ar2.2, Ar2.3, Ar2.4, Ar2.5, Ar2.6, Ar2.7, Ar2.8, Ar2.9, Ar2.10, Ar2.11, Ar2.12, Ar2.13, Ar2.14, Ar2.15, Ar2.16, Ar2.17, Ar2.18, Ar2.19, Ar2.20, Ar2.21, Ar2.22, Ar2′.1, Ar2′.2, Ar2′.3, Ar2′.4, Ar2′.5, Ar2′.6, Ar2′.7, Ar2′.8, Ar2′.9, Ar2′.10, Ar2′.11, Ar2′.12, Ar2′.13, Ar2′.14 and Ar2′.15 with particular preference given to the formulae Ar2.1, Ar2.2, Ar2.3, Ar2.4, Ar2′.2 and Ar2′.6:

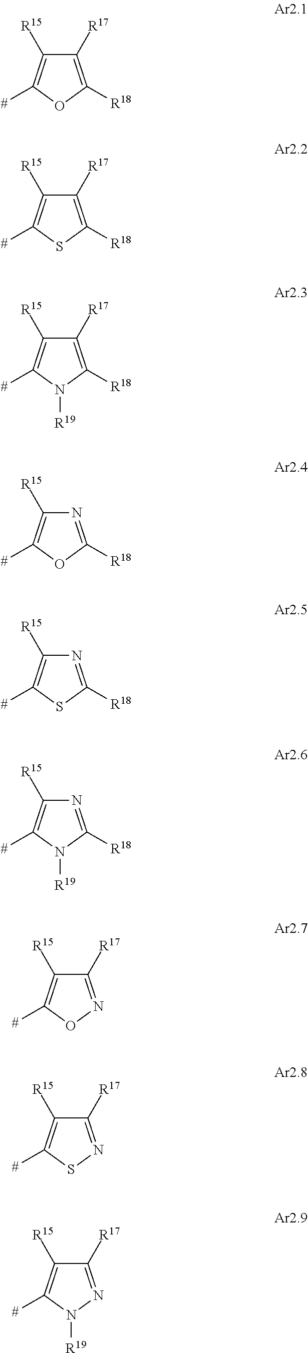

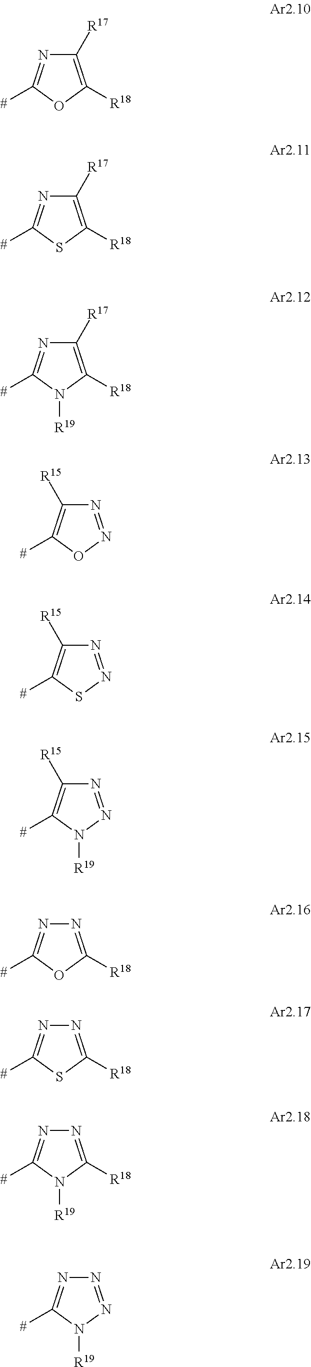

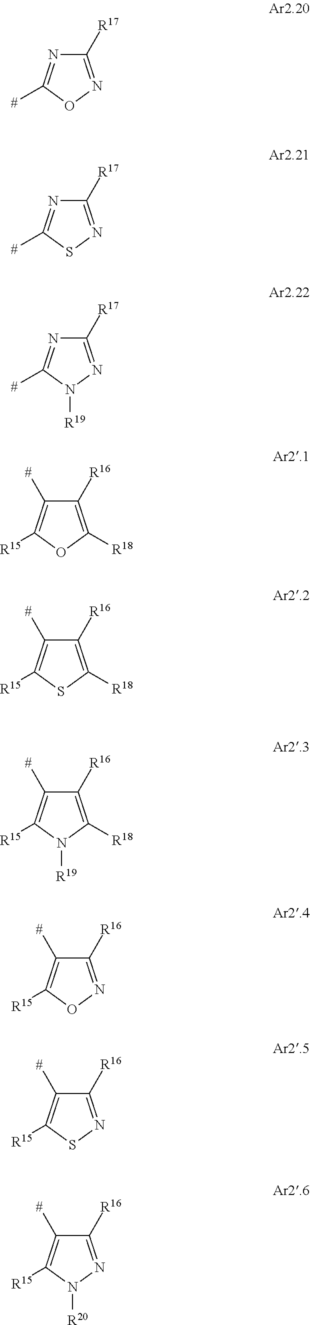

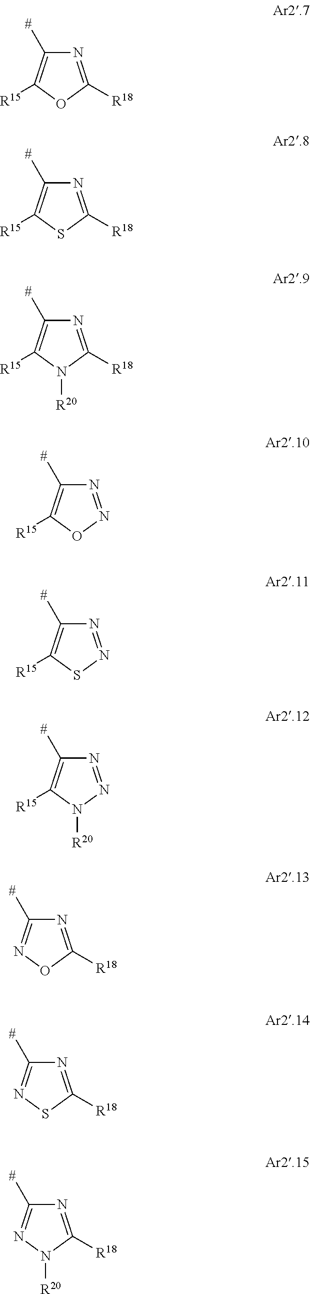

wherein # indicates the point of attachment of R¹ to the imidazo[1,2-a]pyridine core of (I), and
wherein R¹⁵, R¹⁶, R¹⁷, R¹⁸, R¹⁹ and R²⁰ have the meanings given above. In the compounds of the formulae I, I.1, I.2 or I.3, where R¹ is selected from radicals of the formulae Ar2.1, Ar2.2, Ar2.3, Ar2.4, Ar2.5, Ar2.6, Ar2.7, Ar2.8, Ar2.9, Ar2.10, Ar2.11, Ar2.12, Ar2.13, Ar2.14, Ar2.15, Ar2.16, Ar2.17, Ar2.18, Ar2.19, Ar2.20, Ar2.21, Ar2.22, Ar2′.1, Ar2′.2, Ar2′.3, Ar2′.4, Ar2′.5, Ar2′.6, Ar2′.7, Ar2′.8, Ar2′.9, Ar2′.10, Ar2′.11, Ar2′.12, Ar2′.13, Ar2′.14 and Ar2′.1 5, particular embodiments of the invention relate to compounds, wherein, the variables R¹⁵, R¹⁶, R¹⁷, R¹⁸, R¹⁹ and R²⁰, if present, individually or in particular in combination have the following meanings:

R¹⁵, is hydrogen;

R¹⁶, is hydrogen;

R¹⁷, R¹⁸ are independently of each other selected from the group consisting of hydrogen, halogen, in particular fluorine or chlorine, CN, NO₂, C₁-C₄ alkyl, especially methyl, ethyl or isopropyl, C₂-C₄ alkenyl, especially ethenyl, C₂-C₄ alkynyl, especially ethynyl, C₁-C₄-alkoxy, especially methoxy or ethoxy, C₁-C₄-alkylthio, especially methylthio or ethylthio, fluorinated C₁-alkyl, especially difluoromethyl or trifluoromethyl, fluorinated C₁-alkoxy, especially difluoromethoxy or trifluoromethoxy, NH₂, hydroxy-C₁-C₄-alkyl, especially hydroxymethyl, 1-hydroxyethyl or 2-hydroxyethyl, C₁-C₄-alkoxy-C₁-C₄-alkyl, especially methoxymethyl, ethoxymethyl, 1-methoxyethyl or 2-methoxyethyl, C(O)R⁴, especially acetyl or propionyl, and NR⁴R⁵, especially methylamino or dimethylamino;

R¹⁹, R²⁰ are independently of each other selected from the group consisting of hydrogen, CN, C₁-C₄ alkyl, especially methyl, ethyl or isopropyl, C₂-C₄ alkenyl, especially ethenyl, C₂-C₄ alkynyl, especially ethynyl, fluorinated C₁-alkyl, especially difluoromethyl or trifluoromethyl, C₁-C₄-alkoxy-C₁-C₄-alkyl, especially methoxymethyl, ethoxymethyl, 1-methoxyethyl or 2-methoxyethyl, and hydroxy-C₁-C₄-alkyl, especially hydroxymethyl, 1-hydroxyethyl or 2-hydroxyethyl. In particular, R¹⁹, R²⁰ are independently of each other selected from hydrogen and methyl.

Examples of radicals R¹ are given in the following tables A, which are particular embodiments according to the present invention.

TABLE A
Meanings of R¹

| | R¹ |
|---|---|
| 1. | Phenyl |
| 2. | 2-methylphenyl |
| 3. | 3-methylphenyl |
| 4. | 3-methoxyphenyl |
| 5. | 3-chlorophenyl |
| 6. | 3-nitrophenyl |
| 7. | 3-cyanophenyl |
| 8. | 3-trifluoromethylphenyl |
| 9. | 3-trifluoromethoxyphenyl |
| 10. | 4-acetylphenyl |
| 11. | 4-methylphenyl |
| 12. | 4-methoxyphenyl |
| 13. | 4-trifluoromethylphenyl |
| 14. | 4-trifluoromethoxyphenyl |
| 15. | 4-bromophenyl |
| 16. | 4-chlorophenyl |
| 17. | 4-fluorophenyl |
| 18. | 4-(methylthio)phenyl |
| 19. | 4-cyanophenyl |
| 20. | 4-aminophenyl |
| 21. | 4-nitrophenyl |
| 22. | 4-(dimethylamino)phenyl |
| 23. | 4-methylamino-phenyl |
| 24. | 4-aminocarbonylphenyl |
| 25. | 3-chloro-4-methylamino-phenyl |
| 26. | 3-chloro-4-methylphenyl |
| 27. | 3-chloro-4-methoxyphenyl |
| 28. | 3-methyl-4-chlorophenyl |
| 29. | 4-chloro-3-cyanophenyl |
| 30. | 3,4-dimethylphenyl |
| 31. | 3,4-dimethoxyphenyl |
| 32. | 3,4-dicyanophenyl |
| 33. | 3-methyl-4-cyanophenyl |
| 34. | 3-fluoro-4-cyanophenyl |
| 35. | 3-methoxy-4-cyanophenyl, |
| 36. | 3-chloro-4-cyanophenyl |
| 37. | 3,5-chloro-4-cyanophenyl |
| 38. | 4-pentafluorosulfanylphenyl, |
| 39. | 3-pyridyl |
| 40. | 4-pyridyl |
| 41. | 5-cyanopyridine-3-yl |
| 42. | 6-isopropylpyridine-3-yl |
| 43. | 5-fluoropyridine-3-yl |
| 44. | 5-chloropyridine-3-yl |
| 45. | 5-methoxypyridine-3-yl |
| 46. | 5-trifluoromethoxypyridine-3-yl |
| 47. | 5-methylpyridine-3-yl |
| 48. | 4-methylpyridine-2-yl |
| 49. | 5-methylpyridine-2-yl |
| 50. | 6-methylpyridine-2-yl |
| 51. | 5-trifluoromethylpyridine-3-yl |
| 52. | 6-cyanopyridine-3-yl |
| 53. | 6-fluoropyridine-3-yl |
| 54. | 6-chloropyridine-3-yl |
| 55. | 6-methylpyridine-3-yl |
| 56. | 6-methylthiopyridine-3-yl |
| 57. | 6-trifluoromethylpyridine-3-yl |
| 58. | 6-methoxypyridine-3-yl |
| 59. | 6-ethoxypyridine-3-yl |
| 60. | 6-trifluoromethoxypyridine-3-yl |
| 61. | 6-dimethylaminopyridine-3-yl |
| 62. | 2-chloropyridine-4-yl |
| 63. | 2-cyanopyridine-4-yl |
| 64. | 2-methylpyridine-4-yl |
| 65. | 2-methoxypyridine-4-yl |
| 66. | 2-trifluoromethylpyridine-4-yl |
| 67. | 2-trifluoromethoxypyridine-4-yl |
| 68. | 5-cyano-4-methylpyridine-3-yl |
| 69. | 2-cyano-6-methylpyridine-4-yl |
| 70. | pyrimidine-5-yl |
| 71. | 2-methylthiopyrimidine-5-yl |
| 72. | 2-trifluoromethoxypyrimidine-5-yl |
| 73. | 5-methoxypyridazin-3-yl |
| 74. | 5-methylpyridazin-3-yl |
| 75. | 5-chloropyridazin-3-yl |
| 76. | 5-cyanopyridazin-3-yl, |
| 77. | furan-2-yl |
| 78. | furan-3-yl |
| 79. | 5-methylfuran-2-yl |
| 80. | 4-methylfuran-2-yl |
| 81. | 5-methylfuran-3-yl |
| 82. | 5-cyanofuran-2-yl |
| 83. | 4-cyanofuran-2-yl |
| 84. | 5-cyanofuran-3-yl |
| 85. | 5-nitrofuran-2-yl |
| 86. | 2-thienyl |
| 87. | 3-thienyl |
| 88. | 5-methyl-2-thienyl |
| 89. | 4-methyl-2-thienyl |
| 90. | 5-methyl-3-thienyl |
| 91. | 5-cyano-2-thienyl |
| 92. | 4-cyano-2-thienyl |
| 93. | 5-cyano-3-thienyl |
| 94. | 4-cyano-3-thienyl |
| 95. | 5-trifluoromethoxy-3-thienyl |
| 96. | 4-chloro-5-methyl-2-thienyl |
| 97. | 5-cyano-4-fluoro-2-thienyl |
| 98. | pyrrol-2-yl |
| 99. | pyrrol-3-yl |
| 100. | 1-methylpyrrol-2-yl |
| 101. | 4-methylpyrrol-2-yl |
| 102. | 5-methylpyrrol-2-yl |
| 103. | 5-methylpyrrol-3-yl |
| 104. | 5-cyanopyrrol-2-yl |
| 105. | 4-cyanopyrrol-2-yl |
| 106. | 5-cyanopyrrol-3-yl |
| 107. | 1,5-dimethylpyrrol-2-yl |
| 108. | pyrazol-3-yl |
| 109. | pyrazol-4-yl |
| 110. | 1-methylpyrazol-3-yl |
| 111. | 1-methylpyrazol-4-yl |
| 112. | 1-ethylpyrazol-4-yl |
| 113. | 1-oxopyrazol-4-yl |
| 114. | 1-methylimidazol-4-yl |
| 115. | 1-methylimidazol-5-yl |
| 116. | 1,2-dimethylimidazol-4-yl |
| 117. | 1,2-dimethylimidazol-5-yl |
| 118. | 4-methylthiazol-2-yl |
| 119. | isothiazol-3-yl |
| 120. | isothiazol-4-yl |
| 121. | isothiazol-5-yl |
| 122. | 5-methylisothiazol-3-yl |
| 123. | 3-methylisothiazol-5-yl |
| 124. | 5-methylisothiazol-3-yl |
| 125. | 3-cyanoisothiazol-5-yl |
| 126. | 2-methyloxazol-5-yl |
| 127. | oxazol-5-yl |
| 128. | 1,2,3-thiadiazol-4-yl |
| 129. | 1,2,3-thiadiazol-5-yl |
| 130. | 4-methyl-1,2,3-thiadiazol-5-yl |
| 131. | 5-methyl-1,2,3-thiadiazol-4-yl |
| 132. | 4-cyano-1,2,3-thiadiazol-5-yl |
| 133. | 1,3,4-thiadiazol-2-yl |
| 134. | 1,3,4-oxadiazol-2-yl |
| 135. | 5-cyano-1,3,4-thiadiazol-2-yl |
| 136. | 5-cyano-1,3,4-oxadiazol-2-yl |
| 137. | 5-methyl-1,3,4-oxadiazol-2-yl |
| 138. | 5-methyl-1,3,4-thiadiazol-2-yl |
| 139. | 1,2,4-[4H]triazol-3-yl |
| 140. | 1,2,3,4-tetrazol-5-yl |
| 141. | 2-methyl-1,2,3,4-tetrazol-5-yl |
| 142. | 4-((trifluoromethyl)thio)-phenyl |
| 143. | 4-((trifluoromethyl)sulfonyl))-phenyl |

According to a particular embodiment, R² is phenyl or a monocyclic 6-membered heteroaryl, wherein phenyl and 6-membered heteroaryl are unsubstituted or carry 1, 2 or 3, in particular 1 or 2 radicals R^{2a} which are identical or different.

According to a particular embodiment, the present invention relates to the compounds of the formulae I, I.1, I.2 and I.3, to their salts, to their N-oxides and to the salts of the N-oxides, wherein R² is phenyl, which is unsubstituted or carries 1, 2 or 3, in particular 1 or 2 radicals R^{2a} which are identical or different. Amongst these compounds, those are preferred, wherein the carbon ring atom(s) which are located in the ortho position with respect to the point of attachment to the imidazo[1,2-a]pyridine core of (I) do not carry a radical R^{2a}. Preferably, one of the radicals R^{2a}, if present, is located in the para-position with respect to the point of attachment to the imidazo[1,2-a]pyridine core of (I).

According to another particular embodiment, the present invention relates to the compounds of the formulae I, I.1, I.2 and I.3, to their salts, to their N-oxides and to the salts of the N-oxides, wherein R² is a 6-membered heteroaryl, which is unsubstituted or carries 1, 2 or 3, in particular 1 or 2 radicals R^{2a} which are identical or different. Amongst these compounds, those are preferred, wherein the one or more hetero ring atom(s) are not located in the ortho position with respect to the point of attachment to the imidazo[1,2-a]pyridine core of (I). Amongst these compounds, those are preferred, wherein the carbon ring atom(s) which are located in the ortho position with respect to the point of attachment to the imidazo[1,2-a]pyridine core of (I) do not carry a radical R^{2a}. Preferably, one of the radicals R^{2a}, if present, is located in the para-position with respect to the point of attachment to the imidazo[1,2-a]pyridine core of (I).

According to a further particular embodiment, the present invention relates to the compounds of the formulae I, I.1, I.2 and I.3, to their salts, to their N-oxides and to the salts of the N-oxides, wherein R² is 5-membered C-bound heteroaryl, which is unsubstituted or carries 1, 2 or 3, in particular 1 or 2 radicals R^{2a} which are identical or different. Amongst these compounds, those are preferred, wherein the carbon ring atom(s) which are located in the ortho position with respect to the imidazo[1,2-a]pyridine moiety do not carry a radical R^{2a}.

In these embodiments, the radicals R^{2a}, if present, have the aforementioned meanings and are preferably selected from halogen, OH, SH, CN, NO₂, C₁-C₄ alkyl, especially methyl, ethyl or isopropyl, C₂-C₄ alkenyl, especially ethenyl, C₂-C₄ alkynyl, especially ethynyl, C₁-C₄-alkoxy, especially methoxy or ethoxy, C₁-C₄-alkylthio, especially methylthio or ethylthio, fluorinated C₁-C₂-alkyl, especially difluoromethyl or trifluoromethyl, SF₅, fluorinated C₁-C₂-alkoxy, especially difluoromethoxy or trifluoromethoxy, NH₂, hydroxy-C₁-C₄-alkyl, especially hydroxymethyl, 1-hydroxyethyl or 2-hydroxyethyl, C₁-C₄-alkoxy-C₁-C₄-alkyl, especially methoxymethyl, ethoxymethyl, 1-methoxyethyl or 2-methoxyethyl, C₁-C₄-alkyl-sulfonyl, especially methylsulfonyl or ethylsulfonyl, C(O)R⁴, especially acetyl or trifluoroacetyl, NR⁵R⁶, especially methylamino or dimethylamino, C(O)NR⁵R⁶, especially aminocarbonyl, and C(O)OR⁹, especially methoxycarbonyl, ethoxycarbonyl, difluoromethoxycarbonyl or trifluoromethoxycarbonyl,

or two radicals R^{2a}, which are bound to adjacent carbon atoms, together may also form a moiety O-Alk′-O, wherein Alk′ is selected from CH₂, CH₂CH₂, CHF and CF₂.

Especially, the radicals R^{2a}, if present, are selected from the group consisting of halogen, in particular fluorine or chlorine, CN (=cyano), NO₂ (nitro), OH, SH, C₁-C₄-alkyl, in particular methyl, ethyl or isopropyl, C₁-C₄-alkoxy, in particular methoxy or ethoxy, NH₂, NR⁴R⁵, in particular NH(CH₃), or N(CH₃)₂, fluorinated C₁-alkyl, in particular difluoromethyl or trifluoromethyl, SF₅, fluorinated C₁-alkoxy, in particular difluoromethoxy or trifluoromethoxy, C(O)NR⁵R⁶, especially aminocarbonyl, C₁-C₂-alkyl-sulfonyl, especially methylsulfonyl, C(O)R⁴, especially acetyl or trifluoroacetyl. Particularly preferably, R^{2a} is selected from the group consisting of hydrogen, halogen, in particular chlorine, CN, NO₂, methyl, methoxy, difluoromethyl, trifluoromethyl, aminocarbonyl, methylsulfonyl and trifluoroacetyl.

Amongst the compounds of the present invention, where R² in the formulae I, I.1, I.2 or I.3 is phenyl or 6-membered heteroaryl, which are unsubstituted or which carry 1, 2 or 3 radicals R^{2a} which are identical or different, preference is given to those compounds, wherein R² in the formulae I, I.1, I.2 or I.3 is a radical of the formula Ar3:

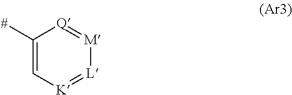

wherein # indicates the point of attachment to the imidazo[1,2-a]pyridine core of (I),
K′ is N or C—R²¹,
L′ is N or C—R²²,
M′ is N or C—R²³,
Q′ is N or C—R^{23a},
wherein R²¹, R²², R²³ and R^{23a}, independently of each other, are hydrogen or have one of the meanings given for R^{2a}.

Amongst the compounds of the present invention, where R² in the formulae I, I.1, I.2, I.3 or I.4 is phenyl or 6-membered heteroaryl, which are unsubstituted or which carry 1, 2 or 3 radicals R^{2a} which are identical or different, preference is given to those compounds, wherein R² in the formulae I, I.1, I.2, I.3 or I.4 is selected from radicals of the formulae Ar3.1, Ar3.2, Ar3.3, Ar3.4, Ar3.5, Ar3.6, Ar3.7, Ar3.8, Ar3.9, Ar3.10 and Ar3.11, with particular preference given to the formulae Ar3.1, Ar3.3 and Ar3.4:

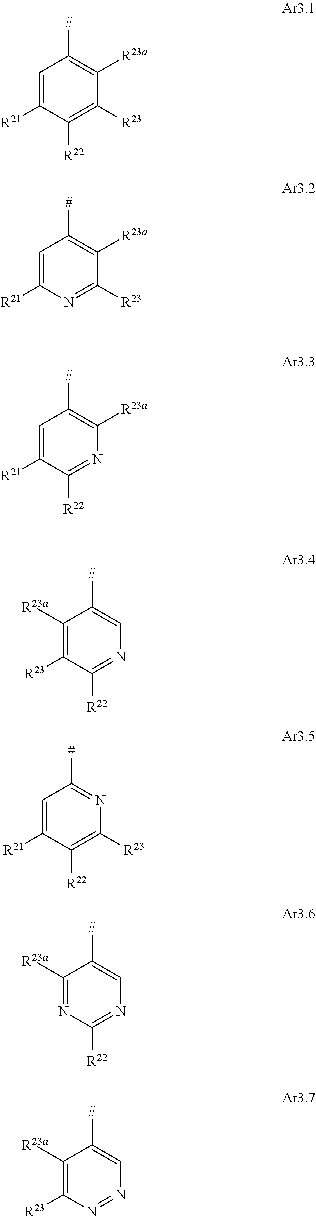

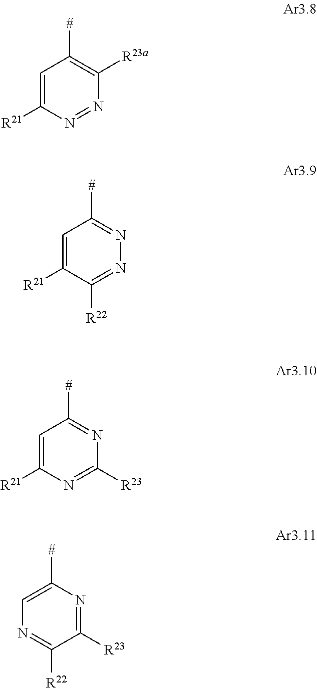

wherein # indicates the point of attachment to the imidazo[1,2-a]pyridine core of (I), and wherein R²¹, R²², R²³ and R^{23a} independently of each other, are hydrogen or have one of the meanings given for R^{2a}.

In the compounds of the formulae I, I.1, I.2 or I.3, where R² is a radical of the formulae Ar3.1, Ar3.2, Ar3.3, Ar3.4, Ar3.5, Ar3.6, Ar3.7, Ar3.8, Ar3.9, Ar3.10 or Ar3.11, preferred embodiments of the invention relate to compounds, wherein, the variables R²¹, R²², R²³ and R^{23a}, if present, individually or in particular in combination have the following meanings.

R²¹, R²³, are dependently of each other selected from hydrogen, halogen, especially fluorine or chlorine, OH, CN, NO₂, C₁-C₄ alkyl, especially methyl or ethyl, C₁-C₄-alkoxy, especially methoxy or ethoxy, C₁-C₄-alkylthio, especially methylthio or ethylthio, fluorinated C₁-C₂-alkyl, especially difluoromethyl or trifluoromethyl, fluorinated C₁-C₂-alkoxy, especially difluoromethoxy or trifluoromethoxy, hydroxy-C₁-C₄-alkyl, especially hydroxymethyl, 1-hydroxyethyl or 2-hydroxyethyl, and C₁-C₄-alkoxy-C₁-C₄-alkyl, especially methoxymethyl, ethoxymethyl, 1-methoxyethyl or 2-methoxyethyl.

R²², is selected from hydrogen, halogen, especially fluorine or chlorine, OH, CN, NO₂, C₁-C₄ alkyl, especially methyl, ethyl or isopropyl, C₁-C₄-alkoxy, especially methoxy or ethoxy, C₁-C₄-alkylthio, especially methylthio or ethylthio, fluorinated C₁-C₂-alkyl, especially difluoromethyl or trifluoromethyl, fluorinated C₁-C₂-alkoxy, especially difluoromethoxy or trifluoromethoxy, hydroxy-C₁-C₄-alkyl, especially hydroxymethyl, 1-hydroxyethyl or 2-hydroxyethyl, C₁-C₄-alkoxy-C₁-C₄-alkyl, especially methoxymethyl, ethoxymethyl, 1-methoxyethyl or 2-methoxyethyl, C₁-C₂-alkyl-sulfonyl, especially methylsulfonyl, C(O)NR⁷R⁸, especially aminocarbonyl, and C(O)R⁴, especially trifluoroacetyl.

R^{23a}, is selected from hydrogen, halogen, especially fluorine or chlorine, OH, CN, C₁-C₂ alkyl, especially methyl or ethyl, C₁-C₂-alkoxy, especially methoxy, fluorinated C₁-alkyl, especially difluoromethyl or trifluoromethyl, and fluorinated C₁-alkoxy, especially difluoromethoxy or trifluoromethoxy.

In the compounds of the formulae I, I.1, I.2 or I.3, where R² is a radical of the formulae Ar3.1, Ar3.2, Ar3.3, Ar3.4, Ar3.5, Ar3.6, Ar3.7, Ar3.8, Ar3.9, Ar3.10 or Ar3.11, more preferred embodiments of the invention relate to compounds, wherein, the variables R²¹, R²² and R²³, if present, individually or in particular in combination have the following meanings:

R²¹, R²³ are independently of each other selected from the group consisting of hydrogen, halogen, CN, NO₂, methyl, methoxy, difluoromethyl, trifluoromethyl, difluoromethoxy and trifluoromethoxy;

R²² is selected from the group consisting of hydrogen, halogen, OH, CN, NO₂, methyl, ethyl, isopropyl, methoxy, ethoxy, difluoromethyl, trifluoromethyl, difluoromethoxy trifluoromethoxy, methylsulfonyl, aminocarbonyl and trifluoroacetyl; and

R^{23a} is selected from the group consisting of hydrogen, halogen, CN, methyl, methoxy, difluoromethyl and trifluoromethyl.

In the compounds of the formulae I, I.1, I.2 or I.3, where R² is a radical of the formulae Ar3.1, Ar3.3, Ar3.4, Ar3.5, Ar3.6, Ar3.9 or Ar3.11, particular embodiments of the invention relate to compounds, wherein, the variables R²¹, R²² and R²³, if present, individually or in particular in combination have the following meanings:

R²¹, R²³, are independently of each other hydrogen, halogen, NO₂, CN, C₁-C₂-alkyl, especially methyl or ethyl, C₁-C₂-alkoxy, especially methoxy or ethoxy, fluorinated C₁-alkyl, especially difluoromethyl or trifluoromethyl and fluorinated C₁-alkoxy, especially difluoromethoxy or trifluoromethoxy.

R²², is selected from hydrogen, halogen, especially fluorine or chlorine, OH, CN, NO₂, C₁-C₄ alkyl, especially methyl, ethyl or isopropyl, C₁-C₂-alkoxy, especially methoxy or ethoxy, fluorinated C₁-C₂-alkyl, especially difluoromethyl or trifluoromethyl, fluorinated C₁-C₂-alkoxy, especially difluoromethoxy or trifluoromethoxy, hydroxy-C₁-C₂-alkyl, especially hydroxymethyl, 1-hydroxyethyl or 2-hydroxyethyl, C₁-C₂-alkoxy-C₁-C₂-alkyl, especially methoxymethyl, ethoxymethyl, 1-methoxyethyl or 2-methoxyethyl, C₁-C₂-alkyl-sulfonyl, especially methylsulfonyl, C(O)NR⁷R⁸, especially aminocarbonyl, and C(O)OR⁹, especially trifluoroacetyl.

R^{23a}, is selected from hydrogen, halogen, CN, C₁-C₂ alkyl, especially methyl or ethyl, C₁-C₂-alkoxy, especially methoxy or ethoxy, fluorinated C₁-alkyl, especially difluoromethyl or trifluoromethyl, and fluorinated C₁-alkoxy, especially difluoromethoxy or trifluoromethoxy.

In the compounds of the formulae I, I.1, I.2 or I.3, where R² is a radical of the formulae Ar3.1, Ar3.3, Ar3.4, Ar3.5, Ar3.6, Ar3.9 or Ar3.11, special embodiments of the invention relate to compounds, wherein, the variables R²¹, R²², R²³ and R^{23a}, if present, individually or in particular in combination have the following meanings:

R²¹, R²³ are independently of each other selected from the group consisting of hydrogen, halogen, CN, NO₂, methyl, methoxy, difluoromethyl and trifluoromethyl, and in particular selected from hydrogen, NO₂, CN and methyl.

R²² is selected from the group consisting of hydrogen, halogen, CN, NO₂, methyl, ethyl, isopropyl, methoxy, ethoxy, difluoromethyl, trifluoromethyl, difluoromethoxy trifluoromethoxy, methylsulfonyl, aminocarbonyl and trifluoroacetyl, and in particular selected from hydrogen, methoxy, chloride, NO₂, CN, methyl, methylsulfonyl, trifluoromethyl, trifluoroacetyl and aminocarbonyl.

R^{23a} is selected from the group consisting of hydrogen, chlorine, CN, methyl and methoxy, and in particular is hydrogen.

Amongst the compounds of the present invention, where R² in the formulae I, I.1, I.2 or I.3 is C-bound 5-membered heteroaryl, which is unsubstituted or carries 1, 2 or 3 radicals R^{2a} which are identical or different, preference is given to those compounds, wherein R¹ in the formulae I, I.1, I.2 or I.3 is a radical of the formulae Ar4 or Ar5:

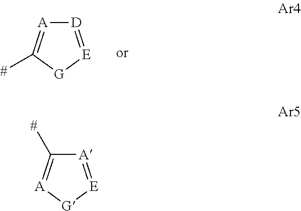

wherein # indicates the point of attachment of R² to the imidazo[1,2-a]pyridine core of (I),
A is N or C—R²⁴,
A′ is N or C—R²⁵,
D is N or C—R²⁶,
E is N or C—R²⁷,
G is O, S or N—R²⁸,
G′ is O, S or N—R²⁹,

wherein R²⁴, R²⁵, R²⁶ and R²⁷, independently of each other, are hydrogen or have one of the meanings given for R^{2a}, and wherein R²⁸ and R²⁹ are selected from the group consisting of hydrogen, CN, C₁-C₁₀-alkyl, in particular C₁-C₄-alkyl, especially methyl, ethyl or isopropyl, C₂-C₁₀-alkenyl, in particular C₂-C₄-alkenyl, especially ethenyl or 3-propenyl, C₂-C₁₀-alkynyl, in particular C₂-C₄-alkynyl, especially ethynyl or 3-propynyl, C₁-C₆-alkoxy, in particular C₁-C₄-alkoxy, especially methoxy or ethoxy, fluorinated C₁-C₂-alkyl, in particular fluorinated C₁-alkyl, especially difluoromethyl or trifluoromethyl, fluorinated C₁-C₂-alkoxy, in particular fluorinated C₁-alkoxy, especially difluoromethoxy or trifluoromethoxy, C(O)R⁴, in particular acetyl or propionyl, NR⁵R⁶, especially methylamino or dimethylamino, and C(O)OR⁹, especially methoxycarbonyl or ethoxycarbonyl.

Amongst the compounds of the present invention, where R² in the formulae I, I.1, I.2 or I.3 is a C-bound 5-membered heteroaryl, which is unsubstituted or carries 1, 2 or 3 radicals R^{2a} which are identical or different, preference is given to those compounds, wherein R² in the formulae I, I.1, I.2, I.3 or I.4 is selected from radicals of the formulae Ar4.1, Ar4.2, Ar4.3, Ar4.4, Ar4.5, Ar4.6, Ar4.7, Ar4.8, Ar4.9, Ar4.10, Ar4.11, Ar4.12, Ar4.13, Ar4.14, Ar4.15, Ar5.1, Ar5.2, Ar5.3, Ar5.4, Ar5.5, Ar5.6, Ar5.7, Ar5.8, Ar5.9, Ar5.10, Ar5.11 and Ar5.12, with particular preference given to the formulae Ar4.1, Ar4.2, Ar4.3, Ar5.1, Ar5.2 and Ar5.3:

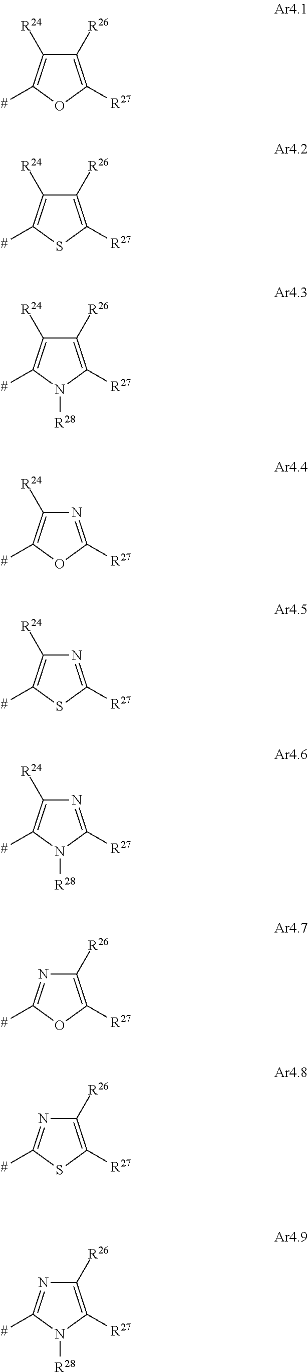

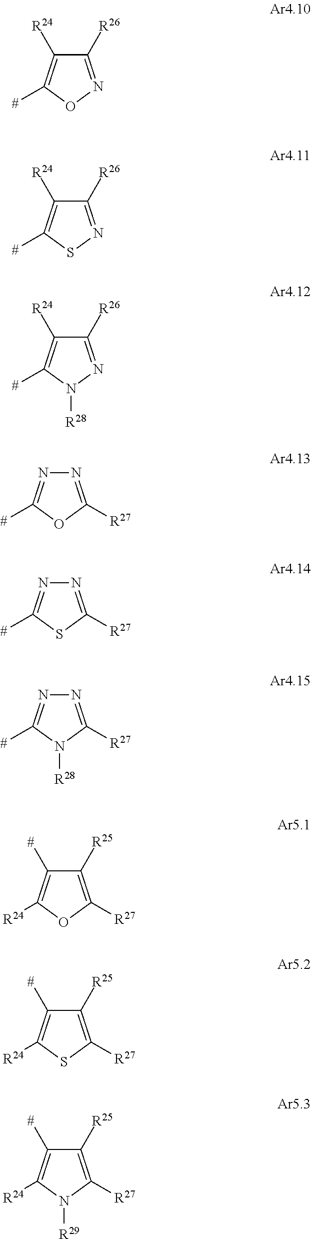

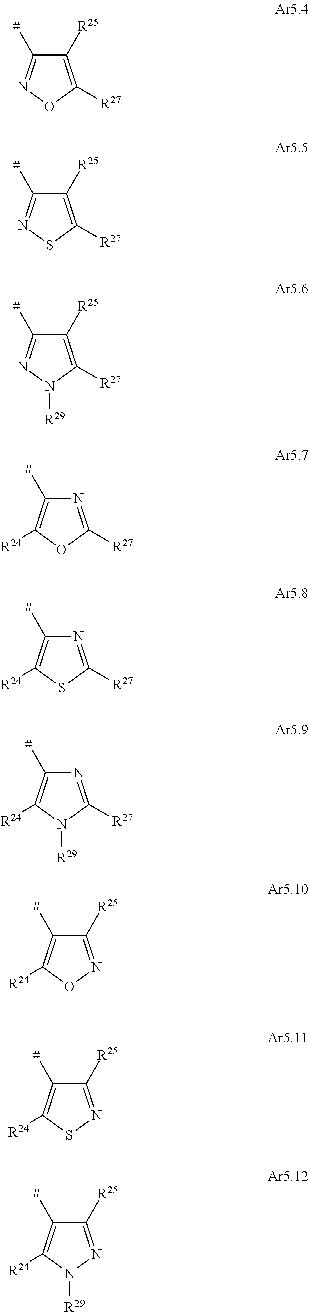

wherein # indicates the point of attachment of R² to the imidazo[1,2-a]pyridine core of (I), and wherein R²⁴, R²⁵, R²⁶, R²⁷, R²⁸ and R²⁹ have the meanings given above.

In the compounds of the formulae I, I.1, I.2 or I.3, where R² is selected from radicals of the formulae Ar4.1, Ar4.2, Ar4.3, Ar4.4, Ar4.5, Ar4.6, Ar4.7, Ar4.8, Ar4.9, Ar4.10, Ar4.11, Ar4.12, Ar4.13, Ar4.14, Ar4.15, Ar5.1, Ar5.2, Ar5.3, Ar5.4, Ar5.5, Ar5.6, Ar5.7, Ar5.8, Ar5.9, Ar5.10, Ar5.11 and Ar5.12, particular embodiments of the invention relate to compounds, wherein, the variables R²⁴, R²⁵, R²⁶, R²⁷, R²⁸ and R²⁹, if present, individually or in particular in combination have the following meanings:

R²⁴, R²⁵, are independently of each other selected from the group consisting of hydrogen and methyl, and more preferably are hydrogen;

R²⁶, R²⁷, are independently of each other selected from the group consisting of hydrogen, halogen, in particular fluorine or chlorine, CN, NO₂, C₁-C₄-alkyl, in particular methyl, ethyl or isopropyl, C₁-C₂-alkoxy, in particular methoxy or ethoxy, NH₂, NR⁵R⁶, in particular NH(CH₃), or N(CH₃)₂, fluorinated C₁-alkyl, in particular difluoromethyl or trifluoromethyl, fluorinated C₁-alkoxy, in particular difluoromethoxy or trifluoromethoxy.

R²⁸, R²⁹, are independently of each other selected from the group consisting of hydrogen, C₁-C₄ alkyl, especially methyl, ethyl or isopropyl, C₂-C₄ alkenyl, especially ethenyl, C₁-C₂-alkoxy, especially methoxy or ethoxy, fluorinated C₁-alkyl, especially difluoromethyl or trifluoromethyl, and fluorinated C₁-alkoxy, especially difluoromethoxy or trifluoromethoxy.

In the compounds of the formulae I, I.1, I.2 or I.3, where R² is selected from radicals of the formulae Ar4.1, Ar4.2, Ar4.3, Ar4.4, Ar4.5, Ar4.6, Ar4.7, Ar4.8, Ar4.9, Ar4.10, Ar4.11, Ar4.12, Ar4.13, Ar4.14, Ar4.15, Ar5.1, Ar5.2, Ar5.3, Ar5.4, Ar5.5, Ar5.6, Ar5.7, Ar5.8, Ar5.9, Ar5.10, Ar5.11 and Ar5.12, particular embodiments of the invention relate to compounds, wherein, the variables R²⁴, R²⁵, R²⁶, R²⁷, R²⁸ and R²⁹, if present, individually or in particular in combination have the following meanings:

R²⁴, R²⁵ are both hydrogen.

R²⁶, R²⁷ are independently of each other selected from the group consisting of hydrogen, halogen, in particular fluorine or chlorine, OH, CN, methyl, methoxy, difluoromethyl, trifluoromethyl, difluoromethoxy and trifluoromethoxy; and especially selected from hydrogen, CN and methyl.

R²⁸, R²⁹ are independently of each other selected from the group consisting of hydrogen and C₁-C₄ alkyl; and especially selected from hydrogen and methyl.

Examples of radicals R² are given in the following tables B, which are particular embodiments according to the present invention.

TABLE B
Meanings of R²

| | R² |
|---|---|
| 1. | phenyl |
| 2. | 3-cyanophenyl |
| 3. | 4-cyanophenyl |
| 4. | 4-methoxyphenyl |
| 5. | 3-methoxyphenyl |
| 6. | 4-chlorphenyl |
| 7. | 3-chlorphenyl |
| 8. | 3,4-dicyanophenyl |
| 9. | 3-methoxy-4-cyanophenyl |
| 10. | 4-methoxy-3-cyanophenyl |
| 11. | 3-chloro-4-cyanophenyl |
| 12. | 4-chloro-3-cyanophenyl |
| 13. | 3,4-dimethoxyphenyl |
| 14. | 3-chloro-4-methoxyphenyl |
| 15. | 4-chloro-3-methoxyphenyl |
| 16. | 3,4-dichlorophenyl |
| 17. | 4-methylphenyl |
| 18. | 3-methylphenyl |
| 19. | 3,4-dimethylphenyl |
| 20. | 3-methyl-4-cyanophenyl |
| 21. | 4-methyl-3-cyanophenyl |
| 22. | 3-methyl-4-chlorophenyl |
| 23. | 4-methyl-3-chlorophenyl |
| 24. | 3-methyl-4-methoxyphenyl |
| 25. | 4-methyl-3-methoxyphenyl |
| 26. | 4-trichloroacetylphenyl |
| 27. | 3-trichloroacetylphenyl |
| 28. | 4-aminocarbonylphenyl |
| 29. | 3-aminocarbonylphenyl |
| 30. | 4-methylsulfonylphenyl |
| 31. | 3-methylsulfonylphenyl |
| 32. | 4-nitrophenyl |
| 33. | 3-nitrophenyl |
| 34. | 4-trifluoromethylphenyl |
| 35. | 3-trifluoromethylphenyl |
| 36. | pyridine-2-yl |
| 37. | pyridine-3-yl |
| 38. | pyridine-4-yl |
| 39. | 6-cyanopyridine-3-yl |
| 40. | 6-methoxypyridine-3-yl |
| 41. | 6-chloropyridine-3-yl |
| 42. | 6-methlypyridine-3-yl |
| 43. | 5-cyanopyridine-2-yl |
| 44. | 5-methoxypyridine-2-yl |
| 45. | 5-chloropyridine-2-yl |
| 46. | 5-methlypyridine-2-yl |
| 47. | pyrimidine-2-yl |
| 48. | pyrimidine-4-yl |
| 49. | pyrimidine-5-yl |
| 50. | 5-cyanopyrimidine-2-yl |
| 51. | 2-cyanopyrimidine-5-yl |
| 52. | 5-methoxypyrimidine-2-yl |
| 53. | 2-methoxypyrimidine-5-yl |
| 54. | 5-chloropyrimidine-2-yl |
| 55. | 2-chloropyrimidine-5-yl |
| 56. | 5-methylpyrimidine-2-yl |
| 57. | 2-methylpyrimidine-5-yl |
| 58. | pyrazin-2-yl |
| 59. | 5-cyano-pyrazin-2-yl |
| 60. | 5-methoxy-pyrazin-2-yl |
| 61. | 5-chloro-pyrazin-2-yl |
| 62. | pyridazin-3-yl |
| 63. | pyridazin-4-yl |
| 64. | 6-cyanopyridazin-3-yl |
| 65. | 6-methoxypyridazin-3-yl |
| 66. | 6-chloropyridazin-3-yl |
| 67. | furan-2-yl |
| 68. | furan-3-yl |
| 69. | 5-cyanofuran-2-yl |
| 70. | 4-cyanofuran-2-yl |
| 71. | 5-cyanofuran-3-yl |
| 72. | 2-thienyl |
| 73. | 3-thienyl |
| 74. | 5-cyano-2-thienyl |
| 75. | 4-cyano-2-thienyl |
| 76. | 5-cyano-3-thienyl |
| 77. | pyrrol-2-yl |
| 78. | pyrrol-3-yl |
| 79. | 5-cyanopyrrol-2-yl |
| 80. | 4-cyanopyrrol-2-yl |
| 81. | 5-cyanopyrrol-3-yl |
| 82. | 5-methylpyrrol-2-yl |
| 83. | 4-methylpyrrol-2-yl |
| 84. | 5-methylpyrrol-3-yl |
| 85. | 1,5-dimethylpyrrol-2-yl |
| 86. | 1,4-dimethylpyrrol-2-yl |
| 87. | 1,5-dimethylpyrrol-3-yl |
| 88. | 1-methyl-5-cyanopyrrol-2-yl |
| 89. | 1-methyl-4-cyanopyrrol-2-yl |
| 90. | 1-methyl-5-cyanopyrrol-3-yl |
| 91. | oxazol-4-yl |
| 92. | oxazol-5-yl |
| 93. | 2-cyanooxazol-4-yl |
| 94. | 2-cyanooxazol-5-yl |
| 95. | 3-methyl-2-cyanooxazol-4-yl |
| 96. | 3-methyl-2-cyanooxazol-5-yl |
| 97. | imidazol-4-yl |
| 98. | imidazol-5-yl |
| 99. | 2-cyanoimidazol-4-yl |
| 100. | 2-cyanoimidazol-5-yl |
| 101. | 2-cyano-1-methylimidazol-4-yl |
| 102. | 2-cyano-1-methylimidazol-5-yl |
| 103. | thiazol-4-yl |
| 104. | thiazol-5-yl |
| 105. | 2-cyanothiazol-4-yl |
| 106. | 2-cyanothiazol-5-yl |
| 107. | 3-methyl-2-cyanothiazol-4-yl |
| 108. | 3-methyl-2-cyanothiazol-5-yl |
| 109. | 4-cyanooxazol-2-yl |
| 110. | 5-cyanooxazol-2-yl |
| 111. | 4-cyanoimidazol-2-yl |
| 112. | 5-cyanoimidazol-2-yl |
| 113. | 4-cyano-1-methylimidazol-2-yl |
| 114. | 5-cyano-1-methylimidazol-2-yl |
| 115. | 5-cyanothiazol-2-yl |
| 116. | 4-cyanothiazol-2-yl |
| 117. | 3-cyanoisoxazol-5-yl |
| 118. | 5-cyanoisoxazol-3-yl |
| 119. | 3-cyanoisothiazol-5-yl |
| 120. | 5-cyanoisothiazol-3-yl |
| 121. | 3-cyanopyrazol-5-yl |
| 122. | 5-cyanopyrazol-3-yl |
| 123. | 3-cyano-1-methylpyrazol-5-yl |
| 124. | 5-cyano-1-methylpyrazol-3-yl |
| 125. | 5-cyano-1,3,4-oxadiazol-2-yl |
| 126. | 5-cyano-1,3,4-thiadiazol-2-yl |
| 127. | 5-cyano-1,2,4-triazol-3-yl |
| 128. | 5-cyano-4-methyl-1,2,4-triazol-3-yl |

According to a particular embodiment, R³ is selected from the group consisting of hydrogen, C₁-C₄-alkyl, especially methyl, ethyl or n-propyl, C₁-C₂-alkoxy-C₁-C₂-alkyl, especially methoxymethyl, ethoxymethyl, 1-methoxyethyl or 2-methoxyethyl, fluorinated C₁-alkyl, especially difluoromethyl or trifluoromethyl, and C(O)R⁴, especially acetyl, trifluoroacetyl or trifluoroacetyl.

According to a particular preferred embodiment, R³ is selected from the group consisting of hydrogen, C₁-C₄-alkyl, difluoromethyl, trifluoromethyl, acetyl, difluoroacetyl and trifluoroacetyl, and especially selected from the group consisting of hydrogen, methyl, ethyl, n-propyl, trifluoromethyl, acetyl or trifluoroactetyl.

A particular embodiment of the invention relates to compounds of the formulae I, I.1, I.2 and I.3, to their salts, to their N-oxides and to the salts of the N-oxides, wherein R¹ is a radical of the formula Ar1 and wherein R² is a radical of the formula Ar3, in particular a radical of the formulae Ar3.1, Ar3.2, Ar3.3, Ar3.4, Ar3.5, Ar3.6, Ar3.7, Ar3.8, Ar3.9, Ar3.10 or Ar3.11, especially a radical of the formulae Ar3.1, Ar3.3 or Ar3.4.

Another particular embodiment of the invention relates to compounds of the formulae I, I.1, I.2 and I.3, to their salts, to their N-oxides and to the salts of the N-oxides, wherein R¹ is a radical of the formula Ar1 and wherein R² is a radical of the formulae Ar4 or Ar5, in particular a radical of the formulae Ar4.1, Ar4.2, Ar4.3, Ar4.4, Ar4.5, Ar4.6, Ar4.7, Ar4.8, Ar4.9, Ar4.10, Ar4.11, Ar4.12, Ar4.13, Ar4.14, Ar4.15, Ar5.1, Ar5.2, Ar5.3, Ar5.4, Ar5.5, Ar5.6, Ar5.7, Ar5.8, Ar5.9, Ar5.10, Ar5.11 or Ar5.12, especially a radical of the formulae Ar4.1, Ar4.2, Ar4.3, Ar5.1, Ar5.2 or Ar5.3.

A further particular embodiment of the invention relates to compounds of the formulae I, I.1, I.2 and I.3, to their salts, to their N-oxides and to the salts of the N-oxides, wherein R¹ is a radical of the formulae Ar2 or Ar2′ and wherein R² is a radical of the formula Ar3, in particular a radical of the formulae Ar3.1, Ar3.2, Ar3.3, Ar3.4, Ar3.5, Ar3.6, Ar3.7, Ar3.8, Ar3.9, Ar3.10 or Ar3.11, especially a radical of the formulae Ar3.1, Ar3.3 or Ar3.4.

A further particular embodiment of the invention relates to compounds of the formulae I, I.1, I.2 and I.3, to their salts, to their N-oxides and to the salts of the N-oxides, wherein R¹ is a radical of the formulae Ar2 or Ar2′ and wherein R² is a radical of the formulae Ar4 or Ar5, in particular a radical of the formulae Ar4.1, Ar4.2, Ar4.3, Ar4.4, Ar4.5, Ar4.6, Ar4.7, Ar4.8, Ar4.9, Ar4.10, Ar4.11, Ar4.12, Ar4.13, Ar4.14, Ar4.15, Ar5.1, Ar5.2, Ar5.3, Ar5.4, Ar5.5, Ar5.6, Ar5.7, Ar5.8, Ar5.9, Ar5.10, Ar5.11 or Ar5.12, especially a radical of the formulae Ar4.1, Ar4.2, Ar4.3, Ar5.1, Ar5.2 or Ar5.3.

A further particular embodiment of the invention relates to compounds of the formulae I, I.1, I.2 and I.3, to their salts, to their N-oxides and to the salts of the N-oxides, wherein R¹ is a radical of the formula Ar1.1 to Ar1.7, in particular a radical Ar1.1, and wherein R² is a radical of the formula Ar3, in particular a radical of the formulae Ar3.1, Ar3.2, Ar3.3, Ar3.4, Ar3.5, Ar3.6, Ar3.7, Ar3.8, Ar3.9, Ar3.10 or Ar3.11, especially a radical of the formulae Ar3.1, Ar3.3 or Ar3.4.

A further particular embodiment of the invention relates to compounds of the formulae I, I.1, I.2 and I.3, to their salts, to their N-oxides and to the salts of the N-oxides, wherein R¹ is a radical of the formula Ar1.1 to Ar1.7, in particular a radical Ar1.1, and wherein R² is a radical of the formulae Ar4 or Ar5, in particular a radical of the formulae Ar4.1, Ar4.2, Ar4.3, Ar4.4, Ar4.5, Ar4.6, Ar4.7, Ar4.8, Ar4.9, Ar4.10, Ar4.11, Ar4.12, Ar4.13, Ar4.14, Ar4.15, Ar5.1, Ar5.2, Ar5.3, Ar5.4, Ar5.5, Ar5.6, Ar5.7, Ar5.8, Ar5.9, Ar5.10, Ar5.11 or Ar5.12, especially a radical of the formulae Ar4.1, Ar4.2, Ar4.3, Ar5.1, Ar5.2 or Ar5.3.

A further particular embodiment of the invention relates to compounds of the formulae I, I.1, I.2 and I.3, to their salts, to their N-oxides and to the salts of the N-oxides, wherein R¹ is selected from radicals of the formulae Ar2.1, Ar2.2, Ar2.3, Ar2.4, Ar2.5, Ar2.6, Ar2.7, Ar2.8, Ar2.9, Ar2.10, Ar2.11, Ar2.12, Ar2.13, Ar2.14, Ar2.15, Ar2.16, Ar2.17, Ar2.18, Ar2.19, Ar2.20, Ar2.21, Ar2.22, Ar2′.1, Ar2′.2, Ar2′.3, Ar2′.4, Ar2′.5, Ar2′.6, Ar2′.7, Ar2′.8, Ar2′.9, Ar2′.10, Ar2′.11, Ar2′.12, Ar2′.13, Ar2′.14 and Ar2′.15, in particular from radicals of the formulae Ar2.1, Ar2.2, Ar2.3, Ar2.4, Ar2′.2 and Ar2′.6, and wherein R² is a radical of the formula Ar3, in particular a radical of the formulae Ar3.1, Ar3.2, Ar3.3, Ar3.4, Ar3.5, Ar3.6, Ar3.7, Ar3.8, Ar3.9, Ar3.10 or Ar3.11, especially a radical of the formulae Ar3.1, Ar3.3 or Ar3.4.

A further particular embodiment of the invention relates to compounds of the formulae I, I.1, I.2 and I.3, to their salts, to their N-oxides and to the salts of the N-oxides, wherein R¹ is selected from radicals of the formulae Ar2.1, Ar2.2, Ar2.3, Ar2.4, Ar2.5, Ar2.6, Ar2.7, Ar2.8, Ar2.9, Ar2.10, Ar2.11, Ar2.12, Ar2.13, Ar2.14, Ar2.15, Ar2.16, Ar2.17, Ar2.18, Ar2.19, Ar2.20, Ar2.21, Ar2.22, Ar2′.1, Ar2′.2, Ar2′.3, Ar2′.4, Ar2′.5, Ar2′.6, Ar2′.7, Ar2′.8, Ar2′.9, Ar2′.10, Ar2′.11, Ar2′.12, Ar2′.13, Ar2′.14 and Ar2′.15, in particular from radicals of the formulae Ar2.1, Ar2.2, Ar2.3, Ar2.4, Ar2′.2 and Ar2′.6, and wherein R² is a radical of the formulae Ar4 or Ar5, in particular a radical of the formulae Ar4.1, Ar4.2, Ar4.3, Ar4.4, Ar4.5, Ar4.6, Ar4.7, Ar4.8, Ar4.9, Ar4.10, Ar4.11, Ar4.12, Ar4.13, Ar4.14, Ar4.15, Ar5.1, Ar5.2, Ar5.3, Ar5.4, Ar5.5, Ar5.6, Ar5.7, Ar5.8, Ar5.9, Ar5.10, Ar5.11 or Ar5.12, especially a radical of the formulae Ar4.1, Ar4.2, Ar4.3, Ar5.1, Ar5.2 or Ar5.3.

A further particular embodiment of the invention relates to compounds of the formulae I, I.1, I.2 and I.3, to their salts, to their N-oxides and to the salts of the N-oxides, wherein R¹ is a radical of the formulae Ar1.1, and wherein R² is a radical of the formulae Ar3.1, Ar3.2, Ar3.3, Ar3.4, Ar3.5, Ar3.6, Ar3.7, Ar3.8, Ar3.9, Ar3.10, Ar3.11 or Ar3.12, especially a radical of the formulae Ar3.1, Ar3.3 or Ar3.4.

A further particular embodiment of the invention relates to compounds of the formulae I, I.1, I.2 and I.3, to their salts, to their N-oxides and to the salts of the N-oxides, wherein R¹ is a radical of the formulae Ar1.1, and wherein R² is a radical of the formulae Ar4.1, Ar4.2, Ar4.3, Ar5.1, Ar5.2 or Ar5.3.

A further particular embodiment of the invention relates to compounds of the formulae I, I.1, I.2 and I.3, to their salts, to their N-oxides and to the salts of the N-oxides, wherein R¹ is selected from radicals of the formulae Ar2.1, Ar2.2, Ar2.3, Ar2.4, Ar2′.2 and Ar2′.6, and wherein R² is a radical of the formula Ar3, in particular a radical of the formulae Ar3.1, Ar3.3 or Ar3.4.

A further particular embodiment of the invention relates to compounds of the formulae I, I.1, I.2 and I.3, to their salts, to their N-oxides and to the salts of the N-oxides, wherein R¹ is selected from radicals of the formulae Ar2.1, Ar2.2, Ar2.3, Ar2.4, Ar2′.2 and Ar2′.6, and wherein R² is a radical of the formulae Ar4.1, Ar4.2, Ar4.3, Ar5.1, Ar5.2 or Ar5.3.

With regard to the radical C(O)R⁴ the following meanings are particular embodiments of R⁴: methyl, ethyl, n-propyl, isopropyl, difluoromethyl and trifluoromethyl. In particular the radical C(O)R⁴ is selected from acetyl or trifluoroacetyl.

With regard to the radical NR⁵R⁶ the following meanings are particular embodiments of R⁵: hydrogen or C₁-C₄-alkyl, in particular hydrogen, methyl, ethyl, n-propyl, isopropyl or n-butyl. The following meanings are particular embodiments of R⁶: C₁-C₄-alkyl, in particular methyl, ethyl, n-propyl, isopropyl or n-butyl. In other embodiments, R⁵, R⁶ together with the nitrogen atom, to which they are bound, form an N-bound, 5- or 6-membered saturated nitrogen heterocycle, such as pyrrolidin-1-yl, morpholin-4-yl, thiomorpholin-4-yl, piperazin-1-yl or 4-methylpiperazin-1-yl. In particular the radical NR⁴R⁵ is selected from methylamino, ethylamino, n-propylamino, isopropylamino, dimethylamino, diethylamino, N-methyl-N-ethylamino, N-methyl-N-isopropylamino, N-methyl-N-propylamino, pyrrolidin-1-yl, morpholin-4-yl, thiomorpholin-4-yl, piperazin-1-yl and 4-methylpiperazin-1-yl.

With regard to the radical C(O)NR⁷R⁸ the following meanings are particular embodiments of R⁷: hydrogen or C₁-C₄-alkyl, in particular hydrogen, methyl, ethyl, n-propyl, isopropyl or n-butyl. The following meanings are particular embodiments of R⁸: hydrogen or C₁-C₄-alkyl, in particular hydrogen, methyl, ethyl, n-propyl, isopropyl or n-butyl.

With regard to the radical C(O)OR⁹ the following meanings are particular embodiments of R⁹: C₁-C₄-alkyl, in particular methyl, ethyl, n-propyl, isopropyl, n-butyl, isobutyl or tert.-butyl. In particular the radical C(O)OR⁹ is selected from methoxycarbonyl, ethoxycarbonyl, n-propoxycarbonyl, n-butoxycarbonyl or tert.-butoxycarbonyl.

Examples of suitable compounds according to the present invention are the compounds of the formula (I) as given in the following tables 1 to 128, 129 to 256, 257 to 384, 385 to 512, 641 to 768, 769 to 896 and 897 to 1024, their pharmaceutically acceptable salts, their N-oxides and the pharmaceutically acceptable salts of said N-oxides.
Table 1 Compounds of the formula (I), wherein X is CH₂, R³ is hydrogen and R² is phenyl and wherein R¹ has one of the meanings given in rows 1 to 141 of table A (Compounds I-1 to I-143);
Table 2 Compounds of the formula (I), wherein X is CH₂, R³ is hydrogen and R² is 3-cyanophenyl and wherein R¹ has one of the meanings given in rows 1 to 141 of table A (Compounds I-144 to I-286)
Table 3 Compounds of the formula (I), wherein X is CH₂, R³ is hydrogen and R² is 4-cyanophenyl and wherein R¹ has one of the meanings given in rows 1 to 141 of table A (Compounds I-287 to I-429);
Table 4 Compounds of the formula (I), wherein X is CH₂, R³ is hydrogen and R² is 4-methoxyphenyl and wherein R¹ has one of the meanings given in rows 1 to 141 of table A (Compounds I-430 to I-572);
Table 5 Compounds of the formula (I), wherein X is CH₂, R³ is hydrogen and R² is 3-methoxyphenyl and wherein R¹ has one of the meanings given in rows 1 to 141 of table A (Compounds I-573 to I-715);
Table 6 Compounds of the formula (I), wherein X is CH₂, R³ is hydrogen and R² is 4-chlorphenyl and wherein R¹ has one of the meanings given in rows 1 to 141 of table A (Compounds I-716 to I-858);
Table 7 Compounds of the formula (I), wherein X is CH₂, R³ is hydrogen and R² is 3-chlorphenyl, and wherein R¹ has one of the meanings given in rows 1 to 141 of table A (Compounds I-859 to I-1001);
Table 8 Compounds of the formula (I), wherein X is CH₂, R³ is hydrogen and R² is 3,4-dicyanophenyl and wherein R¹ has one of the meanings given in rows 1 to 141 of table A (Compounds I-1002 to I-1144);
Table 9 Compounds of the formula (I), wherein X is CH₂, R³ is hydrogen and R² is 3-methoxy-4-cyanophenyl and wherein R¹ has one of the meanings given in rows 1 to 141 of table A (Compounds I-1145 to I-1287);
Table 10 Compounds of the formula (I), wherein X is CH₂, R³ is hydrogen and R² is 4-methoxy-3-cyanophenyl and wherein R¹ has one of the meanings given in rows 1 to 141 of table A (Compounds I-1288 to I-1430);
Table 11 Compounds of the formula (I), wherein X is CH₂, R³ is hydrogen and R² is 3-chloro-4-cyanophenyl and wherein R¹ has one of the meanings given in rows 1 to 141 of table A (Compounds I-1431 to I-1573);
Table 12 Compounds of the formula (I), wherein X is CH₂, R³ is hydrogen and R² is 4-chloro-3-cyanophenyl and wherein R¹ has one of the meanings given in rows 1 to 141 of table A (Compounds I-1574 to I-1716);
Table 13 Compounds of the formula (I), wherein X is CH₂, R³ is hydrogen and R² is 3,4-dimethoxyphenyl and wherein R¹ has one of the meanings given in rows 1 to 141 of table A (Compounds I-1717 to I-1859);
Table 14 Compounds of the formula (I), wherein X is CH₂, R³ is hydrogen and R² is 3-chloro-4-methoxyphenyl and wherein R¹ has one of the meanings given in rows 1 to 141 of table A (Compounds I-1860 to I-2002);
Table 15 Compounds of the formula (I), wherein X is CH₂, R³ is hydrogen and R² is 4-chloro-3-methoxyphenyl and wherein R¹ has one of the meanings given in rows 1 to 141 of table A (Compounds I-2003 to I-2145);
Table 16 Compounds of the formula (I), wherein X is CH₂, R³ is hydrogen and R² is 3,4-dichlorophenyl and wherein R¹ has one of the meanings given in rows 1 to 141 of table A (Compounds I-2146 to I-2288);
Table 17 Compounds of the formula (I), wherein X is CH₂, R³ is hydrogen and R² is 4-methylphenyl and wherein R¹ has one of the meanings given in rows 1 to 141 of table A (Compounds I-2289 to I-2431);
Table 18 Compounds of the formula (I), wherein X is CH₂, R³ is hydrogen and R² is 3-methylphenyl, and wherein R¹ has one of the meanings given in rows 1 to 141 of table A (Compounds I-2432 to I-2574);
Table 19 Compounds of the formula (I), wherein X is CH₂, R³ is hydrogen and R² is 3,4-dimethylphenyl and wherein R¹ has one of the meanings given in rows 1 to 141 of table A (Compounds I-2575 to I-2717);
Table 20 Compounds of the formula (I), wherein X is CH₂, R³ is hydrogen and R² is 3-methyl-4-cyanophenyl and wherein R¹ has one of the meanings given in rows 1 to 141 of table A (Compounds I-2718 to I-2860);
Table 21 Compounds of the formula (I), wherein X is CH₂, R³ is hydrogen and R² is 4-methyl-3-cyanophenyl, and wherein R¹ has one of the meanings given in rows 1 to 141 of table A (Compounds I-2861 to I-3003);
Table 22 Compounds of the formula (I), wherein X is CH₂, R³ is hydrogen and R² is 3-methyl-4-chlorophenyl and wherein R¹ has one of the meanings given in rows 1 to 141 of table A (Compounds I-3004 to I-3146);
Table 23 Compounds of the formula (I), wherein X is CH₂, R³ is hydrogen and R² is 4-methyl-3-chlorophenyl and wherein R¹ has one of the meanings given in rows 1 to 141 of table A (Compounds I-3147 to I-3289);
Table 24 Compounds of the formula (I), wherein X is CH₂, R³ is hydrogen and R² is 3-methyl-4-methoxyphenyl and wherein R¹ has one of the meanings given in rows 1 to 141 of table A (Compounds I-3290 to I-3432);
Table 25 Compounds of the formula (I), wherein X is CH₂, R³ is hydrogen and R² is 4-methyl-3-methoxyphenyl and wherein R¹ has one of the meanings given in rows 1 to 141 of table A (Compounds I-3433 to I-3575);
Table 26 Compounds of the formula (I), wherein X is CH₂, R³ is hydrogen and R² is 4-trichloroacetylphenyl and wherein R¹ has one of the meanings given in rows 1 to 141 of table A (Compounds I-3576 to I-3718);
Table 27 Compounds of the formula (I), wherein X is CH₂, R³ is hydrogen and R² is 3-trichloroacetylphenyl and wherein R¹ has one of the meanings given in rows 1 to 141 of table A (Compounds I-3719 to I-3861);
Table 28 Compounds of the formula (I), wherein X is CH₂, R³ is hydrogen and R² is 4-aminocarbonylphenyl and wherein R¹ has one of the meanings given in rows 1 to 141 of table A (Compounds I-3862 to I-4004);
Table 29 Compounds of the formula (I), wherein X is CH₂, R³ is hydrogen and R² is 3-aminocarbonylphenyl and wherein R¹ has one of the meanings given in rows 1 to 141 of table A (Compounds I-4005 to I-4147);
Table 30 Compounds of the formula (I), wherein X is CH₂, R³ is hydrogen and R² is 4-methylsulfonylphenyl and wherein R¹ has one of the meanings given in rows 1 to 141 of table A (Compounds I-4148 to I-4290);
Table 31 Compounds of the formula (I), wherein X is CH₂, R³ is hydrogen and R² is 3-methylsulfonylphenyl and wherein R¹ has one of the meanings given in rows 1 to 141 of table A (Compounds I-4291 to I-4433);
Table 32 Compounds of the formula (I), wherein X is CH₂, R³ is hydrogen and R² is 4-nitrophenyl and wherein R¹ has one of the meanings given in rows 1 to 141 of table A (Compounds I-4434 to I-4576);
Table 33 Compounds of the formula (I), wherein X is CH₂, R³ is hydrogen and R² is 3-nitrophenyl and wherein R¹ has one of the meanings given in rows 1 to 141 of table A (Compounds I-4577 to I-4719);
Table 34 Compounds of the formula (I), wherein X is CH₂, R³ is hydrogen and R² is 4-trifluoromethylphenyl and wherein R¹ has one of the meanings given in rows 1 to 141 of table A (Compounds I-4720 to I-4862);
Table 35 Compounds of the formula (I), wherein X is CH₂, R³ is hydrogen and R² is 3-trifluoromethylphenyl and wherein R¹ has one of the meanings given in rows 1 to 141 of table A (Compounds I-4863 to I-5005);
Table 36 Compounds of the formula (I), wherein X is CH₂, R³ is hydrogen and R² is pyridine-2-yl and wherein R¹ has one of the meanings given in rows 1 to 141 of table A (Compounds I-5006 to I-5148);
Table 37 Compounds of the formula (I), wherein X is CH₂, R³ is hydrogen and R² is pyridine-3-yl and wherein R¹ has one of the meanings given in rows 1 to 141 of table A (Compounds I-5149 to I-5291);
Table 38 Compounds of the formula (I), wherein X is CH₂, R³ is hydrogen and R² is pyridine-4-yl and wherein R¹ has one of the meanings given in rows 1 to 141 of table A (Compounds I-5292 to I-5434);
Table 39 Compounds of the formula (I), wherein X is CH₂, R³ is hydrogen and R² is 6-cyanopyridine-3-yl and wherein R¹ has one of the meanings given in rows 1 to 141 of table A (Compounds I-5435 to I-5577);
Table 40 Compounds of the formula (I), wherein X is CH₂, R³ is hydrogen and R² is 6-methoxypyridine-3-yl and wherein R¹ has one of the meanings given in rows 1 to 141 of table A (Compounds I-5578 to I-5720);
Table 41 Compounds of the formula (I), wherein X is CH₂, R³ is hydrogen and R² is 6-chloropyridine-3-yl and wherein R¹ has one of the meanings given in rows 1 to 141 of table A (Compounds I-5721 to I-5863);
Table 42 Compounds of the formula (I), wherein X is CH₂, R³ is hydrogen and R² is 6-methlypyridine-3-yl and wherein R¹ has one of the meanings given in rows 1 to 141 of table A (Compounds I-5864 to I-6006);
Table 43 Compounds of the formula (I), wherein X is CH₂, R³ is hydrogen and R² is 5-cyanopyridine-2-yl and wherein R¹ has one of the meanings given in rows 1 to 141 of table A (Compounds I-6007 to I-6149);
Table 44 Compounds of the formula (I), wherein X is CH₂, R³ is hydrogen and R² is 5-methoxypyridine-2-yl and wherein R¹ has one of the meanings given in rows 1 to 141 of table A (Compounds I-6150 to I-6292);
Table 45 Compounds of the formula (I), wherein X is CH₂, R³ is hydrogen and R² is 5-chloropyridine-2-yl and wherein R¹ has one of the meanings given in rows 1 to 141 of table A (Compounds I-6293 to I-6435);
Table 46 Compounds of the formula (I), wherein X is CH₂, R³ is hydrogen and R² is 5-methlypyridine-2-yl and wherein R¹ has one of the meanings given in rows 1 to 141 of table A (Compounds I-6436 to I-6578);
Table 47 Compounds of the formula (I), wherein X is CH₂, R³ is hydrogen and R² is pyrimidine-2-yl and wherein R¹ has one of the meanings given in rows 1 to 141 of table A (Compounds I-6579 to I-6721);
Table 48 Compounds of the formula (I), wherein X is CH₂, R³ is hydrogen and R² is pyrimidine-4-yl and wherein R¹ has one of the meanings given in rows 1 to 141 of table A (Compounds I-6722 to I-6864);
Table 49 Compounds of the formula (I), wherein X is CH₂, R³ is hydrogen and R² is pyrimidine-5-yl and wherein R¹ has one of the meanings given in rows 1 to 141 of table A (Compounds I-6865 to I-7007);
Table 50 Compounds of the formula (I), wherein X is CH₂, R³ is hydrogen and R² is 5-cyanopyrimidine-2-yl and wherein R¹ has one of the meanings given in rows 1 to 141 of table A (Compounds I-7007 to I-7150);
Table 51 Compounds of the formula (I), wherein X is CH₂, R³ is hydrogen and R² is 2-cyanopyrimidine-5-yl and wherein R¹ has one of the meanings given in rows 1 to 141 of table A (Compounds I-7151 to I-7293);
Table 52 Compounds of the formula (I), wherein X is CH₂, R³ is hydrogen and R² is 5-methoxypyrimidine-2-yl and wherein R¹ has one of the meanings given in rows 1 to 141 of table A (Compounds I-7294 to I-7436);
Table 53 Compounds of the formula (I), wherein X is CH₂, R³ is hydrogen and R² is 2-methoxypyrimidine-5-yl and wherein R¹ has one of the meanings given in rows 1 to 141 of table A (Compounds I-7437 to I-7579);
Table 54 Compounds of the formula (I), wherein X is CH₂, R³ is hydrogen and R² is 5-chloropyrimidine-2-yl and wherein R¹ has one of the meanings given in rows 1 to 141 of table A (Compounds I-7580 to I-7722);
Table 55 Compounds of the formula (I), wherein X is CH₂, R³ is hydrogen and R² is 2-chloropyrimidine-5-yl and wherein R¹ has one of the meanings given in rows 1 to 141 of table A (Compounds I-7723 to I-7865);
Table 56 Compounds of the formula (I), wherein X is CH₂, R³ is hydrogen and R² is 5-methylpyrimidine-2-yl and wherein R¹ has one of the meanings given in rows 1 to 141 of table A (Compounds I-7866 to I-8008);
Table 57 Compounds of the formula (I), wherein X is CH₂, R³ is hydrogen and R² is 2-methylpyrimidine-5-yl and wherein R¹ has one of the meanings given in rows 1 to 141 of table A (Compounds I-8009 to I-8151);
Table 58 Compounds of the formula (I), wherein X is CH₂, R³ is hydrogen and R² is pyrazin-2-yl and wherein R¹ has one of the meanings given in rows 1 to 141 of table A (Compounds I-8152 to I-8294);
Table 59 Compounds of the formula (I), wherein X is CH₂, R³ is hydrogen and R² is 5-cyano-pyrazin-2-yl and wherein R¹ has one of the meanings given in rows 1 to 141 of table A (Compounds I-8295 to I-8437);
Table 60 Compounds of the formula (I), wherein X is CH₂, R³ is hydrogen and R² is 5-methoxy-pyrazin-2-yl and wherein R¹ has one of the meanings given in rows 1 to 141 of table A (Compounds I-8438 to I-8580);
Table 61 Compounds of the formula (I), wherein X is CH₂, R³ is hydrogen and R² is 5-chloro-pyrazin-2-yl, and wherein R¹ has one of the meanings given in rows 1 to 141 of table A (Compounds I-8581 to I-8723);
Table 62 Compounds of the formula (I), wherein X is CH₂, R³ is hydrogen and R² is pyridazin-3-yl, and wherein R¹ has one of the meanings given in rows 1 to 141 of table A (Compounds I-8724 to I-8866);
Table 63 Compounds of the formula (I), wherein X is CH₂, R³ is hydrogen and R² is pyridazin-4-yl, and wherein R¹ has one of the meanings given in rows 1 to 141 of table A (Compounds I-8867 to I-9009);
Table 64 Compounds of the formula (I), wherein X is CH₂, R³ is hydrogen and R² is 6-cyanopyridazin-3-yl, and wherein R¹ has one of the meanings given in rows 1 to 141 of table A (Compounds I-9010 to I-9152);
Table 65 Compounds of the formula (I), wherein X is CH₂, R³ is hydrogen and R² is 6-methoxypyridazin-3-yl, and wherein R¹ has one of the meanings given in rows 1 to 141 of table A (Compounds I-9153 to I-9295);
Table 66 Compounds of the formula (I), wherein X is CH₂, R³ is hydrogen and R² is 6-chloropyridazin-3-yl, and wherein R¹ has one of the meanings given in rows 1 to 141 of table A (Compounds I-9296 to I-9438);
Table 67 Compounds of the formula (I), wherein X is CH₂, R³ is hydrogen and R² is furan-2-yl, and wherein R¹ has one of the meanings given in rows 1 to 141 of table A (Compounds I-9439 to I-9581);
Table 68 Compounds of the formula (I), wherein X is CH₂ and R² is furan-3-yl, and wherein R¹ has one of the meanings given in rows 1 to 141 of table A (Compounds I-9582 to I-9724);
Table 69 Compounds of the formula (I), wherein X is CH₂, R³ is hydrogen and R² is 5-cyanofuran-2-yl, and wherein R¹ has one of the meanings given in rows 1 to 141 of table A (Compounds I-9725 to I-9867);
Table 70 Compounds of the formula (I), wherein X is CH₂, R³ is hydrogen and R² is 4-cyanofuran-2-yl, and wherein R¹ has one of the meanings given in rows 1 to 141 of table A (Compounds I-9868 to I-10010);
Table 71 Compounds of the formula (I), wherein X is CH₂, R³ is hydrogen and R² is 5-cyanofuran-3-yl and wherein R¹ has one of the meanings given in rows 1 to 141 of table A (Compounds I-100 μl to I-10153);
Table 72 Compounds of the formula (I), wherein X is CH₂, R³ is hydrogen and R² is 2-thienyl and wherein R¹ has one of the meanings given in rows 1 to 141 of table A (Compounds I-10154 to I-10296);
Table 73 Compounds of the formula (I), wherein X is CH₂, R³ is hydrogen and R² is 3-thienyl and wherein R¹ has one of the meanings given in rows 1 to 141 of table A (Compounds I-10297 to I-10439);
Table 74 Compounds of the formula (I), wherein X is CH₂, R³ is hydrogen and R² is 5-cyano-2-thienyl and wherein R¹ has one of the meanings given in rows 1 to 141 of table A (Compounds I-10440 to I-10582);
Table 75 Compounds of the formula (I), wherein X is CH₂, R³ is hydrogen and R² is 4-cyano-2-thienyl and wherein R¹ has one of the meanings given in rows 1 to 141 of table A (Compounds I-10583 to I-10725);
Table 76 Compounds of the formula (I), wherein X is CH₂, R³ is hydrogen and R² is 5-cyano-3-thienyl and wherein R¹ has one of the meanings given in rows 1 to 141 of table A (Compounds I-10726 to I-10868);
Table 77 Compounds of the formula (I), wherein X is CH₂, R³ is hydrogen and R² is pyrrol-2-yl and wherein R¹ has one of the meanings given in rows 1 to 141 of table A (Compounds I-10869 to I-11011);
Table 78 Compounds of the formula (I), wherein X is CH₂, R³ is hydrogen and R² is pyrrol-3-yl and wherein R¹ has one of the meanings given in rows 1 to 141 of table A (Compounds I-11012 to I-11154);
Table 79 Compounds of the formula (I), wherein X is CH₂, R³ is hydrogen and R² is 5-cyanopyrrol-2-yl and wherein R¹ has one of the meanings given in rows 1 to 141 of table A (Compounds I-11155 to I-11297);
Table 80 Compounds of the formula (I), wherein X is CH₂, R³ is hydrogen and R² is 4-cyanopyrrol-2-yl and wherein R¹ has one of the meanings given in rows 1 to 141 of table A (Compounds I-11298 to I-11440);
Table 81 Compounds of the formula (I), wherein X is CH₂, R³ is hydrogen and R² is 5-cyanopyrrol-3-yl and wherein R¹ has one of the meanings given in rows 1 to 141 of table A (Compounds I-11441 to I-11583);
Table 82 Compounds of the formula (I), wherein X is CH₂, R³ is hydrogen and R² is 5-methylpyrrol-2-yl and wherein R¹ has one of the meanings given in rows 1 to 141 of table A (Compounds I-11584 to I-11726);
Table 83 Compounds of the formula (I), wherein X is CH₂, R³ is hydrogen and R² is 4-methylpyrrol-2-yl and wherein R¹ has one of the meanings given in rows 1 to 141 of table A (Compounds I-11727 to I-11869);
Table 84 Compounds of the formula (I), wherein X is CH₂, R³ is hydrogen and R² is 5-methylpyrrol-3-yl and wherein R¹ has one of the meanings given in rows 1 to 141 of table A (Compounds I-11870 to I-12012);
Table 85 Compounds of the formula (I), wherein X is CH₂, R³ is hydrogen and R² is 1,5-dimethylpyrrol-2-yl and wherein R¹ has one of the meanings given in rows 1 to 141 of table A (Compounds I-12013 to I-12155);
Table 86 Compounds of the formula (I), wherein X is CH₂, R³ is hydrogen and R² is 1,4-dimethylpyrrol-2-yl and wherein R¹ has one of the meanings given in rows 1 to 141 of table A (Compounds I-12156 to I-12298);
Table 87 Compounds of the formula (I), wherein X is CH₂, R³ is hydrogen and R² is 1,5-dimethylpyrrol-3-yl and wherein R¹ has one of the meanings given in rows 1 to 141 of table A (Compounds I-12299 to I-12441);
Table 88 Compounds of the formula (I), wherein X is CH₂, R³ is hydrogen and R² is 1-methyl-5-cyanopyrrol-2-yl and wherein R¹ has one of the meanings given in rows 1 to 141 of table A (Compounds I-12442 to I-12584);
Table 89 Compounds of the formula (I), wherein X is CH₂, R³ is hydrogen and R² is 1-methyl-4-cyanopyrrol-2-yl and wherein R¹ has one of the meanings given in rows 1 to 141 of table A (Compounds I-12585 to I-12727);
Table 90 Compounds of the formula (I), wherein X is CH₂, R³ is hydrogen and R² is 1-methyl-5-cyanopyrrol-3-yl and wherein R¹ has one of the meanings given in rows 1 to 141 of table A (Compounds I-12728 to I-12870);
Table 91 Compounds of the formula (I), wherein X is CH₂, R³ is hydrogen and R² is oxazol-4-yl and wherein R¹ has one of the meanings given in rows 1 to 141 of table A (Compounds I-12871 to I-13013);
Table 92 Compounds of the formula (I), wherein X is CH₂, R³ is hydrogen and R² is oxazol-5-yl and wherein R¹ has one of the meanings given in rows 1 to 141 of table A (Compounds I-13014 to I-13156);
Table 93 Compounds of the formula (I), wherein X is CH₂, R³ is hydrogen and R² is 2-cyanooxazol-4-yl and wherein R¹ has one of the meanings given in rows 1 to 141 of table A (Compounds I-13157 to I-13299);
Table 94 Compounds of the formula (I), wherein X is CH₂, R³ is hydrogen and R² is 2-cyanooxazol-5-yl and wherein R¹ has one of the meanings given in rows 1 to 141 of table A (Compounds I-13300 to I-13442).
Table 95 Compounds of the formula (I), wherein X is CH₂, R³ is hydrogen and R² is 3-methyl-2-cyanooxazol-4-yl and wherein R¹ has one of the meanings given in rows 1 to 141 of table A (Compounds I-13443 to I-13585).
Table 96 Compounds of the formula (I), wherein X is CH₂, R³ is hydrogen and R² is 3-methyl-2-cyanooxazol-5-yl and wherein R¹ has one of the meanings given in rows 1 to 141 of table A (Compounds I-13586 to I-13728).
Table 97 Compounds of the formula (I), wherein X is CH₂, R³ is hydrogen and R² is 2 imidazol-4-yl and wherein R¹ has one of the meanings given in rows 1 to 141 of table A (Compounds I-13729 to I-13871).
Table 98 Compounds of the formula (I), wherein X is CH₂, R³ is hydrogen and R² is imidazol-5-yl and wherein R¹ has one of the meanings given in rows 1 to 141 of table A (Compounds I-13872 to I-14014).
Table 99 Compounds of the formula (I), wherein X is CH₂, R³ is hydrogen and R² is 2-cyanoimidazol-4-yl and wherein R¹ has one of the meanings given in rows 1 to 141 of table A (Compounds I-14015 to I-14157).
Table 100 Compounds of the formula (I), wherein X is CH₂, R³ is hydrogen and R² is 2-cyanoimidazol-5-yl and wherein R¹ has one of the meanings given in rows 1 to 141 of table A (Compounds I-14158 to I-14300).
Table 101 Compounds of the formula (I), wherein X is CH₂, R³ is hydrogen and R² is 2-cyano-1-methylimidazol-4-yl and wherein R¹ has one of the meanings given in rows 1 to 141 of table A (Compounds I-14301 to I-14443).
Table 102 Compounds of the formula (I), wherein X is CH₂, R³ is hydrogen and R² is 2-cyano-1-methylimidazol-5-yl and wherein R¹ has one of the meanings given in rows 1 to 141 of table A (Compounds I-14444 to I-14586).
Table 103 Compounds of the formula (I), wherein X is CH₂, R³ is hydrogen and R² is thiazol-4-yl and wherein R¹ has one of the meanings given in rows 1 to 141 of table A (Compounds I-14587 to I-14729).
Table 104 Compounds of the formula (I), wherein X is CH₂, R³ is hydrogen and R² is thiazol-5-yl and wherein R¹ has one of the meanings given in rows 1 to 141 of table A (Compounds I-14730 to I-14872).
Table 105 Compounds of the formula (I), wherein X is CH₂, R³ is hydrogen and R² is 2-cyanothiazol-4-yl and wherein R¹ has one of the meanings given in rows 1 to 141 of table A (Compounds I-14873 to I-15015).
Table 106 Compounds of the formula (I), wherein X is CH₂, R³ is hydrogen and R² is 2-cyanothiazol-5-yl and wherein R¹ has one of the meanings given in rows 1 to 141 of table A (Compounds I-15015 to I-15158).
Table 107 Compounds of the formula (I), wherein X is CH₂, R³ is hydrogen and R² is 3-methyl-2-cyanothiazol-4-yl and wherein R¹ has one of the meanings given in rows 1 to 141 of table A (Compounds I-15159 to I-15301).
Table 108 Compounds of the formula (I), wherein X is CH₂, R³ is hydrogen and R² is 3-methyl-2-cyanothiazol-5-yl and wherein R¹ has one of the meanings given in rows 1 to 141 of table A (Compounds I-15302 to I-15444).
Table 109 Compounds of the formula (I), wherein X is CH₂, R³ is hydrogen and R² is 4-cyanooxazol-2-yl and wherein R¹ has one of the meanings given in rows 1 to 141 of table A (Compounds I-15445 to I-15587).
Table 110 Compounds of the formula (I), wherein X is CH₂, R³ is hydrogen and R² is 5-cyanooxazol-2-yl and wherein R¹ has one of the meanings given in rows 1 to 141 of table A (Compounds I-15588 to I-15730).
Table 111 Compounds of the formula (I), wherein X is CH₂, R³ is hydrogen and R² is 4-cyanoimidazol-2-yl and wherein R¹ has one of the meanings given in rows 1 to 141 of table A (Compounds I-15731 to I-15873).
Table 112 Compounds of the formula (I), wherein X is CH₂, R³ is hydrogen and R² is 5-cyanoimidazol-2-yl and wherein R¹ has one of the meanings given in rows 1 to 141 of table A (Compounds I-15874 to I-16016).
Table 113 Compounds of the formula (I), wherein X is CH₂, R³ is hydrogen and R² is 4-cyano-1-methylimidazol-2-yl and wherein R¹ has one of the meanings given in rows 1 to 141 of table A (Compounds I-16017 to I-16159).
Table 114 Compounds of the formula (I), wherein X is CH₂, R³ is hydrogen and R² is 5-cyano-1-methylimidazol-2-yl and wherein R¹ has one of the meanings given in rows 1 to 141 of table A (Compounds I-16160 to I-16302).
Table 115 Compounds of the formula (I), wherein X is CH₂, R³ is hydrogen and R² is 5-cyanothiazol-2-yl and wherein R¹ has one of the meanings given in rows 1 to 141 of table A (Compounds I-16303 to I-16445).
Table 116 Compounds of the formula (I), wherein X is CH₂, R³ is hydrogen and R² is 4-cyanothiazol-2-yl and wherein R¹ has one of the meanings given in rows 1 to 141 of table A (Compounds I-16446 to I-16588).
Table 117 Compounds of the formula (I), wherein X is CH₂, R³ is hydrogen and R² is 3-cyanoisoxazol-5-yl and wherein R¹ has one of the meanings given in rows 1 to 141 of table A (Compounds I-16589 to I-16731).
Table 118 Compounds of the formula (I), wherein X is CH₂, R³ is hydrogen and R² is 5-cyanoisoxazol-3-yl and wherein R¹ has one of the meanings given in rows 1 to 141 of table A (Compounds I-16732 to I-16874).
Table 119 Compounds of the formula (I), wherein X is CH₂, R³ is hydrogen and R² is 3-cyanoisothiazol-5-yl and wherein R¹ has one of the meanings given in rows 1 to 141 of table A (Compounds I-16875 to I-17017).
Table 120 Compounds of the formula (I), wherein X is CH₂, R³ is hydrogen and R² is 5-cyanoisothiazol-3-yl and wherein R¹ has one of the meanings given in rows 1 to 141 of table A (Compounds I-17018 to I-17160).
Table 121 Compounds of the formula (I), wherein X is CH₂, R³ is hydrogen and R² is 3-cyanopyrazol-5-yl and wherein R¹ has one of the meanings given in rows 1 to 141 of table A (Compounds I-17161 to I-17303).
Table 122 Compounds of the formula (I), wherein X is CH₂, R³ is hydrogen and R² is 5-cyanopyrazol-3-yl and wherein R¹ has one of the meanings given in rows 1 to 141 of table A (Compounds I-17304 to I-17446).
Table 123 Compounds of the formula (I), wherein X is CH₂, R³ is hydrogen and R² is 3-cyano-1-methylpyrazol-5-yl and wherein R¹ has one of the meanings given in rows 1 to 141 of table A (Compounds I-17447 to I-17589).
Table 124 Compounds of the formula (I), wherein X is CH₂, R³ is hydrogen and R² is 5-cyano-1-methylpyrazol-3-yl and wherein R¹ has one of the meanings given in rows 1 to 141 of table A (Compounds I-17590 to I-17732).
Table 125 Compounds of the formula (I), wherein X is CH₂, R³ is hydrogen and R² is 5-cyano-1,3,4-oxadiazol-2-yl and wherein R¹ has one of the meanings given in rows 1 to 141 of table A (Compounds I-17733 to I-17875).
Table 126 Compounds of the formula (I), wherein X is CH₂, R³ is hydrogen and R² is 5-cyano-1,3,4-thiadiazol-2-yl and wherein R¹ has one of the meanings given in rows 1 to 141 of table A (Compounds I-17876 to I-18018).
Table 127 Compounds of the formula (I), wherein X is CH₂, R³ is hydrogen and R² is 5-cyano-1,2,4-triazol-3-yl and wherein R¹ has one of the meanings given in rows 1 to 141 of table A (Compounds I-18019 to I-18161).
Table 128 Compounds of the formula (I), wherein X is CH₂, R³ is hydrogen and R² is 5-cyano-4-methyl-1,2,4-triazol-3-yl and wherein R¹ has one of the meanings given in rows 1 to 141 of table A (Compounds I-18162 to I-18304).
Tables 129 to 256: Compounds of the formula (I), which correspond to the compounds of tables 1 to 128 wherein X═CH₂ has been replaced by X═CH₂CH₂ (Compounds I-18305 to I-36608).
Tables 257 to 384: Compounds of the formula (I), which correspond to the compounds of tables 1 to 128 wherein X is CH₂ and R³=hydrogen has been replaced by R³=methyl (Compounds I-36609 to I-54912).
Tables 385 to 512: Compounds of the formula (I), which correspond to the compounds of tables 1 to 128 wherein X is CH₂ and R³=hydrogen has been replaced by R³=ethyl (Compounds I-54913 to I-73216).
Tables 513 to 640: Compounds of the formula (I), which correspond to the compounds of tables 1 to 128 wherein X is CH₂ and R³=hydrogen has been replaced by R³=n-propyl (Compounds I-73217 to I-91520).
Tables 641 to 768: Compounds of the formula (I), which correspond to the compounds of tables 129 to 256 wherein X is CH₂CH₂ and R³=hydrogen has been replaced by R³=methyl (Compounds I-91521 to I-109824).
Tables 769 to 896: Compounds of the formula (I), which correspond to the compounds of tables 129 to 256 wherein X is CH₂CH₂ and R³=hydrogen has been replaced by R³=ethyl (Compounds I-109825 to I-128128).
Tables 897 to 1024: Compounds of the formula (I), which correspond to the compounds of tables 129 to 256 wherein X is CH₂CH₂ and R³=hydrogen has been replaced by R³=n-propyl (Compounds I-128129 to I-146432).

Examples of particular preferred compounds according to the present invention are the compounds listed below, their pharmaceutically acceptable salts, their N-oxides and the pharmaceutically acceptable salts of said N-oxides:
2-(4-nitrophenyl)-6-phenyl-3-(piperazin-1-ylmethyl)imidazo[1,2-a]pyridine,
1-(4-((2-(3-nitrophenyl)-6-phenylimidazo[1,2-a]pyridin-3-yl)methyl)piperazin-1-yl)ethanone,
1-(4-((2-(4-nitrophenyl)-6-phenylimidazo[1,2-a]pyridin-3-yl)methyl)piperazin-1-yl)ethanone,
3-((1,4-diazepan-1-yl)methyl)-2-(4-nitrophenyl)-6-phenylimidazo[1,2-a]pyridine,
2-(4-nitrophenyl)-6-phenyl-3-((4-propylpiperazin-1-yl)ethyl)imidazo[1,2-a]-pyridine,
3-((4-ethylpiperazin-1-yl)methyl)-2-(4-nitrophenyl)-6-phenylimidazo[1,2-a]pyridine,
3-((4-methylpiperazin-1-yl)methyl)-2-(4-nitrophenyl)-6-phenylimidazo[1,2-a]-pyridine,
3-((1,4-diazepan-1-yl)methyl)-2-(4-(methylsulfonyl)phenyl)-6-phenylimidazo[1,2-a]pyridine,
4-(3-((1,4-diazepan-1-yl)methyl)-6-phenylimidazo[1,2-a]pyridin-2-yl)benzonitrile,
4-(3-((1,4-diazepan-1-yl)methyl)-6-phenylimidazo[1,2-a]pyridin-2-yl)benzamide,
3-((1,4-diazepan-1-yl)methyl)-6-phenyl-2-(pyridin-4-yl)imidazo[1,2-a]pyridine,
3-((4-methyl-1,4-diazepan-1-yl)methyl)-2-(4-nitrophenyl)-6-phenylimidazo[1,2-a]-pyridine,
4-((2-(4-nitrophenyl)-6-phenylimidazo[1,2-a]pyridin-3-yl)methyl)piperazin-2-one,
3-((1,4-diazepan-1-yl)methyl)-2-(4-chlorophenyl)-6-phenylimidazo[1,2-a]pyridine,
3-((1,4-diazepan-1-yl)methyl)-6-phenyl-2-(pyridin-3-yl)imidazo[1,2-a]pyridine,
5-(3-((1,4-diazepan-1-yl)methyl)-6-phenylimidazo[1,2-a]pyridin-2-yl)picolinonitrile,
3-((1,4-diazepan-1-yl)methyl)-6-phenyl-2-(p-tolyl)imidazo[1,2-a]pyridine,
3-((1,4-diazepan-1-yl)methyl)-2-(4-methoxyphenyl)-6-phenylimidazo[1,2-a]-pyridine,
3-((1,4-diazepan-1-yl)methyl)-2,6-diphenylimidazo[1,2-a]pyridine,
5-(3-((1,4-diazepan-1-yl)methyl)-6-phenylimidazo[1,2-a]pyridin-2-yl)thiophene-3-carbonitrile,
5-(3-((1,4-diazepan-1-yl)methyl)-6-phenylimidazo[1,2-a]pyridin-2-yl)thiophene-2-carbonitrile,
3-(3-((1,4-diazepan-1-yl)methyl)-6-phenylimidazo[1,2-a]pyridin-2-yl)benzonitrile,
4-(3-((1,4-diazepan-1-yl)methyl)-6-(p-tolyl)imidazo[1,2-a]pyridin-2-yl)benzonitrile,
4-(3-((1,4-diazepan-1-yl)methyl)-6-(m-tolyl)imidazo[1,2-a]pyridin-2-yl)benzonitrile,
4-(3-((1,4-diazepan-1-yl)methyl)-6-(o-tolyl)imidazo[1,2-a]pyridin-2-yl)benzonitrile,
4-(3-((1,4-diazepan-1-yl)methyl)-6-(pyrimidin-5-yl)imidazo[1,2-a]pyridin-2-yl)benzonitrile,
4-(3-((1,4-diazepan-1-yl)methyl)-6-(pyridin-4-yl)imidazo[1,2-a]pyridin-2-yl)benzonitrile,
4-(3-((1,4-diazepan-1-yl)methyl)-6-(2-methylpyridin-4-yl)imidazo[1,2-a]pyridin-2-yl)benzonitrile,
4-(3-((1,4-diazepan-1-yl)methyl)-6-(pyridin-3-yl)imidazo[1,2-a]pyridin-2-yl)benzonitrile,
4-((2-(4-methoxyphenyl)-6-phenylimidazo[1,2-a]pyridin-3-yl)methyl)piperazin-2-one,
2-(4-chlorophenyl)-3-((4-methyl-1,4-diazepan-1-yl)methyl)-6-phenylimidazo[1,2-a]pyridine,
4-((2-(4-chlorophenyl)-6-phenylimidazo[1,2-a]pyridin-3-yl)methyl)piperazin-2-one,
4-(3-((3-oxopiperazin-1-yl)methyl)-6-phenylimidazo[1,2-a]pyridin-2-yl)benzonitrile,
4-(3-((4-methyl-1,4-diazepan-1-yl)methyl)-6-phenylimidazo[1,2-a]pyridin-2-yl)benzonitrile,
5-(3-((3-oxopiperazin-1-yl)methyl)-6-phenylimidazo[1,2-a]pyridin-2-yl)thiophene-2-carbonitrile,
5-(3-((4-methyl-1,4-diazepan-1-yl)methyl)-6-phenylimidazo[1,2-a]pyridin-2-yl)thiophene-2-carbonitrile,
4-(3-((1,4-diazepan-1-yl)methyl)-6-(1H-pyrazol-4-yl)imidazo[1,2-a]pyridin-2-yl)benzonitrile,
4-(3-((1,4-diazepan-1-yl)methyl)-6-(1-methyl-1H-pyrrol-2-yl)imidazo[1,2-a]pyridin-2-yl)benzonitrile,
4-(3-((1,4-diazepan-1-yl)methyl)-6-(oxazol-5-yl)imidazo[1,2-a]pyridin-2-yl)benzonitrile,
4-(3-((1,4-diazepan-1-yl)methyl)-6-(4-chlorophenyl)imidazo[1,2-a]pyridin-2-yl)benzonitrile,
4-(3-((1,4-diazepan-1-yl)methyl)-6-(4-methoxyphenyl)imidazo[1,2-a]pyridin-2-yl)benzonitrile,
4-(3-((1,4-diazepan-1-yl)methyl)-2-(4-cyanophenyl)imidazo[1,2-a]pyridin-6-yl)benzamide,
4,4′-(3-((1,4-diazepan-1-yl)ethyl)imidazo[1,2-a]pyridine-2,6-diyl)dibenzonitrile,
4-(3-((1,4-diazepan-1-yl)methyl)-6-(thiophen-3-yl)imidazo[1,2-a]pyridin-2-yl)benzonitrile.

Examples of particular preferred compounds according to the present invention are the compounds listed below, their pharmaceutically acceptable salts, their N-oxides and the pharmaceutically acceptable salts of said N-oxides:
2-(4-methoxyphenyl)-3-((4-methyl-1,4-diazepan-1-yl)methyl)-6-(pyridin-3-yl)imidazo[1,2-a]pyridine,
3-((1,4-diazepan-1-yl)methyl)-2-(4-methoxyphenyl)-6-(pyridin-3-yl)imidazo[1,2-a]-pyridine,
3-((1,4-diazepan-1-yl)methyl)-2-(4-methoxyphenyl)-6-(4-(trifluoromethyl)phenyl)imidazo[1,2-a]pyridine,
2-(4-methoxyphenyl)-3-((4-methyl-1,4-diazepan-1-yl)methyl)-6-(4-(trifluoromethyl)phenyl)imidazo[1,2-a]pyridine,
4-(3-((1,4-diazepan-1-yl)methyl)-2-(4-methoxyphenyl)imidazo[1,2-a]pyridin-6-yl)benzamide,
4-(2-(4-methoxyphenyl)-3-((4-methyl-1,4-diazepan-1-yl)ethyl)imidazo[1,2-a]-pyridin-6-yl)benzamide,
5-(3-((1,4-diazepan-1-yl)methyl)-2-(4-methoxyphenyl)imidazo[1,2-a]pyridin-6-yl)thiophene-2-carbonitrile,
5-(2-(4-methoxyphenyl)-3-((4-methyl-1,4-diazepan-1-yl)ethyl)imidazo[1,2-a]-pyridin-6-yl)thiophene-2-carbonitrile,
3-((1,4-diazepan-1-yl)methyl)-2-(4-methoxyphenyl)-6-(4-(pentafluorothio)phenyl)imidazo[1,2-a]pyridine,
2-(4-methoxyphenyl)-3-((4-methyl-1,4-diazepan-1-yl)methyl)-6-(4-(pentafluorothio)phenyl)imidazo[1,2-a]pyridine,
2-(4-methoxyphenyl)-3-((4-methyl-1,4-diazepan-1-yl)methyl)-6-(1-methyl-1H-pyrrol-2-yl)imidazo[1,2-a]pyridine,
3-((1,4-diazepan-1-yl)methyl)-2-(4-methoxyphenyl)-6-(1-methyl-1H-pyrrol-2-yl)imidazo[1,2-a]pyridine,
4-(3-((1,4-diazepan-1-yl)methyl)-2-(4-methoxyphenyl)imidazo[1,2-a]pyridin-6-yl)-2-methylbenzonitrile,
4-(2-(4-methoxyphenyl)-3-((4-methyl-1,4-diazepan-1-yl)ethyl)imidazo[1,2-a]-pyridin-6-yl)-2-methylbenzonitrile,
3-((1,4-diazepan-1-yl)methyl)-2-(4-methoxyphenyl)-6-(pyrimidin-5-yl)imidazo[1,2-a]pyridine,
2-(4-methoxyphenyl)-3-((4-methyl-1,4-diazepan-1-yl)methyl)-6-(pyrimidin-5-yl)imidazo[1,2-a]pyridine,
5-(2-(4-methoxyphenyl)-3-((4-methyl-1,4-diazepan-1-yl)ethyl)imidazo[1,2-a]-pyridin-6-yl)oxazole,
5-(3-((1,4-diazepan-1-yl)methyl)-2-(4-methoxyphenyl)imidazo[1,2-a]pyridin-6-yl)oxazole,
3-((1,4-diazepan-1-yl)methyl)-2-(4-methoxyphenyl)-6-(p-tolyl)imidazo[1,2-a]-pyridine,
2-(4-methoxyphenyl)-3-((4-methyl-1,4-diazepan-1-yl)methyl)-6-(p-tolyl)imidazo[1,2-a]pyridine,
4-(3-((1,4-diazepan-1-yl)methyl)-2-(4-methoxyphenyl)imidazo[1,2-a]pyridin-6-yl)benzonitrile,
4-(2-(4-methoxyphenyl)-3-((4-methyl-1,4-diazepan-1-yl)ethyl)imidazo[1,2-a]-pyridin-6-yl)benzonitrile,
5-(3-((1,4-diazepan-1-yl)methyl)-2-(4-methoxyphenyl)imidazo[1,2-a]pyridin-6-yl)thiophene-3-carbonitrile,
5-(2-(4-methoxyphenyl)-3-((4-methyl-1,4-diazepan-1-yl)ethyl)imidazo[1,2-a]-pyridin-6-yl)thiophene-3-carbonitrile,
6-(4-chlorophenyl)-2-(4-methoxyphenyl)-3-((4-methyl-1,4-diazepan-1-yl)methyl)imidazo[1,2-a]pyridine,
3-((1,4-diazepan-1-yl)methyl)-6-(4-chlorophenyl)-2-(4-methoxyphenyl)imidazo[1,2-a]pyridine,
4-(3-((1,4-diazepan-1-yl)methyl)-2-(4-methoxyphenyl)imidazo[1,2-a]pyridin-6-yl)thiophene-2-carbonitrile,
4-(2-(4-methoxyphenyl)-3-((4-methyl-1,4-diazepan-1-yl)ethyl)imidazo[1,2-a]-pyridin-6-yl)thiophene-2-carbonitrile,
2-(4-methoxyphenyl)-3-((4-methyl-1,4-diazepan-1-yl)methyl)-6-(o-tolyl)imidazo[1,2-a]pyridine,
3-((1,4-diazepan-1-yl)methyl)-2-(4-methoxyphenyl)-6-(o-tolyl)imidazo[1,2-a]-pyridine,
4-(2-(4-methoxyphenyl)-3-((4-methyl-1,4-diazepan-1-yl)methyl)imidazo[1,2-a]-pyridin-6-yl)-N,N-dimethylaniline,
4-(3-((1,4-diazepan-1-yl)methyl)-2-(4-methoxyphenyl)imidazo[1,2-a]pyridin-6-yl)N,N-dimethylaniline,
2,6-bis(4-methoxyphenyl)-3-((4-methyl-1,4-diazepan-1-yl)methyl)imidazo[1,2-a]-pyridine,
3-((1,4-diazepan-1-yl)methyl)-2,6-bis(4-methoxyphenyl)imidazo[1,2-a]pyridine,
3-((1,4-diazepan-1-yl)methyl)-2-(4-methoxyphenyl)-6-(m-tolyl)imidazo[1,2-a]-pyridine,
2-(4-methoxyphenyl)-3-((4-methyl-1,4-diazepan-1-yl)methyl)-6-(m-tolyl)imidazo[1,2-a]pyridine,
2-(4-methoxyphenyl)-3-((4-methyl-1,4-diazepan-1-yl)methyl)-6-(2-methylpyridin-4-yl)imidazo[1,2-a]pyridine,
3-((1,4-diazepan-1-yl)methyl)-2-(4-methoxyphenyl)-6-(2-methylpyridin-4-yl)imidazo[1,2-a]pyridine,
2-(4-methoxyphenyl)-3-((4-methyl-1,4-diazepan-1-yl)methyl)-6-(thiophen-3-yl)imidazo[1,2-a]pyridine,
3-((1,4-diazepan-1-yl)methyl)-2-(4-methoxyphenyl)-6-(thiophen-3-yl)imidazo[1,2-a]pyridine,
6-(3,4-dimethoxyphenyl)-2-(4-methoxyphenyl)-3-((4-methyl-1,4-diazepan-1-yl)methyl)imidazo[1,2-a]pyridine,
3-((1,4-diazepan-1-yl)methyl)-6-(3,4-dimethoxyphenyl)-2-(4-methoxyphenyl)imidazo[1,2-a]pyridine,
6-(1,5-dimethyl-1H-pyrrol-2-yl)-2-(4-methoxyphenyl)-3-((4-methyl-1,4-diazepan-1-yl)ethyl)imidazo[1,2-a]pyridine,
3-((1,4-diazepan-1-yl)methyl)-6-(1,5-dimethyl-1H-pyrrol-2-yl)-2-(4-methoxyphenyl)imidazo[1,2-a]pyridine,
5-(3-((1,4-diazepan-1-yl)methyl)-2-(4-methoxyphenyl)imidazo[1,2-a]pyridin-6-yl)-1H-pyrrole-2-carbonitrile,
5-(2-(4-methoxyphenyl)-3-((4-methyl-1,4-diazepan-1-yl)methyl)imidazo[1,2-a]-pyridin-6-yl)-1H-pyrrole-2-carbonitrile,
2-(4-methoxyphenyl)-3-((4-methyl-1,4-diazepan-1-yl)methyl)-6-(5-methyl-1H-pyrrol-2-yl)imidazo[1,2-a]pyridine,
3-((1,4-diazepan-1-yl)methyl)-2-(4-methoxyphenyl)-6-(5-methyl-1H-pyrrol-2-yl)imidazo[1,2-a]pyridine,
2-(4-methoxyphenyl)-3-((4-methyl-1,4-diazepan-1-yl)methyl)-6-phenylimidazo[1,2-a]pyridine,
6-(furan-2-yl)-2-(4-methoxyphenyl)-3-((4-methyl-1,4-diazepan-1-yl)methyl)imidazo[1,2-a]pyridine,
3-((1,4-diazepan-1-yl)methyl)-6-(furan-2-yl)-2-(4-methoxyphenyl)imidazo[1,2-a]-pyridine,
3-((1,4-diazepan-1-yl)methyl)-2-(4-methoxyphenyl)-6-(1H-pyrrol-2-yl)imidazo[1,2-a]pyridine,
2-(4-methoxyphenyl)-3-((4-methyl-1,4-diazepan-1-yl)methyl)-6-(1H-pyrrol-2-yl)imidazo[1,2-a]pyridine,
2-(4-methoxyphenyl)-3-((4-methyl-1,4-diazepan-1-yl)methyl)-6-(pyridin-4-yl)imidazo[1,2-a]pyridine,
3-((1,4-diazepan-1-yl)methyl)-2-(4-methoxyphenyl)-6-(pyridin-4-yl)imidazo[1,2-a]-pyridine,
3-((1,4-diazepan-1-yl)methyl)-6-(3,4-dimethylphenyl)-2-(4-methoxyphenyl)imidazo[1,2-a]pyridine,
6-(3,4-dimethylphenyl)-2-(4-methoxyphenyl)-3-((4-methyl-1,4-diazepan-1-yl)methyl)imidazo[1,2-a]pyridine,
3-((1,4-diazepan-1-yl)methyl)-2-(4-methoxyphenyl)-6-(1H-pyrazol-4-yl)imidazo[1,2-a]pyridine,
2-(4-methoxyphenyl)-3-((4-methyl-1,4-diazepan-1-yl)methyl)-6-(1H-pyrazol-4-yl)imidazo[1,2-a]pyridine,
2-(4-chlorophenyl)-3-((4-methyl-1,4-diazepan-1-yl)methyl)-6-(4-(pentafluorothio)phenyl)imidazo[1,2-a]pyridine,
3-((1,4-diazepan-1-yl)methyl)-2-(4-chlorophenyl)-6-(4-(pentafluorothio)phenyl)imidazo[1,2-a]pyridine,
3-((1,4-diazepan-1-yl)methyl)-2-(4-chlorophenyl)-6-(pyrimidin-5-yl)imidazo[1,2-a]-pyridine,
2-(4-chlorophenyl)-3-((4-methyl-1,4-diazepan-1-yl)methyl)-6-(pyrimidin-5-yl)imidazo[1,2-a]pyridine,
4-(2-(4-chlorophenyl)-3-((4-methyl-1,4-diazepan-1-yl)methyl)imidazo[1,2-a]-pyridin-6-yl)-2-methylbenzonitrile,
4-(3-((1,4-diazepan-1-yl)methyl)-2-(4-chlorophenyl)imidazo[1,2-a]pyridin-6-yl)-2-methylbenzonitrile,
3-((1,4-diazepan-1-yl)methyl)-2-(4-chlorophenyl)-6-(1H-pyrrol-2-yl)imidazo[1,2-a]-pyridine,
2-(4-chlorophenyl)-3-((4-methyl-1,4-diazepan-1-yl)methyl)-6-(1H-pyrrol-2-yl)imidazo[1,2-a]pyridine,
3-((1,4-diazepan-1-yl)methyl)-2-(4-chlorophenyl)-6-(m-tolyl)imidazo[1,2-a]-pyridine,
2-(4-chlorophenyl)-3-((4-methyl-1,4-diazepan-1-yl)methyl)-6-(m-tolyl)imidazo[1,2-a]pyridine,
2-(4-chlorophenyl)-6-(3,4-dimethylphenyl)-3-((4-methyl-1,4-diazepan-1-yl)methyl)imidazo[1,2-a]pyridine,
3-((1,4-diazepan-1-yl)methyl)-2-(4-chlorophenyl)-6-(3,4-dimethylphenyl)imidazo[1,2-a]pyridine,
5-(3-((1,4-diazepan-1-yl)methyl)-2-(4-chlorophenyl)imidazo[1,2-a]pyridin-6-yl)-1H-pyrrole-2-carbonitrile,
5-(2-(4-chlorophenyl)-3-((4-methyl-1,4-diazepan-1-yl)methyl)imidazo[1,2-a]-pyridin-6-yl)-1H-pyrrole-2-carbonitrile,
3-((1,4-diazepan-1-yl)methyl)-2-(4-chlorophenyl)-6-(o-tolyl)imidazo[1,2-a]pyridine,
2-(4-chlorophenyl)-3-((4-methyl-1,4-diazepan-1-yl)methyl)-6-(o-tolyl)imidazo[1,2-a]pyridine,
2-(4-chlorophenyl)-6-(3,4-dimethoxyphenyl)-3-((4-methyl-1,4-diazepan-1-yl)methyl)imidazo[1,2-a]pyridine,
3-((1,4-diazepan-1-yl)methyl)-2-(4-chlorophenyl)-6-(3,4-dimethoxyphenyl)imidazo[1,2-a]pyridine,
2-(4-chlorophenyl)-6-(1,5-dimethyl-1H-pyrrol-2-yl)-3-((4-methyl-1,4-diazepan-1-yl)methyl)imidazo[1,2-a]pyridine,
3-((1,4-diazepan-1-yl)methyl)-2-(4-chlorophenyl)-6-(1,5-dimethyl-1H-pyrrol-2-yl)imidazo[1,2-a]pyridine,
2-(4-chlorophenyl)-3-((4-methyl-1,4-diazepan-1-yl)methyl)-6-(5-methyl-1H-pyrrol-2-yl)imidazo[1,2-a]pyridine,
3-((1,4-diazepan-1-yl)methyl)-2-(4-chlorophenyl)-6-(5-methyl-1H-pyrrol-2-yl)imidazo[1,2-a]pyridine,
3-((1,4-diazepan-1-yl)methyl)-2-(4-chlorophenyl)-6-(pyridin-3-yl)imidazo[1,2-a]-pyridine,
2-(4-chlorophenyl)-3-((4-methyl-1,4-diazepan-1-yl)methyl)-6-(pyridin-3-yl)imidazo[1,2-a]pyridine,
2-(4-chlorophenyl)-6-(furan-2-yl)-3-((4-methyl-1,4-diazepan-1-yl)methyl)imidazo[1,2-a]pyridine,
3-((1,4-diazepan-1-yl)methyl)-2-(4-chlorophenyl)-6-(furan-2-yl)imidazo[1,2-a]-pyridine,
3-((1,4-diazepan-1-yl)methyl)-2-(4-chlorophenyl)-6-(1H-pyrazol-4-yl)imidazo[1,2-a]pyridine,
2-(4-chlorophenyl)-3-((4-methyl-1,4-diazepan-1-yl)methyl)-6-(1H-pyrazol-4-yl)imidazo[1,2-a]pyridine,
4-(2-(4-chlorophenyl)-3-((4-methyl-1,4-diazepan-1-yl)methyl)imidazo[1,2-a]-pyridin-6-yl)thiophene-2-carbonitrile,
4-(3-((1,4-diazepan-1-yl)methyl)-2-(4-chlorophenyl)imidazo[1,2-a]pyridin-6-yl)thiophene-2-carbonitrile,
5-(2-(4-chlorophenyl)-3-((4-methyl-1,4-diazepan-1-yl)methyl)imidazo[1,2-a]-pyridin-6-yl)thiophene-3-carbonitrile,
5-(3-((1,4-diazepan-1-yl)methyl)-2-(4-chlorophenyl)imidazo[1,2-a]pyridin-6-yl)thiophene-3-carbonitrile,
3-((1,4-diazepan-1-yl)methyl)-2-(4-chlorophenyl)-6-(1-methyl-1H-pyrrol-2-yl)imidazo[1,2-a]pyridine,
2-(4-chlorophenyl)-3-((4-methyl-1,4-diazepan-1-yl)methyl)-6-(1-methyl-1H-pyrrol-2-yl)imidazo[1,2-a]pyridine,
2-(4-chlorophenyl)-3-((4-methyl-1,4-diazepan-1-yl)methyl)-6-(2-methylpyridin-4-yl)imidazo[1,2-a]pyridine,
3-((1,4-diazepan-1-yl)methyl)-2-(4-chlorophenyl)-6-(2-methylpyridin-4-yl)imidazo[1,2-a]pyridine,
5-(2-(4-chlorophenyl)-3-((4-methyl-1,4-diazepan-1-yl)methyl)imidazo[1,2-a]-pyridin-6-yl)thiophene-2-carbonitrile,
5-(3-((1,4-diazepan-1-yl)methyl)-2-(4-chlorophenyl)imidazo[1,2-a]pyridin-6-yl)thiophene-2-carbonitrile,
5-(3-((1,4-diazepan-1-yl)methyl)-2-(4-chlorophenyl)imidazo[1,2-a]pyridin-6-yl)oxazole,
5-(2-(4-chlorophenyl)-3-((4-methyl-1,4-diazepan-1-yl)methyl)imidazo[1,2-a]-pyridin-6-yl)oxazole,
4-(2-(4-chlorophenyl)-3-((4-methyl-1,4-diazepan-1-yl)methyl)imidazo[1,2-a]-pyridin-6-yl)benzamide,
4-(3-((1,4-diazepan-1-yl)methyl)-2-(4-chlorophenyl)imidazo[1,2-a]pyridin-6-yl)benzamide,
2-(4-chlorophenyl)-6-(4-methoxyphenyl)-3-((4-methyl-1,4-diazepan-1-yl)methyl)imidazo[1,2-a]pyridine,
3-((1,4-diazepan-1-yl)methyl)-2-(4-chlorophenyl)-6-(4-methoxyphenyl)imidazo[1,2-a]pyridine,
2-(4-chlorophenyl)-3-((4-methyl-1,4-diazepan-1-yl)methyl)-6-(pyridin-4-yl)imidazo[1,2-a]pyridine,
3-((1,4-diazepan-1-yl)methyl)-2-(4-chlorophenyl)-6-(pyridin-4-yl)imidazo[1,2-a]-pyridine,
3-((1,4-diazepan-1-yl)methyl)-2-(4-chlorophenyl)-6-(thiophen-3-yl)imidazo[1,2-a]-pyridine,
2-(4-chlorophenyl)-3-((4-methyl-1,4-diazepan-1-yl)methyl)-6-(thiophen-3-yl)imidazo[1,2-a]pyridine,
3-((1,4-diazepan-1-yl)methyl)-2,6-bis(4-chlorophenyl)imidazo[1,2-a]pyridine,
2,6-bis(4-chlorophenyl)-3-((4-methyl-1,4-diazepan-1-yl)ethyl)imidazo[1,2-a]-pyridine,
4-(3-((1,4-diazepan-1-yl)methyl)-2-(4-chlorophenyl)imidazo[1,2-a]pyridin-6-yl)benzonitrile,
4-(2-(4-chlorophenyl)-3-((4-methyl-1,4-diazepan-1-yl)ethyl)imidazo[1,2-a]-pyridin-6-yl)benzonitrile,
4-(2-(4-chlorophenyl)-3-((4-methyl-1,4-diazepan-1-yl)ethyl)imidazo[1,2-a]-pyridin-6-yl)-N,N-dimethylaniline, 4-(3-((1,4-diazepan-1-yl)methyl)-2-(4-chlorophenyl)imidazo[1,2-a]pyridin-6-yl)-N,N-dimethylaniline, 3-((1,4-diazepan-1-yl)methyl)-2-(4-chlorophenyl)-6-(p-tolyl)imidazo[1,2-a]pyridine,
2-(4-chlorophenyl)-3-((4-methyl-1,4-diazepan-1-yl)methyl)-6-(p-tolyl)imidazo[1,2-a]pyridine,
3-((1,4-diazepan-1-yl)methyl)-2-(4-chlorophenyl)-6-(4-(trifluoromethyl)phenyl)imidazo[1,2-a]pyridine,
2-(4-chlorophenyl)-3-((4-methyl-1,4-diazepan-1-yl)methyl)-6-(4-(trifluoromethyl)phenyl)imidazo[1,2-a]pyridine,
5-(3-((1,4-diazepan-1-yl)methyl)-2-(4-cyanophenyl)imidazo[1,2-a]pyridin-6-yl)thiophene-2-carbonitrile,
5-(2-(4-cyanophenyl)-3-((4-methyl-1,4-diazepan-1-yl)ethyl)imidazo[1,2-a]-pyridin-6-yl)thiophene-2-carbonitrile,
5-(3-((1,4-diazepan-1-yl)methyl)-2-(4-cyanophenyl)imidazo[1,2-a]pyridin-6-yl)thiophene-3-carbonitrile,
5-(2-(4-cyanophenyl)-3-((4-methyl-1,4-diazepan-1-yl)ethyl)imidazo[1,2-a]-pyridin-6-yl)thiophene-3-carbonitrile,
4-(3-((4-methyl-1,4-diazepan-1-yl)methyl)-6-(1H-pyrazol-4-yl)imidazo[1,2-a]-pyridin-2-yl)benzonitrile,
4-(3-((1,4-diazepan-1-yl)methyl)-6-(furan-2-yl)imidazo[1,2-a]pyridin-2-yl)benzonitrile,
4-(6-(furan-2-yl)-3-((4-methyl-1,4-diazepan-1-yl)ethyl)imidazo[1,2-a]pyridin-2-yl)benzonitrile,
4-(6-(3,4-dimethylphenyl)-3-((4-methyl-1,4-diazepan-1-yl)ethyl)imidazo[1,2-a]-pyridin-2-yl)benzonitrile,
4-(3-((1,4-diazepan-1-yl)methyl)-6-(3,4-dimethylphenyl)imidazo[1,2-a]pyridin-2-yl)benzonitrile,
4-(3-((4-methyl-1,4-diazepan-1-yl)methyl)-6-(o-tolyl)imidazo[1,2-a]pyridin-2-yl)benzonitrile,
4-(3-((1,4-diazepan-1-yl)methyl)-6-(5-methyl-1H-pyrrol-2-yl)imidazo[1,2-a]pyridin-2-yl)benzonitrile,
4-(3-((4-methyl-1,4-diazepan-1-yl)methyl)-6-(5-methyl-1H-pyrrol-2-yl)imidazo[1,2-a]pyridin-2-yl)benzonitrile,
4-(3-((4-methyl-1,4-diazepan-1-yl)methyl)-6-(pyridin-3-yl)imidazo[1,2-a]pyridin-2-yl)benzonitrile,
4-(3-((1,4-diazepan-1-yl)methyl)-6-(1,5-dimethyl-1H-pyrrol-2-yl)imidazo[1,2-a]-pyridin-2-yl)benzonitrile,
4-(6-(1,5-dimethyl-1H-pyrrol-2-yl)-3-((4-methyl-1,4-diazepan-1-yl)methyl)imidazo[1,2-a]pyridin-2-yl)benzonitrile,
4-(6-(3,4-dimethoxyphenyl)-3-((4-methyl-1,4-diazepan-1-yl)ethyl)imidazo[1,2-a]-pyridin-2-yl)benzonitrile,
4-(3-((1,4-diazepan-1-yl)methyl)-6-(3,4-dimethoxyphenyl)imidazo[1,2-a]pyridin-2-yl)benzonitrile,
4-(3-((4-methyl-1,4-diazepan-1-yl)methyl)-6-(pyridin-4-yl)imidazo[1,2-a]pyridin-2-yl)benzonitrile,
4-(3-((1,4-diazepan-1-yl)methyl)-6-(1H-pyrrol-2-yl)imidazo[1,2-a]pyridin-2-yl)benzonitrile,
4-(3-((4-methyl-1,4-diazepan-1-yl)methyl)-6-(1H-pyrrol-2-yl)imidazo[1,2-a]pyridin-2-yl)benzonitrile,
4-(3-((4-methyl-1,4-diazepan-1-yl)methyl)-6-(pyrimidin-5-yl)imidazo[1,2-a]pyridin-2-yl)benzonitrile,
4-(3-((1,4-diazepan-1-yl)methyl)-6-(4-(trifluoromethyl)phenyl)imidazo[1,2-a]-pyridin-2-yl)benzonitrile,
4-(3-((4-methyl-1,4-diazepan-1-yl)methyl)-6-(4-(trifluoromethyl)phenyl)imidazo[1,2-a]pyridin-2-yl)benzonitrile,
4-(3-((1,4-diazepan-1-yl)methyl)-2-(4-cyanophenyl)imidazo[1,2-a]pyridin-6-yl)-2-methylbenzonitrile,
4-(2-(4-cyanophenyl)-3-((4-methyl-1,4-diazepan-1-yl)ethyl)imidazo[1,2-a]-pyridin-6-yl)-2-methylbenzonitrile,
4-(6-(4-methoxyphenyl)-3-((4-methyl-1,4-diazepan-1-yl)ethyl)imidazo[1,2-a]-pyridin-2-yl)benzonitrile,
5-(2-(4-cyanophenyl)-3-((4-methyl-1,4-diazepan-1-yl)ethyl)imidazo[1,2-a]-pyridin-6-yl)-1H-pyrrole-2-carbonitrile,
5-(3-((1,4-diazepan-1-yl)methyl)-2-(4-cyanophenyl)imidazo[1,2-a]pyridin-6-yl)-1H-pyrrole-2-carbonitrile,
4-(2-(4-cyanophenyl)-3-((4-methyl-1,4-diazepan-1-yl)ethyl)imidazo[1,2-a]-pyridin-6-yl)thiophene-2-carbonitrile,
4-(3-((1,4-diazepan-1-yl)methyl)-2-(4-cyanophenyl)imidazo[1,2-a]pyridin-6-yl)thiophene-2-carbonitrile,
4-(6-(4-chlorophenyl)-3-((4-methyl-1,4-diazepan-1-yl)ethyl)imidazo[1,2-a]-pyridin-2-yl)benzonitrile,
4-(2-(4-cyanophenyl)-3-((4-methyl-1,4-diazepan-1-yl)ethyl)imidazo[1,2-a]-pyridin-6-yl)benzamide,
4,4′-(3-((4-methyl-1,4-diazepan-1-yl)methyl)imidazo[1,2-a]pyridine-2,6-diyl)dibenzonitrile,
4-(6-(4-(dimethylamino)phenyl)-3-((4-methyl-1,4-diazepan-1-yl)methyl)imidazo[1,2-a]pyridin-2-yl)benzonitrile,
4-(3-((1,4-diazepan-1-yl)methyl)-6-(4-(dimethylamino)phenyl)imidazo[1,2-a]-pyridin-2-yl)benzonitrile,
4-(3-((4-methyl-1,4-diazepan-1-yl)methyl)-6-(2-methylpyridin-4-yl)imidazo[1,2-a]-pyridin-2-yl)benzonitrile,
4-(3-((4-methyl-1,4-diazepan-1-yl)methyl)-6-(1-methyl-1H-pyrrol-2-yl)imidazo[1,2-a]pyridin-2-yl)benzonitrile,
4-(3-((4-methyl-1,4-diazepan-1-yl)methyl)-6-(p-tolyl)imidazo[1,2-a]pyridin-2-yl)benzonitrile,
4-(3-((4-methyl-1,4-diazepan-1-yl)methyl)-6-(m-tolyl)imidazo[1,2-a]pyridin-2-yl)benzonitrile,
4-(3-((4-methyl-1,4-diazepan-1-yl)methyl)-6-(oxazol-5-yl)imidazo[1,2-a]pyridin-2-yl)benzonitrile,
4-(3-((4-methyl-1,4-diazepan-1-yl)methyl)-6-(4-(pentafluorothio)phenyl)imidazo[1,2-a]pyridin-2-yl)benzonitrile,
4-(3-((1,4-diazepan-1-yl)methyl)-6-(4-(pentafluorothio)phenyl)imidazo[1,2-a]-pyridin-2-yl)benzonitrile,
4-(3-((4-methyl-1,4-diazepan-1-yl)methyl)-6-(thiophen-3-yl)imidazo[1,2-a]pyridin-2-yl)benzonitrile,
4-(2-(4-chlorophenyl)-3-((4-methylpiperazin-1-yl)ethyl)imidazo[1,2-a]pyridin-6-yl)benzonitrile,
4-(2-(4-methoxyphenyl)-3-((3-oxopiperazin-1-yl)ethyl)imidazo[1,2-a]pyridin-6-yl)benzonitrile,
4-(2-(4-methoxyphenyl)-3-((4-(2,2,2-trifluoroacetyl)-1,4-diazepan-1-yl)methyl)imidazo[1,2-a]pyridin-6-yl)benzonitrile,
4-(2-(4-methoxyphenyl)-3-((4-(trifluoromethyl)-1,4-diazepan-1-yl)methyl)imidazo[1,2-a]pyridin-6-yl)benzonitrile,
4-(6-(3,4-dimethylphenyl)-3-((4-propylpiperazin-1-yl)ethyl)imidazo[1,2-a]pyridin-2-yl)benzonitrile,
4-((2-(4-chlorophenyl)-6-(3,4-dimethoxyphenyl)imidazo[1,2-a]pyridin-3-yl)methyl)piperazin-2-one,
4-(6-(3,4-dimethylphenyl)-3-((3-oxopiperazin-1-yl)ethyl)imidazo[1,2-a]pyridin-2-yl)benzonitrile,
4-((2-(4-methoxyphenyl)-6-(oxazol-5-yl)imidazo[1,2-a]pyridin-3-yl)methyl)piperazin-2-one,
4-(3-((4-methylpiperazin-1-yl)methyl)-6-(pyrimidin-5-yl)imidazo[1,2-a]pyridin-2-yl)benzonitrile,
4-(3-((4-methylpiperazin-1-yl)methyl)-6-(4-(trifluoromethyl)phenyl)imidazo[1,2-a]-pyridin-2-yl)benzonitrile,
4-(6-(3,4-dimethylphenyl)-3-((4-(trifluoromethyl)piperazin-1-yl)methyl)imidazo[1,2-a]pyridin-2-yl)benzonitrile,
4-(6-(3,4-dimethoxyphenyl)-3-((4-propylpiperazin-1-yl)ethyl)imidazo[1,2-a]-pyridin-2-yl)benzonitrile,
4-(2-(4-cyanophenyl)-3-((4-(trifluoromethyl)-1,4-diazepan-1-yl)methyl)imidazo[1,2-a]pyridin-6-yl)thiophene-2-carbonitrile,
4-(3-((4-methylpiperazin-1-yl)methyl)-6-(2-methylpyridin-4-yl)imidazo[1,2-a]-pyridin-2-yl)benzonitrile,
2-(4-methoxyphenyl)-6-(pyrimidin-5-yl)-3-((4-(trifluoromethyl)piperazin-1-yl)methyl)imidazo[1,2-a]pyridine,
4-(3-((4-methylpiperazin-1-yl)methyl)-6-(oxazol-5-yl)imidazo[1,2-a]pyridin-2-yl)benzonitrile,
1-(4-((2-(4-methoxyphenyl)-6-(pyridin-3-yl)imidazo[1,2-a]pyridin-3-yl)methyl)-1,4-diazepan-1-yl)ethanone,
4-(2-(4-methoxyphenyl)-3-((4-propylpiperazin-1-yl)ethyl)imidazo[1,2-a]pyridin-6-yl)-N,N-dimethylaniline, 4-(3-((4-acetylpiperazin-1-yl)methyl)-6-(5-methyl-1H-pyrrol-2-yl)imidazo[1,2-a]-pyridin-2-yl)benzonitrile,
1-(4-((2-(4-methoxyphenyl)-6-(1-methyl-1H-pyrrol-2-yl)imidazo[1,2-a]pyridin-3-yl)methyl)piperazin-1-yl)ethanone,
2-(4-methoxyphenyl)-3-((4-methylpiperazin-1-yl)methyl)-6-(4-(trifluoromethyl)phenyl)imidazo[1,2-a]pyridine,
2-(4-chlorophenyl)-6-(m-tolyl)-3-((4-(trifluoromethyl)-1,4-diazepan-1-yl)methyl)imidazo[1,2-a]pyridine,
4-(6-(4-(dimethylamino)phenyl)-3-((4-(2,2,2-trifluoroacetyl)-1,4-diazepan-1-yl)methyl)imidazo[1,2-a]pyridin-2-yl)benzonitrile,
4-(6-(4-(dimethylamino)phenyl)-3-((4-(trifluoromethyl)-1,4-diazepan-1-yl)methyl)imidazo[1,2-a]pyridin-2-yl)benzonitrile,
4-(6-(1-methyl-1H-pyrrol-2-yl)-3-((4-methylpiperazin-1-yl)ethyl)imidazo[1,2-a]-pyridin-2-yl)benzonitrile,
4-(6-(furan-2-yl)-3-((4-(2,2,2-trifluoroacetyl)-1,4-diazepan-1-yl)methyl)imidazo[1,2-a]pyridin-2-yl)benzonitrile,
2-(4-methoxyphenyl)-3-((4-methylpiperazin-1-yl)methyl)-6-(pyridin-4-yl)imidazo[1,2-a]pyridine,
4-(6-(1-methyl-1H-pyrrol-2-yl)-3-((4-(2,2,2-trifluoroacetyl)piperazin-1-yl)methyl)imidazo[1,2-a]pyridin-2-yl)benzonitrile,
4-(6-(oxazol-5-yl)-3-((4-(2,2,2-trifluoroacetyl)-1,4-diazepan-1-yl)methyl)imidazo[1,2-a]pyridin-2-yl)benzonitrile,
4-(3-((4-acetyl-1,4-diazepan-1-yl)methyl)-6-(4-(pentafluorothio)phenyl)imidazo[1,2-a]pyridin-2-yl)benzonitrile,
4-(6-(3,4-dimethylphenyl)-3-((4-(trifluoromethyl)-1,4-diazepan-1-yl)methyl)imidazo[1,2-a]pyridin-2-yl)benzonitrile,
4-(6-(1-methyl-1H-pyrrol-2-yl)-3-((4-(trifluoromethyl)-1,4-diazepan-1-yl)methyl)imidazo[1,2-a]pyridin-2-yl)benzonitrile,
4-(3-((4-methylpiperazin-1-yl)methyl)-6-(thiophen-3-yl)imidazo[1,2-a]pyridin-2-yl)benzonitrile,
2-(4-methoxyphenyl)-3-((4-methylpiperazin-1-yl)methyl)-6-(1H-pyrrol-2-yl)imidazo[1,2-a]pyridine,
5-(2-(4-cyanophenyl)-3-((4-methylpiperazin-1-yl)ethyl)imidazo[1,2-a]pyridin-6-yl)thiophene-3-carbonitrile,
4-((6-(furan-2-yl)-2-(4-methoxyphenyl)imidazo[1,2-a]pyridin-3-yl)methyl)piperazin-2-one,
4-(3-((4-methylpiperazin-1-yl)methyl)-6-(1H-pyrazol-4-yl)imidazo[1,2-a]pyridin-2-yl)benzonitrile,
4-(6-(furan-2-yl)-3-((4-propylpiperazin-1-yl)ethyl)imidazo[1,2-a]pyridin-2-yl)benzonitrile,
4-(3-((4-acetyl-1,4-diazepan-1-yl)methyl)-2-(4-(2,2,2-trifluoroacetyl)phenyl)imidazo[1,2-a]pyridin-6-yl)benzonitrile,
6-phenyl-2-(pyridin-3-yl)-3-((4-(trifluoromethyl)piperazin-1-yl)methyl)imidazo[1,2-a]pyridine,
4-((6-phenyl-2-(1H-pyrrol-2-yl)imidazo[1,2-a]pyridin-3-yl)methyl)piperazin-2-one,
5-(6-phenyl-3-((4-(trifluoromethyl)-1,4-diazepan-1-yl)ethyl)imidazo[1,2-a]pyridin-2-yl)picolinonitrile,
4-(2-(p-tolyl)-3-((4-(2,2,2-trifluoroacetyl)piperazin-1-yl)ethyl)imidazo[1,2-a]-pyridin-6-yl)benzonitrile,
4-(6-(4-cyanophenyl)-3-((4-propylpiperazin-1-yl)methyl)imidazo[1,2-a]pyridin-2-yl)benzamide,
4-(3-((4-acetylpiperazin-1-yl)methyl)-2-(p-tolyl)imidazo[1,2-a]pyridin-6-yl)benzonitrile,
4-(2-(1,5-dimethyl-1H-pyrrol-2-yl)-3-((3-oxopiperazin-1-yl)methyl)imidazo[1,2-a]-pyridin-6-yl)benzonitrile,
4-(2-(1,5-dimethyl-1H-pyrrol-2-yl)-3-((4-(2,2,2-trifluoroacetyl)-1,4-diazepan-1-yl)methyl)imidazo[1,2-a]pyridin-6-yl)benzonitrile,
4-(2-(4-(methylsulfonyl)phenyl)-3-((4-(2,2,2-trifluoroacetyl)-1,4-diazepan-1-yl)methyl)imidazo[1,2-a]pyridin-6-yl)benzonitrile,
4-(3-((4-acetyl-1,4-diazepan-1-yl)methyl)-2-(furan-2-yl)imidazo[1,2-a]pyridin-6-yl)benzonitrile,
5-(6-phenyl-3-((4-(trifluoromethyl)-1,4-diazepan-1-yl)methyl)imidazo[1,2-a]pyridin-2-yl)thiophene-2-carbonitrile,
4-(2-(3-nitrophenyl)-3-(piperazin-1-ylmethyl)imidazo[1,2-a]pyridin-6-yl)benzonitrile

4-(2-(4-(methylsulfonyl)phenyl)-3-((3-oxopiperazin-1-yl)methyl)imidazo[1,2-a]-pyridin-6-yl)benzonitrile,
4-(2-(furan-2-yl)-3-((4-(2,2,2-trifluoroacetyl)-1,4-diazepan-1-yl)methyl)imidazo[1,2-a]pyridin-6-yl)benzonitrile,
2-(furan-2-yl)-6-phenyl-3-((4-(trifluoromethyl)-1,4-diazepan-1-yl)methyl)imidazo[1,2-a]pyridine,
4-(3-((4-acetyl-1,4-diazepan-1-yl)methyl)-6-(3,4-dimethylphenyl)imidazo[1,2-a]-pyridin-2-yl)benzamide,
5-(6-(m-tolyl)-3-((4-(trifluoromethyl)-1,4-diazepan-1-yl)methyl)imidazo[1,2-a]-pyridin-2-yl)-1H-pyrrole-2-carbonitrile,
3-((4-methylpiperazin-1-yl)methyl)-6-(1H-pyrrol-2-yl)-2-(p-tolyl)imidazo[1,2-a]-pyridine,
3-(3-(piperazin-1-ylmethyl)-6-(p-tolyl)imidazo[1,2-a]pyridin-2-yl)benzonitrile,
1-(4-((2-(pyridin-3-yl)-6-(p-tolyl)imidazo[1,2-a]pyridin-3-yl)methyl)piperazin-1-yl)ethanone,
4-(3-((4-methylpiperazin-1-yl)methyl)-6-(p-tolyl)imidazo[1,2-a]pyridin-2-yl)thiophene-2-carbonitrile,
4-(3-((4-methylpiperazin-1-yl)methyl)-2-(p-tolyl)imidazo[1,2-a]pyridin-6-yl)thiophene-2-carbonitrile,
2-methyl-4-(3-((4-methylpiperazin-1-yl)methyl)-6-(pyridin-3-yl)imidazo[1,2-a]-pyridin-2-yl)benzonitrile,
1-(4-((6-(pyridin-4-yl)-2-(p-tolyl)imidazo[1,2-a]pyridin-3-yl)methyl)piperazin-1-yl)ethanone,
2,2,2-trifluoro-1-(4-(3-((4-methylpiperazin-1-yl)methyl)-6-(m-tolyl)imidazo[1,2-a]-pyridin-2-yl)phenyl)ethanone,
2,2,2-trifluoro-1-(4-((6-(2-methylpyridin-4-yl)-2-(p-tolyl)imidazo[1,2-a]pyridin-3-yl)methyl)-1,4-diazepan-1-yl)ethanone,
4-(2-(p-tolyl)-3-((4-(trifluoromethyl)-1,4-diazepan-1-yl)methyl)imidazo[1,2-a]-pyridin-6-yl)benzamide,
2-phenyl-3-((4-propylpiperazin-1-yl)methyl)-6-(p-tolyl)imidazo[1,2-a]pyridine,
5-(3-((4-acetyl-1,4-diazepan-1-yl)methyl)-6-(3,4-dimethylphenyl)imidazo[1,2-a]-pyridin-2-yl)picolinonitrile,
4-(6-(3,4-dimethylphenyl)-3-((4-(2,2,2-trifluoroacetyl)-1,4-diazepan-1-yl)methyl)imidazo[1,2-a]pyridin-2-yl)benzamide,
3-(3-((4-acetyl-1,4-diazepan-1-yl)methyl)-6-(1,5-dimethyl-1H-pyrrol-2-yl)imidazo[1,2-a]pyridin-2-yl)benzonitrile,
5-(3-((4-methylpiperazin-1-yl)methyl)-2-(p-tolyl)imidazo[1,2-a]pyridin-6-yl)thiophene-2-carbonitrile,
1-(4-((6-(4-(dimethylamino)phenyl)-2-(p-tolyl)imidazo[1,2-a]pyridin-3-yl)methyl)-1,4-diazepan-1-yl)ethanone,
1-(4-((2-phenyl-6-(4-(trifluoromethyl)phenyl)imidazo[1,2-a]pyridin-3-yl)methyl)-1,4-diazepan-1-yl)ethanone,
6-(4-methoxyphenyl)-3-((4-methylpiperazin-1-yl)methyl)-2-(p-tolyl)imidazo[1,2-a]-pyridine,
1-(4-((6-(4-methoxyphenyl)-2-(p-tolyl)imidazo[1,2-a]pyridin-3-yl)methyl)-1,4-diazepan-1-yl)ethanone,
5-(3-((4-methylpiperazin-1-yl)methyl)-6-(p-tolyl)imidazo[1,2-a]pyridin-2-yl)thiophene-2-carbonitrile,
N,N-dimethyl-4-(3-((4-methylpiperazin-1-yl)methyl)-2-(p-tolyl)imidazo[1,2-a]pyridin-6-yl)aniline, 6-(2-methylpyridin-4-yl)-2-(p-tolyl)-3-((4-(trifluoromethyl)-1,4-diazepan-1-yl)methyl)imidazo[1,2-a]pyridine,
4-(3-((4-methylpiperazin-1-yl)methyl)-6-(m-tolyl)imidazo[1,2-a]pyridin-2-yl)benzamide,
2,2,2-trifluoro-1-(4-(3-((4-methylpiperazin-1-yl)methyl)-6-(1H-pyrrol-2-yl)imidazo[1,2-a]pyridin-2-yl)phenyl)ethanone,
4-(3-((4-acetylpiperazin-1-yl)methyl)-6-(p-tolyl)imidazo[1,2-a]pyridin-2-yl)thiophene-2-carbonitrile,
2,2,2-trifluoro-1-(4-((6-(pyridin-3-yl)-2-(p-tolyl)imidazo[1,2-a]pyridin-3-yl)methyl)piperazin-1-yl)ethanone,
5-(3-((4-acetyl-1,4-diazepan-1-yl)methyl)-6-(p-tolyl)imidazo[1,2-a]pyridin-2-yl)thiophene-3-carbonitrile,
2-(1,5-dimethyl-1H-pyrrol-2-yl)-3-((4-propylpiperazin-1-yl)methyl)-6-(m-tolyl)imidazo[1,2-a]pyridine,
4-(3-((3-oxopiperazin-1-yl)methyl)-6-(1H-pyrrol-2-yl)imidazo[1,2-a]pyridin-2-yl)benzamide,
6-(4-methoxyphenyl)-3-((4-methylpiperazin-1-yl)methyl)-2-(1H-pyrrol-2-yl)imidazo[1,2-a]pyridine,
3-((4-methylpiperazin-1-yl)methyl)-6-(pyridin-4-yl)-2-(4-(trifluoromethyl)phenyl)imidazo[1,2-a]pyridine,
4-(6-(4-(dimethylamino)phenyl)-3-(piperazin-1-ylmethyl)imidazo[1,2-a]pyridin-2-yl)-2-methylbenzonitrile,
1-(4-((6-(4-chlorophenyl)-2-(p-tolyl)imidazo[1,2-a]pyridin-3-yl)methyl)-1,4-diazepan-1-yl)ethanone,
3-((4-methylpiperazin-1-yl)methyl)-6-(1H-pyrrol-2-yl)-2-(4-(trifluoromethyl)phenyl)imidazo[1,2-a]pyridine,
4-(2-(3,4-dimethylphenyl)-3-((4-(2,2,2-trifluoroacetyl)-1,4-diazepan-1-yl)methyl)imidazo[1,2-a]pyridin-6-yl)-2-methylbenzonitrile,
1-(4-((2-(5-methyl-1H-pyrrol-2-yl)-6-(pyridin-4-yl)imidazo[1,2-a]pyridin-3-yl)methyl)-1,4-diazepan-1-yl)ethanone,
3-((4-methylpiperazin-1-yl)methyl)-2-(4-nitrophenyl)-6-(1H-pyrrol-2-yl)imidazo[1,2-a]pyridine,
3-((4-methylpiperazin-1-yl)methyl)-2-(1H-pyrrol-2-yl)-6-(m-tolyl)imidazo[1,2-a]-pyridine,
4-(3-(piperazin-1-ylmethyl)-6-(4-(trifluoromethyl)phenyl)imidazo[1,2-a]pyridin-2-yl)benzamide,
3-((4-methylpiperazin-1-yl)methyl)-2-(1H-pyrrol-2-yl)-6-(4-(trifluoromethyl)phenyl)imidazo[1,2-a]pyridine,
5-(3-((4-methylpiperazin-1-yl)methyl)-6-(p-tolyl)imidazo[1,2-a]pyridin-2-yl)picolinonitrile,
5-(3-((4-methylpiperazin-1-yl)methyl)-2-(p-tolyl)imidazo[1,2-a]pyridin-6-yl)-1H-pyrrole-2-carbonitrile,
4,4′-(3-((3-oxopiperazin-1-yl)ethyl)imidazo[1,2-a]pyridine-2,6-diyl)dibenzamide,
5-(3-((4-acetyl-1,4-diazepan-1-yl)methyl)-2-(3-cyanophenyl)imidazo[1,2-a]pyridin-6-yl)thiophene-2-carbonitrile,
4-(3-((1,4-diazepan-1-yl)methyl)-6-(4-((trifluoromethyl)thio)phenyl)imidazo[1,2-a]-pyridin-2-yl)benzonitrile,
4-(3-((1,4-diazepan-1-yl)methyl)-6-(4-((trifluoromethyl)sulfonyl)phenyl)imidazo[1,2-a]pyridin-2-yl)benzonitrile.

In a further aspect the present invention relates to Imidazo[1,2-a]pyridine compounds of the formula (I),

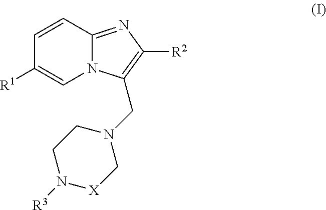

wherein X, R¹, R² and R³ are as defined herein above, their pharmaceutically acceptable salts, their N-oxides and the pharmaceutically acceptable salts of said N-oxides, except for the following compounds:
2-(3-nitrophenyl)-6-phenyl-3-(1-piperazinylmethyl)-imidazo[1,2-a]pyridine,
2-(4-nitrophenyl)-6-phenyl-3-(1-piperazinylmethyl)-imidazo[1,2-a]pyridine,
2,6-diphenyl-3-(1-piperazinylmethyl)-imidazo[1,2-a]pyridine,
2-(4-chlorophenyl)-6-phenyl-3-(piperazin-1-ylmethyl)-imidazo[1,2-a]pyridine,
2-(4-fluorophenyl)-6-phenyl-3-(piperazin-1-ylmethyl)-imidazo[1,2-a]pyridine,
2-(4-methoxyphenyl)-6-phenyl-3-(piperazin-1-ylmethyl)-imidazo[1,2-a]pyridine,
2-(3-methoxyphenyl)-6-phenyl-3-(piperazin-1-ylmethyl)-imidazo[1,2-a]pyridine,
2-(4-methylphenyl)-6-phenyl-3-(piperazin-1-ylmethyl)-imidazo[1,2-a]pyridine,
2-(4-chlorophenyl)-3-[(4-ethyl-piperazin-1-yl)methyl]-6-phenyl-imidazo[1,2-a]-pyridine,
3-[(4-ethyl-1-piperazinyl)methyl]-2,6-diphenyl-imidazo[1,2-a]pyridine,
3-[(4-ethyl-1-piperazinyl)methyl]-2-(4-methylphenyl)-6-phenyl-imidazo[1,2-a]-pyridine,
1-[4-[[2-(3-nitrophenyl)-6-phenylimidazo[1,2-a]pyridin-3-yl]methyl]-piperazin-1-yl]-ethanone,
1-[4-[[2-(4-nitrophenyl)-6-phenylimidazo[1,2-a]pyridin-3-yl]methyl]-piperazin-1-yl]-ethanone,
1-[4-[[2-(4-fluorophenyl)-6-phenylimidazo[1,2-a]pyridin-3-yl]methyl]-piperazin-1-yl]-ethanone,
1-[4-[(2,6-diphenylimidazo[1,2-a]pyridin-3-yl)methyl]-piperazin-1-yl]-ethanone,
1-[4-[(2-(3-methoxyphenyl)-6-phenylimidazo[1,2-a]pyridin-3-yl)methyl]-piperazin-1-yl]-ethanone,
1-[4-[[2-(4-chlorophenyl)-6-phenylimidazo[1,2-a]pyridin-3-yl]methyl]-piperazin-1-yl]-ethanone,
1-[4-[[2-(4-nitrophenyl)-6-phenylimidazo[1,2-a]pyridin-3-yl]methyl]-piperazin-1-yl]-ethanone,
1-[4-[[2-(3-nitrophenyl)-6-phenylimidazo[1,2-a]pyridin-3-yl]methyl]-piperazin-1-yl]-ethanone,
2-methyl-1-[4-[[2-(4-methylphenyl)-6-phenylimidazo[1,2-a]pyridin-3-yl]methyl]-piperazin-1-yl]-propan-1-one,
1-[4-[(2-(3-methoxyphenyl)-6-phenylimidazo[1,2-a]pyridin-3-yl)methyl]-piperazin-1-yl]-propanone,
1-[4-[[2-(3-nitrophenyl)-6-phenylimidazo[1,2-a]pyridin-3-yl]methyl]-piperazin-1-yl]-propan-1-one,
1-[4-[[2-(4-fluorophenyl)-6-phenylimidazo[1,2-a]pyridin-3-yl]methyl]-piperazin-1-yl]-propan-1-one,
1-[4-[[2-(4-methylphenyl)-6-phenylimidazo[1,2-a]pyridin-3-yl]methyl]-piperazin-1-yl]-propan-1-one,
1-[4-[[2-(4-chlorophenyl)-6-phenylimidazo[1,2-a]pyridin-3-yl]methyl]-piperazin-1-yl]-propanone,
1-[4-[[2-(4-methoxyphenyl)-6-phenylimidazo[1,2-a]pyridin-3-yl]methyl]-piperazin-1-yl]-propanone,
1-[4-[[2-(4-nitrophenyl)-6-phenylimidazo[1,2-a]pyridin-3-yl]methyl]-piperazin-1-yl]-propan-1-one,
2-methyl-1-[4-[[2-(4-nitrophenyl)-6-phenylimidazo[1,2-a]pyridin-3-yl]methyl]-piperazin-1-yl]-propan-1-one,
1-[4-[[2-(4-methoxyphenyl)-6-phenylimidazo[1,2-a]pyridin-3-yl]methyl]-piperazin-1-yl]-2-methyl-1-propanone,
1-[4-[[2-(4-chlorophenyl)-6-phenylimidazo[1,2-a]pyridin-3-yl]methyl]-piperazin-1-yl]-2-methyl-propan-1-one,
1-[4-[[2-(4-fluorophenyl)-6-phenylimidazo[1,2-a]pyridin-3-yl]methyl]-piperazin-1-yl]-2-methyl-propan-1-one,
2-methyl-1-[4-[[2-(3-methoxyphenyl)-6-phenylimidazo[1,2-a]pyridin-3-yl]methyl]-piperazin-1-yl]-propan-1-one,
2-methyl-1-[4-[[2-(3-nitrophenyl)-6-phenylimidazo[1,2-a]pyridin-3-yl]methyl]-piperazin-1-yl]-propan-1-one,
3-methyl-1-[4-[[2-(4-nitrophenyl)-6-phenylimidazo[1,2-a]pyridin-3-yl]methyl]-piperazin-1-yl]-butan-1-one,
1-[4-[[2-(4-fluorophenyl)-6-phenylimidazo[1,2-a]pyridin-3-yl]methyl]-piperazin-1-yl]-3-methyl-butan-1-one,
1-[4-[[2-(4-chlorophenyl)-6-phenylimidazo[1,2-a]pyridin-3-yl]methyl]-piperazin-1-yl]-3-methyl-butan-1-one,
3-methyl-1-[4-[[2-(3-methoxyphenyl)-6-phenylimidazo[1,2-a]pyridin-3-yl]methyl]-piperazin-1-yl]-butan-1-one,
3-methyl-1-[4-[[2-(4-methylphenyl)-6-phenylimidazo[1,2-a]pyridin-3-yl]methyl]-piperazin-1-yl]-butan-1-one,
3-methyl-1-[4-[[2-(3-nitrophenyl)-6-phenylimidazo[1,2-a]pyridin-3-yl]methyl]-piperazin-1-yl]-butan-1-one,
cyclopropyl-[4-[[2-(3-methoxyphenyl)-6-phenylimidazo[1,2-a]pyridin-3-yl]methyl]-1-piperazinyl]-methanone,
cyclopropyl-[4-[[2-(4-fluorophenyl)-6-phenylimidazo[1,2-a]pyridin-3-yl]methyl]-piperazin-1-yl]-methanone,
cyclopropyl-[4-[[2-(3-nitrophenyl)-6-phenylimidazo[1,2-a]pyridin-3-yl]methyl]-piperazin-1-yl]-methanone,
cyclopropyl-[4-[[2-(4-nitrophenyl)-6-phenylimidazo[1,2-a]pyridin-3-yl]methyl]-piperazin-1-yl]-methanone,
cyclopropyl-[4-[[2-(4-methoxyphenyl)-6-phenylimidazo[1,2-a]pyridin-3-yl]methyl]-piperazin-1-yl]-methanone,
cyclopropyl-[4-[[2-(4-chlorophenyl)-6-phenylimidazo[1,2-a]pyridin-3-yl]methyl]-piperazin-1-yl]-methanone,
cyclobutyl-[4-[[2-(4-nitrophenyl)-6-phenylimidazo[1,2-a]pyridin-3-yl]methyl]-piperazin-1-yl]-methanone,
cyclobutyl-[4-[[2-(4-methoxyphenyl)-6-phenylimidazo[1,2-a]pyridin-3-yl]methyl]-piperazin-1-yl]-methanone,
cyclobutyl-[4-[[2-(4-fluorophenyl)-6-phenylimidazo[1,2-a]pyridin-3-yl]methyl]-piperazin-1-yl]-methanone,
cyclobutyl-[4-[[2-(4-methylphenyl)-6-phenylimidazo[1,2-a]pyridin-3-yl]methyl]-piperazin-1-yl]-methanone,
cyclobutyl-[4-[[2-(3-methoxyphenyl)-6-phenylimidazo[1,2-a]pyridin-3-yl]methyl]-piperazin-1-yl]-methanone,
cyclobutyl-[4-[[2-(4-chlorophenyl)-6-phenylimidazo[1,2-a]pyridin-3-yl]methyl]-piperazin-1-yl]-methanone,
cyclobutyl-[4-[[2-(3-nitrophenyl)-6-phenylimidazo[1,2-a]pyridin-3-yl]methyl]-piperazin-1-yl]-methanone,
cyclopentyl-[4-[[2-(4-chlorophenyl)-6-phenylimidazo[1,2-a]pyridin-3-yl]methyl]-piperazin-1-yl]-methanone,
cyclopentyl-[4-[[2-(4-fluorophenyl)-6-phenylimidazo[1,2-a]pyridin-3-yl]methyl]-piperazin-1-yl]-methanone,
cyclopentyl-[4-[[2-(3-methoxyphenyl)-6-phenylimidazo[1,2-a]pyridin-3-yl]methyl]-piperazin-1-yl]-methanone,
cyclopentyl-[4-[[2-(4-methoxyphenyl)-6-phenylimidazo[1,2-a]pyridin-3-yl]-methyl]-piperazin-1-yl]-methanone,
cyclopentyl-[4-[[2-(3-nitrophenyl)-6-phenylimidazo[1,2-a]pyridin-3-yl]methyl]-piperazin-1-yl]-methanone,
cyclopentyl-[4-[[2-(4-nitrophenyl)-6-phenylimidazo[1,2-a]pyridin-3-yl]methyl]-piperazin-1-yl]-methanone,
1-[4-[[2-(4-fluorophenyl)-6-phenylimidazo[1,2-a]pyridin-3-yl]methyl]-piperazin-1-yl]-2-methoxy-ethanone,
2-methoxy-1-[4-[[2-(3-nitrophenyl)-6-phenylimidazo[1,2-a]pyridin-3-yl]methyl]-piperazin-1-yl]ethanone,
2-methoxy-1-[4-[[2-(4-methylphenyl)-6-phenylimidazo[1,2-a]pyridin-3-yl]methyl]-piperazin-1-yl]-ethanone, and
2-methoxy-1-[4-[[2-(4-nitrophenyl)-6-phenylimidazo[1,2-a]pyridin-3-yl]methyl]-piperazin-1-yl]-ethanone.

The compounds of the formula I according to the invention can be prepared by analogy to standard methods of organic chemistry, known in the art.

Compounds of the formula I can be prepared e.g. starting from alpha bromo ketone compounds and 2-aminopyridine compounds or the salts thereof as depicted in schemes 1, 2 and 3.

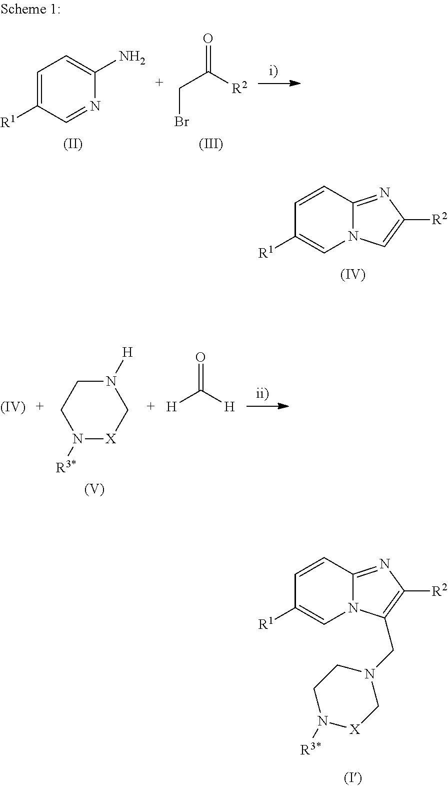

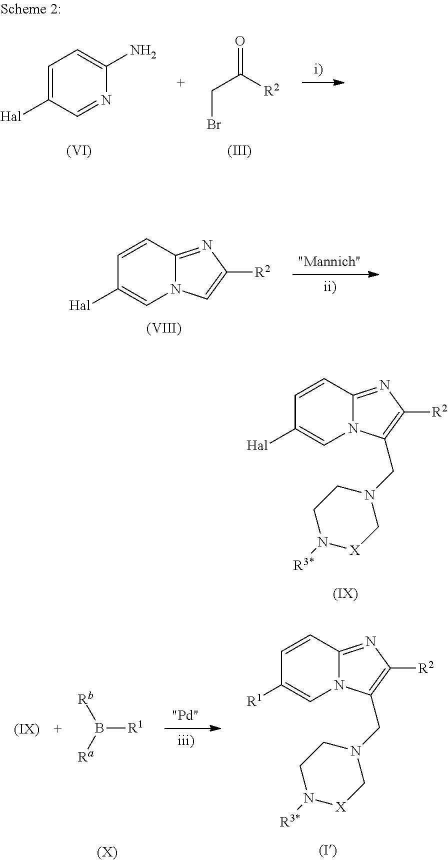

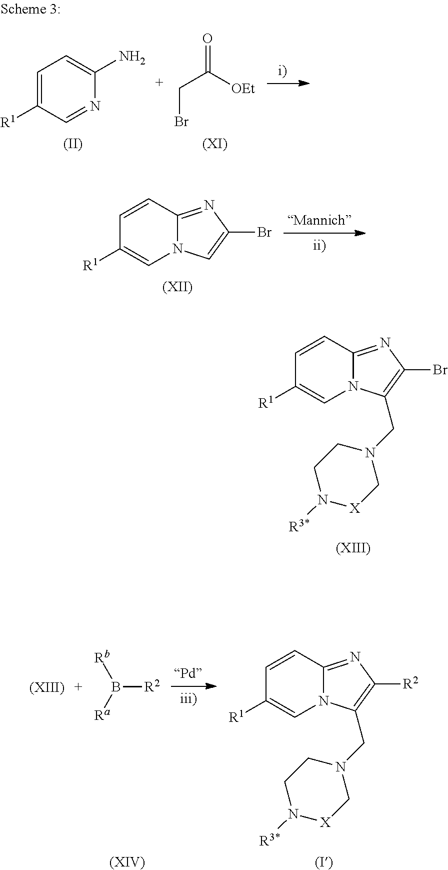

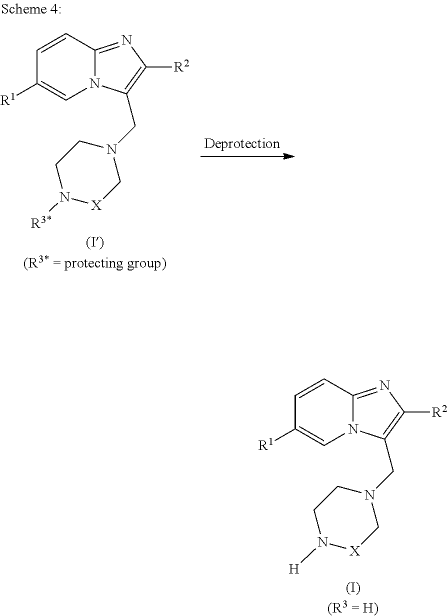

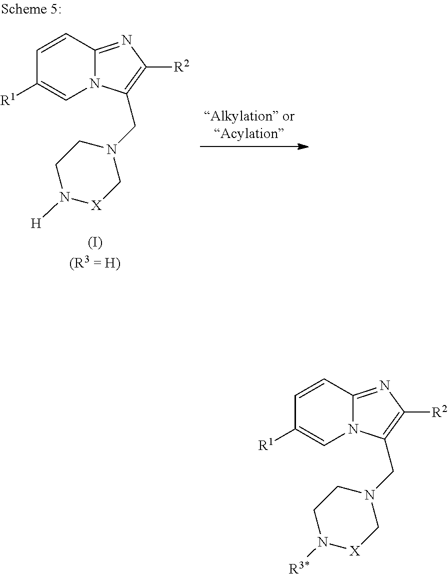

In schemes 1 to 5, R¹, R² and X have the aforementioned meanings. R³* is a radical R³ different from hydrogen or is a suitable N-protecting group. Suitable N-protecting groups are described, for example, in P. J. Kocienski “Protecting Groups”, 2nd ed., Georg Thieme Verlag, Stuttgart 2000, pp 186-237 or in Peter G. M. Wuts and Theodora W. Greene “Greene's Protective Groups in Organic Synthesis”, 4th ed., Wiley-Interscience, New Jersey 2007, pp 696-926, and in the literature cited therein. Preferred examples of N-protecting groups are e.g. oxycarbonyl groups such as C₁₀₆-alkoxycarbonyl, e.g. methoxycarbonyl, ethoxycarbonyl and Boc (tert-butoxycarbonyl) and other oxycarbonyl groups such as benzyloxycarbonyl (Cbz), allyloxycarbonyl, 9-fluorenylmethoxycarbonyl (Fmoc) and 2-trimethylsilylethoxycarbonyl (Teoc), or benzyl. R^{a} and Rb are hydroxyl or C₁-C₄-alkoxy such as methoxy or ethoxy or R^{a} and Rb together form a moiety O—R—O, wherein R is ethan-1,2-diyl, 1,1,2,2-tetramethylethan-1,2-diyl, propan-1,3-diyl, 2,2-dimethylpropan-1,3-diyl or 1,1,3-trimethylpropan-1,3-diyl. Hal is chlorine, bromine or iodine, preferably bromine or chlorine. “Pd” denominates a Pd(0) catalyst or a Pd(0) precursor compound, optionally in combination with a suitable ligand.

According to step i) of scheme 1, a suitable 2-aminopyridine compound II is reacted with an alpha bromo ketone compound III to give the imidazo[1,2-a]pyridine compound IV. Step i) is carried out in accordance with standard methods of organic chemistry and as described in the experimental part of this application. The reaction is usually carried out in the presence of a base. Suitable bases are, in general, inorganic compounds, such as alkali metal and alkaline earth metal carbonates, such as lithium carbonate, sodium carbonate, potassium carbonate, caesium carbonate and calcium carbonate, and also alkali metal bicarbonates, such as sodium bicarbonate.

The reaction is usually carried out in an inert organic solvent. Suitable solvents are C₁-C₆-alkanols such as methanol, ethanol or n-propanol, or mixtures of these solvents, or a mixture of C₁-C₆-alkanol(s) with water.

According to step ii) of scheme 1, the compound IV intermediate is reacted with a cyclic amine V and formaldehyde in the sense of a Mannich reaction in accordance with standard methods of organic chemistry and as described in the experimental part of this application to give compounds I′. Formaldehyde oligomers, e.g. trioxane, or polymers of formaldehyde, such as paraformaldehyde may be used instead of formaldehyde. Paraformaldehyde is preferably used.

If the 2-aminopyridine compound II is not available, compounds I′ can be prepared as depicted in scheme 2 and as described in the experimental part of this application. A skilled person will appreciate that step i) of scheme 2 can be performed under conditions as described for step i) of scheme 1. Similarly, the step ii) of scheme 2 can be performed under conditions as described for step ii) of scheme 1. According to step iii) of scheme 2, the halogen compound IX is treated with a boronic compound X, in a known manner, under conditions of a Suzuki coupling in the presence of a palladium catalyst to give the compound I′. The reaction is usually carried out in the presence of a base and a palladium catalyst, such as for example described in the following literature: Synth. Commun. Vol. 11, p. 513 (1981); Acc. Chem. Res. Vol. 15, pp. 178-184 (1982); Chem. Rev. Vol. 95, pp. 2457-2483 (1995); Organic Letters Vol. 6 (16), p. 2808 (2004); “Metal catalyzed cross coupling reactions”, 2^{nd} Edition, Wiley, VCH 2005 (Eds. De Meijere, Diederich); “Handbook of organopalladium chemistry for organic synthesis” (Eds Negishi), Wiley, Interscience, New York, 2002; “Handbook of functionalized organometallics”, (Ed. P. Knochel), Wiley, VCH, 2005 or in the experimental part of this application.

Suitable catalysts are in tetrakis(triphenylphosphine)palladium(0); bis(triphenylphosphine)palladium(II) chloride; bis(acetonitrile)palladium(II) chloride; [1,1′-bis(diphenylphosphino)ferrocene]-palladium(11) chloride/methylene chloride (1:1) complex; bis[bis-(1,2-diphenylphosphino)ethane]palladium(0); bis(bis-(1,2-diphenylphosphino)butane]-palladium(II) chloride; palladium(II) acetate; palladium(II) chloride; and palladium(II) acetate/tri-o-tolylphosphine complex or mixtures of phosphines and Pd salts or phosphines and Pd-complexes e.g. dibenzylideneacetone-palladium and tri-tert-butylphosphine (or its tetrafluoroborate), triscyclohexylphosphine; or a polymer-bound Pd-triphenylphosphine catalyst system.

Suitable bases are, in general, inorganic compounds, such as alkali metal and alkaline earth metal oxides, such as lithium oxide, sodium oxide, calcium oxide and magnesium oxide, alkali metal and alkaline earth metal carbonates, such as lithium carbonate, sodium carbonate, potassium carbonate, caesium carbonate and calcium carbonate, and also alkali metal bicarbonates, such as sodium bicarbonate, alkali metal and alkaline earth metal alkoxides, such as sodium methoxide, sodium ethoxide, potassium ethoxide and potassium tert.-butoxide, moreover organic bases, for example tertiary amines, such as trimethylamine, triethylamine, diisopropylethylamine and N-methylpiperidine, pyridine, substituted pyridines, such as collidine, lutidine and 4-dimethylaminopyridine, and also bicyclic amines, preferably potassium carbonate.

The reaction is usually carried out in an inert organic solvent. Suitable solvents are aliphatic hydrocarbons, such as pentane, hexane, cyclohexane and petroleum ether, aromatic hydrocarbons, such as toluene, o-, m- and p-xylene, ethers, such as diisopropyl ether, tert.-butyl methyl ether, 1,4-dioxane, anisole and tetrahydrofuran and dimethoxyethane, alkanol, e.g. C₁-C₆-alkanols such as methanol, ethanol or n-propanol, or mixtures of these solvents, particularly preferably ethers, such as dioxane or a mixture of toluene/methanol.

If the alpha bromo ketone compound III is not available, compounds I′ can be prepared as depicted in scheme 3 and as described in the experimental part of this application. In step i) a 2-aminopyridine compound II is reacted with ethyl bromoacetate XI and then with phosphoryl tribromide to give the compound XII. A skilled person will appreciate that step ii) of scheme 3 can be performed under conditions as described for step ii) of scheme 1. Similarly, step iii) of scheme 3 can be performed under conditions as described for step iii) of scheme 2.

If in the resulting imidazo[1,2-a]pyridine compound (I′) the radical R³* is not the desired radical R³ but a protecting group, e.g. benzyl, this substituent may be cleaved to obtain a compound wherein R³ is H, e.g. as depicted in scheme 4. The reaction conditions for the cleavage are known in the art. If R³* is an N-protective group (PG), the protective groups can be removed by standard methods, e.g. by the methods as described in P. J. Kocienski “Protecting Groups”, 2^{nd} ed., Georg Thieme Verlag, Stuttgart 2000, pp 186-237 or in Peter G. M. Wuts and Theodora W. Greene “Greene's Protective Groups in Organic Synthesis”, 4^{th} ed., Wiley-Interscience, New Jersey 2007, pp 696-926 and in the literature cited therein, such as treatment of the protected amine with an acid, e.g. halogen acid, such as HCl or HBr, formic acid or trifluoroacetic acid, or by hydrogenation, optionally in the presence of a Pd catalyst.

The imidazo[1,2-a]pyridine compound (I), wherein R³ is H can be reacted, in a known manner, in the sense of an alkylation (see scheme 5), with a compound R³-Lg. In this compound, R³ is C₁-C₆-alkyl, fluorinated C₁-C₂-alkyl, or C₁-C₄-alkoxy-C₁-C₄-alkyl and Lg is a nucleophilically displaceable leaving group, e.g. halogen, trifluoromethylsulfonate, alkylsulfonate, arylsulfonate, alkyl sulfate and the like.

The alkylation can also be achieved, in the sense of a reductive amination, by reacting the compound (I), wherein R³═H, with a suitable ketone or aldehyde in the presence of a reducing agent, e.g. in the presence of a borohydride such as sodium borohydride, sodium cyanoborohydride or sodium triacetoxyborohydride. The skilled person is familiar with the reaction conditions which are required for a reductive amination.

The imidazo[1,2-a]pyridine compound (I), wherein R³ is H, can also be reacted, in a known manner, in the sense of acylation (see scheme 5) with an acylating agent such as an acylhalide or an acylanhydride to obtain a compound of the formula I, wherein R³ is C₁-C₄-alkylcarbonyl, C₃-C₇-cycloalkylcarbonyl or C₁-C₂-fluoroalkyl-carbonyl.

The N-oxides of compounds of formula I can be prepared by, for example, treating a compound of the formula I with an oxidizing agent, in particular an inorganic or organic peroxide or hydroperoxide, such as hydrogen peroxide, or percarboxylic acids, such as peracetic acid, perbenzoic acid or m-chloroperbenzoic acid.

Besides the guidance provided in the general procedures below, alternative synthetic transformations that may be employed in the synthesis of compounds of formula (I) and in the synthesis of intermediates involved in the synthesis of compounds of formula (I) are known by or accessible to one skilled in the art. Collections of synthetic transformations may be found in compilations, such as: J. March. Advanced Organic Chemistry, 4th ed.; John Wiley: New York (1992) R. C. Larock. Comprehensive Organic Transformations, 2nd ed.; Wiley-VCH: New York (1999); F. A. Carey; R. J. Sundberg. Advanced Organic Chemistry, 2nd ed.; Plenum Press: New York (1984) T. W. Greene; P. G. M. Wuts. Protective Groups in Organic Synthesis, 3rd ed.; John Wiley: New York (1999). L. S. Hegedus, Transition Metals in the Synthesis of Complex Organic Molecules, 2nd ed.; University Science Books: Mill Valley, Calif. (1994) L. A. Paquette, Ed. The Encyclopedia of Reagents for Organic Synthesis; John Wiley: New York (1994). A. R. Katritzky; O. Meth-Cohn; C. W. Rees, Eds. Comprehensive Organic Functional Group Transformations; Pergamon Press: Oxford, UK (1995). G. Wilkinson; F. G A. Stone; E. W. Abel, Eds. Comprehensive Organometallic Chemistry; Pergamon Press: Oxford, UK (1982). B. M. Trost; I. Fleming, Comprehensive Organic Synthesis; Pergamon Press: Oxford, UK (1991) A. R. Katritzky; C. W. Rees Eds. Comprehensive Heterocylic Chemistry; Pergamon Press: Oxford, UK (1984) A. R. Katritzky; C. W. Rees; E. F. V. Scriven, Eds. Comprehensive Heterocylic Chemistry; Pergamon Press: Oxford, UK (1996). C. Hansch; P. G. Sammes; J. B. Taylor, Eds. Comprehensive Medicinal Chemistry: Pergamon Press: Oxford, UK (1990).

In addition, recurring reviews of synthetic methodology and related topics include Organic Reactions; John Wiley: New York; Organic Syntheses; John Wiley: New York; Reagents for Organic Synthesis: John Wiley: New York; The Total Synthesis of Natural Products; John Wiley: New York; The Organic Chemistry of Drug Synthesis; John Wiley: New York; Annual Reports in Organic Synthesis; Academic Press: San Diego Calif.; and Methoden der Organischen Chemie (Houben-Weyl); Thieme: Stuttgart, Germany. Furthermore, databases of synthetic transformations include Chemical Abstracts, which may be searched using either CAS OnLine or SciFinder, Handbuch der Organischen Chemie (Beilstein), which may be searched using Crossfire, and REACCS.

As shown in the examples below, the advantageous properties of the compounds of the invention include their activity as HIF inhibitors. For example, the compounds of the present invention were shown to inhibit the activation of HIF-mediated transcription under hypoxic conditions. Thus, the compounds of the invention can be used for the preparation of a medicament for the treatment of a disorder characterized by pathophysiological HIF signaling. A person skilled in the art of medical, biological and/or pharmacological science can determine with routine methodology if a disorder is characterized by undesirable HIF signaling. Tissues affected by such diseases will overexpress genes that are induced by activation of the HIF responsive element (HRE). HIF-1 acts by binding to HIF-responsive elements (HREs) in promoters that generally contain the sequence NCGTG. The genes affected by HIF activity which are regulated by said promoters are well known in the art and were also described in multiple reviews (see e.g. FIG. 3 of Gregg L. Semenza, Nature Reviews, October 2003, vol. 3).

In animal studies, HIF-1 overexpression is associated with increased tumor growth, increased vascularization, metastasis and fibrosis, e.g. renal fibrosis (see: Semenza, G., Drug Discovery Today, vol. 12, no. 19/20, October 2007; Kimura, Kuniko, et al., American Journal of Physiology (2008), 295(4, Pt. 2), F1023-F1029 and for a review see N. J. Mabjeesh et al., Histol. Histopathol (2007) 22:559-572). Fibrosis is the formation or development of excess fibrous connective tissue in an organ or tissue. Recently, it has become clear that inhibition of HIF-1 activity also acts to prevent inflammation, by virtue of its essential role in the activation and infiltration of macrophages and neutrophils into affected tissues (see e.g. Giaccia et al., Drug Discovery, vol. 2, October 2003).

For the above mentioned reasons, a compound of the present invention can be used to treat an inflammatory disease, a hyperproliferative disease or disorder, a hypoxia related pathology and also diseases characterized by pathophysiological hyper-vascularization. Therefore, as a further aspect, the invention provides a pharmaceutical composition comprising at least one compound of the present invention, optionally together with at least one physiologically acceptable carrier or auxiliary substance.

As a further aspect, the invention provides a therapeutical composition which, in addition to the compound of the invention comprises at least one further pharmaceutically active compound that is useful to treat one of the aforementioned diseases or disorders. Such therapeutical compositions are useful because the therapeutic efficiency of the compounds of the invention can be amplified by the presence of said at least one further pharmaceutically active compound and vice versa. For example, it was shown that inhibiting HIF1α activity via antisense gene therapy enhances the therapeutic efficacy of doxorubicin to combat hepatocellular carcinoma (see Liu, Fengjun et. al., Cancer Science (2008), 99(10), 2055-2061).

In a further aspect, the present invention relates to a pharmaceutical composition comprising a compound according to the invention and a second therapeutic agent useful for the treatment or prevention of a disease or disorder selected from the group consisting of an inflammatory disease, a hyperproliferative disease or disorder, a hypoxia related pathology and a disease characterized by pathophysiological hypervascularization, and, optionally, a pharmaceutically acceptable carrier or excipient. Such compositions are also useful to obtain synergistic therapeutic effects and also to prevent drug resistance of tumor cells, for example. It is also for these reasons, that current chemotherapy generally involves administering a cocktail of different cytotoxic and/or cytostatic compounds to improve the effectiveness of the treatment and reduce the possibility of tumor cell adaptation.

In a further aspect, the present invention relates to a pharmaceutical composition comprising a compound according to the invention in combination with radiation therapies.

Any composition of the present invention may be admixed with a pharmaceutically acceptable diluent, excipient or carrier, or a mixture thereof.

Even though the compounds of the present invention (including their pharmaceutically acceptable salts, their N-oxides, the salts of said N-oxides, ester derivatives and pharmaceutically acceptable solvates) can be administered alone, they will generally be administered in a mixture with a pharmaceutical carrier, excipient or diluent, particularly for human therapy. The pharmaceutical compositions may be for human or animal usage in human and veterinary medicine. Examples of such suitable excipients for the various different forms of pharmaceutical compositions described herein may be found in the “Handbook of Pharmaceutical Excipients”, 2nd Edition, (1994), Edited by A Wade and P J Weller. Acceptable carriers or diluents for therapeutic use are well known in the pharmaceutical art, and are described, for example, in Remington's Pharmaceutical Sciences, Mack Publishing Co. (A. R. Gennaro edit. 1985)

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material.

In powders, the carrier is a finely divided solid, which is in a mixture with the finely divided active component. In tablets, the active component is mixed with the carrier having the necessary binding properties in suitable proportions and compacted in the shape and size desired.

The powders and tablets preferably contain from 1% to 80%, more preferably from 5% to 60% of the active compound or active compounds. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. Liquid forms are particularly preferred for topical applications to the eye. For parenteral injection, liquid preparations can be formulated in solution as in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Interestingly, HIF inhibitors, such as the compounds of the invention, can prevent the development of tumor resistance towards chemotherapeutic drugs and can make cancer cells more sensitive towards radiotherapy (see e.g. Palayoor S T, et al., Int J Cancer. 2008 Nov. 15; 123(10):2430-7 and Gregg L. Semenza, Nature Reviews, October 2003, vol. 3). Thus, useful second therapeutic agents that can be combined with a compound of the invention to produce the pharmaceutical composition of the invention include, without limitation, a (further) HIF-1 inhibitor, a cytotoxic compound and cytostatic compounds.

A HIF-1 inhibitor can be, e.g. selected from the group consisting of PX-478 (S-2-amino-3-[4′-N,N,-bis(2-chloroethyl)amino]phenyl propionic acid N-oxide dihydrochloride); a topoisomerase-1 inhibitor such as 8,9-Dimethoxy-5-(2-N,N-dimethylaminoethyl)-2,3-methylenedioxy-5H-dibenzo[c,h][1,6]naphthyridin-6-one (also known as ARC-111 or topovale) or (S)-10-[(dimethylamino)methyl]-4-ethyl-4,9-dihydroxy-1H-pyrano[3′,′:6,7]indolizino[1,2-b]quinoline-3,14-(4H,12H)-dione monohydrochloride (also referred to as tropotecan); echinomycin; chetomin (NSC289491); cyclosporine A; 3-[2-[4-[bis(4-fluorophenyl)methylene]-1-piperidinyl]-2,3-dihydro-2-thioxo-4(1H)-quinazolinone (R59949); an inhibitor of the PIK3K/Akt/mTor signaling cascade, e.g., LY294002, wortmannin or rapamycin; an inhibitor of the MAPK signaling cascade, e.g. the MEK1 inhibitor PD98059; a soluble guanyl cyclase stimulator such as 3-(5′ hydroxymethyl-2′-furyl)-1-benzylindazole (YC-1); a heat-shock protein 90 inhibitor, in particular radicicol, the radicicol analogue KF58333 or geldanamycin; a microtubule disrupting agent, in particular e.g. taxol, vincristine or 2-methoxyestradiol; a histone deacetylase inhibitor, e.g. FK228; a thioredoxin inhibitor, in particular PX-12 or pleurotin; UCNO-1; diphenylene iodonium, genestein and carboxyamido-triazole.

Many cytotoxic or cytostatic compounds are known to the expert artisan skilled in the therapy of hyperproliferative diseases or disorders such as a tumor or cancer disease. For example, cytotoxic and cytostatic compounds include, but are not limited to, pure or mixed anti-estrogens such as faslodex, tamoxifen or raloxifen; any inhibitors of topoisomerase I or II, such as camptothecin (topo I) or etoposide (topo II); any compound that acts through inhibiting aromatase activity, such as anastrozole or letrozole; any preparation that interferes with HER2 signaling such as herceptin; any compound that intercalates DNA, such as doxorubicin. Particularly preferred cytostatic or cytotoxic drugs, which can be combined with the compounds of the present invention are alkylating substances, anti-metabolites, antibiotics, epothilones, nuclear receptor agonists and antagonists, anti-androgenes, anti-estrogens, platinum compounds, hormones and antihormones, interferons and inhibitors of cell cycle-dependent protein kinases (CDKs), inhibitors of cyclooxygenases and/or lipoxygenases, biogeneic fatty acids and fatty acid derivatives, including prostanoids and leukotrienes, inhibitors of protein kinases, inhibitors of protein phosphatases, inhibitors of lipid kinases, platinum coordination complexes, ethyleneimenes, methylmelamines, trazines, vinca alkaloids, pyrimidine analogs, purine analogs, alkylsulfonates, folic acid analogs, anthracendiones, substituted urea, methylhydrazin derivatives, in particular acediasulfone, aclarubicine, ambazone, aminoglutethimide, L-asparaginase, azathioprine, bleomycin, busulfan, calcium folinate, carboplatin, carpecitabine, carmustine, celecoxib, chlorambucil, cis-platin, cladribine, cyclophosphamide, cytarabine, dacarbazine, dactinomycin dapsone, daunorubicin, dibrompropamidine, diethylstilbestrole, docetaxel, doxorubicin, enediynes, epirubicin, epothilone B, epothilone D, estramucin phosphate, estrogen, ethinylestradiole, etoposide, flavopiridol, floxuridine, fludarabine, fluorouracil, fluoxymesterone, flutamide fosfestrol, furazolidone, gemcitabine, gonadotropin releasing hormone analogue, hexamethylmelamine, hydroxycarbamide, hydroxymethylnitrofurantoin, hydroxyprogesteronecaproate, hydroxyurea, idarubicin, idoxuridine, ifosfamide, interferon γ, irinotecan, leuprolide, lomustine, lurtotecan, mafenide sulfate olamide, mechlorethamine, medroxyprogesterone acetate, megastrolacetate, melphalan, mepacrine, mercaptopurine, methotrexate, metronidazole, mitomycin C, mitopodozide, mitotane, mitoxantrone, mithramycin, nalidixic acid, nifuratel, nifuroxazide, nifuralazine, nifurtimox, nimustine, ninorazole, nitrofurantoin, nitrogen mustards, oleomucin, oxolinic acid, pentamidine, pentostatin, phenazopyridine, phthalylsulfathiazole, pipobroman, prednimustine, prednisone, preussin, procarbazine, pyrimethamine, raltitrexed, rapamycin, rofecoxib, rosiglitazone, salazosulfapyridine, scriflavinium chloride, semustine streptozocine, sulfacarbamide, sulfacetamide, sulfachlopyridazine, sulfadiazine, sulfadicramide, sulfadimethoxine, sulfaethidole, sulfafurazole, sulfaguanidine, sulfaguanole, sulfamethizole, sulfamethoxazole, co-trimoxazole, sulfamethoxydiazine, sulfamethoxypyridazine, sulfamoxole, sulfanilamide, sulfaperin, sulfaphenazole, sulfathiazole, sulfisomidine, staurosporin, tamoxifen, taxol, teniposide, tertiposide, testolactone, testosteronpropionate, thioguanine, thiotepa, tinidazole, topotecan, triaziquone, treosulfan, trimethoprim, trofosfamide, UCN-01, vinblastine, vincristine, vindesine, vinblastine, vinorelbine, and zorubicin, or their respective derivatives or analogs thereof. Several of the above indicated drugs are now administered simultaneously for cancer therapy and, consequently, it is also envisioned that more than one cytostatic and/or cytotoxic drug can be comprised in compositions of the present invention.

As mentioned above, HIF inhibitors render cancer cells more vulnerable to chemotherapy and radiation therapy. Thus, to effectively treat a hyperproliferative disease or disorder, the compounds of the present invention can be co-administered with other active medicinal agents and/or administered in conjunction with other anticancer, antitumor, or antiproliferative disease therapies. In one aspect, the invention provides a method for treating a hyperproliferative disease or disorder comprising administering a compound according to the invention to a patient prior to, during and/or after said patient was subjected to a radiation therapy, a chemotherapy, an immunotherapy, a laser/microwave thermotherapy or a gene therapy using antisense DNA and RNA (for examples see Moeller et al., Cancer Cell 2004 5429-441).

In a further aspect the invention provides, as already outlined above, the use of a compound according to the invention or a composition according to the invention for the preparation of a medicament for the therapy, including the treatment or prevention, of a disease or disorder selected from the group consisting of an inflammatory disease, a hyperproliferative disease or disorder, a hypoxia related pathology such as e.g. diabetic retinopathy, ischemic reperfusion injury, ischemic myocardial and limb disease, ischemic stroke, sepsis and septic shock (see, e.g. Liu F Q, et al., Exp Cell Res. 2008 Apr. 1; 314(6):1327-36); and a disease characterized by pathophysiological hyper-vascularization, such as e.g. angiogenesis in osteosarcoma (see, e.g.: Yang, Qing-cheng et al., Dier Junyi Daxue Xuebao (2008), 29(5), 504-508), macular degeneration, in particular, age-related macular degeneration and vasoproliferative retinopathy (see e.g. Kim J H, et al., J Cell Mol. Med. 2008 Jan. 19).

As was already mentioned above, HIF inhibitors, such as the compounds of the invention, are useful to treat inflammatory disease or disorder. For example, it was shown that oxygen-dependent HIF isoforms are strongly upregulated in psoriatic skin (see e.g. Rosenberger C, et al., J Invest Dermatol. 2007 October; 127(10):2445-52). Furthermore it was shown that a HIF inhibitor, neovastat, inhibits the airway inflammation in asthma (see e.g., Lee S Y, et al., Vascul Pharmacol. 2007 November-December; 47(5-6):313-8). Furthermore, recent evidence also shows that HIF participates under hypoxic conditions in joint inflammation and destruction in rheumatoid arthritis (see e.g., Ahn, J. K., et al., Rheumatology (Oxford, United Kingdom) (2008), 47(6), 834-839). Thus, in a preferred embodiment of the use of the invention, the inflammatory disease is selected form the group consisting of atherosclerosis, rheumatoid arthritis, asthma, inflammatory bowel disease, psoriasis, in particular psoriasis vulgaris, psoriasis capitis, psoriasis guttata, psoriasis inversa; neurodermatitis; ichtyosis; alopecia greata; alopecia totalis; alopecia subtotalis; alopecia universalis; alopecia diffusa; atopic dermatitis; lupus erythematodes of the skin; dermatomyositis of the skin; atopic eczema; morphea; scleroderma; alopecia greata Ophiasis type; androgenic alopecia; allergic dermatitis; irritative contact dermatitis; contact dermatitis; pemphigus vulgaris; pemphigus foliaceus; pemphigus vegetans; scarring mucous membrane pemphigoid; bullous pemphigoid; mucous membrane pemphigoid; dermatitis; dermatitis herpetiformis Duhring; urticaria; necrobiosis lipoidica; erythema nodosum; prurigo simplex; prurigo nodularis; prurigo acuta; linear IgA dermatosis; polymorphic light dermatosis; erythema solaris; exanthema of the skin; drug exanthema; purpura chronica progressiva; dihydrotic eczema; eczema; fixed drug exanthema; photoallergic skin reaction; and perioral dermatitis. Therefore, a further preferred embodiment of the present invention encompasses a combination of one or more compounds of the present invention and medication in current use for treating such inflammatory diseases or conditions, which can be determined by a person skilled in the art of pharmacological sciences. Such therapeutics for combination can be selected e.g. from a group of anti-inflammatory steroids, antioxidants, therapeutic antibodies or fusion proteins that sequester or bind to certain cytokines or cellular epitopes associated with inflammatory processes, or a dihydrofolate reductase inhibitor like methotrexate.

The compounds of the invention show anti-proliferative effects. Furthermore, HIF inhibitors, such as the compounds of the invention are effective medicaments for the treatment of various cancer diseases (see review article by e.g. Gregg L. Semenza, Nature Reviews, October 2003, vol. 3 and also review article by N. J. Mabjeesh et al., Histol. Histopathol (2007), 22:559-572). Thus, also preferred is the use of the invention wherein the hyperproliferative disease is selected from the group consisting of a tumor or cancer disease, precancerosis, dysplasia, histiocytosis, a vascular proliferative disease and a virus-induced proliferative disease. Thus, in one preferred embodiment of the use of the invention the hyperproliferative disease is a tumor or cancer disease selected from the group consisting of diffuse large B-cell lymphoma (DLBCL), T-cell lymphomas or leukemias, e.g., cutaneous T-cell lymphoma (CTCL), noncutaneous peripheral T-cell lymphoma, lymphoma associated with human T-cell lymphotrophic virus (HTLV), adult T-cell leukemia/lymphoma (ATLL), as well as acute lymphocytic leukemia, acute nonlymphocytic leukemia, acute myeloid leukemia, chronic lymphocytic leukemia, chronic myelogenous leukemia, Hodgkin's disease, non-Hodgkin's lymphoma, myeloma, multiple myeloma, mesothelioma, childhood solid tumors, glioma, bone cancer and soft-tissue sarcomas, common solid tumors of adults such as head and neck cancers (e.g., oral, laryngeal and esophageal), genitourinary cancers (e.g., prostate, bladder, renal (in particular malignant renal cell carcinoma (RCC)), uterine, ovarian, testicular, rectal, and colon), lung cancer (e.g., small cell carcinoma and non-small cell lung carcinoma, including squamous cell carcinoma and adenocarcinoma), breast cancer, pancreatic cancer, melanoma and other skin cancers, basal cell carcinoma, metastatic skin carcinoma, squamous cell carcinoma of both ulcerating and papillary type, stomach cancer, brain cancer, liver cancer, adrenal cancer, kidney cancer, thyroid cancer, medullary carcinoma, osteosarcoma, soft-tissue sarcoma, Ewing's sarcoma, veticulum cell sarcoma, and Kaposi's sarcoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, leiomyosarcoma, rhabdomyosarcoma, squamous cell carcinoma, adenocarcinoma, sweat gland carcinoma, sebaceous gland carcinoma, papillary carcinoma, glioblastoma, papillary adenocarcinomas, cystadenocarcinoma, bronchogenic carcinoma, seminoma, embryonal carcinoma, Wilms' tumor, small cell lung carcinoma, epithelial carcinoma, astrocytoma, medulloblastoma, craniopharyngioma, ependymoma, pinealoma, hemangioblastoma, acoustic neuroma, oligodendroglioma, meningioma, neuroblastoma, retinoblastoma, glaucoma, hemangioma, heavy chain disease and metastases.

The precancerosis treatable with the compounds of the present invention are preferably selected from the group consisting of precancerosis, in particular actinic keratosis, cutaneaous horn, actinic cheilitis, tar keratosis, arsenic keratosis, x-ray keratosis, Bowen's disease, bowenoid papulosis, lentigo maligna, lichen sclerosus, and lichen rubber mucosae; precancerosis of the digestive tract, in particular erythroplakia, leukoplakia, Barrett's esophagus, Plummer-Vinson syndrome, crural ulcer, gastropathia hypertrophica gigantea, borderline carcinoma, neoplastic intestinal polyp, rectal polyp, porcelain gallbladder; gynaecological precancerosis, in particular carcinoma ductale in situ (CDIS), cervical intraepithelial neoplasia (CIN), endometrial hyperplasia (grade III), vulvar dystrophy, vulvar intraepithelial neoplasia (VIN), hydatidiform mole; urologic precancerosis, in particular bladder papillomatosis, Queyrat's erythroplasia, testicular intraepithelial neoplasia (TIN), carcinoma in situ (CIS); precancerosis caused by chronic inflammation, in particular pyoderma, osteomyelitis, acne conglobata, lupus vulgaris, and fistula.

Dysplasia is frequently a forerunner of cancer, and is can be found in e.g. the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation. Dysplastic disorders which can be treated with the compounds of the present invention include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis heminelia, dysplasia epiphysialis multiplex, dysplasia epiphysalis punctata, epithelial dysplasia, faciodigitogenital dysplasia, familial fibrous dysplasia of jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysical dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, ophthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

A compound according to the invention can be administered by various well known routes, including oral, rectal, intragastrical, intracranial and parenteral administration, e.g. intravenous, intramuscular, intranasal, intradermal, subcutaneous, and similar administration routes. Parenteral administration and particular intravenous administration, preferably by depot injection, is preferred. Depending on the route of administration different pharmaceutical formulations are required and some of those may require that protective coatings are applied to the drug formulation to prevent degradation of a compound of the invention in, for example, the digestive tract.

Thus, preferably, a compound of the invention is formulated as a syrup, an infusion or injection solution, a tablet, a capsule, a caplet, lozenge, a liposome, a suppository, a plaster, a band-aid, a retard capsule, a powder, or a slow release formulation. Preferably the diluent is water, a buffer, a buffered salt solution or a salt solution and the carrier preferably is selected from the group consisting of cocoa butter and vitebesole.

Particular preferred pharmaceutical forms for the administration of a compound of the invention are forms suitable for injectionable use and include sterile aqueous solutions or dispersions and sterile powders for the extemporaneous preparation of sterile injectable solutions or dispersion. In all cases, the final solution or dispersion form must be sterile and fluid. Typically, such a solution or dispersion will include a solvent or dispersion medium, containing, for example, water-buffered aqueous solutions, e.g. biocompatible buffers, ethanol, polyol, such as glycerol, propylene glycol, polyethylene glycol, suitable mixtures thereof, surfactants or vegetable oils. A compound of the invention can also be formulated into liposomes, in particular for parenteral administration. Liposomes provide the advantage of increased half life in the circulation, if compared to the free drug and a prolonged more even release of the enclosed drug.

Sterilization of infusion or injection solutions can be accomplished by any number of art recognized techniques, including but not limited to, addition of preservatives like anti-bacterial or anti-fungal agents, e.g. parabene, chlorobutanol, phenol, sorbic acid or thimersal. Further, isotonic agents, such as sugars or salts, in particular sodium chloride may be incorporated in infusion or injection solutions.

Production of sterile injectable solutions containing one or several of the compounds of the invention is accomplished by incorporating the respective compound in the required amount in the appropriate solvent with various ingredients enumerated above as required followed by sterilization. To obtain a sterile powder the above solutions are vacuum-dried or freeze-dried as necessary. Preferred diluents of the present invention are water, physiologically acceptable buffers, physiologically acceptable buffer salt solutions or salt solutions. Preferred carriers are cocoa butter and vitebesole. Besides the preferred excipients mentioned already above, also the following excipients can be chosen, without limitation, to be used with the various pharmaceutical forms of a compound of the invention:
a) binders such as lactose, mannitol, crystalline sorbitol, dibasic phosphates, calcium phosphates, sugars, microcrystalline cellulose, carboxymethyl cellulose, hydroxyethyl cellulose, polyvinyl pyrrolidone and the like;
b) lubricants such as magnesium stearate, talc, calcium stearate, zinc stearate, stearic acid, hydrogenated vegetable oil, leucine, glycerids and sodium stearyl fumarates,
c) disintegrants such as starches, croscarmellose, sodium methyl cellulose, agar, bentonite, alginic acid, carboxymethyl cellulose, polyvinyl pyrrolidone and the like.

Other suitable excipients can be found in the Handbook of Pharmaceutical Excipients, published by the American Pharmaceutical Association, which is herein incorporated by reference.

It is to be understood that depending on the severity and the particular type of the disorder which is treatable with one of the compounds of the invention, as well as on the respective patient to be treated, e.g. the general health status of the patient, etc., different doses of the respective compound are required to elicit a therapeutic or prophylactic effect. The determination of the appropriate dose lies within the discretion of the attending physician. It is contemplated that the average daily dosage of a compound of the invention in the therapeutic or prophylactic use of the invention should be in the range of about 0.1 mg to about 3 g. However, in a preferred use of the present invention a compound of the invention is administered to a subject in need thereof in an amount ranging from 1.0 to 1000 mg, more preferably ranging from 10 to 500 mg, most preferably ranging from 20 to 200 mg. The duration of therapy and the dosing frequency with a compound of the invention will vary, depending on the severity of the disease being treated and the condition and idiosyncratic response of each individual patient.

As is known in the art, the pharmaceutically effective amount of a given composition will also depend on the administration route. In general the required amount will be higher, if the administration is through the gastrointestinal tract; e.g. by suppository, rectal, or by an intragastric probe, and lower if the route of administration is parenteral, e.g. intravenous. Typically, a compound of the invention will be administered in ranges of 20 mg to 3 g, preferably 20 mg to 500 mg, if rectal or intragastric administration is used and in ranges of 10 to 500 mg, if parenteral administration is used.

If a person is known to be at risk of developing a disorder treatable with a compound of the invention, a prophylactic administration of the pharmaceutical composition according to the invention may be possible. In these cases, the respective compound of the invention is preferably administered in above outlined preferred and particular preferred doses on a daily basis. This administration can be continued until the risk of developing the respective disorder has lessened. In most instances, however, a compound of the invention will be administered once a disease/disorder has been diagnosed. In these cases it is preferred that a first dose of a compound of the invention is administered one, two, three or four times daily. Preferably the administration is discontinued for one day, one week or one month and then repeated until the symptoms of the respective disease are no longer worsening or until they are improving.

Within the meaning of this invention, a combination of substituents or variables is permissible only if such a combination results in a stable or chemically feasible compound. A stable compound or chemically feasible compound is one that is not substantially altered when kept at a temperature of 40° C. or less, in the absence of moisture or other chemically reactive conditions, for at least a week. This invention also envisions the quaternization of any basic nitrogen-containing groups of the compounds disclosed herein. Water or oil-soluble or dispersible products may be obtained by such quaternization.

Various modifications and variations of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

The following examples and figures are merely illustrative of the present invention and should not be construed to limit the scope of the invention, as indicated by the appended claims, in any way.

### Preparation Examples

### Synthesis of Building Blocks

The starting materials used in the examples are either commercially available or can be synthesized by the average skilled person trained in organic chemistry without undue burden following routine laboratory practice or as outlined for example below. For example, 2-aminopyridine compounds in question are either commercially available or have been synthesized according to literature procedures or as described below; alpha-bromo ketone compounds in question are either commercially available or have been synthesized according to literature procedures or as described below

The following abbreviations are used hereinafter:

Boc: tert. butoxycarbonyl

DCM: Dichloromethane

DMF: Dimethylformamide

Et: Ethyl

TFA: Trifluoroacetic acid

Pd(dppf)Cl₂: 1,1′-Bis(diphenylphosphino)ferrocene]dichloropalladium

Imidazo[1,2-a]pyridine compounds of the formula (I) according to the present invention can be prepared for example according to the following approaches 1 to 4. A skilled person will readily understand that compounds 4, wherein R¹ is R³ correspond to compounds I and that in compounds 4, wherein R¹ is a protecting group, such as Boc, the protecting group can be removed as shown in the extra step 1 to give compounds I, wherein R³ is H. The skilled person will also readily understand that a compound 5 correspond to a compound I with R³═H. The skilled person will also readily understand that compounds 12, wherein R″ is lower alkyl, cycloalkyl or lower fluoroalkyl correspond to compounds I, where R³ is C(O)R⁴ with R⁴ being C₁-C₄-alkyl, C₃-C₇-cycloalkyl or fluorinated C₁-C₂-alkyl.

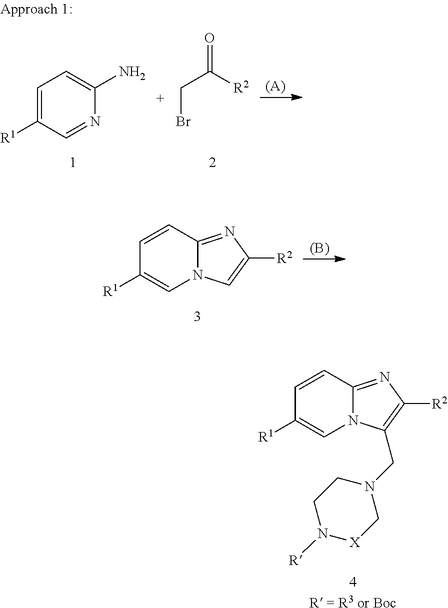

Approach 1 may include an additional step, i.e. the extra step 1, in order to deprotect compounds 4 with R¹ being the N-protecting group, such as Boc.

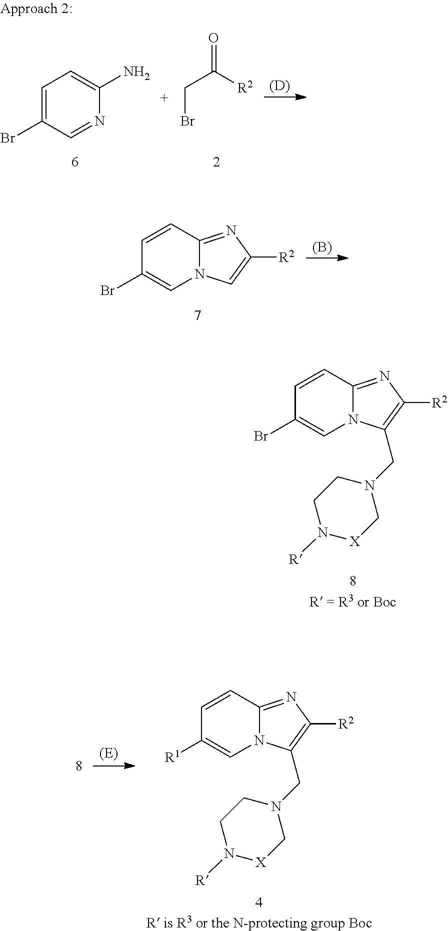

Approach 2 may include the extra step 1 to deprotect compounds 4 with R¹ being the N-protecting group Boc.

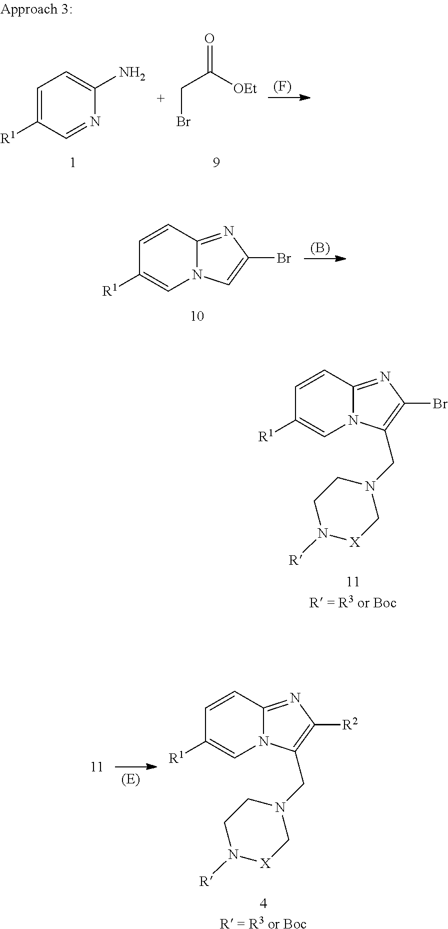

Approach 3 may include the extra step 1 to deprotect compounds 4 with R¹ being the N-protecting group Boc.

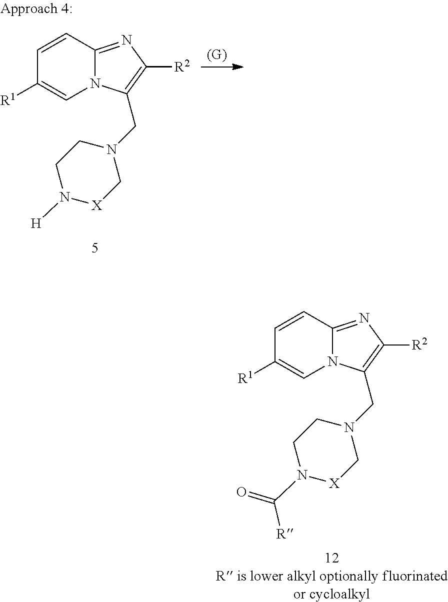

Extra Step 1: Deprotection of compounds 4, where R¹ is Boc

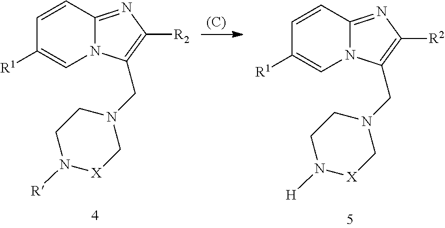

The methods A, B, C, D, E, F and G depicted in the schemes “Approach 1”, “Approach 2”, “Approach 3, “Approach 4” and “Extra step 1” indicated above can be performed as follows:

General Procedure A:

Compounds 1 (9.31 mmol, 1.0 equiv.) and 2 (10.24 mmol, 1.1 equiv.) were dissolved in ethanol (10 ml) and water (10 ml). NaHCO₃ (10.24 mmol, 1.1 equiv.) was then added to the reaction mixture and it was heated to reflux for 18 h. The reaction mixture was poured into ethyl acetate (150 ml) and was washed with water (3×150 ml). The organic phase was dried over Na₂SO₄, filtered and evaporated. The residue was purified by automated column chromatography using the ISCO Combi Flash system eluting with ethyl acetate in cyclohexane (0-100%). This afforded compound 3 in 15-62% yield.

General Procedure B:

To a suitably substituted cyclic amine (2.44 mmol, 2.0 equiv.), dissolved in DMF (2 ml) with paraformaldehyde (2.44 mmol, 2.0 equiv.), acetic acid (2.44 mmol, 2.0 equiv.) was added. The reaction mixture was stirred at room temperature for 1 h. A solution of 3, 7 or 10 (1.22 mmol, 1.0 equiv.) in DMF (3 m l) was added and the reaction mixture was heated to 60° C. for 1-4 h. The reaction mixture was poured into ethyl acetate (100 ml) and washed with saturated NaHCO₃ solution (3×100 ml). The organic phase was dried over Na₂SO₄, filtered and evaporated. The crude product was purified by automated column chromatography using the ISCO Combi Flash system eluting with ethyl acetate in cyclohexane (0-100%) or by preparative HPLC-MS. This afforded compounds 4, 8 or 11 in 43-78% yield.

General Procedure C (Extra Step 1):

Compound 4 (0.041 mmol, 1.0 equiv.) was dissolved in DCM (1 ml) and TFA (1 ml) was added. The reaction mixture was stirred at room temperature for 20 min. The solvent was removed in vacuo and the residue was dissolved in ethyl acetate (10 ml) and washed with saturated NaHCO₃ (aq) (3×10 ml). The organic phase was dried over Na₂SO₄, filtered and evaporated. This afforded compound 5 in 63-100% yield.

General Procedure D:

Compounds 6 (7.301 mmol, 1.0 equiv.) and 2 (8.03 mmol, 1,1 equiv.) were suspended in ethanol (20 ml) and the reaction mixture was heated to reflux for 18 h. Upon reaction completion the reaction mixture was cooled and poured into ethyl acetate (100 ml) and the solution was washed with saturated NaHCO₃ solution (2×100 ml). The combined aqueous phases were extracted with ethyl acetate (200 ml). The combined organic phases were dried over Na₂SO₄, filtered and evaporated. This afforded compound 7 in 63-100% yield.

General Procedure E:

Compound 8 or 11 (0.058 mmol, 1.0 equiv.), a suitably substituted boronic acid or ester (0.117 mmol, 2.0 equiv.) and Pd(dppf)Cl₂ (0.005 mmol, 0.1 equiv.) were dissolved in dioxane (1 ml). A 2M aqueous solution of K₂CO₃ (0.147 mmol, 2.5 equiv.) was added and the reaction mixture was heated to 110° C. in a microwave apparatus for 30 min. The reaction mixture was poured into ethyl acetate (20 ml) and washed with saturated NaHCO₃ solution (3×20 ml). The organic phase was dried over Na₂SO₄, filtered and evaporated. The crude product was purified by automated column chromatography using the ISCO Combi Flash system eluting with ethyl acetate in cyclohexane (0-100%) or by preparative HPLC-MS. This afforded compound 4 in 27-58% yield.

General Procedure F:

Compound 1 (11.75 mmol) was dissolved in compound 9 (10 ml) and the reaction mixture was stirred at room temperature for 18 h. The excess 9 was removed in vacuo. To fully remove 9, the mixture was evaporated with toluene three times. The solid was washed with diethyl ether and collected by filtration. This solid (1.48 mmol, 1.0 equiv.) was then suspended in acetonitrile (7 ml) and POBr₃ (2.96 mmol, 2.0 equiv.) was added and the reaction mixture was heated to reflux for 18 h. It was then poured into ice cooled 1M NaOH (aq) (100 ml). This suspension was stirred for 15 min and then extracted with ethyl acetate (3×100 ml). The combined organic phases were dried over Na₂SO₄, filtered and evaporated. The crude product was purified by automated column chromatography using the ISCO Combi Flash system eluting with ethyl acetate in cyclohexane (0-100%). This afforded compound 10 in 10-36% yield.

General Procedure G:

Compound 5 (0.048 mmol, 1 equiv.) was dissolved in DCM (1 ml) and to this solution was added a suitably substituted acid chloride (0.058 mmol, 1.2 equiv.), followed by triethylamine (0.058 mmol, 1.2 equiv.). The reaction mixture was stirred at room temperature for up to 18 h. Upon reaction completion it was poured into ethyl acetate (5 ml) and washed with saturated NaHCO₃ (aq) (3×5 ml). The organic phase was dried over Na₂SO₄, filtered and evaporated. The residue was purified by preparative HPLC-MS to afford compound 12 in 27-37% yield.

Analytical Procedures:

Compounds I were analyzed as follows:

Measured via HPLC/MS, using a Waters X-bridge C₁₈-column, 5 μm particle size, 4.6×150 mm (diameter×length) at a flow rate of 1.75 ml/min with a linear gradient (water to acetonitrile, 0.2% formic acid as modifier) from initially 99:1 to 1:99 over 9.10 min, then held for 1.80 min. Mass signals were determined using a Waters 3100 Mass Detector.

The following compounds of the formula (I) listed in table I below were prepared using the standard operation procedures described above.

TABLE I

| Example | IUPAC-Name | Rₜ^{#} [min] | m/z + 1 |
|---|---|---|---|
| 1 | 2-(4-nitrophenyl)-6-phenyl-3-(piperazin-1- | | |
| | ylmethyl)imidazo[1,2-a]pyridine | | |
| 2 | 1-(4-((2-(3-nitrophenyl)-6-phenylimidazo[1,2-a]pyridin-3- | | |
| | yl)methyl)piperazin-1-yl)ethanone | | |
| 3 | 1-(4-((2-(4-nitrophenyl)-6-phenylimidazo[1,2-a]pyridin-3- | | |
| | yl)methyl)piperazin-1-yl)ethanone | | |
| 4 | 3-((1,4-diazepan-1-yl)methyl)-2-(4-nitrophenyl)-6- | 4.60 | 428 |
| | phenylimidazo[1,2-a]pyridine | | |
| 5 | 2-(4-nitrophenyl)-6-phenyl-3-((4-propylpiperazin-1- | 5.12 | 456 |
| | yl)methyl)imidazo[1,2-a]pyridine | | |
| 6 | 3-((4-ethylpiperazin-1-yl)methyl)-2-(4-nitrophenyl)-6- | 5.04 | 442 |
| | phenylimidazo[1,2-a]pyridine | | |
| 7 | 3-((4-methylpiperazin-1-yl)methyl)-2-(4-nitrophenyl)-6- | 4.39 | 428 |
| | phenylimidazo[1,2-a]pyridine | | |
| 8 | 3-((1,4-diazepan-1-yl)methyl)-2-(4-(methylsulfonyl)phenyl)- | 4.12 | 461 |
| | 6-phenylimidazo[1,2-a]pyridine | | |
| 9 | 4-(3-((1,4-diazepan-1-yl)methyl)-6-phenylimidazo[1,2- | 4.25 | 408 |
| | a]pyridin-2-yl)benzonitrile | | |
| 10 | 4-(3-((1,4-diazepan-1-yl)methyl)-6-phenylimidazo[1,2- | 3.67 | 426 |
| | a]pyridin-2-yl)benzamide | | |
| 11 | 3-((1,4-diazepan-1-yl)methyl)-6-phenyl-2-(pyridin-4- | 3.85 | 384 |
| | yl)imidazo[1,2-a]pyridine | | |
| 12 | 3-((4-methyl-1,4-diazepan-1-yl)methyl)-2-(4-nitrophenyl)-6- | 4.75 | 442 |
| | phenylimidazo[1,2-a]pyridine | | |
| 13 | 4-((2-(4-nitrophenyl)-6-phenylimidazo[1,2-a]pyridin-3- | 5.11 | 428 |
| | yl)methyl)piperazin-2-one | | |
| 14 | 3-((1,4-diazepan-1-yl)methyl)-2-(4-chlorophenyl)-6- | 4.76 | 417 |
| | phenylimidazo[1,2-a]pyridine | | |
| 15 | 3-((1,4-diazepan-1-yl)methyl)-6-phenyl-2-(pyridin-3- | 3.60 | 384 |
| | yl)imidazo[1,2-a]pyridine | | |
| 16 | 5-(3-((1,4-diazepan-1-yl)methyl)-6-phenylimidazo[1,2- | 4.49 | 409 |
| | a]pyridin-2-yl)picolinonitrile | | |
| 17 | 3-((1,4-diazepan-1-yl)methyl)-6-phenyl-2-(p- | 4.34 | 397 |
| | tolyl)imidazo[1,2-a]pyridine | | |
| 18 | 3-((1,4-diazepan-1-yl)methyl)-2-(4-methoxyphenyl)-6- | 4.11 | 413 |
| | phenylimidazo[1,2-a]pyridine | | |
| 19 | 3-((1,4-diazepan-1-yl)methyl)-2,6-diphenylimidazo[1,2- | 3.95 | 383 |
| | a]pyridine | | |
| 20 | 5-(3-((1,4-diazepan-1-yl)methyl)-6-phenylimidazo[1,2- | 5.13 | 414 |
| | a]pyridin-2-yl)thiophene-3-carbonitrile | | |
| 21 | 5-(3-((1,4-diazepan-1-yl)methyl)-6-phenylimidazo[1,2- | 5.28 | 414 |
| | a]pyridin-2-yl)thiophene-2-carbonitrile | | |
| 22 | 3-(3-((1,4-diazepan-1-yl)methyl)-6-phenylimidazo[1,2- | 4.89 | 408 |
| | a]pyridin-2-yl)benzonitrile | | |
| 23 | 4-(3-((1,4-diazepan-1-yl)methyl)-6-(p-tolyl)imidazo[1,2- | 5.23 | 422 |
| | a]pyridin-2-yl)benzonitrile | | |
| 24 | 4-(3-((1,4-diazepan-1-yl)methyl)-6-(m-tolyl)imidazo[1,2- | 4.84 | 422 |
| | a]pyridin-2-yl)benzonitrile | | |
| 25 | 4-(3-((1,4-diazepan-1-yl)methyl)-6-(o-tolyl)imidazo[1,2- | 4.50 | 422 |
| | a]pyridin-2-yl)benzonitrile | | |
| 26 | 4-(3-((1,4-diazepan-1-yl)methyl)-6-(pyrimidin-5- | 3.58 | 410 |
| | yl)imidazo[1,2-a]pyridin-2-yl)benzonitrile | | |
| 27 | 4-(3-((1,4-diazepan-1-yl)methyl)-6-(pyridin-4- | 3.33 | 409 |
| | yl)imidazo[1,2-a]pyridin-2-yl)benzonitrile | | |
| 28 | 4-(3-((1,4-diazepan-1-yl)methyl)-6-(2-methylpyridin-4- | 3.98 | 423 |
| | yl)imidazo[1,2-a]pyridin-2-yl)benzonitrile | | |
| 29 | 4-(3-((1,4-diazepan-1-yl)methyl)-6-(pyridin-3- | 3.57 | 409 |
| | yl)imidazo[1,2-a]pyridin-2-yl)benzonitrile | | |
| 30 | 4-((2-(4-methoxyphenyl)-6-phenylimidazo[1,2-a]pyridin-3- | 4.64 | 413 |
| | yl)methyl)piperazin-2-one | | |
| 31 | 2-(4-chlorophenyl)-3-((4-methyl-1,4-diazepan-1-yl)methyl)- | 4.58 | 431 |
| | 6-phenylimidazo[1,2-a]pyridine | | |
| 32 | 4-((2-(4-chlorophenyl)-6-phenylimidazo[1,2-a]pyridin-3- | 4.99 | 417 |
| | yl)methyl)piperazin-2-one | | |
| 33 | 4-(3-((3-oxopiperazin-1-yl)methyl)-6-phenylimidazo[1,2- | 4.65 | 408 |
| | a]pyridin-2-yl)benzonitrile | | |
| 34 | 4-(3-((4-methyl-1,4-diazepan-1-yl)methyl)-6- | 5.96 | 422 |
| | phenylimidazo[1,2-a]pyridin-2-yl)benzonitrile | | |
| 35 | 5-(3-((3-oxopiperazin-1-yl)methyl)-6-phenylimidazo[1,2- | 5.85 | 414 |
| | a]pyridin-2-yl)thiophene-2-carbonitrile | | |
| 36 | 5-(3-((4-methyl-1,4-diazepan-1-yl)methyl)-6- | 5.72 | 428 |
| | phenylimidazo[1,2-a]pyridin-2-yl)thiophene-2-carbonitrile | | |
| 37 | 4-(3-((1,4-diazepan-1-yl)methyl)-6-(1H-pyrazol-4- | 3.47 | 398 |
| | yl)imidazo[1,2-a]pyridin-2-yl)benzonitrile | | |
| 38 | 4-(3-((1,4-diazepan-1-yl)methyl)-6-(1-methyl-1H-pyrrol-2- | 4.25 | 411 |
| | yl)imidazo[1,2-a]pyridin-2-yl)benzonitrile | | |
| 39 | 4-(3-((1,4-diazepan-1-yl)methyl)-6-(oxazol-5- | 3.83 | 399 |
| | yl)imidazo[1,2-a]pyridin-2-yl)benzonitrile | | |
| 40 | 4-(3-((1,4-diazepan-1-yl)methyl)-6-(4- | 5.62 | 442 |
| | chlorophenyl)imidazo[1,2-a]pyridin-2-yl)benzonitrile | | |
| 41 | 4-(3-((1,4-diazepan-1-yl)methyl)-6-(4- | 4.63 | 438 |
| | methoxyphenyl)imidazo[1,2-a]pyridin-2-yl)benzonitrile | | |
| 42 | 4-(3-((1,4-diazepan-1-yl)methyl)-2-(4- | 4.26 | 451 |
| | cyanophenyl)imidazo[1,2-a]pyridin-6-yl)benzamide | | |
| 43 | 4,4′-(3-((1,4-diazepan-1-yl)methyl)imidazo[1,2-a]pyridine- | 4.90 | 433 |
| | 2,6-diyl)dibenzonitrile | | |
| 44 | 4-(3-((1,4-diazepan-1-yl)methyl)-6-(thiophen-3- | 4.40 | 414 |
| | yl)imidazo[1,2-a]pyridin-2-yl)benzonitrile | | |

Rₜ^{#} retention time, (HPLC) as described above

Compounds of the formula (I), listed in table II below, can be prepared using the standard operation procedures described above.

TABLE II

| Example | IUPAC-Name | Rₜ^{#} [min] | m/z + 1 |
|---|---|---|---|
| 45 | 2-(4-methoxyphenyl)-3-((4-methyl-1,4-diazepan-1- | | |
| | yl)methyl)-6-(pyridin-3-yl)imidazo[1,2-a]pyridine | | |
| 46 | 3-((1,4-diazepan-1-yl)methyl)-2-(4-methoxyphenyl)-6- | | |
| | (pyridin-3-yl)imidazo[1,2-a]pyridine | | |
| 47 | 3-((1,4-diazepan-1-yl)methyl)-2-(4-methoxyphenyl)-6-(4- | | |
| | (trifluoromethyl)phenyl)imidazo[1,2-a]pyridine | | |
| 48 | 2-(4-methoxyphenyl)-3-((4-methyl-1,4-diazepan-1- | | |
| | yl)methyl)-6-(4-(trifluoromethyl)phenyl)imidazo[1,2- | | |
| | a]pyridine | | |
| 49 | 4-(3-((1,4-diazepan-1-yl)methyl)-2-(4-methoxy- | | |
| | phenyl)imidazo[1,2-a]pyridin-6-yl)benzamide | | |
| 50 | 4-(2-(4-methoxyphenyl)-3-((4-methyl-1,4-diazepan-1- | | |
| | yl)methyl)imidazo[1,2-a]pyridin-6-yl)benzamide | | |
| 51 | 5-(3-((1,4-diazepan-1-yl)methyl)-2-(4-methoxy- | | |
| | phenyl)imidazo[1,2-a]pyridin-6-yl)thiophene-2- | | |
| | carbonitrile | | |
| 52 | 5-(2-(4-methoxyphenyl)-3-((4-methyl-1,4-diazepan-1- | | |
| | yl)methyl)imidazo[1,2-a]pyridin-6-yl)thiophene-2- | | |
| | carbonitrile | | |
| 53 | 3-((1,4-diazepan-1-yl)methyl)-2-(4-methoxyphenyl)-6-(4- | | |
| | (pentafluorothio)phenyl)imidazo[1,2-a]pyridine | | |
| 54 | 2-(4-methoxyphenyl)-3-((4-methyl-1,4-diazepan-1- | | |
| | yl)methyl)-6-(4-(pentafluorothio)phenyl)imidazo[1,2- | | |
| | a]pyridine | | |
| 55 | 2-(4-methoxyphenyl)-3-((4-methyl-1,4-diazepan-1- | | |
| | yl)methyl)-6-(1-methyl-1H-pyrrol-2-yl)imidazo[1,2- | | |
| | a]pyridine | | |
| 56 | 3-((1,4-diazepan-1-yl)methyl)-2-(4-methoxyphenyl)-6-(1- | | |
| | methyl-1H-pyrrol-2-yl)imidazo[1,2-a]pyridine | | |
| 57 | 4-(3-((1,4-diazepan-1-yl)methyl)-2-(4-methoxyphenyl)- | | |
| | imidazo[1,2-a]pyridin-6-yl)-2-methylbenzonitrile | | |
| 58 | 4-(2-(4-methoxyphenyl)-3-((4-methyl-1,4-diazepan-1- | | |
| | yl)methyl)imidazo[1,2-a]pyridin-6-yl)-2-methylbenzonitrile | | |
| 59 | 3-((1,4-diazepan-1-yl)methyl)-2-(4-methoxyphenyl)-6- | | |
| | (pyrimidin-5-yl)imidazo[1,2-a]pyridine | | |
| 60 | 2-(4-methoxyphenyl)-3-((4-methyl-1,4-diazepan-1- | | |
| | yl)methyl)-6-(pyrimidin-5-yl)imidazo[1,2-a]pyridine | | |
| 61 | 5-(2-(4-methoxyphenyl)-3-((4-methyl-1,4-diazepan-1- | | |
| | yl)methyl)imidazo[1,2-a]pyridin-6-yl)oxazole | | |
| 62 | 5-(3-((1,4-diazepan-1-yl)methyl)-2-(4- | | |
| | methoxyphenyl)imidazo[1,2-a]pyridin-6-yl)oxazole | | |
| 63 | 3-((1,4-diazepan-1-yl)methyl)-2-(4-methoxyphenyl)-6-(p- | | |
| | tolyl)imidazo[1,2-a]pyridine | | |
| 64 | 2-(4-methoxyphenyl)-3-((4-methyl-1,4-diazepan-1- | | |
| | yl)methyl)-6-(p-tolyl)imidazo[1,2-a]pyridine | | |
| 65 | 4-(3-((1,4-diazepan-1-yl)methyl)-2-(4-methoxy- | | |
| | phenyl)imidazo[1,2-a]pyridin-6-yl)benzonitrile | | |
| 66 | 4-(2-(4-methoxyphenyl)-3-((4-methyl-1,4-diazepan-1- | | |
| | yl)methyl)imidazo[1,2-a]pyridin-6-yl)benzonitrile | | |
| 67 | 5-(3-((1,4-diazepan-1-yl)methyl)-2-(4-methoxy- | | |
| | phenyl)imidazo[1,2-a]pyridin-6-yl)thiophene-3- | | |
| | carbonitrile | | |
| 68 | 5-(2-(4-methoxyphenyl)-3-((4-methyl-1,4-diazepan-1- | | |
| | yl)methyl)imidazo[1,2-a]pyridin-6-yl)thiophene-3- | | |
| | carbonitrile | | |
| 69 | 6-(4-chlorophenyl)-2-(4-methoxyphenyl)-3-((4-methyl- | | |
| | 1,4-diazepan-1-yl)methyl)imidazo[1,2-a]pyridine | | |
| 70 | 3-((1,4-diazepan-1-yl)methyl)-6-(4-chlorophenyl)-2-(4- | | |
| | methoxyphenyl)imidazo[1,2-a]pyridine | | |
| 71 | 4-(3-((1,4-diazepan-1-yl)methyl)-2-(4-methoxy- | | |
| | phenyl)imidazo[1,2-a]pyridin-6-yl)thiophene-2- | | |
| | carbonitrile | | |
| 72 | 4-(2-(4-methoxyphenyl)-3-((4-methyl-1,4-diazepan-1- | | |
| | yl)methyl)imidazo[1,2-a]pyridin-6-yl)thiophene-2- | | |
| | carbonitrile | | |
| 73 | 2-(4-methoxyphenyl)-3-((4-methyl-1,4-diazepan-1- | | |
| | yl)methyl)-6-(o-tolyl)imidazo[1,2-a]pyridine | | |
| 74 | 3-((1,4-diazepan-1-yl)methyl)-2-(4-methoxyphenyl)-6-(o- | | |
| | tolyl)imidazo[1,2-a]pyridine | | |
| 75 | 4-(2-(4-methoxyphenyl)-3-((4-methyl-1,4-diazepan-1- | | |
| | yl)methyl)imidazo[1,2-a]pyridin-6-yl)-N,N-dimethylaniline | | |
| 76 | 4-(3-((1,4-diazepan-1-yl)methyl)-2-(4-methoxy- | | |
| | phenyl)imidazo[1,2-a]pyridin-6-yl)-N,N-dimethylaniline | | |
| 77 | 2,6-bis(4-methoxyphenyl)-3-((4-methyl-1,4-diazepan-1- | | |
| | yl)methyl)imidazo[1,2-a]pyridine | | |
| 78 | 3-((1,4-diazepan-1-yl)methyl)-2,6-bis(4-methoxy- | | |
| | phenyl)imidazo[1,2-a]pyridine | | |
| 79 | 3-((1,4-diazepan-1-yl)methyl)-2-(4-methoxyphenyl)-6-(m- | | |
| | tolyl)imidazo[1,2-a]pyridine | | |
| 80 | 2-(4-methoxyphenyl)-3-((4-methyl-1,4-diazepan-1- | | |
| | yl)methyl)-6-(m-tolyl)imidazo[1,2-a]pyridine | | |
| 81 | 2-(4-methoxyphenyl)-3-((4-methyl-1,4-diazepan-1- | | |
| | yl)methyl)-6-(2-methylpyridin-4-yl)imidazo[1,2-a]pyridine | | |
| 82 | 3-((1,4-diazepan-1-yl)methyl)-2-(4-methoxyphenyl)-6-(2- | | |
| | methylpyridin-4-yl)imidazo[1,2-a]pyridine | | |
| 83 | 2-(4-methoxyphenyl)-3-((4-methyl-1,4-diazepan-1- | | |
| | yl)methyl)-6-(thiophen-3-yl)imidazo[1,2-a]pyridine | | |
| 84 | 3-((1,4-diazepan-1-yl)methyl)-2-(4-methoxyphenyl)-6- | | |
| | (thiophen-3-yl)imidazo[1,2-a]pyridine | | |
| 85 | 6-(3,4-dimethoxyphenyl)-2-(4-methoxyphenyl)-3-((4- | | |
| | methyl-1,4-diazepan-1-yl)methyl)imidazo[1,2-a]pyridine | | |
| 86 | 3-((1,4-diazepan-1-yl)methyl)-6-(3,4-dimethoxyphenyl)- | | |
| | 2-(4-methoxyphenyl)imidazo[1,2-a]pyridine | | |
| 87 | 6-(1,5-dimethyl-1H-pyrrol-2-yl)-2-(4-methoxyphenyl)-3- | | |
| | ((4-methyl-1,4-diazepan-1-yl)methyl)imidazo[1,2- | | |
| | a]pyridine | | |
| 88 | 3-((1,4-diazepan-1-yl)methyl)-6-(1,5-dimethyl-1H-pyrrol- | | |
| | 2-yl)-2-(4-methoxyphenyl)imidazo[1,2-a]pyridine | | |
| 89 | 5-(3-((1,4-diazepan-1-yl)methyl)-2-(4-methoxyphenyl)- | | |
| | imidazo[1,2-a]pyridin-6-yl)-1H-pyrrole-2-carbonitrile | | |
| 90 | 5-(2-(4-methoxyphenyl)-3-((4-methyl-1,4-diazepan-1- | | |
| | yl)methyl)imidazo[1,2-a]pyridin-6-yl)-1H-pyrrole-2- | | |
| | carbonitrile | | |
| 91 | 2-(4-methoxyphenyl)-3-((4-methyl-1,4-diazepan-1- | | |
| | yl)methyl)-6-(5-methyl-1H-pyrrol-2-yl)imidazo[1,2- | | |
| | a]pyridine | | |
| 92 | 3-((1,4-diazepan-1-yl)methyl)-2-(4-methoxyphenyl)-6-(5- | | |
| | methyl-1H-pyrrol-2-yl)imidazo[1,2-a]pyridine | | |
| 93 | 2-(4-methoxyphenyl)-3-((4-methyl-1,4-diazepan-1- | | |
| | yl)methyl)-6-phenylimidazo[1,2-a]pyridine | | |
| 94 | 6-(furan-2-yl)-2-(4-methoxyphenyl)-3-((4-methyl-1,4- | | |
| | diazepan-1-yl)methyl)imidazo[1,2-a]pyridine | | |
| 95 | 3-((1,4-diazepan-1-yl)methyl)-6-(furan-2-yl)-2-(4- | | |
| | methoxyphenyl)imidazo[1,2-a]pyridine | | |
| 96 | 3-((1,4-diazepan-1-yl)methyl)-2-(4-methoxyphenyl)-6- | | |
| | (1H-pyrrol-2-yl)imidazo[1,2-a]pyridine | | |
| 97 | 2-(4-methoxyphenyl)-3-((4-methyl-1,4-diazepan-1- | | |
| | yl)methyl)-6-(1H-pyrrol-2-yl)imidazo[1,2-a]pyridine | | |
| 98 | 2-(4-methoxyphenyl)-3-((4-methyl-1,4-diazepan-1- | | |
| | yl)methyl)-6-(pyridin-4-yl)imidazo[1,2-a]pyridine | | |
| 99 | 3-((1,4-diazepan-1-yl)methyl)-2-(4-methoxyphenyl)-6- | | |
| | (pyridin-4-yl)imidazo[1,2-a]pyridine | | |
| 100 | 3-((1,4-diazepan-1-yl)methyl)-6-(3,4-dimethylphenyl)-2- | | |
| | (4-methoxyphenyl)imidazo[1,2-a]pyridine | | |
| 101 | 6-(3,4-dimethylphenyl)-2-(4-methoxyphenyl)-3-((4- | | |
| | methyl-1,4-diazepan-1-yl)methyl)imidazo[1,2-a]pyridine | | |
| 102 | 3-((1,4-diazepan-1-yl)methyl)-2-(4-methoxyphenyl)-6- | | |
| | (1H-pyrazol-4-yl)imidazo[1,2-a]pyridine | | |
| 103 | 2-(4-methoxyphenyl)-3-((4-methyl-1,4-diazepan-1- | | |
| | yl)methyl)-6-(1H-pyrazol-4-yl)imidazo[1,2-a]pyridine | | |
| 104 | 2-(4-chlorophenyl)-3-((4-methyl-1,4-diazepan-1- | | |
| | yl)methyl)-6-(4-(pentafluorothio)phenyl)imidazo[1,2- | | |
| | a]pyridine | | |
| 105 | 3-((1,4-diazepan-1-yl)methyl)-2-(4-chlorophenyl)-6-(4- | | |
| | (pentafluorothio)phenyl)imidazo[1,2-a]pyridine | | |
| 106 | 3-((1,4-diazepan-1-yl)methyl)-2-(4-chlorophenyl)-6- | | |
| | (pyrimidin-5-yl)imidazo[1,2-a]pyridine | | |
| 107 | 2-(4-chlorophenyl)-3-((4-methyl-1,4-diazepan-1- | | |
| | yl)methyl)-6-(pyrimidin-5-yl)imidazo[1,2-a]pyridine | | |
| 108 | 4-(2-(4-chlorophenyl)-3-((4-methyl-1,4-diazepan-1- | | |
| | yl)methyl)imidazo[1,2-a]pyridin-6-yl)-2-methylbenzonitrile | | |
| 109 | 4-(3-((1,4-diazepan-1-yl)methyl)-2-(4-chlorophenyl)- | | |
| | imidazo[1,2-a]pyridin-6-yl)-2-methylbenzonitrile | | |
| 110 | 3-((1,4-diazepan-1-yl)methyl)-2-(4-chlorophenyl)-6-(1H- | | |
| | pyrrol-2-yl)imidazo[1,2-a]pyridine | | |
| 111 | 2-(4-chlorophenyl)-3-((4-methyl-1,4-diazepan-1- | | |
| | yl)methyl)-6-(1H-pyrrol-2-yl)imidazo[1,2-a]pyridine | | |
| 112 | 3-((1,4-diazepan-1-yl)methyl)-2-(4-chlorophenyl)-6-(m- | | |
| | tolyl)imidazo[1,2-a]pyridine | | |
| 113 | 2-(4-chlorophenyl)-3-((4-methyl-1,4-diazepan-1- | | |
| | yl)methyl)-6-(m-tolyl)imidazo[1,2-a]pyridine | | |
| 114 | 2-(4-chlorophenyl)-6-(3,4-dimethylphenyl)-3-((4-methyl- | | |
| | 1,4-diazepan-1-yl)methyl)imidazo[1,2-a]pyridine | | |
| 115 | 3-((1,4-diazepan-1-yl)methyl)-2-(4-chlorophenyl)-6-(3,4- | | |
| | dimethylphenyl)imidazo[1,2-a]pyridine | | |
| 116 | 5-(3-((1,4-diazepan-1-yl)methyl)-2-(4-chlorophenyl)- | | |
| | imidazo[1,2-a]pyridin-6-yl)-1H-pyrrole-2-carbonitrile | | |
| 117 | 5-(2-(4-chlorophenyl)-3-((4-methyl-1,4-diazepan-1- | | |
| | yl)methyl)imidazo[1,2-a]pyridin-6-yl)-1H-pyrrole-2- | | |
| | carbonitrile | | |
| 118 | 3-((1,4-diazepan-1-yl)methyl)-2-(4-chlorophenyl)-6-(o- | | |
| | tolyl)imidazo[1,2-a]pyridine | | |
| 119 | 2-(4-chlorophenyl)-3-((4-methyl-1,4-diazepan-1- | | |
| | yl)methyl)-6-(o-tolyl)imidazo[1,2-a]pyridine | | |
| 120 | 2-(4-chlorophenyl)-6-(3,4-dimethoxyphenyl)-3-((4- | | |
| | methyl-1,4-diazepan-1-yl)methyl)imidazo[1,2-a]pyridine | | |
| 121 | 3-((1,4-diazepan-1-yl)methyl)-2-(4-chlorophenyl)-6-(3,4- | | |
| | dimethoxyphenyl)imidazo[1,2-a]pyridine | | |
| 122 | 2-(4-chlorophenyl)-6-(1,5-dimethyl-1H-pyrrol-2-yl)-3-((4- | | |
| | methyl-1,4-diazepan-1-yl)methyl)imidazo[1,2-a]pyridine | | |
| 123 | 3-((1,4-diazepan-1-yl)methyl)-2-(4-chlorophenyl)-6-(1,5- | | |
| | dimethyl-1H-pyrrol-2-yl)imidazo[1,2-a]pyridine | | |
| 124 | 2-(4-chlorophenyl)-3-((4-methyl-1,4-diazepan-1- | | |
| | yl)methyl)-6-(5-methyl-1H-pyrrol-2-yl)imidazo[1,2- | | |
| | a]pyridine | | |
| 125 | 3-((1,4-diazepan-1-yl)methyl)-2-(4-chlorophenyl)-6-(5- | | |
| | methyl-1H-pyrrol-2-yl)imidazo[1,2-a]pyridine | | |
| 126 | 3-((1,4-diazepan-1-yl)methyl)-2-(4-chlorophenyl)-6- | | |
| | (pyridin-3-yl)imidazo[1,2-a]pyridine | | |
| 127 | 2-(4-chlorophenyl)-3-((4-methyl-1,4-diazepan-1- | | |
| | yl)methyl)-6-(pyridin-3-yl)imidazo[1,2-a]pyridine | | |
| 128 | 2-(4-chlorophenyl)-6-(furan-2-yl)-3-((4-methyl-1,4- | | |
| | diazepan-1-yl)methyl)imidazo[1,2-a]pyridine | | |
| 129 | 3-((1,4-diazepan-1-yl)methyl)-2-(4-chlorophenyl)-6- | | |
| | (furan-2-yl)imidazo[1,2-a]pyridine | | |
| 130 | 3-((1,4-diazepan-1-yl)methyl)-2-(4-chlorophenyl)-6-(1H- | | |
| | pyrazol-4-yl)imidazo[1,2-a]pyridine | | |
| 131 | 2-(4-chlorophenyl)-3-((4-methyl-1,4-diazepan-1- | | |
| | yl)methyl)-6-(1H-pyrazol-4-yl)imidazo[1,2-a]pyridine | | |
| 132 | 4-(2-(4-chlorophenyl)-3-((4-methyl-1,4-diazepan-1- | | |
| | yl)methyl)imidazo[1,2-a]pyridin-6-yl)thiophene-2- | | |
| | carbonitrile | | |
| 133 | 4-(3-((1,4-diazepan-1-yl)methyl)-2-(4-chlorophenyl)- | | |
| | imidazo[1,2-a]pyridin-6-yl)thiophene-2-carbonitrile | | |
| 134 | 5-(2-(4-chlorophenyl)-3-((4-methyl-1,4-diazepan-1- | | |
| | yl)methyl)imidazo[1,2-a]pyridin-6-yl)thiophene-3- | | |
| | carbonitrile | | |
| 135 | 5-(3-((1,4-diazepan-1-yl)methyl)-2-(4-chlorophenyl)- | | |
| | imidazo[1,2-a]pyridin-6-yl)thiophene-3-carbonitrile | | |
| 136 | 3-((1,4-diazepan-1-yl)methyl)-2-(4-chlorophenyl)-6-(1- | | |
| | methyl-1H-pyrrol-2-yl)imidazo[1,2-a]pyridine | | |
| 137 | 2-(4-chlorophenyl)-3-((4-methyl-1,4-diazepan-1- | | |
| | yl)methyl)-6-(1-methyl-1H-pyrrol-2-yl)imidazo[1,2- | | |
| | a]pyridine | | |
| 138 | 2-(4-chlorophenyl)-3-((4-methyl-1,4-diazepan-1- | | |
| | yl)methyl)-6-(2-methylpyridin-4-yl)imidazo[1,2-a]pyridine | | |
| 139 | 3-((1,4-diazepan-1-yl)methyl)-2-(4-chlorophenyl)-6-(2- | | |
| | methylpyridin-4-yl)imidazo[1,2-a]pyridine | | |
| 140 | 5-(2-(4-chlorophenyl)-3-((4-methyl-1,4-diazepan-1- | | |
| | yl)methyl)imidazo[1,2-a]pyridin-6-yl)thiophene-2- | | |
| | carbonitrile | | |
| 141 | 5-(3-((1,4-diazepan-1-yl)methyl)-2-(4- | | |
| | chlorophenyl)imidazo[1,2-a]pyridin-6-yl)thiophene-2- | | |
| | carbonitrile | | |
| 142 | 5-(3-((1,4-diazepan-1-yl)methyl)-2-(4- | | |
| | chlorophenyl)imidazo[1,2-a]pyridin-6-yl)oxazole | | |
| 143 | 5-(2-(4-chlorophenyl)-3-((4-methyl-1,4-diazepan-1- | | |
| | yl)methyl)imidazo[1,2-a]pyridin-6-yl)oxazole | | |
| 144 | 4-(2-(4-chlorophenyl)-3-((4-methyl-1,4-diazepan-1- | | |
| | yl)methyl)imidazo[1,2-a]pyridin-6-yl)benzamide | | |
| 145 | 4-(3-((1,4-diazepan-1-yl)methyl)-2-(4- | | |
| | chlorophenyl)imidazo[1,2-a]pyridin-6-yl)benzamide | | |
| 146 | 2-(4-chlorophenyl)-6-(4-methoxyphenyl)-3-((4-methyl- | | |
| | 1,4-diazepan-1-yl)methyl)imidazo[1,2-a]pyridine | | |
| 147 | 3-((1,4-diazepan-1-yl)methyl)-2-(4-chlorophenyl)-6-(4- | | |
| | methoxyphenyl)imidazo[1,2-a]pyridine | | |
| 148 | 2-(4-chlorophenyl)-3-((4-methyl-1,4-diazepan-1- | | |
| | yl)methyl)-6-(pyridin-4-yl)imidazo[1,2-a]pyridine | | |
| 149 | 3-((1,4-diazepan-1-yl)methyl)-2-(4-chlorophenyl)-6- | | |
| | (pyridin-4-yl)imidazo[1,2-a]pyridine | | |
| 150 | 3-((1,4-diazepan-1-yl)methyl)-2-(4-chlorophenyl)-6- | | |
| | (thiophen-3-yl)imidazo[1,2-a]pyridine | | |
| 151 | 2-(4-chlorophenyl)-3-((4-methyl-1,4-diazepan-1- | | |
| | yl)methyl)-6-(thiophen-3-yl)imidazo[1,2-a]pyridine | | |
| 152 | 3-((1,4-diazepan-1-yl)methyl)-2,6-bis(4- | | |
| | chlorophenyl)imidazo[1,2-a]pyridine | | |
| 153 | 2,6-bis(4-chlorophenyl)-3-((4-methyl-1,4-diazepan-1- | | |
| | yl)methyl)imidazo[1,2-a]pyridine | | |
| 154 | 4-(3-((1,4-diazepan-1-yl)methyl)-2-(4- | | |
| | chlorophenyl)imidazo[1,2-a]pyridin-6-yl)benzonitrile | | |
| 155 | 4-(2-(4-chlorophenyl)-3-((4-methyl-1,4-diazepan-1- | | |
| | yl)methyl)imidazo[1,2-a]pyridin-6-yl)benzonitrile | | |
| 156 | 4-(2-(4-chlorophenyl)-3-((4-methyl-1,4-diazepan-1- | | |
| | yl)methyl)imidazo[1,2-a]pyridin-6-yl)-N,N-dimethylaniline | | |
| 157 | 4-(3-((1,4-diazepan-1-yl)methyl)-2-(4-chlorophenyl)- | | |
| | imidazo[1,2-a]pyridin-6-yl)-N,N-dimethylaniline | | |
| 158 | 3-((1,4-diazepan-1-yl)methyl)-2-(4-chlorophenyl)-6-(p- | | |
| | tolyl)imidazo[1,2-a]pyridine | | |
| 159 | 2-(4-chlorophenyl)-3-((4-methyl-1,4-diazepan-1- | | |
| | yl)methyl)-6-(p-tolyl)imidazo[1,2-a]pyridine | | |
| 160 | 3-((1,4-diazepan-1-yl)methyl)-2-(4-chlorophenyl)-6-(4- | | |
| | (trifluoromethyl)phenyl)imidazo[1,2-a]pyridine | | |
| 161 | 2-(4-chlorophenyl)-3-((4-methyl-1,4-diazepan-1- | | |
| | yl)methyl)-6-(4-(trifluoromethyl)phenyl)imidazo[1,2- | | |
| | a]pyridine | | |
| 162 | 5-(3-((1,4-diazepan-1-yl)methyl)-2-(4- | | |
| | cyanophenyl)imidazo[1,2-a]pyridin-6-yl)thiophene-2- | | |
| | carbonitrile | | |
| 163 | 5-(2-(4-cyanophenyl)-3-((4-methyl-1,4-diazepan-1- | | |
| | yl)methyl)imidazo[1,2-a]pyridin-6-yl)thiophene-2- | | |
| | carbonitrile | | |
| 164 | 5-(3-((1,4-diazepan-1-yl)methyl)-2-(4-cyanophenyl)- | | |
| | imidazo[1,2-a]pyridin-6-yl)thiophene-3-carbonitrile | | |
| 165 | 5-(2-(4-cyanophenyl)-3-((4-methyl-1,4-diazepan-1- | | |
| | yl)methyl)imidazo[1,2-a]pyridin-6-yl)thiophene-3- | | |
| | carbonitrile | | |
| 166 | 4-(3-((4-methyl-1,4-diazepan-1-yl)methyl)-6-(1H-pyrazol- | | |
| | 4-yl)imidazo[1,2-a]pyridin-2-yl)benzonitrile | | |
| 167 | 4-(3-((1,4-diazepan-1-yl)methyl)-6-(furan-2- | | |
| | yl)imidazo[1,2-a]pyridin-2-yl)benzonitrile | | |
| 168 | 4-(6-(furan-2-yl)-3-((4-methyl-1,4-diazepan-1- | | |
| | yl)methyl)imidazo[1,2-a]pyridin-2-yl)benzonitrile | | |
| 169 | 4-(6-(3,4-dimethylphenyl)-3-((4-methyl-1,4-diazepan-1- | | |
| | yl)methyl)imidazo[1,2-a]pyridin-2-yl)benzonitrile | | |
| 170 | 4-(3-((1,4-diazepan-1-yl)methyl)-6-(3,4- | | |
| | dimethylphenyl)imidazo[1,2-a]pyridin-2-yl)benzonitrile | | |
| 171 | 4-(3-((4-methyl-1,4-diazepan-1-yl)methyl)-6-(o- | | |
| | tolyl)imidazo[1,2-a]pyridin-2-yl)benzonitrile | | |
| 172 | 4-(3-((1,4-diazepan-1-yl)methyl)-6-(5-methyl-1H-pyrrol-2- | | |
| | yl)imidazo[1,2-a]pyridin-2-yl)benzonitrile | | |
| 173 | 4-(3-((4-methyl-1,4-diazepan-1-yl)methyl)-6-(5-methyl- | | |
| | 1H-pyrrol-2-yl)imidazo[1,2-a]pyridin-2-yl)benzonitrile | | |
| 174 | 4-(3-((4-methyl-1,4-diazepan-1-yl)methyl)-6-(pyridin-3- | | |
| | yl)imidazo[1,2-a]pyridin-2-yl)benzonitrile | | |
| 175 | 4-(3-((1,4-diazepan-1-yl)methyl)-6-(1,5-dimethyl-1H- | | |
| | pyrrol-2-yl)imidazo[1,2-a]pyridin-2-yl)benzonitrile | | |
| 176 | 4-(6-(1,5-dimethyl-1H-pyrrol-2-yl)-3-((4-methyl-1,4- | | |
| | diazepan-1-yl)methyl)imidazo[1,2-a]pyridin-2- | | |
| | yl)benzonitrile | | |
| 177 | 4-(6-(3,4-dimethoxyphenyl)-3-((4-methyl-1,4-diazepan-1- | | |
| | yl)methyl)imidazo[1,2-a]pyridin-2-yl)benzonitrile | | |
| 178 | 4-(3-((1,4-diazepan-1-yl)methyl)-6-(3,4- | | |
| | dimethoxyphenyl)imidazo[1,2-a]pyridin-2-yl)benzonitrile | | |
| 179 | 4-(3-((4-methyl-1,4-diazepan-1-yl)methyl)-6-(pyridin-4- | | |
| | yl)imidazo[1,2-a]pyridin-2-yl)benzonitrile | | |
| 180 | 4-(3-((1,4-diazepan-1-yl)methyl)-6-(1H-pyrrol-2- | | |
| | yl)imidazo[1,2-a]pyridin-2-yl)benzonitrile | | |
| 181 | 4-(3-((4-methyl-1,4-diazepan-1-yl)methyl)-6-(1H-pyrrol-2- | | |
| | yl)imidazo[1,2-a]pyridin-2-yl)benzonitrile | | |
| 182 | 4-(3-((4-methyl-1,4-diazepan-1-yl)methyl)-6-(pyrimidin-5- | | |
| | yl)imidazo[1,2-a]pyridin-2-yl)benzonitrile | | |
| 183 | 4-(3-((1,4-diazepan-1-yl)methyl)-6-(4- | | |
| | (trifluoromethyl)phenyl)imidazo[1,2-a]pyridin-2- | | |
| | yl)benzonitrile | | |
| 184 | 4-(3-((4-methyl-1,4-diazepan-1-yl)methyl)-6-(4- | | |
| | (trifluoromethyl)phenyl)imidazo[1,2-a]pyridin-2- | | |
| | yl)benzonitrile | | |
| 185 | 4-(3-((1,4-diazepan-1-yl)methyl)-2-(4- | | |
| | cyanophenyl)imidazo[1,2-a]pyridin-6-yl)-2- | | |
| | methylbenzonitrile | | |
| 186 | 4-(2-(4-cyanophenyl)-3-((4-methyl-1,4-diazepan-1- | | |
| | yl)methyl)imidazo[1,2-a]pyridin-6-yl)-2-methylbenzonitrile | | |
| 187 | 4-(6-(4-methoxyphenyl)-3-((4-methyl-1,4-diazepan-1- | | |
| | yl)methyl)imidazo[1,2-a]pyridin-2-yl)benzonitrile | | |
| 188 | 5-(2-(4-cyanophenyl)-3-((4-methyl-1,4-diazepan-1- | | |
| | yl)methyl)imidazo[1,2-a]pyridin-6-yl)-1H-pyrrole-2- | | |
| | carbonitrile | | |
| 189 | 5-(3-((1,4-diazepan-1-yl)methyl)-2-(4- | | |
| | cyanophenyl)imidazo[1,2-a]pyridin-6-yl)-1H-pyrrole-2- | | |
| | carbonitrile | | |
| 190 | 4-(2-(4-cyanophenyl)-3-((4-methyl-1,4-diazepan-1- | | |
| | yl)methyl)imidazo[1,2-a]pyridin-6-yl)thiophene-2- | | |
| | carbonitrile | | |
| 191 | 4-(3-((1,4-diazepan-1-yl)methyl)-2-(4- | | |
| | cyanophenyl)imidazo[1,2-a]pyridin-6-yl)thiophene-2- | | |
| | carbonitrile | | |
| 192 | 4-(6-(4-chlorophenyl)-3-((4-methyl-1,4-diazepan-1- | | |
| | yl)methyl)imidazo[1,2-a]pyridin-2-yl)benzonitrile | | |
| 193 | 4-(2-(4-cyanophenyl)-3-((4-methyl-1,4-diazepan-1- | | |
| | yl)methyl)imidazo[1,2-a]pyridin-6-yl)benzamide | | |
| 194 | 4,4′-(3-((4-methyl-1,4-diazepan-1-yl)methyl)imidazo[1,2- | | |
| | a]pyridine-2,6-diyl)dibenzonitrile | | |
| 195 | 4-(6-(4-(dimethylamino)phenyl)-3-((4-methyl-1,4- | | |
| | diazepan-1-yl)methyl)imidazo[1,2-a]pyridin-2- | | |
| | yl)benzonitrile | | |
| 196 | 4-(3-((1,4-diazepan-1-yl)methyl)-6-(4- | | |
| | (dimethylamino)phenyl)imidazo[1,2-a]pyridin-2- | | |
| | yl)benzonitrile | | |
| 197 | 4-(3-((4-methyl-1,4-diazepan-1-yl)methyl)-6-(2- | | |
| | methylpyridin-4-yl)imidazo[1,2-a]pyridin-2-yl)benzonitrile | | |
| 198 | 4-(3-((4-methyl-1,4-diazepan-1-yl)methyl)-6-(1-methyl- | | |
| | 1H-pyrrol-2-yl)imidazo[1,2-a]pyridin-2-yl)benzonitrile | | |
| 199 | 4-(3-((4-methyl-1,4-diazepan-1-yl)methyl)-6-(p- | | |
| | tolyl)imidazo[1,2-a]pyridin-2-yl)benzonitrile | | |
| 200 | 4-(3-((4-methyl-1,4-diazepan-1-yl)methyl)-6-(m- | | |
| | tolyl)imidazo[1,2-a]pyridin-2-yl)benzonitrile | | |
| 201 | 4-(3-((4-methyl-1,4-diazepan-1-yl)methyl)-6-(oxazol-5- | | |
| | yl)imidazo[1,2-a]pyridin-2-yl)benzonitrile | | |
| 202 | 4-(3-((4-methyl-1,4-diazepan-1-yl)methyl)-6-(4- | | |
| | (pentafluorothio)phenyl)imidazo[1,2-a]pyridin-2- | | |
| | yl)benzonitrile | | |
| 203 | 4-(3-((1,4-diazepan-1-yl)methyl)-6-(4- | | |
| | (pentafluorothio)phenyl)imidazo[1,2-a]pyridin-2- | | |
| | yl)benzonitrile | | |
| 204 | 4-(3-((4-methyl-1,4-diazepan-1-yl)methyl)-6-(thiophen-3- | | |
| | yl)imidazo[1,2-a]pyridin-2-yl)benzonitrile | | |
| 205 | 4-(2-(4-chlorophenyl)-3-((4-methylpiperazin-1- | | |
| | yl)methyl)imidazo[1,2-a]pyridin-6-yl)benzonitrile | | |
| 206 | 4-(2-(4-methoxyphenyl)-3-((3-oxopiperazin-1- | | |
| | yl)methyl)imidazo[1,2-a]pyridin-6-yl)benzonitrile | | |
| 207 | 4-(2-(4-methoxyphenyl)-3-((4-(2,2,2-trifluoroacetyl)-1,4- | | |
| | diazepan-1-yl)methyl)imidazo[1,2-a]pyridin-6- | | |
| | yl)benzonitrile | | |
| 208 | 4-(2-(4-methoxyphenyl)-3-((4-(trifluoromethyl)-1,4- | | |
| | diazepan-1-yl)methyl)imidazo[1,2-a]pyridin-6- | | |
| | yl)benzonitrile | | |
| 209 | 4-(6-(3,4-dimethylphenyl)-3-((4-propylpiperazin-1- | | |
| | yl)methyl)imidazo[1,2-a]pyridin-2-yl)benzonitrile | | |
| 210 | 4-((2-(4-chlorophenyl)-6-(3,4- | | |
| | dimethoxyphenyl)imidazo[1,2-a]pyridin-3- | | |
| | yl)methyl)piperazin-2-one | | |
| 211 | 4-(6-(3,4-dimethylphenyl)-3-((3-oxopiperazin-1- | | |
| | yl)methyl)imidazo[1,2-a]pyridin-2-yl)benzonitrile | | |
| 212 | 4-((2-(4-methoxyphenyl)-6-(oxazol-5-yl)imidazo[1,2- | | |
| | a]pyridin-3-yl)methyl)piperazin-2-one | | |
| 213 | 4-(3-((4-methylpiperazin-1-yl)methyl)-6-(pyrimidin-5- | | |
| | yl)imidazo[1,2-a]pyridin-2-yl)benzonitrile | | |
| 214 | 4-(3-((4-methylpiperazin-1-yl)methyl)-6-(4- | | |
| | (trifluoromethyl)phenyl)imidazo[1,2-a]pyridin-2- | | |
| | yl)benzonitrile | | |
| 215 | 4-(6-(3,4-dimethylphenyl)-3-((4- | | |
| | (trifluoromethyl)piperazin-1-yl)methyl)imidazo[1,2- | | |
| | a]pyridin-2-yl)benzonitrile | | |
| 216 | 4-(6-(3,4-dimethoxyphenyl)-3-((4-propylpiperazin-1- | | |
| | yl)methyl)imidazo[1,2-a]pyridin-2-yl)benzonitrile | | |
| 217 | 4-(2-(4-cyanophenyl)-3-((4-(trifluoromethyl)-1,4- | | |
| | diazepan-1-yl)methyl)imidazo[1,2-a]pyridin-6- | | |
| | yl)thiophene-2-carbonitrile | | |
| 218 | 4-(3-((4-methylpiperazin-1-yl)methyl)-6-(2-methylpyridin- | | |
| | 4-yl)imidazo[1,2-a]pyridin-2-yl)benzonitrile | | |
| 219 | 2-(4-methoxyphenyl)-6-(pyrimidin-5-yl)-3-((4- | | |
| | (trifluoromethyl)piperazin-1-yl)methyl)imidazo[1,2- | | |
| | a]pyridine | | |
| 220 | 4-(3-((4-methylpiperazin-1-yl)methyl)-6-(oxazol-5- | | |
| | yl)imidazo[1,2-a]pyridin-2-yl)benzonitrile | | |
| 221 | 1-(4-((2-(4-methoxyphenyl)-6-(pyridin-3-yl)imidazo[1,2- | | |
| | a]pyridin-3-yl)methyl)-1,4-diazepan-1-yl)ethanone | | |
| 222 | 4-(2-(4-methoxyphenyl)-3-((4-propylpiperazin-1- | | |
| | yl)methyl)imidazo[1,2-a]pyridin-6-yl)-N,N-dimethylaniline | | |
| 223 | 4-(3-((4-acetylpiperazin-1-yl)methyl)-6-(5-methyl-1H- | | |
| | pyrrol-2-yl)imidazo[1,2-a]pyridin-2-yl)benzonitrile | | |
| 224 | 1-(4-((2-(4-methoxyphenyl)-6-(1-methyl-1H-pyrrol-2- | | |
| | yl)imidazo[1,2-a]pyridin-3-yl)methyl)piperazin-1- | | |
| | yl)ethanone | | |
| 225 | 2-(4-methoxyphenyl)-3-((4-methylpiperazin-1-yl)methyl)- | | |
| | 6-(4-(trifluoromethyl)phenyl)imidazo[1,2-a]pyridine | | |
| 226 | 2-(4-chlorophenyl)-6-(m-tolyl)-3-((4-(trifluoromethyl)-1,4- | | |
| | diazepan-1-yl)methyl)imidazo[1,2-a]pyridine | | |
| 227 | 4-(6-(4-(dimethylamino)phenyl)-3-((4-(2,2,2- | | |
| | trifluoroacetyl)-1,4-diazepan-1-yl)methyl)imidazo[1,2- | | |
| | a]pyridin-2-yl)benzonitrile | | |
| 228 | 4-(6-(4-(dimethylamino)phenyl)-3-((4-(trifluoromethyl)- | | |
| | 1,4-diazepan-1-yl)methyl)imidazo[1,2-a]pyridin-2- | | |
| | yl)benzonitrile | | |
| 229 | 4-(6-(1-methyl-1H-pyrrol-2-yl)-3-((4-methylpiperazin-1- | | |
| | yl)methyl)imidazo[1,2-a]pyridin-2-yl)benzonitrile | | |
| 230 | 4-(6-(furan-2-yl)-3-((4-(2,2,2-trifluoroacetyl)-1,4- | | |
| | diazepan-1-yl)methyl)imidazo[1,2-a]pyridin-2- | | |
| | yl)benzonitrile | | |
| 231 | 2-(4-methoxyphenyl)-3-((4-methylpiperazin-1-yl)methyl)- | | |
| | 6-(pyridin-4-yl)imidazo[1,2-a]pyridine | | |
| 232 | 4-(6-(1-methyl-1H-pyrrol-2-yl)-3-((4-(2,2,2- | | |
| | trifluoroacetyl)piperazin-1-yl)methyl)imidazo[1,2- | | |
| | a]pyridin-2-yl)benzonitrile | | |
| 233 | 4-(6-(oxazol-5-yl)-3-((4-(2,2,2-trifluoroacetyl)-1,4- | | |
| | diazepan-1-yl)methyl)imidazo[1,2-a]pyridin-2- | | |
| | yl)benzonitrile | | |
| 234 | 4-(3-((4-acetyl-1,4-diazepan-1-yl)methyl)-6-(4- | | |
| | (pentafluorothio)phenyl)imidazo[1,2-a]pyridin-2- | | |
| | yl)benzonitrile | | |
| 235 | 4-(6-(3,4-dimethylphenyl)-3-((4-(trifluoromethyl)-1,4- | | |
| | diazepan-1-yl)methyl)imidazo[1,2-a]pyridin-2- | | |
| | yl)benzonitrile | | |
| 236 | 4-(6-(1-methyl-1H-pyrrol-2-yl)-3-((4-(trifluoromethyl)-1,4- | | |
| | diazepan-1-yl)methyl)imidazo[1,2-a]pyridin-2- | | |
| | yl)benzonitrile | | |
| 237 | 4-(3-((4-methylpiperazin-1-yl)methyl)-6-(thiophen-3- | | |
| | yl)imidazo[1,2-a]pyridin-2-yl)benzonitrile | | |
| 238 | 2-(4-methoxyphenyl)-3-((4-methylpiperazin-1-yl)methyl)- | | |
| | 6-(1H-pyrrol-2-yl)imidazo[1,2-a]pyridine | | |
| 239 | 5-(2-(4-cyanophenyl)-3-((4-methylpiperazin-1- | | |
| | yl)methyl)imidazo[1,2-a]pyridin-6-yl)thiophene-3- | | |
| | carbonitrile | | |
| 240 | 4-((6-(furan-2-yl)-2-(4-methoxyphenyl)imidazo[1,2- | | |
| | a]pyridin-3-yl)methyl)piperazin-2-one | | |
| 241 | 4-(3-((4-methylpiperazin-1-yl)methyl)-6-(1H-pyrazol-4- | | |
| | yl)imidazo[1,2-a]pyridin-2-yl)benzonitrile | | |
| 242 | 4-(6-(furan-2-yl)-3-((4-propylpiperazin-1- | | |
| | yl)methyl)imidazo[1,2-a]pyridin-2-yl)benzonitrile | | |
| 243 | 4-(3-((4-acetyl-1,4-diazepan-1-yl)methyl)-2-(4-(2,2,2- | | |
| | trifluoroacetyl)phenyl)imidazo[1,2-a]pyridin-6- | | |
| | yl)benzonitrile | | |
| 244 | 6-phenyl-2-(pyridin-3-yl)-3-((4-(trifluoromethyl)piperazin- | | |
| | 1-yl)methyl)imidazo[1,2-a]pyridine | | |
| 245 | 4-((6-phenyl-2-(1H-pyrrol-2-yl)imidazo[1,2-a]pyridin-3- | | |
| | yl)methyl)piperazin-2-one | | |
| 246 | 5-(6-phenyl-3-((4-(trifluoromethyl)-1,4-diazepan-1- | | |
| | yl)methyl)imidazo[1,2-a]pyridin-2-yl)picolinonitrile | | |
| 247 | 4-(2-(p-tolyl)-3-((4-(2,2,2-trifluoroacetyl)piperazin-1- | | |
| | yl)methyl)imidazo[1,2-a]pyridin-6-yl)benzonitrile | | |
| 248 | 4-(6-(4-cyanophenyl)-3-((4-propylpiperazin-1- | | |
| | yl)methyl)imidazo[1,2-a]pyridin-2-yl)benzamide | | |
| 249 | 4-(3-((4-acetylpiperazin-1-yl)methyl)-2-(p- | | |
| | tolyl)imidazo[1,2-a]pyridin-6-yl)benzonitrile | | |
| 250 | 4-(2-(1,5-dimethyl-1H-pyrrol-2-yl)-3-((3-oxopiperazin-1- | | |
| | yl)methyl)imidazo[1,2-a]pyridin-6-yl)benzonitrile | | |
| 251 | 4-(2-(1,5-dimethyl-1H-pyrrol-2-yl)-3-((4-(2,2,2- | | |
| | trifluoroacetyl)-1,4-diazepan-1-yl)methyl)imidazo[1,2- | | |
| | a]pyridin-6-yl)benzonitrile | | |
| 252 | 4-(2-(4-(methylsulfonyl)phenyl)-3-((4-(2,2,2- | | |
| | trifluoroacetyl)-1,4-diazepan-1-yl)methyl)imidazo[1,2- | | |
| | a]pyridin-6-yl)benzonitrile | | |
| 253 | 4-(3-((4-acetyl-1,4-diazepan-1-yl)methyl)-2-(furan-2- | | |
| | yl)imidazo[1,2-a]pyridin-6-yl)benzonitrile | | |
| 254 | 5-(6-phenyl-3-((4-(trifluoromethyl)-1,4-diazepan-1- | | |
| | yl)methyl)imidazo[1,2-a]pyridin-2-yl)thiophene-2- | | |
| | carbonitrile | | |
| 255 | 4-(2-(3-nitrophenyl)-3-(piperazin-1-ylmethyl)imidazo[1,2- | | |
| | a]pyridin-6-yl)benzonitrile | | |
| 256 | 4-(2-(4-(methylsulfonyl)phenyl)-3-((3-oxopiperazin-1- | | |
| | yl)methyl)imidazo[1,2-a]pyridin-6-yl)benzonitrile | | |
| 257 | 4-(2-(furan-2-yl)-3-((4-(2,2,2-trifluoroacetyl)-1,4- | | |
| | diazepan-1-yl)methyl)imidazo[1,2-a]pyridin-6- | | |
| | yl)benzonitrile | | |
| 258 | 2-(furan-2-yl)-6-phenyl-3-((4-(trifluoromethyl)-1,4- | | |
| | diazepan-1-yl)methyl)imidazo[1,2-a]pyridine | | |
| 259 | 4-(3-((4-acetyl-1,4-diazepan-1-yl)methyl)-6-(3,4- | | |
| | dimethylphenyl)imidazo[1,2-a]pyridin-2-yl)benzamide | | |
| 260 | 5-(6-(m-tolyl)-3-((4-(trifluoromethyl)-1,4-diazepan-1- | | |
| | yl)methyl)imidazo[1,2-a]pyridin-2-yl)-1H-pyrrole-2- | | |
| | carbonitrile | | |
| 261 | 3-((4-methylpiperazin-1-yl)methyl)-6-(1H-pyrrol-2-yl)-2- | | |
| | (p-tolyl)imidazo[1,2-a]pyridine | | |
| 262 | 3-(3-(piperazin-1-ylmethyl)-6-(p-tolyl)imidazo[1,2- | | |
| | a]pyridin-2-yl)benzonitrile | | |
| 263 | 1-(4-((2-(pyridin-3-yl)-6-(p-tolyl)imidazo[1,2-a]pyridin-3- | | |
| | yl)methyl)piperazin-1-yl)ethanone | | |
| 264 | 4-(3-((4-methylpiperazin-1-yl)methyl)-6-(p- | | |
| | tolyl)imidazo[1,2-a]pyridin-2-yl)thiophene-2-carbonitrile | | |
| 265 | 4-(3-((4-methylpiperazin-1-yl)methyl)-2-(p- | | |
| | tolyl)imidazo[1,2-a]pyridin-6-yl)thiophene-2-carbonitrile | | |
| 266 | 2-methyl-4-(3-((4-methylpiperazin-1-yl)methyl)-6- | | |
| | (pyridin-3-yl)imidazo[1,2-a]pyridin-2-yl)benzonitrile | | |
| 267 | 1-(4-((6-(pyridin-4-yl)-2-(p-tolyl)imidazo[1,2-a]pyridin-3- | | |
| | yl)methyl)piperazin-1-yl)ethanone | | |
| 268 | 2,2,2-trifluoro-1-(4-(3-((4-methylpiperazin-1-yl)methyl)-6- | | |
| | (m-tolyl)imidazo[1,2-a]pyridin-2-yl)phenyl)ethanone | | |
| 269 | 2,2,2-trifluoro-1-(4-((6-(2-methylpyridin-4-yl)-2-(p- | | |
| | tolyl)imidazo[1,2-a]pyridin-3-yl)methyl)-1,4-diazepan-1- | | |
| | yl)ethanone | | |
| 270 | 4-(2-(p-tolyl)-3-((4-(trifluoromethyl)-1,4-diazepan-1- | | |
| | yl)methyl)imidazo[1,2-a]pyridin-6-yl)benzamide | | |
| 271 | 2-phenyl-3-((4-propylpiperazin-1-yl)methyl)-6-(p- | | |
| | tolyl)imidazo[1,2-a]pyridine | | |
| 272 | 5-(3-((4-acetyl-1,4-diazepan-1-yl)methyl)-6-(3,4- | | |
| | dimethylphenyl)imidazo[1,2-a]pyridin-2-yl)picolinonitrile | | |
| 273 | 4-(6-(3,4-dimethylphenyl)-3-((4-(2,2,2-trifluoroacetyl)-1,4- | | |
| | diazepan-1-yl)methyl)imidazo[1,2-a]pyridin-2- | | |
| | yl)benzamide | | |
| 274 | 3-(3-((4-acetyl-1,4-diazepan-1-yl)methyl)-6-(1,5- | | |
| | dimethyl-1H-pyrrol-2-yl)imidazo[1,2-a]pyridin-2- | | |
| | yl)benzonitrile | | |
| 275 | 5-(3-((4-methylpiperazin-1-yl)methyl)-2-(p- | | |
| | tolyl)imidazo[1,2-a]pyridin-6-yl)thiophene-2-carbonitrile | | |
| 276 | 1-(4-((6-(4-(dimethylamino)phenyl)-2-(p- | | |
| | tolyl)imidazo[1,2-a]pyridin-3-yl)methyl)-1,4-diazepan-1- | | |
| | yl)ethanone | | |
| 277 | 1-(4-((2-phenyl-6-(4-(trifluoromethyl)phenyl)imidazo[1,2- | | |
| | a]pyridin-3-yl)methyl)-1,4-diazepan-1-yl)ethanone | | |
| 278 | 6-(4-methoxyphenyl)-3-((4-methylpiperazin-1-yl)methyl)- | | |
| | 2-(p-tolyl)imidazo[1,2-a]pyridine | | |
| 279 | 1-(4-((6-(4-methoxyphenyl)-2-(p-tolyl)imidazo[1,2- | | |
| | a]pyridin-3-yl)methyl)-1,4-diazepan-1-yl)ethanone | | |
| 280 | 5-(3-((4-methylpiperazin-1-yl)methyl)-6-(p- | | |
| | tolyl)imidazo[1,2-a]pyridin-2-yl)thiophene-2-carbonitrile | | |
| 281 | N,N-dimethyl-4-(3-((4-methylpiperazin-1-yl)methyl)-2-(p- | | |
| | tolyl)imidazo[1,2-a]pyridin-6-yl)aniline | | |
| 282 | 6-(2-methylpyridin-4-yl)-2-(p-tolyl)-3-((4-(trifluoromethyl)- | | |
| | 1,4-diazepan-1-yl)methyl)imidazo[1,2-a]pyridine | | |
| 283 | 4-(3-((4-methylpiperazin-1-yl)methyl)-6-(m-tolyl)- | | |
| | imidazo[1,2-a]pyridin-2-yl)benzamide | | |
| 284 | 2,2,2-trifluoro-1-(4-(3-((4-methylpiperazin-1-yl)methyl)-6- | | |
| | (1H-pyrrol-2-yl)imidazo[1,2-a]pyridin-2- | | |
| | yl)phenyl)ethanone | | |
| 285 | 4-(3-((4-acetylpiperazin-1-yl)methyl)-6-(p- | | |
| | tolyl)imidazo[1,2-a]pyridin-2-yl)thiophene-2-carbonitrile | | |
| 286 | 2,2,2-trifluoro-1-(4-((6-(pyridin-3-yl)-2-(p- | | |
| | tolyl)imidazo[1,2-a]pyridin-3-yl)methyl)piperazin-1- | | |
| | yl)ethanone | | |
| 287 | 5-(3-((4-acetyl-1,4-diazepan-1-yl)methyl)-6-(p- | | |
| | tolyl)imidazo[1,2-a]pyridin-2-yl)thiophene-3-carbonitrile | | |
| 288 | 2-(1,5-dimethyl-1H-pyrrol-2-yl)-3-((4-propylpiperazin-1- | | |
| | yl)methyl)-6-(m-tolyl)imidazo[1,2-a]pyridine | | |
| 289 | 4-(3-((3-oxopiperazin-1-yl)methyl)-6-(1H-pyrrol-2- | | |
| | yl)imidazo[1,2-a]pyridin-2-yl)benzamide | | |
| 290 | 6-(4-methoxyphenyl)-3-((4-methylpiperazin-1-yl)methyl)- | | |
| | 2-(1H-pyrrol-2-yl)imidazo[1,2-a]pyridine | | |
| 291 | 3-((4-methylpiperazin-1-yl)methyl)-6-(pyridin-4-yl)-2-(4- | | |
| | (trifluoromethyl)phenyl)imidazo[1,2-a]pyridine | | |
| 292 | 4-(6-(4-(dimethylamino)phenyl)-3-(piperazin-1- | | |
| | ylmethyl)imidazo[1,2-a]pyridin-2-yl)-2-methylbenzonitrile | | |
| 293 | 1-(4-((6-(4-chlorophenyl)-2-(p-tolyl)imidazo[1,2-a]pyridin- | | |
| | 3-yl)methyl)-1,4-diazepan-1-yl)ethanone | | |
| 294 | 3-((4-methylpiperazin-1-yl)methyl)-6-(1H-pyrrol-2-yl)-2- | | |
| | (4-(trifluoromethyl)phenyl)imidazo[1,2-a]pyridine | | |
| 295 | 4-(2-(3,4-dimethylphenyl)-3-((4-(2,2,2-trifluoroacetyl)-1,4- | | |
| | diazepan-1-yl)methyl)imidazo[1,2-a]pyridin-6-yl)-2- | | |
| | methylbenzonitrile | | |
| 296 | 1-(4-((2-(5-methyl-1H-pyrrol-2-yl)-6-(pyridin-4- | | |
| | yl)imidazo[1,2-a]pyridin-3-yl)methyl)-1,4-diazepan-1- | | |
| | yl)ethanone | | |
| 297 | 3-((4-methylpiperazin-1-yl)methyl)-2-(4-nitrophenyl)-6- | | |
| | (1H-pyrrol-2-yl)imidazo[1,2-a]pyridine | | |
| 298 | 3-((4-methylpiperazin-1-yl)methyl)-2-(1H-pyrrol-2-yl)-6- | | |
| | (m-tolyl)imidazo[1,2-a]pyridine | | |
| 299 | 4-(3-(piperazin-1-ylmethyl)-6-(4-(trifluoromethyl)- | | |
| | phenyl)imidazo[1,2-a]pyridin-2-yl)benzamide | | |
| 300 | 3-((4-methylpiperazin-1-yl)methyl)-2-(1H-pyrrol-2-yl)-6- | | |
| | (4-(trifluoromethyl)phenyl)imidazo[1,2-a]pyridine | | |
| 301 | 5-(3-((4-methylpiperazin-1-yl)methyl)-6-(p- | | |
| | tolyl)imidazo[1,2-a]pyridin-2-yl)picolinonitrile | | |
| 302 | 5-(3-((4-methylpiperazin-1-yl)methyl)-2-(p- | | |
| | tolyl)imidazo[1,2-a]pyridin-6-yl)-1H-pyrrole-2-carbonitrile | | |
| 303 | 4,4′-(3-((3-oxopiperazin-1-yl)methyl)imidazo[1,2- | | |
| | a]pyridine-2,6-diyl)dibenzamide | | |
| 304 | 5-(3-((4-acetyl-1,4-diazepan-1-yl)methyl)-2-(3- | | |
| | cyanophenyl)imidazo[1,2-a]pyridin-6-yl)thiophene-2- | | |
| | carbonitrile | | |
| 305 | 4-(3-((1,4-diazepan-1-yl)methyl)-6-(4- | | |
| | ((trifluoromethyl)thio)phenyl)imidazo[1,2-a]pyridin-2- | | |
| | yl)benzonitrile | | |
| 306 | 4-(3-((1,4-diazepan-1-yl)methyl)-6-(4- | | |
| | ((trifluoromethyl)sulfonyl)phenyl)imidazo[1,2-a]pyridin-2- | | |
| | yl)benzonitrile | | |

Rₜ^{#} retention time, HPLC

### Biology Examples

### HIF/HRE Reporter Assay

Inhibition of an activated HIF signaling response under chemically-induced hypoxic conditions due to compound treatment was determined using the CellSensor® HRE-bla HCT-116 stably transfected reporter cell line from Invitrogen according to the manufacturer's instructions.

Cells were maintained as described previously and seeded into 384-well, clear-bottom plates (Corning 3712) at 15000 cells/well in 32 μl assay medium (Opti-MEM [Invitrogen], 0.5% FBS, 100 U/ml penicillin, 100 μg/ml streptomycin, 0.1 mM non-essential amino acids [NEAA], 1 mM sodium pyruvate, 5 mM HEPES [pH 7.3]). Following a 2 h incubation period, compounds (4 μl) were subsequently added to the cells at 10× concentrations in 5% DMSO and incubated under normal conditions for 30 min. To induce hypoxic conditions, 4 μl of a 2 mM deferoxamine (DFO) solution was added to the cells followed by 24 h incubation under standard assay conditions (as described). Control wells included wells containing only medium (no cells) and wells treated with 0.5% DMSO instead of compound.

Prior to the readout, the Substrate Loading Solution was prepared as described in the manufacturer's protocol and 10 μl added to each well. Following a further 2 h incubation period at room temperature and in the dark, fluorescence was measured at two wavelengths (blue channel: ex. 409 nm, em. 460 nm; green channel: ex. 409 nm, em. 530 nm) on a PerkinElmer Envision HTS. For the analysis, the average signal of the cell-free wells at 460 nm and 530 nm was first subtracted from the blue and green channel data, respectively. The blue/green emission ratios were then calculated for each well, dividing the background-corrected blue emission values by the background-corrected green emission values. 1050 values were determined from these ratios using GraphPad Prism (Prism 5, GraphPad software, Inc.). The results are summarized in the following table.

| Example | Evaluation |
|---|---|
| 1 | +++ |
| 2 | + |
| 3 | ++ |
| 4 | +++ |
| 5 | ++ |
| 6 | ++ |
| 7 | +++ |
| 8 | + |
| 9 | +++ |
| 10 | + |
| 11 | +++ |
| 12 | +++ |
| 13 | +++ |
| 14 | +++ |
| 15 | +++ |
| 16 | ++ |
| 17 | +++ |
| 18 | +++ |
| 19 | ++ |
| 20 | +++ |
| 21 | +++ |
| 22 | +++ |
| 23 | +++ |
| 24 | +++ |
| 25 | +++ |
| 26 | + |
| 27 | +++ |
| 28 | ++ |
| 29 | + |
| 30 | + |
| 31 | +++ |
| 32 | + |
| 33 | + |
| 34 | +++ |
| 35 | + |
| 36 | +++ |
| 37 | + |
| 38 | ++ |
| 39 | + |
| 40 | +++ |
| 41 | +++ |
| 42 | + |
| 43 | +++ |
| 44 | +++ |

Reference:
EC₅₀ < 500 nM: +++
500-1000 nM: ++
>1000 nM: +

The results of these experiments show that the compounds of the invention are capable of inhibiting hypoxia regulated element-mediated transcriptional activity under hypoxic conditions.

1. A method for treating a disorder or disease selected from the group consisting of an inflammatory disease, a hyperproliferative disease or disorder, a hypoxia related pathology and a disease characterized by pathophysiological hypervascularization in a subject in need thereof, comprising administering to the subject an effective amount of an imidazo[1,2-a]pyridine compound of formula (I): 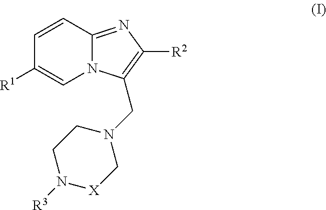
wherein
X is CH₂, CH₂CH₂ or C═O;
R¹ is phenyl or C-bound monocyclic 5- or 6-membered heteroaryl, wherein phenyl and monocyclic 5- or 6-membered heteroaryl are unsubstituted or carry 1, 2, 3, 4 or 5 radicals R^{1a} which are identical or different;
R^{1a} is selected from the group consisting of halogen, CN, NO₂, NH₂, OH, SH, C₁-C₁₀-alkenyl, C₂-C₁₀-alkenyl, C₂-C₁₀-alkynyl, C₁-C₆-alkoxy, C₁-C₆-alkylthio, hydroxy-C₁-C₆-alkyl, C₁-C₄-alkoxy-C₁-C₄-alkyl, fluorinated C₁-C₂-alkyl, SF₅, fluorinated C₁-C₂-alkoxy, fluorinated C₁-C₂-alkylsulfonyl, fluorinated C₁-C₂-alkylthio, C(O)R⁴, NR⁵R⁶, C(O)NR⁷R⁸ and C(O)OR⁹, or two radicals R^{1a}, which are bound to adjacent carbon atoms, may also form a bridging moiety O-Alk-O, wherein Alk is selected from CH₂, CH₂CH₂, CHF, CF₂ and CF₂CF₂;
R² is phenyl or C-bound monocyclic 5- or 6-membered heteroaryl, wherein phenyl and monocyclic 5- or 6-membered heteroaryl are unsubstituted or carry 1, 2, 3, 4 or 5 radicals R^{2a} which are identical or different;
R^{2a} is selected from the group consisting of halogen, CN, NO₂, NH₂, OH, SH, C₁-C₁₀-alkyl, C₂-C₁₀-alkenyl, C₂-C₁₀-alkynyl, C₁-C₆-alkoxy, C₁-C₆-alkylthio, hydroxy-C₁-C₆-alkyl, C₁-C₄-alkoxy-C₁-C₄-alkyl, fluorinated C₁-C₂-alkyl, SF₅, C₁-C₄-alkyl-sulfonyl, fluorinated C₁-C₂-alkoxy, C(O)R⁴, NR⁵R⁶, C(O)NR⁷R⁸ and C(O)OR⁹, or two radicals R^{2a}, which are bound to adjacent carbon atoms, may also form a bridging moiety O-Alk′-O, wherein Alk′ is selected from CH₂, CH₂CH₂, CHF, CF₂ and CF₂CF₂;
R³ is hydrogen, C₁-C₆-alkyl, C₁-C₄-alkoxy-C₁-C₄-alkyl, fluorinated C₁-C₂-alkyl or C(O)R⁴;
R⁴ is selected from the group consisting of hydrogen, C₁-C₄-alkyl, C₁-C₄-alkoxy-C₁-C₄-alkyl, C₃-C₇-cycloalkyl, fluorinated C₁-C₂-alkyl, C₁-C₄-alkoxy, C₁-C₄-alkoxy-C₁-C₄-alkoxy, C₃-C₇-cycloalkoxy, fluorinated C₁-C₂-alkoxy;
R⁵ is hydrogen or C₁-C₆-alkyl;
R⁶ is C₁-C₆-alkyl, hydroxy-C₂-C₆-alkyl, C₁-C₄-alkoxy-C₂-C₄-alkyl, OH, C₁-C₄-alkoxy or a radical C(O)R^{x}, wherein R^{x} is C₁-C₄-alkyl; or
R⁵, R⁶ together with the nitrogen atom, to which they are bound, form an N-bound, 5- or 6-membered saturated nitrogen heterocycle;
R⁷ and R⁸ independently of one another are hydrogen, OH, C₁-C₄-alkoxy or C₁-C₆-alkyl;
R⁹ is hydrogen, C₁-C₆-alkyl, hydroxy-C₂-C₆-alkyl or C₁-C₄-alkoxy-C₂-C₄-alkyl;
or a pharmaceutically acceptable salt thereof, an N-oxide thereof or a pharmaceutically acceptable salt of said N-oxide.

2. The method of claim 1, wherein R¹ is phenyl or a 6-membered heteroaryl, which are unsubstituted or carry 1, 2 or 3 radicals R^{1a} being identical or different.

3. The method of claim 2, wherein R¹ is a radical of the formula Ar1 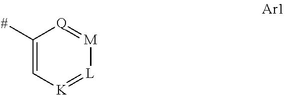
wherein # indicates the point of attachment to the imidazo[1,2-a]pyridine core of (I),
K is N or C—R¹¹,
L is N or C—R¹²,
M is N or C—R¹³,
Q is N or C—R¹⁴,
wherein R¹¹, R¹², R¹³ and R¹⁴, independently of each other, are hydrogen or have one of the meanings given for R^{1a}.

4. The method of claim 3, wherein R¹ is selected from radicals of the formulae Ar1.1 to Ar1.7: 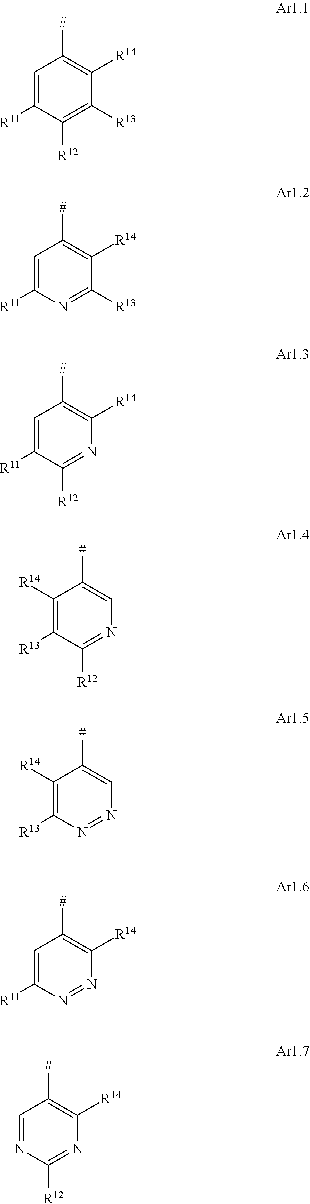
wherein # indicates the point of attachment to the imidazo[1,2-a]pyridine core of (I), and
wherein R¹¹, R¹², R¹³ and R¹⁴, independently of each other, are hydrogen or have one of the meanings given for R^{1a}.

5. The method of claim 3, wherein
R¹¹, R¹³ if present, are independently of each other selected from the group consisting of hydrogen, halogen, OH, CN, NO₂, SH, C₁-C₄-alkyl, C₂-C₄-alkenyl, C₂-C₄-alkynyl, C₁-C₄-alkoxy, C₁-C₄-alkylthio, fluorinated C₁-alkyl, fluorinated C₁-alkoxy, NH₂, NR⁵R⁶, hydroxy-C₁-C₄-alkyl and C₁-C₄-alkoxy-C₁-C₄-alkyl;
R¹² if present, is selected from the group consisting of hydrogen, halogen, OH, SH, CN, NO₂, C₁-C₄-alkyl, C₂-C₄-alkenyl, C₂-C₄-alkynyl, C₁-C₄-alkoxy, C₁-C₄-alkylthio, fluorinated C₁-C₂-alkyl, SF₅, fluorinated C₁-C₂-alkoxy, NH₂, hydroxy-C₁-C₄-alkyl, C₁-C₄-alkoxy-C₁-C₄-alkyl, C(O)R⁴, NR⁵R⁶, C(O)NH₂ and C(O)OR⁹;
R¹⁴ if present, is selected from the group consisting of hydrogen, halogen, OH, C₁-C₄-alkyl, C₂-C₄-alkenyl, C₂-C₄-alkynyl, C₁-C₄-alkoxy, fluorinated C₁-C₂-alkyl and fluorinated C₁-C₂-alkoxy.

6. The method of claim 4, wherein R¹ is a radical of the formulae Ar1.1.

7. The method of claim 6, wherein
R¹¹ is selected from the group consisting of hydrogen, halogen, methyl, ethyl, difluoromethyl, trifluoromethyl, methoxy, difluoromethoxy and trifluoromethoxy;
R¹² is selected from the group consisting of hydrogen, halogen, CN, NO₂, C₁-C₄-alkyl, C₂-C₄-alkenyl, C₂-C₄-alkynyl, C₁-C₄-alkoxy, C₁-C₄-alkylthio, fluorinated C₁-alkyl, SF₅, fluorinated C₁-alkoxy, NH₂, hydroxy-C₁-C₄-alkyl, C₁-C₄-alkoxy-C₁-C₄-alkyl, C(O)R⁴, NR⁵R⁶ and C(O)NH₂;
R¹³ is hydrogen;
and R¹⁴ is hydrogen.

8. The method of claim 6, wherein R¹¹, R¹², R¹³ and R¹⁴ are hydrogen.

9. The method of claim 1, wherein R¹ is a 5-membered C-bound heteroaryl, which is unsubstituted or which carries 1, 2, 3 or 4 radicals R^{1a} which are identical or different.

10. The method of claim 9, wherein R¹ is a radical of the formulae 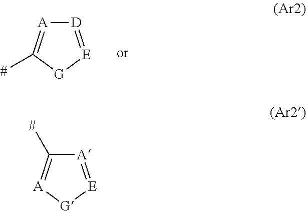
wherein # indicates the point of attachment of R¹ to the imidazo[1,2-a]pyridine core of (I),
A is N or C—R¹⁵,
A′ is N or C—R¹⁶,
D is N or C—R¹⁷,
E is N or C—R¹⁸,
is O, S or N—R¹⁹, G′ is O, S or N—R²⁰,
wherein R¹⁵, R¹⁶, R¹⁷ and R¹⁸, independently of each other, are hydrogen or have one of the meanings given for R^{1a} and wherein R¹⁹ and R²⁰ are selected from the group consisting of hydrogen, CN, NH₂, OH, C₁-C₁₀-alkyl, C₂-C₁₀-alkenyl, C₂-C₁₀-alkynyl, C₁-C₆-alkoxy, hydroxy-C₂-C₆-alkyl, C₁-C₄-alkoxy-C₂-C₄-alkyl, fluorinated C₁-C₂-alkyl, fluorinated C₁-C₂-alkoxy, C(O)R⁴, NR⁵R⁶, and C(O)OR⁹.

11. The method of claim 10, wherein R¹ is selected from radicals of the formulae Ar2.1, Ar2.2, Ar2.3, Ar2.4, Ar2.5, Ar2.6, Ar2.7, Ar2.8, Ar2.9, Ar2.10, Ar2.11, Ar2.12, Ar2.13, Ar2.14, Ar2.15, Ar2.16, Ar2.17, Ar2.18, Ar2.19, Ar2.20, Ar2.21, Ar2.22, Ar2′.1, Ar2′.2, Ar2′.3, Ar2′.4, Ar2′.5, Ar2′.6, Ar2′.7, Ar2′.8, Ar2′.9, Ar2′.10, Ar2′.11, Ar2′.12, Ar2′.13, Ar2′.14 and Ar2′.15: 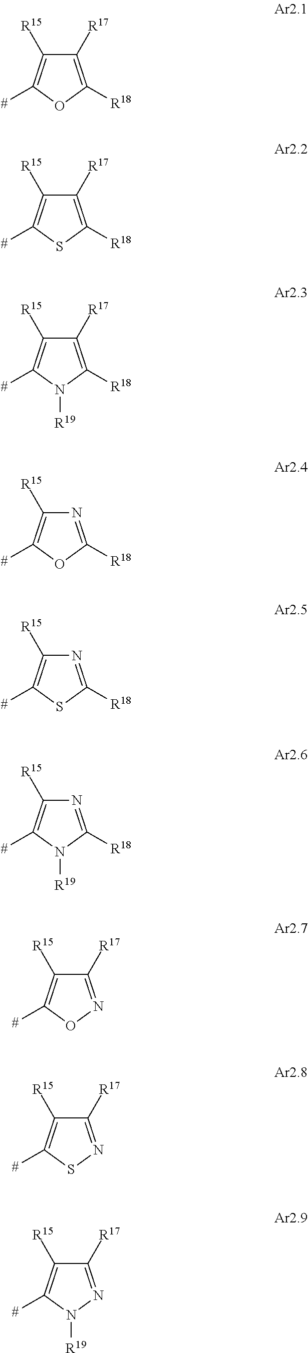 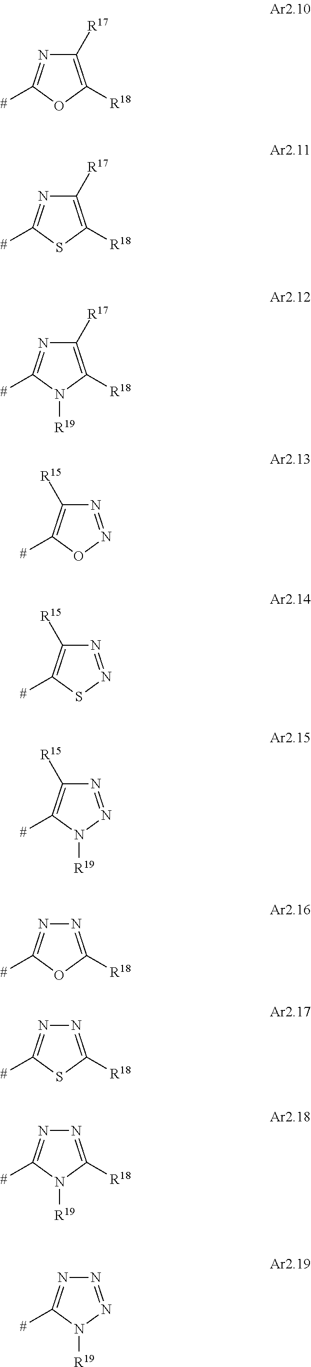 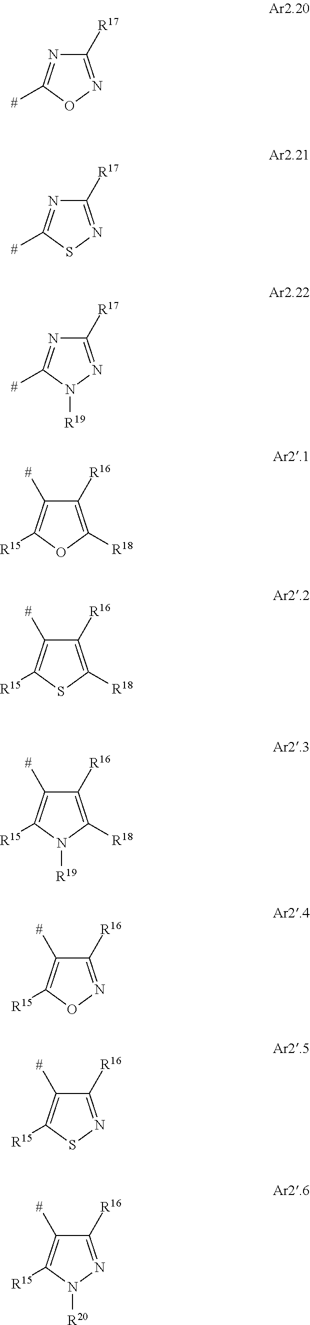 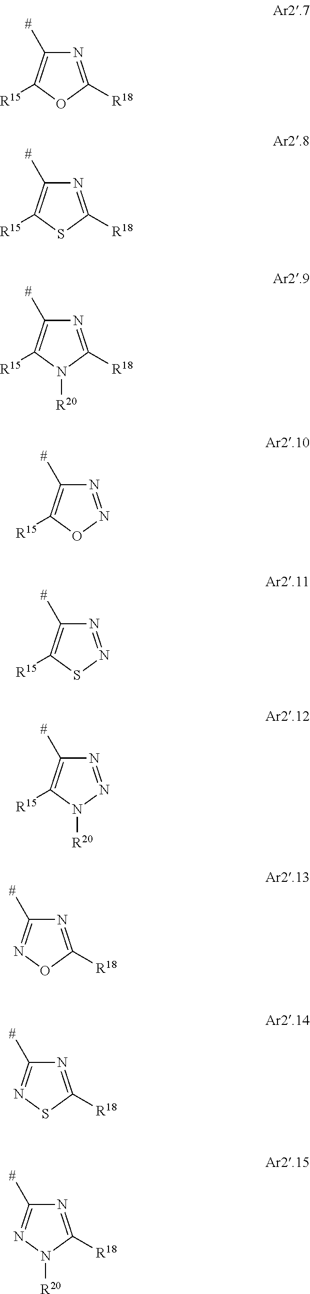
wherein # indicates the point of attachment of R¹ to the imidazo[1,2-a]pyridine core of (I), and
wherein R¹⁵, R¹⁶, R¹⁷, R¹⁸, R¹⁹ and R²⁰ have the meanings given in claim 9.

12. The method of claim 10, wherein
R¹⁵ if present, is hydrogen;
R¹⁶ if present, is hydrogen;
R¹⁷ if present, is selected from the group consisting of hydrogen, halogen, CN, NO₂, C₁-C₄-alkyl, C₂-C₄-alkenyl, C₂-C₄-alkynyl, C₁-C₄-alkoxy, C₁-C₄-alkylthio, fluorinated C₁-alkyl, fluorinated C₁-alkoxy, NH₂, hydroxy-C₁-C₄-alkyl, C₁-C₄-alkoxy-C₁-C₄-alkyl, C(O)R⁴ and NR⁵R⁶;
R¹⁸ if present, is selected from the group consisting of hydrogen, halogen, CN, NO₂, C₁-C₄-alkyl, C₂-C₄-alkenyl, C₂-C₄-alkynyl, C₁-C₄-alkoxy, C₁-C₄-alkylthio, fluorinated C₁-alkyl, fluorinated C₁-alkoxy, NH₂, hydroxy-C₁-C₄-alkyl, C₁-C₄-alkoxy-C₁-C₄-alkyl, C(O)R⁴ and NR⁵R⁶;
R¹⁹ if present, is selected from the group consisting of hydrogen, CN, C₁-C₄-alkyl, C₂-C₄-alkenyl, C₂-C₄-alkynyl, fluorinated C₁-alkyl, C₁-C₄-alkoxy-C₂-C₄-alkyl and hydroxy-C₂-C₄-alkyl; and
R²⁰ if present, is selected from the group consisting of hydrogen, CN, C₁-C₄-alkyl, C₂-C₄-alkenyl, C₂-C₄-alkynyl, fluorinated C₁-alkyl, C₁-C₄-alkoxy-C₂-C₄-alkyl and hydroxy-C₂-C₄-alkyl.

13. The method of claim 1, wherein R¹ is selected from the group consisting of phenyl, 2-methylphenyl, 3-methylphenyl, 3-methoxyphenyl, 3-chlorophenyl, 3-nitrophenyl, 3-cyanophenyl, 3-trifluoromethylphenyl, 3-trifluoromethoxyphenyl, 4-acetylphenyl, 4-methylphenyl, 4-methoxyphenyl, 4-trifluoromethylphenyl, 4-trifluoromethoxyphenyl, 4-bromophenyl, 4-chlorophenyl, 4-fluorophenyl, 4-(methylthio)phenyl, 4-cyanophenyl, 4-aminophenyl, 4-nitrophenyl, 4-(dimethylamino)phenyl, 4-methylamino-phenyl, 4-aminocarbonylphenyl, 3-chloro-4-methylamino-phenyl, 3-chloro-4-methylphenyl, 3-chloro-4-methoxyphenyl, 3-methyl-4-chlorophenyl, 4-chloro-3-cyanophenyl, 3,4-dimethylphenyl, 3,4-dimethoxyphenyl, 3,4-dicyanophenyl, 3-methyl-4-cyanophenyl, 3-fluoro-4-cyanophenyl, 3-methoxy-4-cyanophenyl, 3-chloro-4-cyanophenyl, 3,5-chloro-4-cyanophenyl, 4-pentafluorosulfanylphenyl, 3-pyridyl, 4-pyridyl, 5-cyanopyridine-3-yl, 6-isopropylpyridine-3-yl, 5-fluoropyridine-3-yl, 5-chloropyridine-3-yl, 5-methoxypyridine-3-yl, 5-trifluoromethoxypyridine-3-yl, 5-methylpyridine-3-yl, 4-methylpyridine-2-yl, 5-methylpyridine-2-yl, 6-methylpyridine-2-yl, 5-trifluoromethylpyridine-3-yl, 6-cyanopyridine-3-yl, 6-fluoropyridine-3-yl, 6-chloropyridine-3-yl, 6-methylpyridine-3-yl, 6-methylthiopyridine-3-yl, 6-trifluoromethylpyridine-3-yl, 6-methoxypyridine-3-yl, 6-ethoxypyridine-3-yl, 6-trifluoromethoxypyridine-3-yl, 6-dimethylaminopyridine-3-yl, 2-chloropyridine-4-yl, 2-cyanopyridine-4-yl, 2-methylpyridine-4-yl, 2-methoxypyridine-4-yl, 2-trifluoromethylpyridine-4-yl, 2-trifluoromethoxypyridine-4-yl, 5-cyano-4-methylpyridine-3-yl, 2-cyano-6-methylpyridine-4-yl, pyrimidine-5-yl, 2-methylthiopyrimidine-5-yl, 2-trifluoromethoxypyrimidine-5-yl, 5-methoxypyridazin-3-yl, 5-methylpyridazin-3-yl, 5-chloropyridazin-3-yl, 5-cyanopyridazin-3-yl, furan-2-yl, furan-3-yl, 5-methylfuran-2-yl, 4-methylfuran-2-yl, 5-methylfuran-3-yl, 5-cyanofuran-2-yl, 4-cyanofuran-2-yl, 5-cyanofuran-3-yl, 5-nitrofuran-2-yl, 2-thienyl, 3-thienyl, 5-methyl-2-thienyl, 4-methyl-2-thienyl, 5-methyl-3-thienyl, 5-cyano-2-thienyl, 4-cyano-2-thienyl, 5-cyano-3-thienyl, 4-cyano-3-thienyl, 5-trifluoromethoxy-3-thienyl, 4-chloro-5-methyl-2-thienyl, 5-cyano-4-fluoro-2-thienyl, pyrrol-2-yl, pyrrol-3-yl, 1-methylpyrrol-2-yl, 4-methylpyrrol-2-yl, 5-methylpyrrol-2-yl, 5-methylpyrrol-3-yl, 5-cyanopyrrol-2-yl, 4-cyanopyrrol-2-yl, 5-cyanopyrrol-3-yl, 1,5-dimethylpyrrol-2-yl, pyrazol-3-yl, pyrazol-4-yl, 1-methylpyrazol-3-yl, 1-methylpyrazol-4-yl, 1-ethylpyrazol-4-yl, 1-oxopyrazol-4-yl, 1-methylimidazol-4-yl, 1-methylimidazol-5-yl, 1,2-dimethylimidazol-4-yl, 1,2-dimethylimidazol-5-yl, 4-methylthiazol-2-yl, isothiazol-3-yl, isothiazol-4-yl, isothiazol-5-yl, 5-methylisothiazol-3-yl, 3-methylisothiazol-5-yl, 5-methylisothiazol-3-yl, 3-cyanoisothiazol-5-yl, 2-methyloxazol-5-yl, oxazol-5-yl, 1,2,3-thiadiazol-4-yl, 1,2,3-thiadiazol-5-yl, 4-methyl-1,2,3-thiadiazol-5-yl, 5-methyl-1,2,3-thiadiazol-4-yl, 4-cyano-1,2,3-thiadiazol-5-yl, 1,3,4-thiadiazol-2-yl, 1,3,4-oxadiazol-2-yl, 5-cyano-1,3,4-thiadiazol-2-yl, 5-cyano-1,3,4-oxadiazol-2-yl, 5-methyl-1,3,4-oxadiazol-2-yl, 5-methyl-1,3,4-thiadiazol-2-yl, 1,2,4-[4H]triazol-3-yl, 1,2,3,4-tetrazol-5-yl and 2-methyl-1,2,3,4-tetrazol-5-yl.

14. The method of claim 1, wherein R² is phenyl or a 6-membered heteroaryl, which are unsubstituted or carry 1, 2 or 3 radicals R^{2a} being identical or different.

15. The method of claim 14, wherein R² is a radical of the formula Ar3 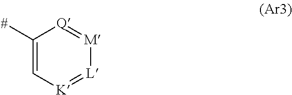
wherein # indicates the point of attachment to the imidazo[1,2-a]pyridine core of (I),
K′ is N or C—R²¹,
L′ is N or C—R²²,
M′ is N or C—R²³,
Q′ is N or C—R^{23a},
wherein R²¹, R²², R²³ and R^{23a}, independently of each other, are hydrogen or have one of the meanings given for R^{2a}.

16. The method of claim 14, wherein R² is selected from radicals of the formulae Ar3.1, Ar3.2, Ar3.3, Ar3.4, Ar3.5, Ar3.6, Ar3.7, Ar3.8, Ar3.9, Ar3.10 and Ar3.11, 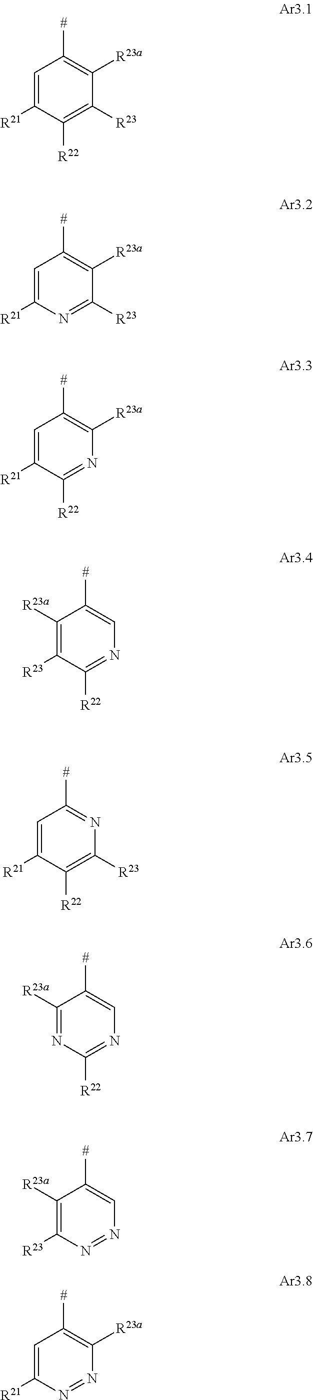 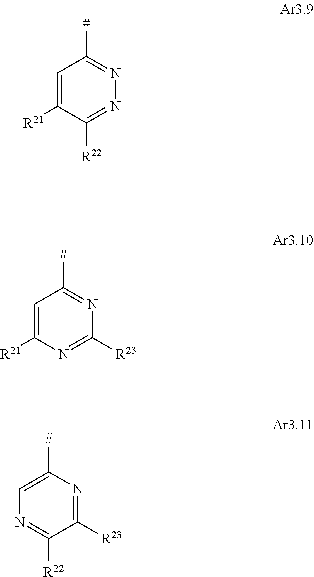
wherein # indicates the point of attachment to the imidazo[1,2-a]pyridine core of (I), and
wherein R²¹, R²², R²³ and R^{23a} independently of each other, are hydrogen or have one of the meanings given for R^{2a}.

17. The method of claim 15, wherein R²¹, R²², R²³ and R^{23a} if present, are independently of each other selected from the group consisting of hydrogen, halogen, NO₂, CN, methyl, methoxy, methylsulfonyl, difluoromethyl, trifluoromethyl, difluoromethoxy, trifluoromethoxy, aminocarbonyl, acetyl and trifluoroacetyl.

18. The method of claim 16, wherein R² is a radical Ar3.1, Ar3.3 or Ar3.4.

19. The method of claim 17, wherein R²² is selected from the group consisting of hydrogen, methoxy, chloride, NO₂, CN, methyl, methylsulfonyl, trifluoromethyl, trifluoroacetyl and aminocarbonyl, and wherein R²¹ and R²³ are independently of each other selected from the group consisting of hydrogen, NO₂, CN and methyl.

20. The method of claim 1, wherein R² is 5-membered heteroaryl, which is unsubstituted or carries 1, 2 or 3 radicals R^{2a} which are identical or different.

21. The method of claim 20, wherein R² is a radical of the formulae Ar4 or Ar5 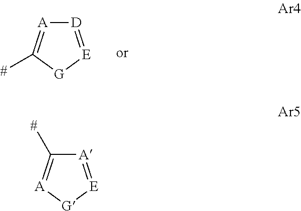
wherein # indicates the point of attachment of R² to the imidazo[1,2-a]pyridine core of (I),
A is N or C—R²⁴,
A′ is N or C—R²⁵,
D is N or C—R²⁶,
E is N or C—R²⁷,
G is O, S or N—R²⁸,
G′ is O, S or N—R²⁹,
wherein R²⁴, R²⁵, R²⁶ and R²⁷, independently of each other, are hydrogen or have one of the meanings given for R^{2a}, and wherein R²⁸ and R²⁹ are selected from the group consisting of hydrogen, CN, C₁-C₁₀-alkyl, C₂-C₁₀-alkenyl, C₂-C₁₀-alkynyl, C₁-C₆-alkoxy, fluorinated C₁-C₂-alkyl, fluorinated C₁-C₂-alkoxy, C(O)R⁴, NR⁵R⁶, and C(O)OR⁹.

22. The method of claim 21, wherein R² is selected from radicals of the formulae Ar4.1, Ar4.2, Ar4.3, Ar4.4, Ar4.5, Ar4.6, Ar4.7, Ar4.8, Ar4.9, Ar4.10, Ar4.11, Ar4.12, Ar4.13, Ar4.14, Ar4.15, Ar5.1, Ar5.2, Ar5.3, Ar5.4, Ar5.5, Ar5.6, Ar5.7, Ar5.8, Ar5.9, Ar5.10, Ar5.11 and Ar5.12, 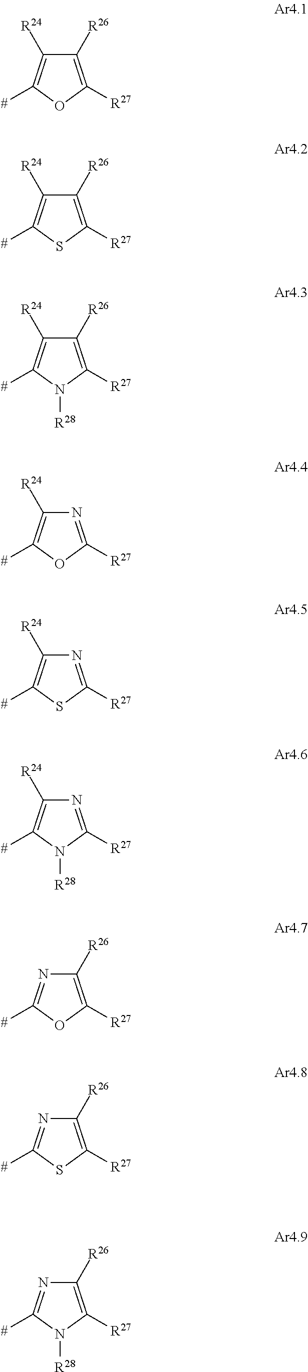 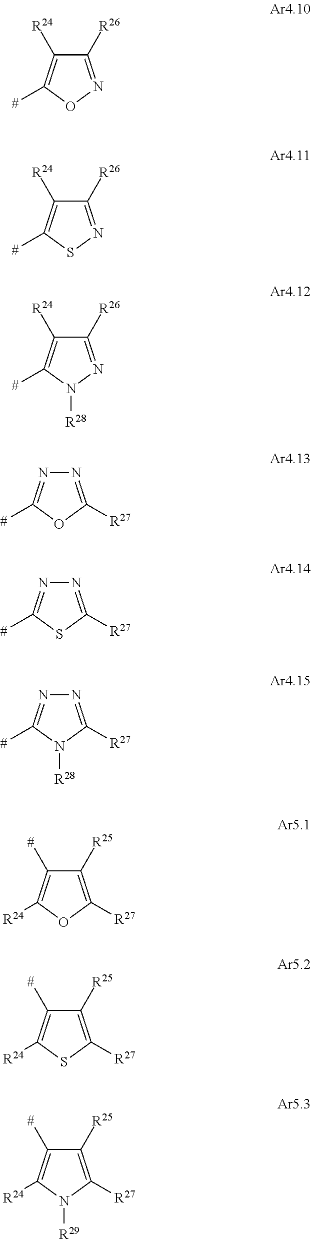 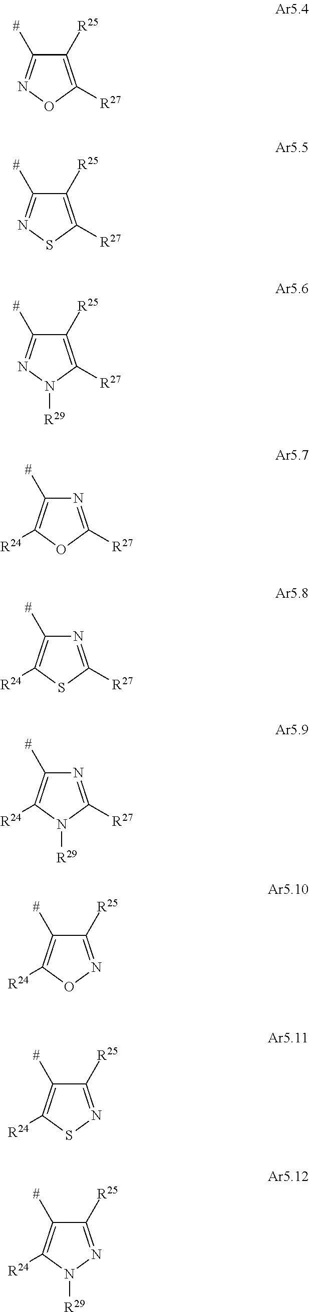
wherein # indicates the point of attachment of R² to the imidazo[1,2-a]pyridine core of (I),
and wherein R²⁴, R²⁵, R²⁶, R²⁷, R²⁸ and R²⁹ have the meanings given in claim 21.

23. The method of claim 22, wherein R² is selected from the radicals Ar4.1, Ar4.2, Ar4.3, Ar5.1, Ar5.2 and Ar5.3.

24. The method of claim 21, wherein
R²⁴, R²⁵, R²⁶ and R²⁷, if present, independently of each other are selected from the group consisting of hydrogen, methyl and CN;
R²⁸ and R²⁹, if present, independently of each other are selected from the group consisting of hydrogen and methyl.

25. The method of claim 1, wherein R² is selected from the group consisting of phenyl, 3-cyanophenyl, 4-cyanophenyl, 4-methoxyphenyl, 3-methoxyphenyl, 4-chlorphenyl, 3-chlorphenyl, 3,4-dicyanophenyl, 3-methoxy-4-cyanophenyl, 4-methoxy-3-cyanophenyl, 3-chloro-4-cyanophenyl, 4-chloro-3-cyanophenyl, 3,4-dimethoxyphenyl, 3-chloro-4-methoxyphenyl, 4-chloro-3-methoxyphenyl, 3,4-dichlorophenyl, 4-methylphenyl, 3-methylphenyl, 3,4-dimethylphenyl, 3-methyl-4-cyanophenyl, 4-methyl-3-cyanophenyl, 3-methyl-4-chlorophenyl, 4-methyl-3-chlorophenyl, 3-methyl-4-methoxyphenyl, 4-methyl-3-methoxyphenyl, 4-trichloroacetylphenyl, 3-trichloroacetylphenyl, 4-aminocarbonylphenyl, 3-aminocarbonylphenyl, 4-methylsulfonylphenyl, 3-methylsulfonylphenyl, 4-nitrophenyl, 3-nitrophenyl, 4-trifluoromethylphenyl, 3-trifluoromethylphenyl, pyridine-2-yl, pyridine-3-yl, pyridine-4-yl, 6-cyanopyridine-3-yl, 6-methoxypyridine-3-yl, 6-chloropyridine-3-yl, 6-methlypyridine-3-yl, 5-cyanopyridine-2-yl, 5-methoxypyridine-2-yl, 5-chloropyridine-2-yl, 5-methlypyridine-2-yl, pyrimidine-2-yl, pyrimidine-4-yl, pyrimidine-5-yl, 5-cyanopyrimidine-2-yl, 2-cyanopyrimidine-5-yl, 5-methoxypyrimidine-2-yl, 2-methoxypyrimidine-5-yl, 5-chloropyrimidine-2-yl, 2-chloropyrimidine-5-yl, 5-methylpyrimidine-2-yl, 2-methylpyrimidine-5-yl, pyrazin-2-yl, 5-cyano-pyrazin-2-yl, 5-methoxy-pyrazin-2-yl, 5-chloro-pyrazin-2-yl, pyridazin-3-yl, pyridazin-4-yl, 6-cyanopyridazin-3-yl, 6-methoxypyridazin-3-yl, 6-chloropyridazin-3-yl, furan-2-yl, furan-3-yl, 5-cyanofuran-2-yl, 4-cyanofuran-2-yl, 5-cyanofuran-3-yl, 2-thienyl, 3-thienyl, 5-cyano-2-thienyl, 4-cyano-2-thienyl, 5-cyano-3-thienyl, pyrrol-2-yl, pyrrol-3-yl, 5-cyanopyrrol-2-yl, 4-cyanopyrrol-2-yl, 5-cyanopyrrol-3-yl, 5-methylpyrrol-2-yl, 4-methylpyrrol-2-yl, 5-methylpyrrol-3-yl, 1,5-dimethylpyrrol-2-yl, 1,4-dimethylpyrrol-2-yl, 1,5-dimethylpyrrol-3-yl, 1-methyl-5-cyanopyrrol-2-yl, 1-methyl-4-cyanopyrrol-2-yl, 1-methyl-5-cyanopyrrol-3-yl, oxazol-4-yl, oxazol-5-yl, 2-cyanooxazol-4-yl, 2-cyanooxazol-5-yl, 3-methyl-2-cyanooxazol-4-yl, 3-methyl-2-cyanooxazol-5-yl, imidazol-4-yl, imidazol-5-yl, 2-cyanoimidazol-4-yl, 2-cyanoimidazol-5-yl, 2-cyano-1-methylimidazol-4-yl, 2-cyano-1-methylimidazol-5-yl, thiazol-4-yl, thiazol-5-yl, 2-cyanothiazol-4-yl, 2-cyanothiazol-5-yl, 3-methyl-2-cyanothiazol-4-yl, 3-methyl-2-cyanothiazol-5-yl, 4-cyanooxazol-2-yl, 5-cyanooxazol-2-yl, 4-cyanoimidazol-2-yl, 5-cyanoimidazol-2-yl, 4-cyano-1-methylimidazol-2-yl, 5-cyano-1-methylimidazol-2-yl, 5-cyanothiazol-2-yl, 4-cyanothiazol-2-yl, 3-cyanoisoxazol-5-yl, 5-cyanoisoxazol-3-yl, 3-cyanoisothiazol-5-yl, 5-cyanoisothiazol-3-yl, 3-cyanopyrazol-5-yl, 5-cyanopyrazol-3-yl, 3-cyano-1-methylpyrazol-5-yl, 5-cyano-1-methylpyrazol-3-yl, 5-cyano-1,3,4-oxadiazol-2-yl, 5-cyano-1,3,4-thiadiazol-2-yl, 5-cyano-1,2,4-triazol-3-yl, 5-cyano-4-methyl-1,2,4-triazol-3-yl.

26. The method of claim 1, wherein R³ is selected from the group consisting of hydrogen, C₁-C₄-alkyl, difluoromethyl, trifluoromethyl, acetyl, difluoroacetyl and trifluoroacetyl.

27. The method of claim 26, wherein R³ is selected from the group consisting of hydrogen, methyl, ethyl or n-propyl.

28. The method of claim 1, wherein X is CH₂CH₂.

29. The method of claim 1, wherein the compound of formula (I) is selected from:
2-(4-nitrophenyl)-6-phenyl-3-(piperazin-1-ylmethyl)imidazo[1,2-a]pyridine,
1-(4-((2-(3-nitrophenyl)-6-phenylimidazo[1,2-a]pyridin-3-yl)methyl)piperazin-1-yl)ethanone,
1-(4-((2-(4-nitrophenyl)-6-phenylimidazo[1,2-a]pyridin-3-yl)methyl)piperazin-1-yl)ethanone,
3-((1,4-diazepan-1-yl)methyl)-2-(4-nitrophenyl)-6-phenylimidazo[1,2-a]pyridine,
2-(4-nitrophenyl)-6-phenyl-3-((4-propylpiperazin-1-yl)methyl)imidazo[1,2-a]pyridine,
3-((4-ethylpiperazin-1-yl)methyl)-2-(4-nitrophenyl)-6-phenylimidazo[1,2-a]pyridine,
3-((4-methylpiperazin-1-yl)methyl)-2-(4-nitrophenyl)-6-phenylimidazo[1,2-a]pyridine,
3-((1,4-diazepan-1-yl)methyl)-2-(4-(methylsulfonyl)phenyl)-6-phenylimidazo[1,2-a]-pyridine,
4-(3-((1,4-diazepan-1-yl)methyl)-6-phenylimidazo[1,2-a]pyridin-2-yl)benzonitrile,
4-(3-((1,4-diazepan-1-yl)methyl)-6-phenylimidazo[1,2-a]pyridin-2-yl)benzamide,
3-((1,4-diazepan-1-yl)methyl)-6-phenyl-2-(pyridin-4-yl)imidazo[1,2-a]pyridine,
3-((4-methyl-1,4-diazepan-1-yl)methyl)-2-(4-nitrophenyl)-6-phenylimidazo[1,2-a]pyridine,
4-((2-(4-nitrophenyl)-6-phenylimidazo[1,2-a]pyridin-3-yl)methyl)piperazin-2-one,
3-((1,4-diazepan-1-yl)methyl)-2-(4-chlorophenyl)-6-phenylimidazo[1,2-a]pyridine,
3-((1,4-diazepan-1-yl)methyl)-6-phenyl-2-(pyridin-3-yl)imidazo[1,2-a]pyridine,
5-(3-((1,4-diazepan-1-yl)methyl)-6-phenylimidazo[1,2-a]pyridin-2-yl)picolinonitrile,
3-((1,4-diazepan-1-yl)methyl)-6-phenyl-2-(p-tolyl)imidazo[1,2-a]pyridine,
3-((1,4-diazepan-1-yl)methyl)-2-(4-methoxyphenyl)-6-phenylimidazo[1,2-a]pyridine,
3-((1,4-diazepan-1-yl)methyl)-2,6-diphenylimidazo[1,2-a]pyridine,
5-(3-((1,4-diazepan-1-yl)methyl)-6-phenylimidazo[1,2-a]pyridin-2-yl)thiophene-3-carbonitrile,
5-(3-((1,4-diazepan-1-yl)methyl)-6-phenylimidazo[1,2-a]pyridin-2-yl)thiophene-2-carbonitrile,
3-(3-((1,4-diazepan-1-yl)methyl)-6-phenylimidazo[1,2-a]pyridin-2-yl)benzonitrile,
4-(3-((1,4-diazepan-1-yl)methyl)-6-(p-tolyl)imidazo[1,2-a]pyridin-2-yl)benzonitrile,
4-(3-((1,4-diazepan-1-yl)methyl)-6-(m-tolyl)imidazo[1,2-a]pyridin-2-yl)benzonitrile,
4-(3-((1,4-diazepan-1-yl)methyl)-6-(o-tolyl)imidazo[1,2-a]pyridin-2-yl)benzonitrile,
4-(3-((1,4-diazepan-1-yl)methyl)-6-(pyrimidin-5-yl)imidazo[1,2-a]pyridin-2-yl)benzonitrile,
4-(3-((1,4-diazepan-1-yl)methyl)-6-(pyridin-4-yl)imidazo[1,2-a]pyridin-2-yl)benzonitrile,
4-(3-((1,4-diazepan-1-yl)methyl)-6-(2-methylpyridin-4-yl)imidazo[1,2-a]pyridin-2-yl)benzonitrile,
4-(3-((1,4-diazepan-1-yl)methyl)-6-(pyridin-3-yl)imidazo[1,2-a]pyridin-2-yl)benzonitrile,
4-((2-(4-methoxyphenyl)-6-phenylimidazo[1,2-a]pyridin-3-yl)methyl)piperazin-2-one,
2-(4-chlorophenyl)-3-((4-methyl-1,4-diazepan-1-yl)methyl)-6-phenylimidazo[1,2-a]-pyridine,
4-((2-(4-chlorophenyl)-6-phenylimidazo[1,2-a]pyridin-3-yl)methyl)piperazin-2-one,
4-(3-((3-oxopiperazin-1-yl)methyl)-6-phenylimidazo[1,2-a]pyridin-2-yl)benzonitrile,
4-(3-((4-methyl-1,4-diazepan-1-yl)methyl)-6-phenylimidazo[1,2-a]pyridin-2-yl)benzonitrile,
5-(3-((3-oxopiperazin-1-yl)methyl)-6-phenylimidazo[1,2-a]pyridin-2-yl)thiophene-2-carbonitrile,
5-(3-((4-methyl-1,4-diazepan-1-yl)methyl)-6-phenylimidazo[1,2-a]pyridin-2-yl)thiophene-2-carbonitrile,
4-(3-((1,4-diazepan-1-yl)methyl)-6-(1H-pyrazol-4-yl)imidazo[1,2-a]pyridin-2-yl)benzonitrile,
4-(3-((1,4-diazepan-1-yl)methyl)-6-(1-methyl-1H-pyrrol-2-yl)imidazo[1,2-a]pyridin-2-yl)benzonitrile,
4-(3-((1,4-diazepan-1-yl)methyl)-6-(oxazol-5-yl)imidazo[1,2-a]pyridin-2-yl)benzonitrile,
4-(3-((1,4-diazepan-1-yl)methyl)-6-(4-chlorophenyl)imidazo[1,2-a]pyridin-2-yl)benzonitrile,
4-(3-((1,4-diazepan-1-yl)methyl)-6-(4-methoxyphenyl)imidazo[1,2-a]pyridin-2-yl)benzonitrile,
4-(3-((1,4-diazepan-1-yl)methyl)-2-(4-cyanophenyl)imidazo[1,2-a]pyridin-6-yl)benzamide,
4,4′-(3-((1,4-diazepan-1-yl)methyl)imidazo[1,2-a]pyridine-2,6-diyl)dibenzonitrile,
4-(3-((1,4-diazepan-1-yl)methyl)-6-(thiophen-3-yl)imidazo[1,2-a]pyridin-2-yl)benzonitrile,
the pharmaceutically acceptable salts thereof, the N-oxides thereof and the pharmaceutically acceptable salts of said N-oxides.

30. The method of claim 1, wherein the compound of formula (I) is selected from:
2-(4-methoxyphenyl)-3-((4-methyl-1,4-diazepan-1-yl)methyl)-6-(pyridin-3-yl)imidazo[1,2-a]pyridine,
3-((1,4-diazepan-1-yl)methyl)-2-(4-methoxyphenyl)-6-(pyridin-3-yl)imidazo[1,2-a]-pyridine,
3-((1,4-diazepan-1-yl)methyl)-2-(4-methoxyphenyl)-6-(4-(trifluoromethyl)phenyl)imidazo[1,2-a]pyridine,
2-(4-methoxyphenyl)-3-((4-methyl-1,4-diazepan-1-yl)methyl)-6-(4-(trifluoromethyl)phenyl)imidazo[1,2-a]pyridine,
4-(3-((1,4-diazepan-1-yl)methyl)-2-(4-methoxyphenyl)imidazo[1,2-a]pyridin-6-yl)benzamide,
4-(2-(4-methoxyphenyl)-3-((4-methyl-1,4-diazepan-1-yl)methyl)imidazo[1,2-a]pyridin-6-yl)benzamide,
5-(3-((1,4-diazepan-1-yl)methyl)-2-(4-methoxyphenyl)imidazo[1,2-a]pyridin-6-yl)thiophene-2-carbonitrile,
5-(2-(4-methoxyphenyl)-3-((4-methyl-1,4-diazepan-1-yl)methyl)imidazo[1,2-a]pyridin-6-yl)thiophene-2-carbonitrile,
3-((1,4-diazepan-1-yl)methyl)-2-(4-methoxyphenyl)-6-(4-(pentafluorothio)phenyl)imidazo[1,2-a]pyridine,
2-(4-methoxyphenyl)-3-((4-methyl-1,4-diazepan-1-yl)methyl)-6-(4-(pentafluorothio)phenyl)imidazo[1,2-a]pyridine,
2-(4-methoxyphenyl)-3-((4-methyl-1,4-diazepan-1-yl)methyl)-6-(1-methyl-1H-pyrrol-2-yl)imidazo[1,2-a]pyridine,
3-((1,4-diazepan-1-yl)methyl)-2-(4-methoxyphenyl)-6-(1-methyl-1H-pyrrol-2-yl)imidazo[1,2-a]pyridine,
4-(3-((1,4-diazepan-1-yl)methyl)-2-(4-methoxyphenyl)imidazo[1,2-a]pyridin-6-yl)-2-methylbenzonitrile,
4-(2-(4-methoxyphenyl)-3-((4-methyl-1,4-diazepan-1-yl)methyl)imidazo[1,2-a]pyridin-6-yl)-2-methylbenzonitrile,
3-((1,4-diazepan-1-yl)methyl)-2-(4-methoxyphenyl)-6-(pyrimidin-5-yl)imidazo[1,2-a]-pyridine,
2-(4-methoxyphenyl)-3-((4-methyl-1,4-diazepan-1-yl)methyl)-6-(pyrimidin-5-yl)imidazo[1,2-a]pyridine,
5-(2-(4-methoxyphenyl)-3-((4-methyl-1,4-diazepan-1-yl)methyl)imidazo[1,2-a]pyridin-6-yl)oxazole,
5-(3-((1,4-diazepan-1-yl)methyl)-2-(4-methoxyphenyl)imidazo[1,2-a]pyridin-6-yl)oxazole,
3-((1,4-diazepan-1-yl)methyl)-2-(4-methoxyphenyl)-6-(p-tolyl)imidazo[1,2-a]pyridine,
2-(4-methoxyphenyl)-3-((4-methyl-1,4-diazepan-1-yl)methyl)-6-(p-tolyl)imidazo[1,2-a]pyridine,
4-(3-((1,4-diazepan-1-yl)methyl)-2-(4-methoxyphenyl)imidazo[1,2-a]pyridin-6-yl)benzonitrile,
4-(2-(4-methoxyphenyl)-3-((4-methyl-1,4-diazepan-1-yl)methyl)imidazo[1,2-a]pyridin-6-yl)benzonitrile,
5-(3-((1,4-diazepan-1-yl)methyl)-2-(4-methoxyphenyl)imidazo[1,2-a]pyridin-6-yl)thiophene-3-carbonitrile,
5-(2-(4-methoxyphenyl)-3-((4-methyl-1,4-diazepan-1-yl)methyl)imidazo[1,2-a]pyridin-6-yl)thiophene-3-carbonitrile,
6-(4-chlorophenyl)-2-(4-methoxyphenyl)-3-((4-methyl-1,4-diazepan-1-yl)methyl)imidazo[1,2-a]pyridine,
3-((1,4-diazepan-1-yl)methyl)-6-(4-chlorophenyl)-2-(4-methoxyphenyl)imidazo[1,2-a]pyridine,
4-(3-((1,4-diazepan-1-yl)methyl)-2-(4-methoxyphenyl)imidazo[1,2-a]pyridin-6-yl)thiophene-2-carbonitrile,
4-(2-(4-methoxyphenyl)-3-((4-methyl-1,4-diazepan-1-yl)methyl)imidazo[1,2-a]pyridin-6-yl)thiophene-2-carbonitrile,
2-(4-methoxyphenyl)-3-((4-methyl-1,4-diazepan-1-yl)methyl)-6-(o-tolyl)imidazo[1,2-a]pyridine,
3-((1,4-diazepan-1-yl)methyl)-2-(4-methoxyphenyl)-6-(o-tolyl)imidazo[1,2-a]pyridine,
4-(2-(4-methoxyphenyl)-3-((4-methyl-1,4-diazepan-1-yl)methyl)imidazo[1,2-a]pyridin-6-yl)-N,N-dimethylaniline,
4-(3-((1,4-diazepan-1-yl)methyl)-2-(4-methoxyphenyl)imidazo[1,2-a]pyridin-6-yl)-N,N-dimethylaniline,
2,6-bis(4-methoxyphenyl)-3-((4-methyl-1,4-diazepan-1-yl)methyl)imidazo[1,2-a]pyridine,
3-((1,4-diazepan-1-yl)methyl)-2,6-bis(4-methoxyphenyl)imidazo[1,2-a]pyridine,
3-((1,4-diazepan-1-yl)methyl)-2-(4-methoxyphenyl)-6-(m-tolyl)imidazo[1,2-a]pyridine,
2-(4-methoxyphenyl)-3-((4-methyl-1,4-diazepan-1-yl)methyl)-6-(m-tolyl)imidazo[1,2-a]pyridine,
2-(4-methoxyphenyl)-3-((4-methyl-1,4-diazepan-1-yl)methyl)-6-(2-methylpyridin-4-yl)imidazo[1,2-a]pyridine,
3-((1,4-diazepan-1-yl)methyl)-2-(4-methoxyphenyl)-6-(2-methylpyridin-4-yl)imidazo[1,2-a]pyridine,
2-(4-methoxyphenyl)-3-((4-methyl-1,4-diazepan-1-yl)methyl)-6-(thiophen-3-yl)imidazo[1,2-a]pyridine,
3-((1,4-diazepan-1-yl)methyl)-2-(4-methoxyphenyl)-6-(thiophen-3-yl)imidazo[1,2-a]-pyridine,
6-(3,4-dimethoxyphenyl)-2-(4-methoxyphenyl)-3-((4-methyl-1,4-diazepan-1-yl)methyl)imidazo[1,2-a]pyridine,
3-((1,4-diazepan-1-yl)methyl)-6-(3,4-dimethoxyphenyl)-2-(4-methoxyphenyl)imidazo[1,2-a]pyridine,
6-(1,5-dimethyl-1H-pyrrol-2-yl)-2-(4-methoxyphenyl)-3-((4-methyl-1,4-diazepan-1-yl)methyl)imidazo[1,2-a]pyridine,
3-((1,4-diazepan-1-yl)methyl)-6-(1,5-dimethyl-1H-pyrrol-2-yl)-2-(4-methoxyphenyl)imidazo[1,2-a]pyridine,
5-(3-((1,4-diazepan-1-yl)methyl)-2-(4-methoxyphenyl)imidazo[1,2-a]pyridin-6-yl)-1H-pyrrole-2-carbonitrile,
5-(2-(4-methoxyphenyl)-3-((4-methyl-1,4-diazepan-1-yl)methyl)imidazo[1,2-a]pyridin-6-yl)-1H-pyrrole-2-carbonitrile,
2-(4-methoxyphenyl)-3-((4-methyl-1,4-diazepan-1-yl)methyl)-6-(5-methyl-1H-pyrrol-2-yl)imidazo[1,2-a]pyridine,
3-((1,4-diazepan-1-yl)methyl)-2-(4-methoxyphenyl)-6-(5-methyl-1H-pyrrol-2-yl)imidazo[1,2-a]pyridine,
2-(4-methoxyphenyl)-3-((4-methyl-1,4-diazepan-1-yl)methyl)-6-phenylimidazo[1,2-a]pyridine,
6-(furan-2-yl)-2-(4-methoxyphenyl)-3-((4-methyl-1,4-diazepan-1-yl)methyl)imidazo[1,2-a]pyridine,
3-((1,4-diazepan-1-yl)methyl)-6-(furan-2-yl)-2-(4-methoxyphenyl)imidazo[1,2-a]pyridine,
3-((1,4-diazepan-1-yl)methyl)-2-(4-methoxyphenyl)-6-(1H-pyrrol-2-yl)imidazo[1,2-a]-pyridine,
2-(4-methoxyphenyl)-3-((4-methyl-1,4-diazepan-1-yl)methyl)-6-(1H-pyrrol-2-yl)imidazo[1,2-a]pyridine,
2-(4-methoxyphenyl)-3-((4-methyl-1,4-diazepan-1-yl)methyl)-6-(pyridin-4-yl)imidazo[1,2-a]pyridine,
3-((1,4-diazepan-1-yl)methyl)-2-(4-methoxyphenyl)-6-(pyridin-4-yl)imidazo[1,2-a]-pyridine,
3-((1,4-diazepan-1-yl)methyl)-6-(3,4-dimethylphenyl)-2-(4-methoxyphenyl)imidazo[1,2-a]pyridine,
6-(3,4-dimethylphenyl)-2-(4-methoxyphenyl)-3-((4-methyl-1,4-diazepan-1-yl)methyl)imidazo[1,2-a]pyridine,
3-((1,4-diazepan-1-yl)methyl)-2-(4-methoxyphenyl)-6-(1H-pyrazol-4-yl)imidazo[1,2-a]pyridine,
2-(4-methoxyphenyl)-3-((4-methyl-1,4-diazepan-1-yl)methyl)-6-(1H-pyrazol-4-yl)imidazo[1,2-a]pyridine,
2-(4-chlorophenyl)-3-((4-methyl-1,4-diazepan-1-yl)methyl)-6-(4-(pentafluorothio)phenyl)imidazo[1,2-a]pyridine,
3-((1,4-diazepan-1-yl)methyl)-2-(4-chlorophenyl)-6-(4-(pentafluorothio)phenyl)imidazo[1,2-a]pyridine,
3-((1,4-diazepan-1-yl)methyl)-2-(4-chlorophenyl)-6-(pyrimidin-5-yl)imidazo[1,2-a]-pyridine,
2-(4-chlorophenyl)-3-((4-methyl-1,4-diazepan-1-yl)methyl)-6-(pyrimidin-5-yl)imidazo[1,2-a]pyridine,
4-(2-(4-chlorophenyl)-3-((4-methyl-1,4-diazepan-1-yl)methyl)imidazo[1,2-a]pyridin-6-yl)-2-methylbenzonitrile,
4-(3-((1,4-diazepan-1-yl)methyl)-2-(4-chlorophenyl)imidazo[1,2-a]pyridin-6-yl)-2-methylbenzonitrile,
3-((1,4-diazepan-1-yl)methyl)-2-(4-chlorophenyl)-6-(1H-pyrrol-2-yl)imidazo[1,2-a]-pyridine,
2-(4-chlorophenyl)-3-((4-methyl-1,4-diazepan-1-yl)methyl)-6-(1H-pyrrol-2-yl)imidazo[1,2-a]pyridine,
3-((1,4-diazepan-1-yl)methyl)-2-(4-chlorophenyl)-6-(m-tolyl)imidazo[1,2-a]pyridine,
2-(4-chlorophenyl)-3-((4-methyl-1,4-diazepan-1-yl)methyl)-6-(m-tolyl)imidazo[1,2-a]-pyridine,
2-(4-chlorophenyl)-6-(3,4-dimethylphenyl)-3-((4-methyl-1,4-diazepan-1-yl)methyl)imidazo[1,2-a]pyridine,
3-((1,4-diazepan-1-yl)methyl)-2-(4-chlorophenyl)-6-(3,4-dimethylphenyl)imidazo[1,2-a]pyridine,
5-(3-((1,4-diazepan-1-yl)methyl)-2-(4-chlorophenyl)imidazo[1,2-a]pyridin-6-yl)-1H-pyrrole-2-carbonitrile,
5-(2-(4-chlorophenyl)-3-((4-methyl-1,4-diazepan-1-yl)methyl)imidazo[1,2-a]pyridin-6-yl)-1H-pyrrole-2-carbonitrile,
3-((1,4-diazepan-1-yl)methyl)-2-(4-chlorophenyl)-6-(o-tolyl)imidazo[1,2-a]pyridine,
2-(4-chlorophenyl)-3-((4-methyl-1,4-diazepan-1-yl)methyl)-6-(o-tolyl)imidazo[1,2-a]—pyridine,
2-(4-chlorophenyl)-6-(3,4-dimethoxyphenyl)-3-((4-methyl-1,4-diazepan-1-yl)methyl)imidazo[1,2-a]pyridine,
3-((1,4-diazepan-1-yl)methyl)-2-(4-chlorophenyl)-6-(3,4-dimethoxyphenyl)imidazo[1,2-a]pyridine,
2-(4-chlorophenyl)-6-(1,5-dimethyl-1H-pyrrol-2-yl)-3-((4-methyl-1,4-diazepan-1-yl)methyl)imidazo[1,2-a]pyridine,
3-((1,4-diazepan-1-yl)methyl)-2-(4-chlorophenyl)-6-(1,5-dimethyl-1H-pyrrol-2-yl)imidazo[1,2-a]pyridine,
2-(4-chlorophenyl)-3-((4-methyl-1,4-diazepan-1-yl)methyl)-6-(5-methyl-1H-pyrrol-2-yl)imidazo[1,2-a]pyridine,
3-((1,4-diazepan-1-yl)methyl)-2-(4-chlorophenyl)-6-(5-methyl-1H-pyrrol-2-yl)imidazo[1,2-a]pyridine,
3-((1,4-diazepan-1-yl)methyl)-2-(4-chlorophenyl)-6-(pyridin-3-yl)imidazo[1,2-a]pyridine,
2-(4-chlorophenyl)-3-((4-methyl-1,4-diazepan-1-yl)methyl)-6-(pyridin-3-yl)imidazo[1,2-a]pyridine,
2-(4-chlorophenyl)-6-(furan-2-yl)-3-((4-methyl-1,4-diazepan-1-yl)methyl)imidazo[1,2-a]pyridine,
3-((1,4-diazepan-1-yl)methyl)-2-(4-chlorophenyl)-6-(furan-2-yl)imidazo[1,2-a]pyridine,
3-((1,4-diazepan-1-yl)methyl)-2-(4-chlorophenyl)-6-(1H-pyrazol-4-yl)imidazo[1,2-a]pyridine,
2-(4-chlorophenyl)-3-((4-methyl-1,4-diazepan-1-yl)methyl)-6-(1H-pyrazol-4-yl)imidazo[1,2-a]pyridine,
4-(2-(4-chlorophenyl)-3-((4-methyl-1,4-diazepan-1-yl)methyl)imidazo[1,2-a]pyridin-6-yl)thiophene-2-carbonitrile,
4-(3-((1,4-diazepan-1-yl)methyl)-2-(4-chlorophenyl)imidazo[1,2-a]pyridin-6-yl)thiophene-2-carbonitrile,
5-(2-(4-chlorophenyl)-3-((4-methyl-1,4-diazepan-1-yl)methyl)imidazo[1,2-a]pyridin-6-yl)thiophene-3-carbonitrile,
5-(3-((1,4-diazepan-1-yl)methyl)-2-(4-chlorophenyl)imidazo[1,2-a]pyridin-6-yl)thiophene-3-carbonitrile,
3-(((1,4-diazepan-1-yl)methyl)-2-(4-chlorophenyl)-6-(1-methyl-1H-pyrrol-2-yl)imidazo[1,2-a]pyridine,
2-(4-chlorophenyl)-3-((4-methyl-1,4-diazepan-1-yl)methyl)-6-(1-methyl-1H-pyrrol-2-yl)imidazo[1,2-a]pyridine,
2-(4-chlorophenyl)-3-((4-methyl-1,4-diazepan-1-yl)methyl)-6-(2-methylpyridin-4-yl)imidazo[1,2-a]pyridine,
3-((1,4-diazepan-1-yl)methyl)-2-(4-chlorophenyl)-6-(2-methylpyridin-4-yl)imidazo[1,2-a]pyridine,
5-(2-(4-chlorophenyl)-3-((4-methyl-1,4-diazepan-1-yl)methyl)imidazo[1,2-a]pyridin-6-yl)thiophene-2-carbonitrile,
5-(3-((1,4-diazepan-1-yl)methyl)-2-(4-chlorophenyl)imidazo[1,2-a]pyridin-6-yl)thiophene-2-carbonitrile,
5-(3-((1,4-diazepan-1-yl)methyl)-2-(4-chlorophenyl)imidazo[1,2-a]pyridin-6-yl)oxazole,
5-(2-(4-chlorophenyl)-3-((4-methyl-1,4-diazepan-1-yl)methyl)imidazo[1,2-a]pyridin-6-yl)oxazole,
4-(2-(4-chlorophenyl)-3-((4-methyl-1,4-diazepan-1-yl)methyl)imidazo[1,2-a]pyridin-6-yl)benzamide,
4-(3-((1,4-diazepan-1-yl)methyl)-2-(4-chlorophenyl)imidazo[1,2-a]pyridin-6-yl)benzamide,
2-(4-chlorophenyl)-6-(4-methoxyphenyl)-3-((4-methyl-1,4-diazepan-1-yl)methyl)imidazo[1,2-a]pyridine,
3-((1,4-diazepan-1-yl)methyl)-2-(4-chlorophenyl)-6-(4-methoxyphenyl)imidazo[1,2-a]pyridine,
2-(4-chlorophenyl)-3-((4-methyl-1,4-diazepan-1-yl)methyl)-6-(pyridin-4-yl)imidazo[1,2-a]pyridine,
3-((1,4-diazepan-1-yl)methyl)-2-(4-chlorophenyl)-6-(pyridin-4-yl)imidazo[1,2-a]pyridine,
3-((1,4-diazepan-1-yl)methyl)-2-(4-chlorophenyl)-6-(thiophen-3-yl)imidazo[1,2-a]-pyridine,
2-(4-chlorophenyl)-3-((4-methyl-1,4-diazepan-1-yl)methyl)-6-(thiophen-3-yl)imidazo[1,2-a]pyridine,
3-((1,4-diazepan-1-yl)methyl)-2,6-bis(4-chlorophenyl)imidazo[1,2-a]pyridine,
2,6-bis(4-chlorophenyl)-3-((4-methyl-1,4-diazepan-1-yl)methyl)imidazo[1,2-a]pyridine,
4-(3-((1,4-diazepan-1-yl)methyl)-2-(4-chlorophenyl)imidazo[1,2-a]pyridin-6-yl)benzonitrile,
4-(2-(4-chlorophenyl)-3-((4-methyl-1,4-diazepan-1-yl)methyl)imidazo[1,2-a]pyridin-6-yl)benzonitrile,
4-(2-(4-chlorophenyl)-3-((4-methyl-1,4-diazepan-1-yl)methyl)imidazo[1,2-a]pyridin-6-yl)-N,N-dimethylaniline,
4-(3-((1,4-diazepan-1-yl)methyl)-2-(4-chlorophenyl)imidazo[1,2-a]pyridin-6-yl)-N,N-dimethylaniline,
3-((1,4-diazepan-1-yl)methyl)-2-(4-chlorophenyl)-6-(p-tolyl)imidazo[1,2-a]pyridine,
2-(4-chlorophenyl)-3-((4-methyl-1,4-diazepan-1-yl)methyl)-6-(p-tolyl)imidazo[1,2-a]-pyridine,
3-((1,4-diazepan-1-yl)methyl)-2-(4-chlorophenyl)-6-(4-(trifluoromethyl)phenyl)imidazo[1,2-a]pyridine,
2-(4-chlorophenyl)-3-((4-methyl-1,4-diazepan-1-yl)methyl)-6-(4-(trifluoromethyl)phenyl)imidazo[1,2-a]pyridine,
5-(3-((1,4-diazepan-1-yl)methyl)-2-(4-cyanophenyl)imidazo[1,2-a]pyridin-6-yl)thiophene-2-carbonitrile,
5-(2-(4-cyanophenyl)-3-((4-methyl-1,4-diazepan-1-yl)methyl)imidazo[1,2-a]pyridin-6-yl)thiophene-2-carbonitrile,
5-(3-((1,4-diazepan-1-yl)methyl)-2-(4-cyanophenyl)imidazo[1,2-a]pyridin-6-yl)thiophene-3-carbonitrile,
5-(2-(4-cyanophenyl)-3-((4-methyl-1,4-diazepan-1-yl)methyl)imidazo[1,2-a]pyridin-6-yl)thiophene-3-carbonitrile,
4-(3-((4-methyl-1,4-diazepan-1-yl)methyl)-6-(1H-pyrazol-4-yl)imidazo[1,2-a]pyridin-2-yl)benzonitrile,
4-(3-((1,4-diazepan-1-yl)methyl)-6-(furan-2-yl)imidazo[1,2-a]pyridin-2-yl)benzonitrile,
4-(6-(furan-2-yl)-3-((4-methyl-1,4-diazepan-1-yl)methyl)imidazo[1,2-a]pyridin-2-yl)benzonitrile,
4-(6-(3,4-dimethylphenyl)-3-((4-methyl-1,4-diazepan-1-yl)methyl)imidazo[1,2-a]pyridin-2-yl)benzonitrile,
4-(3-((1,4-diazepan-1-yl)methyl)-6-(3,4-dimethylphenyl)imidazo[1,2-a]pyridin-2-yl)benzonitrile,
4-(3-((4-methyl-1,4-diazepan-1-yl)methyl)-6-(o-tolyl)imidazo[1,2-a]pyridin-2-yl)benzonitrile,
4-(3-((1,4-diazepan-1-yl)methyl)-6-(5-methyl-1H-pyrrol-2-yl)imidazo[1,2-a]pyridin-2-yl)benzonitrile,
4-(3-((4-methyl-1,4-diazepan-1-yl)methyl)-6-(5-methyl-1H-pyrrol-2-yl)imidazo[1,2-a]-pyridin-2-yl)benzonitrile,
4-(3-((4-methyl-1,4-diazepan-1-yl)methyl)-6-(pyridin-3-yl)imidazo[1,2-a]pyridin-2-yl)benzonitrile,
4-(3-((1,4-diazepan-1-yl)methyl)-6-(1,5-dimethyl-1H-pyrrol-2-yl)imidazo[1,2-a]pyridin-2-yl)benzonitrile,
4-(6-(1,5-dimethyl-1H-pyrrol-2-yl)-3-((4-methyl-1,4-diazepan-1-yl)methyl)imidazo[1,2-a]pyridin-2-yl)benzonitrile,
4-(6-(3,4-dimethoxyphenyl)-3-((4-methyl-1,4-diazepan-1-yl)methyl)imidazo[1,2-a]-pyridin-2-yl)benzonitrile,
4-(3-((1,4-diazepan-1-yl)methyl)-6-(3,4-dimethoxyphenyl)imidazo[1,2-a]pyridin-2-yl)benzonitrile,
4-(3-((4-methyl-1,4-diazepan-1-yl)methyl)-6-(pyridin-4-yl)imidazo[1,2-a]pyridin-2-yl)benzonitrile,
4-(3-((1,4-diazepan-1-yl)methyl)-6-(1H-pyrrol-2-yl)imidazo[1,2-a]pyridin-2-yl)benzonitrile,
4-(3-((4-methyl-1,4-diazepan-1-yl)methyl)-6-(1H-pyrrol-2-yl)imidazo[1,2-a]pyridin-2-yl)benzonitrile,
4-(3-((4-methyl-1,4-diazepan-1-yl)methyl)-6-(pyrimidin-5-yl)imidazo[1,2-a]pyridin-2-yl)benzonitrile,
4-(3-((1,4-diazepan-1-yl)methyl)-6-(4-(trifluoromethyl)phenyl)imidazo[1,2-a]pyridin-2-yl)benzonitrile,
4-(3-((4-methyl-1,4-diazepan-1-yl)methyl)-6-(4-(trifluoromethyl)phenyl)imidazo[1,2-a]pyridin-2-yl)benzonitrile,
4-(3-((1,4-diazepan-1-yl)methyl)-2-(4-cyanophenyl)imidazo[1,2-a]pyridin-6-yl)-2-methylbenzonitrile,
4-(2-(4-cyanophenyl)-3-((4-methyl-1,4-diazepan-1-yl)methyl)imidazo[1,2-a]pyridin-6-yl)-2-methylbenzonitrile,
4-(6-(4-methoxyphenyl)-3-((4-methyl-1,4-diazepan-1-yl)methyl)imidazo[1,2-a]pyridin-2-yl)benzonitrile,
5-(2-(4-cyanophenyl)-3-((4-methyl-1,4-diazepan-1-yl)methyl)imidazo[1,2-a]pyridin-6-yl)-1H-pyrrole-2-carbonitrile,
5-(3-((1,4-diazepan-1-yl)methyl)-2-(4-cyanophenyl)imidazo[1,2-a]pyridin-6-yl)-1H-pyrrole-2-carbonitrile,
4-(2-(4-cyanophenyl)-3-((4-methyl-1,4-diazepan-1-yl)methyl)imidazo[1,2-a]pyridin-6-yl)thiophene-2-carbonitrile,
4-(3-((1,4-diazepan-1-yl)methyl)-2-(4-cyanophenyl)imidazo[1,2-a]pyridin-6-yl)thiophene-2-carbonitrile,
4-(6-(4-chlorophenyl)-3-((4-methyl-1,4-diazepan-1-yl)methyl)imidazo[1,2-a]pyridin-2-yl)benzonitrile,
4-(2-(4-cyanophenyl)-3-((4-methyl-1,4-diazepan-1-yl)methyl)imidazo[1,2-a]pyridin-6-yl)benzamide,
4,4′-(3-((4-methyl-1,4-diazepan-1-yl)methyl)imidazo[1,2-a]pyridine-2,6-diyl)dibenzonitrile,
4-(6-(4-(dimethylamino)phenyl)-3-((4-methyl-1,4-diazepan-1-yl)methyl)imidazo[1,2-a]pyridin-2-yl)benzonitrile,
4-(3-((1,4-diazepan-1-yl)methyl)-6-(4-(dimethylamino)phenyl)imidazo[1,2-a]pyridin-2-yl)benzonitrile,
4-(3-((4-methyl-1,4-diazepan-1-yl)methyl)-6-(2-methylpyridin-4-yl)imidazo[1,2-a]pyridin-2-yl)benzonitrile,
4-(3-((4-methyl-1,4-diazepan-1-yl)methyl)-6-(1-methyl-1H-pyrrol-2-yl)imidazo[1,2-a]-pyridin-2-yl)benzonitrile,
4-(3-((4-methyl-1,4-diazepan-1-yl)methyl)-6-(p-tolyl)imidazo[1,2-a]pyridin-2-yl)benzonitrile,
4-(3-((4-methyl-1,4-diazepan-1-yl)methyl)-6-(m-tolyl)imidazo[1,2-a]pyridin-2-yl)benzonitrile,
4-(3-((4-methyl-1,4-diazepan-1-yl)methyl)-6-(oxazol-5-yl)imidazo[1,2-a]pyridin-2-yl)benzonitrile,
4-(3-((4-methyl-1,4-diazepan-1-yl)methyl)-6-(4-(pentafluorothio)phenyl)imidazo[1,2-a]pyridin-2-yl)benzonitrile,
4-(3-((1,4-diazepan-1-yl)methyl)-6-(4-(pentafluorothio)phenyl)imidazo[1,2-a]pyridin-2-yl)benzonitrile,
4-(3-((4-methyl-1,4-diazepan-1-yl)methyl)-6-(thiophen-3-yl)imidazo[1,2-a]pyridin-2-yl)benzonitrile,
4-(2-(4-chlorophenyl)-3-((4-methylpiperazin-1-yl)methyl)imidazo[1,2-a]pyridin-6-yl)benzonitrile,
4-(2-(4-methoxyphenyl)-3-((3-oxopiperazin-1-yl)methyl)imidazo[1,2-a]pyridin-6-yl)benzonitrile,
4-(2-(4-methoxyphenyl)-3-((4-(2,2,2-trifluoroacetyl)-1,4-diazepan-1-yl)methyl)imidazo[1,2-a]pyridin-6-yl)benzonitrile,
4-(2-(4-methoxyphenyl)-3-((4-(trifluoromethyl)-1,4-diazepan-1-yl)methyl)imidazo[1,2-a]pyridin-6-yl)benzonitrile,
4-(6-(3,4-dimethylphenyl)-3-((4-propylpiperazin-1-yl)methyl)imidazo[1,2-a]pyridin-2-yl)benzonitrile,
4-((2-(4-chlorophenyl)-6-(3,4-dimethoxyphenyl)imidazo[1,2-a]pyridin-3-yl)methyl)piperazin-2-one,
4-(6-(3,4-dimethylphenyl)-3-((3-oxopiperazin-1-yl)methyl)imidazo[1,2-a]pyridin-2-yl)benzonitrile,
4-((2-(4-methoxyphenyl)-6-(oxazol-5-yl)imidazo[1,2-a]pyridin-3-yl)methyl)piperazin-2-one,
4-(3-((4-methylpiperazin-1-yl)methyl)-6-(pyrimidin-5-yl)imidazo[1,2-a]pyridin-2-yl)benzonitrile,
4-(3-((4-methylpiperazin-1-yl)methyl)-6-(4-(trifluoromethyl)phenyl)imidazo[1,2-a]-pyridin-2-yl)benzonitrile,
4-(6-(3,4-dimethylphenyl)-3-((4-(trifluoromethyl)piperazin-1-yl)methyl)imidazo[1,2-a]-pyridin-2-yl)benzonitrile,
4-(6-(3,4-dimethoxyphenyl)-3-((4-propylpiperazin-1-yl)methyl)imidazo[1,2-a]pyridin-2-yl)benzonitrile,
4-(2-(4-cyanophenyl)-3-((4-(trifluoromethyl)-1,4-diazepan-1-yl)methyl)imidazo[1,2-a]pyridin-6-yl)thiophene-2-carbonitrile,
4-(3-((4-methylpiperazin-1-yl)methyl)-6-(2-methylpyridin-4-yl)imidazo[1,2-a]pyridin-2-yl)benzonitrile,
2-(4-methoxyphenyl)-6-(pyrimidin-5-yl)-3-((4-(trifluoromethyl)piperazin-1-yl)methyl)imidazo[1,2-a]pyridine,
4-(3-((4-methylpiperazin-1-yl)methyl)-6-(oxazol-5-yl)imidazo[1,2-a]pyridin-2-yl)benzonitrile,
1-(4-((2-(4-methoxyphenyl)-6-(pyridin-3-yl)imidazo[1,2-a]pyridin-3-yl)methyl)-1,4-diazepan-1-yl)ethanone,
4-(2-(4-methoxyphenyl)-3-((4-propylpiperazin-1-yl)methyl)imidazo[1,2-a]pyridin-6-yl)-N,N-dimethylaniline, 4-(3-((4-acetylpiperazin-1-yl)methyl)-6-(5-methyl-1H-pyrrol-2-yl)imidazo[1,2-a]pyridin-2-yl)benzonitrile,
1-(4-((2-(4-methoxyphenyl)-6-(1-methyl-1H-pyrrol-2-yl)imidazo[1,2-a]pyridin-3-yl)methyl)piperazin-1-yl)ethanone,
2-(4-methoxyphenyl)-3-((4-methylpiperazin-1-yl)methyl)-6-(4-(trifluoromethyl)phenyl)imidazo[1,2-a]pyridine,
2-(4-chlorophenyl)-6-(m-tolyl)-3-((4-(trifluoromethyl)-1,4-diazepan-1-yl)methyl)imidazo[1,2-a]pyridine,
4-(6-(4-(dimethylamino)phenyl)-3-((4-(2,2,2-trifluoroacetyl)-1,4-diazepan-1-yl)methyl)imidazo[1,2-a]pyridin-2-yl)benzonitrile,
4-(6-(4-(dimethylamino)phenyl)-3-((4-(trifluoromethyl)-1,4-diazepan-1-yl)methyl)imidazo[1,2-a]pyridin-2-yl)benzonitrile,
4-(6-(1-methyl-1H-pyrrol-2-yl)-3-((4-methylpiperazin-1-yl)methyl)imidazo[1,2-a]pyridin-2-yl)benzonitrile,
4-(6-(furan-2-yl)-3-((4-(2,2,2-trifluoroacetyl)-1,4-diazepan-1-yl)methyl)imidazo[1,2-a]-pyridin-2-yl)benzonitrile,
2-(4-methoxyphenyl)-3-((4-methylpiperazin-1-yl)methyl)-6-(pyridin-4-yl)imidazo[1,2-a]pyridine,
4-(6-(1-methyl-1H-pyrrol-2-yl)-3-((4-(2,2,2-trifluoroacetyl)piperazin-1-yl)methyl)imidazo[1,2-a]pyridin-2-yl)benzonitrile,
4-(6-(oxazol-5-yl)-3-((4-(2,2,2-trifluoroacetyl)-1,4-diazepan-1-yl)methyl)imidazo[1,2-a]pyridin-2-yl)benzonitrile,
4-(3-((4-acetyl-1,4-diazepan-1-yl)methyl)-6-(4-(pentafluorothio)phenyl)imidazo[1,2-a]pyridin-2-yl)benzonitrile,
4-(6-(3,4-dimethylphenyl)-3-((4-(trifluoromethyl)-1,4-diazepan-1-yl)methyl)imidazo[1,2-a]pyridin-2-yl)benzonitrile,
4-(6-(1-methyl-1H-pyrrol-2-yl)-3-((4-(trifluoromethyl)-1,4-diazepan-1-yl)methyl)imidazo[1,2-a]pyridin-2-yl)benzonitrile,
4-(3-((4-methylpiperazin-1-yl)methyl)-6-(thiophen-3-yl)imidazo[1,2-a]pyridin-2-yl)benzonitrile,
2-(4-methoxyphenyl)-3-((4-methylpiperazin-1-yl)methyl)-6-(1H-pyrrol-2-yl)imidazo[1,2-a]pyridine,
5-(2-(4-cyanophenyl)-3-((4-methylpiperazin-1-yl)methyl)imidazo[1,2-a]pyridin-6-yl)thiophene-3-carbonitrile,
4-((6-(furan-2-yl)-2-(4-methoxyphenyl)imidazo[1,2-a]pyridin-3-yl)methyl)piperazin-2-one,
4-(3-((4-methylpiperazin-1-yl)methyl)-6-(1H-pyrazol-4-yl)imidazo[1,2-a]pyridin-2-yl)benzonitrile,
4-(6-(furan-2-yl)-3-((4-propylpiperazin-1-yl)methyl)imidazo[1,2-a]pyridin-2-yl)benzonitrile,
4-(3-((4-acetyl-1,4-diazepan-1-yl)methyl)-2-(4-(2,2,2-trifluoroacetyl)phenyl)imidazo[1,2-a]pyridin-6-yl)benzonitrile,
6-phenyl-2-(pyridin-3-yl)-3-((4-(trifluoromethyl)piperazin-1-yl)methyl)imidazo[1,2-a]-pyridine,
4-((6-phenyl-2-(1H-pyrrol-2-yl)imidazo[1,2-a]pyridin-3-yl)methyl)piperazin-2-one,
5-(6-phenyl-3-((4-(trifluoromethyl)-1,4-diazepan-1-yl)methyl)imidazo[1,2-a]pyridin-2-yl)picolinonitrile,
4-(2-(p-tolyl)-3-((4-(2,2,2-trifluoroacetyl)piperazin-1-yl)methyl)imidazo[1,2-a]pyridin-6-yl)benzonitrile,
4-(6-(4-cyanophenyl)-3-((4-propylpiperazin-1-yl)methyl)imidazo[1,2-a]pyridin-2-yl)benzamide,
4-(3-((4-acetylpiperazin-1-yl)methyl)-2-(p-tolyl)imidazo[1,2-a]pyridin-6-yl)benzonitrile,
4-(2-(1,5-dimethyl-1H-pyrrol-2-yl)-3-((3-oxopiperazin-1-yl)methyl)imidazo[1,2-a]pyridin-6-yl)benzonitrile,
4-(2-(1,5-dimethyl-1H-pyrrol-2-yl)-3-((4-(2,2,2-trifluoroacetyl)-1,4-diazepan-1-yl)methyl)imidazo[1,2-a]pyridin-6-yl)benzonitrile,
4-(2-(4-(methylsulfonyl)phenyl)-3-((4-(2,2,2-trifluoroacetyl)-1,4-diazepan-1-yl)methyl)imidazo[1,2-a]pyridin-6-yl)benzonitrile,
4-(3-((4-acetyl-1,4-diazepan-1-yl)methyl)-2-(furan-2-yl)imidazo[1,2-a]pyridin-6-yl)benzonitrile,
5-(6-phenyl-3-((4-(trifluoromethyl)-1,4-diazepan-1-yl)methyl)imidazo[1,2-a]pyridin-2-yl)thiophene-2-carbonitrile,
4-(2-(3-nitrophenyl)-3-(piperazin-1-ylmethyl)imidazo[1,2-a]pyridin-6-yl)benzonitrile
4-(2-(4-(methylsulfonyl)phenyl)-3-((3-oxopiperazin-1-yl)methyl)imidazo[1,2-a]pyridin-6-yl)benzonitrile,
4-(2-(furan-2-yl)-3-((4-(2,2,2-trifluoroacetyl)-1,4-diazepan-1-yl)methyl)imidazo[1,2-a]-pyridin-6-yl)benzonitrile,
2-(furan-2-yl)-6-phenyl-3-((4-(trifluoromethyl)-1,4-diazepan-1-yl)methyl)imidazo[1,2-a]pyridine,
4-(3-((4-acetyl-1,4-diazepan-1-yl)methyl)-6-(3,4-dimethylphenyl)imidazo[1,2-a]pyridin-2-yl)benzamide,
5-(6-(m-tolyl)-3-((4-(trifluoromethyl)-1,4-diazepan-1-yl)methyl)imidazo[1,2-a]pyridin-2-yl)-1H-pyrrole-2-carbonitrile,
3-((4-methylpiperazin-1-yl)methyl)-6-(1H-pyrrol-2-yl)-2-(p-tolyl)imidazo[1,2-a]pyridine,
3-(3-(piperazin-1-ylmethyl)-6-(p-tolyl)imidazo[1,2-a]pyridin-2-yl)benzonitrile,
1-(4-((2-(pyridin-3-yl)-6-(p-tolyl)imidazo[1,2-a]pyridin-3-yl)methyl)piperazin-1-yl)ethanone,
4-(3-((4-methylpiperazin-1-yl)methyl)-6-(p-tolyl)imidazo[1,2-a]pyridin-2-yl)thiophene-2-carbonitrile,
4-(3-((4-methylpiperazin-1-yl)methyl)-2-(p-tolyl)imidazo[1,2-a]pyridin-6-yl)thiophene-2-carbonitrile,
2-methyl-4-(3-((4-methylpiperazin-1-yl)methyl)-6-(pyridin-3-yl)imidazo[1,2-a]pyridin-2-yl)benzonitrile,
1-(4-((6-(pyridin-4-yl)-2-(p-tolyl)imidazo[1,2-a]pyridin-3-yl)methyl)piperazin-1-yl)ethanone,
2,2,2-trifluoro-1-(4-(3-((4-methylpiperazin-1-yl)methyl)-6-(m-tolyl)imidazo[1,2-a]pyridin-2-yl)phenyl)ethanone,
2,2,2-trifluoro-1-(4-((6-(2-methylpyridin-4-yl)-2-(p-tolyl)imidazo[1,2-a]pyridin-3-yl)methyl)-1,4-diazepan-1-yl)ethanone,
4-(2-(p-tolyl)-3-((4-(trifluoromethyl)-1,4-diazepan-1-yl)methyl)imidazo[1,2-a]pyridin-6-yl)benzamide,
2-phenyl-3-((4-propylpiperazin-1-yl)methyl)-6-(p-tolyl)imidazo[1,2-a]pyridine,
5-(3-((4-acetyl-1,4-diazepan-1-yl)methyl)-6-(3,4-dimethylphenyl)imidazo[1,2-a]pyridin-2-yl)picolinonitrile,
4-(6-(3,4-dimethylphenyl)-3-((4-(2,2,2-trifluoroacetyl)-1,4-diazepan-1-yl)methyl)imidazo[1,2-a]pyridin-2-yl)benzamide,
3-(3-((4-acetyl-1,4-diazepan-1-yl)methyl)-6-(1,5-dimethyl-1H-pyrrol-2-yl)imidazo[1,2-a]pyridin-2-yl)benzonitrile,
5-(3-((4-methylpiperazin-1-yl)methyl)-2-(p-tolyl)imidazo[1,2-a]pyridin-6-yl)thiophene-2-carbonitrile,
1-(4-((6-(4-(dimethylamino)phenyl)-2-(p-tolyl)imidazo[1,2-a]pyridin-3-yl)methyl)-1,4-diazepan-1-yl)ethanone,
1-(4-((2-phenyl-6-(4-(trifluoromethyl)phenyl)imidazo[1,2-a]pyridin-3-yl)methyl)-1,4-diazepan-1-yl)ethanone,
6-(4-methoxyphenyl)-3-((4-methylpiperazin-1-yl)methyl)-2-(p-tolyl)imidazo[1,2-a]-pyridine,
1-(4-((6-(4-methoxyphenyl)-2-(p-tolyl)imidazo[1,2-a]pyridin-3-yl)methyl)-1,4-diazepan-1-yl)ethanone,
5-(3-((4-methylpiperazin-1-yl)methyl)-6-(p-tolyl)imidazo[1,2-a]pyridin-2-yl)thiophene-2-carbonitrile,
N,N-dimethyl-4-(3-((4-methylpiperazin-1-yl)methyl)-2-(p-tolyl)imidazo[1,2-a]pyridin-6-yl)aniline,
6-(2-methylpyridin-4-yl)-2-(p-tolyl)-3-((4-(trifluoromethyl)-1,4-diazepan-1-yl)methyl)imidazo[1,2-a]pyridine,
4-(3-((4-methylpiperazin-1-yl)methyl)-6-(m-tolyl)imidazo[1,2-a]pyridin-2-yl)benzamide,
2,2,2-trifluoro-1-(4-(3-((4-methylpiperazin-1-yl)methyl)-6-(1H-pyrrol-2-yl)imidazo[1,2-a]pyridin-2-yl)phenyl)ethanone,
4-(3-((4-acetylpiperazin-1-yl)methyl)-6-(p-tolyl)imidazo[1,2-a]pyridin-2-yl)thiophene-2-carbonitrile,
2,2,2-trifluoro-1-(4-((6-(pyridin-3-yl)-2-(p-tolyl)imidazo[1,2-a]pyridin-3-yl)methyl)piperazin-1-yl)ethanone,
5-(3-((4-acetyl-1,4-diazepan-1-yl)methyl)-6-(p-tolyl)imidazo[1,2-a]pyridin-2-yl)thiophene-3-carbonitrile,
2-(1,5-dimethyl-1H-pyrrol-2-yl)-3-((4-propylpiperazin-1-yl)methyl)-6-(m-tolyl)imidazo[1,2-a]pyridine,
4-(3-((3-oxopiperazin-1-yl)methyl)-6-(1H-pyrrol-2-yl)imidazo[1,2-a]pyridin-2-yl)benzamide,
6-(4-methoxyphenyl)-3-((4-methylpiperazin-1-yl)methyl)-2-(1H-pyrrol-2-yl)imidazo[1,2-a]pyridine,
3-((4-methylpiperazin-1-yl)methyl)-6-(pyridin-4-yl)-2-(4-(trifluoromethyl)phenyl)imidazo[1,2-a]pyridine,
4-(6-(4-(dimethylamino)phenyl)-3-(piperazin-1-ylmethyl)imidazo[1,2-a]pyridin-2-yl)-2-methylbenzonitrile,
1-(4-((6-(4-chlorophenyl)-2-(p-tolyl)imidazo[1,2-a]pyridin-3-yl)methyl)-1,4-diazepan-1-yl)ethanone,
3-((4-methylpiperazin-1-yl)methyl)-6-(1H-pyrrol-2-yl)-2-(4-(trifluoromethyl)phenyl)imidazo[1,2-a]pyridine,
4-(2-(3,4-dimethylphenyl)-3-((4-(2,2,2-trifluoroacetyl)-1,4-diazepan-1-yl)methyl)imidazo[1,2-a]pyridin-6-yl)-2-methylbenzonitrile,
1-(4-((2-(5-methyl-1H-pyrrol-2-yl)-6-(pyridin-4-yl)imidazo[1,2-a]pyridin-3-yl)methyl)-1,4-diazepan-1-yl)ethanone,
3-((4-methylpiperazin-1-yl)methyl)-2-(4-nitrophenyl)-6-(1H-pyrrol-2-yl)imidazo[1,2-a]-pyridine,
3-((4-methylpiperazin-1-yl)methyl)-2-(1H-pyrrol-2-yl)-6-(m-tolyl)imidazo[1,2-a]pyridine,
4-(3-(piperazin-1-ylmethyl)-6-(4-(trifluoromethyl)phenyl)imidazo[1,2-a]pyridin-2-yl)benzamide,
3-((4-methylpiperazin-1-yl)methyl)-24 I H-pyrrol-2-yl)-6-(4-(trifluoromethyl)phenyl)imidazo[1,2-a]pyridine,
5-(3-((4-methylpiperazin-1-yl)methyl)-6-(p-tolyl)imidazo[1,2-a]pyridin-2-yl)picolinonitrile,
5-(3-((4-methylpiperazin-1-yl)methyl)-2-(p-tolyl)imidazo[1,2-a]pyridin-6-yl)-1H-pyrrole-2-carbonitrile,
4,4′-(3-((3-oxopiperazin-1-yl)methyl)imidazo[1,2-a]pyridine-2,6-diyl)dibenzamide,
5-(3-((4-acetyl-1,4-diazepan-1-yl)methyl)-2-(3-cyanophenyl)imidazo[1,2-a]pyridin-6-yl)thiophene-2-carbonitrile,
4-(3-((1,4-diazepan-1-yl)methyl)-6-(4-((trifluoromethyl)thio)phenyl)imidazo[1,2-a]-pyridin-2-yl)benzonitrile,
4-(3-((1,4-diazepan-1-yl)methyl)-6-(4-((trifluoromethyl)sulfonyl)phenyl)imidazo[1,2-a]pyridin-2-yl)benzonitrile,
the pharmaceutically acceptable salts thereof, the N-oxides thereof and the pharmaceutically acceptable salts of said N-oxides.

31. (canceled)

32. A pharmaceutical composition comprising an imidazo[1,2-a]pyridine compound of formula (I): 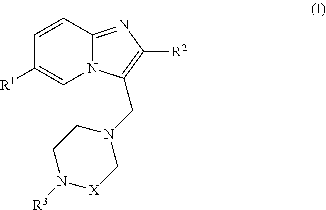
wherein
X is CH₂, CH₂CH₂ or C═O;
R¹ is phenyl or C-bound monocyclic 5- or 6-membered heteroaryl, wherein phenyl and monocyclic 5- or 6-membered heteroaryl are unsubstituted or carry 1, 2, 3, 4 or 5 radicals R^{1a} which are identical or different;
R^{1a} is selected from the group consisting of halogen, CN, NO₂, NH₂, OH, SH, C₁-C₁₀-alkyl, C₂-C₁₀-alkenyl, C₂-C₁₀-alkynyl, C₁-C₆-alkoxy, C₁-C₆-alkylthio, hydroxy-C₁-C₆-alkyl, C₁-C₄-alkoxy-C₁-C₄-alkyl, fluorinated C₁-C₂-alkyl, SF₅, fluorinated C₁-C₂-alkoxy, fluorinated C₁-C₂-alkylsulfonyl, fluorinated C₁-C₂-alkylthio, C(O)R⁴, NR⁵R⁶, C(O)NR⁷R⁸ and C(O)OR⁹, or two radicals R^{1a}, which are bound to adjacent carbon atoms, may also form a bridging moiety O-Alk-O, wherein Alk is selected from CH₂, CH₂CH₂, CHF, CF₂ and CF₂CF₂;
R² is phenyl or C-bound monocyclic 5- or 6-membered heteroaryl, wherein phenyl and monocyclic 5- or 6-membered heteroaryl are unsubstituted or carry 1, 2, 3, 4 or 5 radicals R^{2a} which are identical or different;
R^{2a} is selected from the group consisting of halogen, CN, NO₂, NH₂, OH, SH, C₁-C₁₀-alkyl, C₁-C₁₀-alkenyl, C₂-C₁₀-alkynyl, C₁-C₆-alkoxy, C₁-C₆-alkylthio, hydroxy-C₁-C₆-alkyl, C₁-C₄-alkoxy-C₁-C₄-alkyl, fluorinated C₁-C₂-alkyl, SF₅, C₁-C₄-alkyl-sulfonyl, fluorinated C₁-C₂-alkoxy, C(O)R⁴, NR⁵R⁶, C(O)NR⁷R⁸ and C(O)OR⁹, or two radicals R^{2a}, which are bound to adjacent carbon atoms, may also form a bridging moiety O-Alk′-O, wherein Alk′ is selected from CH₂, CH₂CH₂, CHF, CF₂ and CF₂CF₂;
R³ is hydrogen, C₁-C₆-alkyl, C₁-C₄-alkoxy-C₁-C₄-alkyl, fluorinated C₁-C₂-alkyl or C(O)R⁴;
R⁴ is selected from the group consisting of hydrogen, C₁-C₄-alkyl, C₁-C₄-alkoxy-C₁-C₄-alkyl, C₃-C₇-cycloalkyl, fluorinated C₁-C₂-alkyl, C₁-C₄-alkoxy, C₁-C₄-alkoxy-C₁-C₄-alkoxy, C₃-C₇-cycloalkoxy, fluorinated C₁-C₂-alkoxy;
R⁵ is hydrogen or C₁-C₆-alkyl;
R⁶ is C₁-C₆-alkyl, hydroxy-C₁-C₆-alkyl, C₁-C₄-alkoxy-C₇-C₄-alkyl, OH, C₁-C₄-alkoxy or a radical C(O)R^{x}, wherein Rₓ is C₁-C₄-alkyl; or
R⁵, R⁶ together with the nitrogen atom, to which they are bound, form an N-bound, 5- or 6-membered saturated nitrogen heterocycle;
R⁷ and R⁸ independently of one another are hydrogen, OH, C₁-C₄-alkoxy or C₁-C₆-alkyl;
R⁹ is hydrogen, C₁-C₆-alkyl, hydroxy-C₁-C₆-alkyl or C₁-C₄-alkoxy-C₁-C₄-alkyl;
or a pharmaceutically acceptable salt or N-oxide thereof or the pharmaceutically acceptable salt of said N-oxide together with at least one physiologically acceptable carrier or auxiliary substance.

33. The pharmaceutical composition of claim 32, further comprising at least one second therapeutic agent useful for the treatment or prevention of a disease or disorder selected from the group consisting of an inflammatory disease, a hyperproliferative disease or disorder, a hypoxia related pathology and a disease characterized by pathophysiological hypervascularization.

34. (canceled)

35. The method of claim 1, wherein the disease or disorder is an inflammatory disease which is selected form the group consisting of atherosclerosis, rheumatoid arthritis, asthma, inflammatory bowel disease, psoriasis, in particular psoriasis vulgaris, psoriasis capitis, psoriasis guttata, psoriasis inversa; neurodermatitis; ichthyosis; alopecia greata; alopecia totalis; alopecia subtotalis; alopecia universalis; alopecia diffusa; atopic dermatitis; lupus erythematodes of the skin; dermatomyositis; atopic eczema; morphea; scleroderma; alopecia greata Ophiasis type; androgenic alopecia; allergic dermatitis; irritative contact dermatitis; contact dermatitis; pemphigus vulgaris; pemphigus foliaceus; pemphigus vegetans; scarring mucous membrane pemphigoid; bullous pemphigoid; mucous membrane pemphigoid; dermatitis; dermatitis herpetiformis Duhring; urticaria; necrobiosis lipoidica; erythema nodosum; prurigo simplex; prurigo nodularis; prurigo acuta; linear IgA dermatosis; polymorphic light dermatosis; erythema solaris; exanthema of the skin; drug exanthema; purpura chronica progressiva; dihydrotic eczema; eczema; fixed drug exanthema; photoallergic skin reaction; and perioral dermatitis.

36. The method of claim 1, wherein the disease or disorder is an hyperproliferative disease which is selected from the group consisting of a tumor or cancer disease, precancerosis, dysplasia, histiocytosis, a vascular proliferative disease and a virus-induced proliferative disease.

37. The method of claim 1, wherein the disease or disorder is a tumor or cancer disease which is selected from the group consisting of diffuse large B-cell lymphoma (DLBCL), T-cell lymphomas or leukemias, e.g., cutaneous T-cell lymphoma (CTCL), noncutaneous peripheral T-cell lymphoma, lymphoma associated with human T-cell lymphotrophic virus (HTLV), adult T-cell leukemia/lymphoma (ATLL), as well as acute lymphocytic leukemia, acute nonlymphocytic leukemia, acute myeloid leukemia, chronic lymphocytic leukemia, chronic myelogenous leukemia, Hodgkin's disease, non-Hodgkin's lymphoma, myeloma, multiple myeloma, mesothelioma, childhood solid tumors, glioma, bone cancer and soft-tissue sarcomas, common solid tumors of adults such as head and neck cancers (e.g., oral, laryngeal and esophageal), genitourinary cancers (e.g., prostate, bladder, renal, uterine, ovarian, testicular, rectal, and colon), lung cancer (e.g., small cell carcinoma and non-small cell lung carcinoma, including squamous cell carcinoma and adenocarcinoma), breast cancer, pancreatic cancer, melanoma and other skin cancers, basal cell carcinoma, metastatic skin carcinoma, squamous cell carcinoma of both ulcerating and papillary type, stomach cancer, brain cancer, liver cancer, adrenal cancer, kidney cancer, thyroid cancer, medullary carcinoma, osteosarcoma, soft-tissue sarcoma, Ewing's sarcoma, veticulum cell sarcoma, and Kaposi's sarcoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, leiomyosarcoma, rhabdomyosarcoma, squamous cell carcinoma, adenocarcinoma, sweat gland carcinoma, sebaceous gland carcinoma, papillary carcinoma, glioblastoma, papillary adenocarcinomas, cystadenocarcinoma, bronchogenic carcinoma, seminoma, embryonal carcinoma, Wilms' tumor, small cell lung carcinoma, epithelial carcinoma, astrocytoma, medulloblastoma, craniopharyngioma, ependymoma, pinealoma, hemangioblastoma, acoustic neuroma, oligodendroglioma, meningioma, neuroblastoma, retinoblastoma, glaucoma, hemangioma, heavy chain disease and metastases.

38. The method of claim 1, wherein the disease or disorder is a hyperproliferative disease which method comprises administering the compound of formula (I) to the subject prior to, during and/or after said subject was subjected to a radiation therapy, a chemotherapy, an immunotherapy, a laser/microwave thermotherapy or a gene therapy using antisense DNA and/or RNA.

39. (canceled)

40. An imidazo[1,2-a]pyridine compound of formula (I), 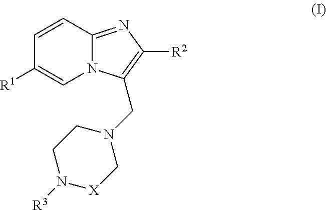
wherein
X is CH₂, CH₂CH₂ or C═O;
R¹ is phenyl or C-bound monocyclic 5- or 6-membered heteroaryl, wherein phenyl and monocyclic 5- or 6-membered heteroaryl are unsubstituted or carry 1, 2, 3, 4 or 5 radicals R^{1a} which are identical or different;
R^{1a} is selected from the group consisting of halogen, CN, NO₂, NH₂, OH, SH, C₁-C₁₀-alkyl, C₂-C₁₀-alkenyl, C₁-C₁₀-alkynyl, C₁-C₆-alkoxy, hydroxy-C₁-C₆-alkyl, C₁-C₄-alkoxy-C₁-C₄-alkyl, fluorinated C₁-C₂-alkyl, SF₅, fluorinated C₁-C₂-alkoxy, fluorinated C₁-C₂-alkylsulfonyl, fluorinated alkylthio, C(O)R⁴, NR⁵R⁶, C(O)NR⁷R⁸ and C(O)OR⁹, or two radicals R^{1a}, which are bound to adjacent carbon atoms, may also form a bridging moiety O-Alk-O, wherein Alk is selected from CH₂, CH₂CH₂, CHF, CF₂ and CF₂CF₂;
R² is phenyl or C-bound monocyclic 5- or 6-membered heteroaryl, wherein phenyl and monocyclic 5- or 6-membered heteroaryl are unsubstituted or carry 1, 2, 3, 4 or 5 radicals R^{2a} which are identical or different;
R^{2a} is selected from the group consisting of halogen, CN, NO₂, NH₂, OH, SH, C₁-C₁₀-alkyl, C₂-C₁₀-alkenyl, C₂-C₁₀-alkynyl, C₁-C₆-alkoxy, C₁-C₆-alkylthio, hydroxy-C₁-C₆-alkyl, C₁-C₄-alkoxy-C₁-C₄-alkyl, fluorinated C₁-C₂-alkyl, SF₅, C₁-C₄-alkyl-sulfonyl, fluorinated C₁-C₂-alkoxy, C(O)R⁴, NR⁵R⁶, C(O)NR⁷R⁸ and C(O)OR⁹, or two radicals R^{2a}, which are bound to adjacent carbon atoms., may also form a bridging moiety O-Alk′-O, wherein Alk′ is selected from CH₂, CH₂CH₂, CHF, CF₂ and CF₂CF₂;
R³ is hydrogen, C₁-C₆-alkyl, C₁-C₄-alkoxy-C₁-C₄-alkyl, fluorinated C₁-C₂-alkyl or C(O)R⁴;
R⁴ is selected from the group consisting of hydrogen, C₁-C₄-alkyl, C₁-C₄-alkoxy-C₁-C₄-alkyl, C₃-C₇-cycloalkyl, fluorinated C₁-C₂-alkyl, C₁-C₄-alkoxy, C₁-C₄-alkoxy-C₁-C₄-alkoxy, C₃-C₇-cycloalkoxy, fluorinated C₁-C₂-alkoxy;
R⁵ is hydrogen or C₁-C₆-alkyl;
R⁶ is C₁-C₆-alkyl, hydroxy-C₁-C₆-alkyl, C₁-C₄-alkoxy-C₂-C₄-alkyl, OH, C₁-C₄-alkoxy or a radical C(O)Rₓ, wherein Rₓ is C₁-C₄-alkyl; or
R⁵, R⁶ together with the nitrogen atom, to which they are bound, form an N-bound, 5- or 6-membered saturated nitrogen heterocycle;
R⁷ and R⁸ independently of one another are hydrogen, OH, C₁-C₄-alkoxy or C₁-C₆-alkyl;
R⁹ is hydrogen, C₁-C₆-alkyl, hydroxy-C₂-C₆-alkyl or C₁-C₄-alkoxy-C₇-C₄-alkyl;
or a pharmaceutically acceptable salt or N-oxide thereof, or a pharmaceutically acceptable salt of said N-oxide;
except for the following compounds:
2-(3-nitrophenyl)-6-phenyl-3-(1-piperazinylmethyl)-imidazo[1,2-a]pyridine,
2-(4-nitrophenyl)-6-phenyl-3-(1-piperazinylmethyl)-imidazo[1,2-a]pyridine,
2,6-diphenyl-3-(1-piperazinylmethyl)-imidazo[1,2-a]pyridine,
2-(4-chlorophenyl)-6-phenyl-3-(piperazin-1-ylmethyl)-imidazo[1,2-a]pyridine,
2-(4-fluorophenyl)-6-phenyl-3-(piperazin-1-ylmethyl)-imidazo[1,2-a]pyridine,
2-(4-methoxyphenyl)-6-phenyl-3-(piperazin-1-ylmethyl)-imidazo[1,2-a]pyridine,
2-(3-methoxyphenyl)-6-phenyl-3-(piperazin-1-ylmethyl)-imidazo[1,2-a]pyridine,
2-(4-methylphenyl)-6-phenyl-3-(piperazin-1-ylmethyl)-imidazo[1,2-a]pyridine,
2-(4-chlorophenyl)-3-[(4-ethyl-piperazin-1-yl)methyl]-6-phenyl-imidazo[1,2-a]pyridine,
3-[(4-ethyl-1-piperazinyl)methyl]-2,6-diphenyl-imidazo[1,2-a]pyridine,
3-[(4-ethyl-1-piperazinyl)methyl]-2-(4-methylphenyl)-6-phenyl-imidazo[1,2-a]pyridine,
1-[4-[[2-(3-nitrophenyl)-6-phenylimidazo[1,2-a]pyridin-3-yl]methyl]-piperazin-1-yl]-ethanone,
1-[4-[[2-(4-nitrophenyl)-6-phenylimidazo[1,2-a]pyridin-3-yl]-methyl]-piperazin-1-yl]-ethanone,
1-[4-[[2-(4-fluorophenyl)-6-phenylimidazo[1,2-a]pyridin-3-yl]methyl]-piperazin-1-yl]-ethanone,
1-[4-[(2,6-diphenylimidazo[1,2-a]pyridin-3-yl)methyl]-piperazin-1-yl]-ethanone,
1-[4-[(2-(3-methoxyphenyl)-6-phenylimidazo[1,2-a]pyridin-3-yl)methyl]-piperazin-1-yl]-ethanone,
1-[4-[[2-(4-chlorophenyl)-6-phenylimidazo[1,2-a]pyridin-3-yl]methyl]-piperazin-1-yl]-ethanone,
1-[4-[[2-(4-nitrophenyl)-6-phenylimidazo[1,2-a]pyridin-3-yl]methyl]-piperazin-1-yl]-ethanone,
1-[4-[[2-(3-nitrophenyl)-6-phenylimidazo[1,2-a]pyridin-3-yl]methyl]-piperazin-1-yl]-ethanone,
2-methyl-1-[4-[[2-(4-methylphenyl)-6-phenylimidazo[1,2-a]pyridin-3-yl]methyl]-piperazin-1-yl]-propan-1-one,
1-[4-[(2-(3-methoxyphenyl)-6-phenylimidazo[1,2-a]pyridin-3-yl)methyl]-piperazin-1-yl]-propanone,
1-[4-[[2-(3-nitrophenyl)-6-phenylimidazo[1,2-a]pyridin-3-yl]methyl]-piperazin-1-yl]-propan-1-one,
1-[4-[[2-(4-fluorophenyl)-6-phenylimidazo[1,2-a]pyridin-3-yl]methyl]-piperazin-1-yl]-propan-1-one,
1-[4-[[2-(4-methylphenyl)-6-phenylimidazo[1,2-a]pyridin-3-yl]methyl]-piperazin-1-yl]-propan-1-one,
1-[4-[[2-(4-chlorophenyl)-6-phenylimidazo[1,2-a]pyridin-3-yl]methyl]-piperazin-1-yl]-propanone,
1-[4-[[2-(4-methoxyphenyl)-6-phenylimidazo[1,2-a]pyridin-3-yl]methyl]-piperazin-1-yl]-propanone,
1-[4-[[2-(4-nitrophenyl)-6-phenylimidazo[1,2-a]pyridin-3-yl]methyl]-piperazin-1-yl]-propan-1-one,
2-methyl-1-[4-[[2-(4-nitrophenyl)-6-phenylimidazo[1,2-a]pyridin-3-yl]methyl]-piperazin-1-yl]-propan-1-one,
1-[4-[[2-(4-methoxyphenyl)-6-phenylimidazo[1,2-a]pyridin-3-yl]methyl]-piperazin-1-yl]-2-methyl-1-propanone,
1-[4-[[2-(4-chlorophenyl)-6-phenylimidazo[1,2-a]pyridin-3-yl]methyl]-piperazin-1-yl]-2-methyl-propan-1-one,
1-[4-[[2-(4-fluorophenyl)-6-phenylimidazo[1,2-a]pyridin-3-yl]methyl]-piperazin-1-yl]-2-methyl-propan-1-one,
2-methyl-1-[4-[[2-(3-methoxyphenyl)-6-phenylimidazo[1,2-a]pyridin-3-yl]methyl]-piperazin-1-yl]-propan-1-one,
2-methyl-1-[4-[[2-(3-nitrophenyl)-6-phenylimidazo[1,2-a]pyridin-3-yl]methyl]-piperazin-1-yl]-propan-1-one,
3-methyl-1-[4-[[2-(4-nitrophenyl)-6-phenylimidazo[1,2-a]pyridin-3-yl]methyl]-piperazin-1-yl]-butan-1-one,
1-[4-[[2-(4-fluorophenyl)-6-phenylimidazo[1,2-a]pyridin-3-yl]methyl]-piperazin-1-yl]-3-methyl-butan-1-one,
1-[4-[[2-(4-chlorophenyl)-6-phenylimidazo[1,2-a]pyridin-3-yl]methyl]-piperazin-1-yl]-3-methyl-butan-1-one,
3-methyl-1-[4-[[2-(3-methoxyphenyl)-6-phenylimidazo[1,2-a]pyridin-3-yl]methyl]-piperazin-1-yl]-butan-1-one,
3-methyl-1-[4-[[2-(4-methylphenyl)-6-phenylimidazo[1,2-a]pyridin-3-yl]methyl]-piperazin-1-yl]-butan-1-one,
3-methyl-1-[4-[[2-(3-nitrophenyl)-6-phenylimidazo[1,2-a]pyridin-3-yl]methyl]-piperazin-1-yl]-butan-1-one,
cyclopropyl-[4-[[2-(3-methoxyphenyl)-6-phenylimidazo[1,2-a]pyridin-3-yl]methyl]-1-piperazinyl]-methanone,
cyclopropyl-[4-[[2-(4-fluorophenyl)-6-phenylimidazo[1,2-a]pyridin-3-yl]methyl]-piperazin-1-yl]-methanone,
cyclopropyl-[4-[[2-(3-nitrophenyl)-6-phenylimidazo[1,2-a]pyridin-3-yl]methyl]-piperazin-1-yl]-methanone,
cyclopropyl-[4-[[2-(4-nitrophenyl)-6-phenylimidazo[1,2-a]pyridin-3-yl]methyl]-piperazin-1-yl]-methanone,
cyclopropyl-[4-[[2-(4-methoxyphenyl)-6-phenylimidazo[1,2-a]pyridin-3-yl]methyl]-piperazin-1-yl]-methanone,
cyclopropyl-[4-[[2-(4-chlorophenyl)-6-phenylimidazo[1,2-a]pyridin-3-yl]methyl]-piperazin-1-yl]-methanone,
cyclobutyl-[4-[[2-(4-nitrophenyl)-6-phenylimidazo[1,2-a]pyridin-3-yl]methyl]-piperazin-1-yl]-methanone,
cyclobutyl-[4-[[2-(4-methoxyphenyl)-6-phenylimidazo[1,2-a]pyridin-3-yl]methyl]-piperazin-1-yl]-methanone,
cyclobutyl-[4-[[2-(4-fluorophenyl)-6-phenylimidazo[1,2-a]pyridin-3-yl]methyl]-piperazin-1-yl]-methanone,
cyclobutyl-[4-[[2-(4-methylphenyl)-6-phenylimidazo[1,2-a]pyridin-3-yl]methyl]-piperazin-1-yl]-methanone,
cyclobutyl-[4-[[2-(3-methoxyphenyl)-6-phenylimidazo[1,2-a]pyridin-3-yl]methyl]-piperazin-1-yl]-methanone,
cyclobutyl-[4-[[2-(4-chlorophenyl)-6-phenylimidazo[1,2-a]pyridin-3-yl]methyl]-piperazin-1-yl]-methanone,
cyclobutyl-[4-[[2-(3-nitrophenyl)-6-phenylimidazo[1,2-a]pyridin-3-yl]methyl]-piperazin-1-yl]-methanone,
cyclopentyl-[4-[[2-(4-chlorophenyl)-6-phenylimidazo[1,2-a]pyridin-3-yl]methyl]-piperazin-1-yl]-methanone,
cyclopentyl-[4-[[2-(4-fluorophenyl)-6-phenylimidazo[1,2-a]pyridin-3-yl]methyl]-piperazin-1-yl]-methanone,
cyclopentyl-[4-[[2-(3-methoxyphenyl)-6-phenylimidazo[1,2-a]pyridin-3-yl]methyl]-piperazin-1-yl]-methanone,
cyclopentyl-[4-[[2-(4-methoxyphenyl)-6-phenylimidazo[1,2-a]pyridin-3-yl]-methyl]-piperazin-1-yl]-methanone,
cyclopentyl-[4-[[2-(3-nitrophenyl)-6-phenylimidazo[1,2-a]pyridin-3-yl]methyl]-piperazin-1-yl]-methanone,
cyclopentyl-[4-[[2-(4-nitrophenyl)-6-phenylimidazo[1,2-a]pyridin-3-yl]methyl]-piperazin-1-yl]-methanone,
1-[4-[[2-(4-fluorophenyl)-6-phenylimidazo[1,2-a]pyridin-3-yl]methyl]-piperazin-1-yl]-2-methoxy-ethanone,
2-methoxy-1-[4-[[2-(3-nitrophenyl)-6-phenylimidazo[1,2-a]pyridin-3-yl]methyl]-piperazin-1-yl]-ethanone,
2-methoxy-1-[4-[[2-(4-methylphenyl)-6-phenylimidazo[1,2-a]pyridin-3-yl]methyl]-piperazin-1-yl]-ethanone, and
2-methoxy-1-[4-[[2-(4-nitrophenyl)-6-phenylimidazo[1,2-a]pyridin-3-yl]methyl]-piperazin-1-yl]-ethanone.
